SEQUENCE LISTING (472 sequences)

SEQ ID NO: 1 - protein, 773 aa, unidentified
  source 1..773: note = Candidatus Altiarchaeales archaeon
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 2 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMVGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 3 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMSGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 4 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMQGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 5 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMPGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 6 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMHGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 7 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMGGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 8 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMFGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 9 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMAGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 10 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 11 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 12 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRALYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 13 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRVIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 14 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRSIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 15 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRKIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 16 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRHIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 17 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRGIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 18 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRAIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 19 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFSSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 20 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 21 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 22 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 23 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYVLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 24 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYSLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 25 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 26 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYGLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 27 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYALHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 28 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 29 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 30 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 31 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLTKSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 32 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLSSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 33 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLSGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 34 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLSASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 35 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 36 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 37 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 38 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGCSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 39 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 40 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLAVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 41 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 42 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 43 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 44 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLSPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 45 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLAPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 46 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLVPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 47 - protein, 773 aa, synthetic construct; identical to SEQ ID NO: 44

SEQ ID NO: 48 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLGPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 49 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVVYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 50 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVSYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 51 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLVPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 52 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLSPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 53 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLYVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 54 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLYSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 55 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 56 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGLYASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 57 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGVYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 58 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGSYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 59 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGGYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 60 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRGAYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 61 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRALGPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 62 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRALAPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 63 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRALYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 64 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRASYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 65 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVYSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 66 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 67 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 68 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 69 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSYVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 70 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 71 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGGPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 72 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGAPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 73 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 74 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGYASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 75 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSASPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 76 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAYSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 77 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAYGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 78 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAYASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 79 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVVSIIISHNIDPSTIDCECCPEDSYRSPTGHYFCKKKRGLIPETLNELVQRRIEVKKGLKNEKNPERRRFLDVKQQALKLLANSMSGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 80 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVGSIIISHNIDPSTIDCECCPEDSYRSPTGHYFCKKKRGLIPETLNELVQRRIEVKKGLKNEKNPERRRFLDVKQQALKLLANSMGGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 81 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLVASIIISHNIDPSTIDCECCPEDSYRSPTGHYFCKKKRGLIPETLNELVQRRIEVKKGLKNEKNPERRRFLDVKQQALKLLANSMRGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 82 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLSVSIIISHNIDPSTIDCECCPEDSYRSPTGHYFCKKKRGLIPETLNELVQRRIEVKKGLKNEKNPERRRFLDVKQQALKLLANSMRGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 83 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLSGSIIISHNIDPSTIDCECCPEDSYRSPTGHYFCKKKRGLIPETLNELVQRRIEVKKGLKNEKNPERRRFLDVKQQALKLLANSMSGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 84 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGGSIIISHNIDPSTIDCECCPEDSYRSPTGHYFCKKKRGLIPETLNELVQRRIEVKKGLKNEKNPERRRFLDVKQQALKLLANSMVGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 85 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGASIIISHNIDPSTIDCECCPEDSYRSPTGHYFCKKKRGLIPETLNELVQRRIEVKKGLKNEKNPERRRFLDVKQQALKLLANSMNGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 86 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLGASIIISHNIDPSTIDCECCPEDSYRSPTGHYFCKKKRGLIPETLNELVQRRIEVKKGLKNEKNPERRRFLDVKQQALKLLANSMGGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 87 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLASSIIISHNIDPSTIDCECCPEDSYRSPTGHYFCKKKRGLIPETLNELVQRRIEVKKGLKNEKNPERRRFLDVKQQALKLLANSMRGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 88 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRAISHNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 89 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLVLLASSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 90 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLVLLAGSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 91 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLVLLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 92 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLSLLAVSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 93 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLSLLASSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 94 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLSLLAGSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 95 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLQLLAVSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 96 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLQLLAGSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 97 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLQLLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 98 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLGLLAVSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 99 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLGLLASSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 100 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLGLLAGSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 101 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLGLLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 102 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLALLAGSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 103 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLALLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 104 - protein, 773 aa, synthetic construct; identical to SEQ ID NO: 94

SEQ ID NO: 105 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 106 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 107 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAISIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 108 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSASASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 109 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSASGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 110 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSASSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 111 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 112 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 113 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 114 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 115 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIIVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 116 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 117 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 118 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSTAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 119 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSTGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 120 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 121 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 122 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSISSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 123 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVVGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 124 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIPDNILLFDFRSLYPSIIISHNIDPSTIDCECCPEDSYRSPTGHYFCKKKRGLIPETLNELVQRRIEVKKGLKNEKNPERRRFLDVKQQVLGLLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 125 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLSLLAASMTGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 126 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLQLLAGSMYGYFGSPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 127 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLGLLAGSIYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 128 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYLLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 129 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 130 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 131 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 132 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 133 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAGSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 134 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGTSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 135 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYLLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 136 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYFLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 137 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 138 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYLLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 139 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSISGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 140 - protein, 773 aa, synthetic construct; identical to SEQ ID NO: 25

SEQ ID NO: 141 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 142 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYFLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 143 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSATSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 144 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 145 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSGVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 146 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSTAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 147 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSTAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 148 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSTGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 149 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 150 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYSLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 151 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYTLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 152 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 153 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 154 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 155 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 156 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 157 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVTSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 158 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMIGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 159 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 160 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 161 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 162 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 163 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSISGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 164 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 165 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAITSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 166 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSGVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 167 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSGGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 168 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 169 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAVSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 170 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVTISIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 171 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVVASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 172 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRVLAPSIIISHNIDPSTIDCECCPEDSYRSPTGHYFCKKKRGLIPETLNELVQRRIEVKKGLKNEKNPERRRFLDVKQQVLGLLASSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 173 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIPDNILLFDFRSLYPSIIISHNIDPSTIDCECCPEDSYRSPTGHYFCKKKRGLIPETLNELVQRRIEVKKGLKNEKNPERRRFLDVKQQVLVLLAASMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 174 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQVLSLLAGSMYGYFGSPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 175 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDEKGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 176 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKAGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 177 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 178 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 179 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 180 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 181 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRTWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 182 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 183 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTIITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 184 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRTWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 185 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 186 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKAGLKNEKNPERRRFLDVKQQALKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 187 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKAGLKNEKNPERRRFLDVKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 188 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKAGLKNEKNPERRRFLDVKQQALKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 189 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRSLKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 190 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQQSLKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 191 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKALANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 192 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQQALKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 193 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLQLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 194 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTIITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 195 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRALKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 196 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTIITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 197 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDEKGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 198 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDEKGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 199 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDEKGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 200 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 201 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQRALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 202 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDIKQQALKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 203 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 204 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGELIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 205 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKGLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 206 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKSIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 207 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPESSYRSPTGHYFCKKKRGLIPETLNELVQRRIEVKKGLKNEKNPERRRFLDVKQQSLKSLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 208 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCWECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 209 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 210 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQCSLKRLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 211 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKAHVIDFVEEGDYDPDYYINNQVLPSVLRILEALGYSEDELKGLGKQMKLGGF

SEQ ID NO: 212 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKGVIRNIKTGKIPLKDLDINTQITRGMAEYKTEGPHIVAAKKAMKRGLEFKQGNIVTYVVTKKGKGISDKARVIDFVEEGDYDPDYYINNQVLPSVLRVLEALGYSEDELKGLGKQMKLGGF

SEQ ID NO: 213 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKSLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 214 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCYECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 215 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQGSLKALANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 216 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRNPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 217 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKGGLEFKQGNIVTYVVTKKGKSISDKARVIDFVEEGDYDPDYYINNQVLPSVLRILEALGYSEDELKGLGKQMKLGGF

SEQ ID NO: 218 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKHLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 219 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQASLKSLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 220 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSVKNLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 221 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIMKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 222 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAGHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 223 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKGISDKARVIDFVEEGDYDPDYYINNQVLPSVLRILEALGYSEDELKGLGKQMKLGGF

SEQ ID NO: 224 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKGISDKARVIDFVEEGDYDPDYYINNQVLPSVLRILEALGYSEDELKGLGKQMKLGGF

SEQ ID NO: 225 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 226 - protein, 773 aa, synthetic construct; identical to SEQ ID NO: 180

SEQ ID NO: 227 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQGSLKRLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 228 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKNAEHFLDKINIELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 229 - protein, 773 aa, synthetic construct
MKKTLLDADYITREEKAVVRLFYKTEEGREIQEVADFRPYMYVLPEEHDLKKLQREIMELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 230 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCPECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 231 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQGSLKILANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 232 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKNLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 233 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCFECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 234 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSYRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQRSLKMLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 235 - protein, 773 aa, synthetic construct; identical to SEQ ID NO: 216

SEQ ID NO: 236 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFDKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 237 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDGLKGLGKQMKLGGF

SEQ ID NO: 238 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQTSLKSLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 239 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKCLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 240 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKILANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 241 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKYLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 242 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRASSGQRVESLLIKKAFEKSILVPNRPSDRVVNERQRKTYIGAYVVEPKRGIHDNILLFDFRSSAGSIIISHNIDPSTIDCECCPEDNYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 243 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQLSLKMLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 244 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 245 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSYAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 246 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSTGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 247 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSMAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 248 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 249 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGSKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 250 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYSLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 251 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDSENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 252 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDRENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 253 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFSKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 254 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLESAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 255 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVSRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 256 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 257 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYLLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 258 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 259 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 260 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 261 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYTLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 262 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSGTSSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 263 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSVAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 264 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSIASSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 265 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAGASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYHLHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 266 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSAAASIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 267 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSASPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 268 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 269 - protein, 135 aa, unidentified
  source 1..135: note = Candidatus Altiarchaeales archaeon
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDL

SEQ ID NO: 270 - protein, 212 aa, unidentified
  source 1..212: note = Candidatus Altiarchaeales archaeon
RIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITREL

SEQ ID NO: 271 - protein, 107 aa, unidentified
  source 1..107: note = Candidatus Altiarchaeales archaeon
IYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKR

SEQ ID NO: 272 - protein, 50 aa, unidentified
  source 1..50: note = Candidatus Altiarchaeales archaeon
GLIPETLNELVQRRIEVKKGLKNEKNPERRRFLDVKQQALKLLANSMYGY

SEQ ID NO: 273 - protein, 120 aa, unidentified
  source 1..120: note = Candidatus Altiarchaeales archaeon
FGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWA

SEQ ID NO: 274 - protein, 149 aa, unidentified
  source 1..149: note = Candidatus Altiarchaeales archaeon
NIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 275 - protein, 876 aa, Geobacillus stearothermophilus
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 276 - protein, 876 aa, synthetic construct
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSSIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 277 - protein, 876 aa, synthetic construct
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSAIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 278 - protein, 876 aa, synthetic construct
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSAAALRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 279 - protein, 876 aa, synthetic construct
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSSAALRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 280 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 281 - protein, 775 aa, Thermococcus sp.; identical to SEQ ID NO: 280
  source 1..775: note = 9oN-7

SEQ ID NO: 282 - protein, 775 aa, Thermococcus sp.
  source 1..775: note = 9oN-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 283 - protein, 1702 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSILPNEWLPIIENGEIKFVKIGEFINSYMEKQKENVKTVENTEVLEVNNLFAFSFNKKIKESEVKKVKALIRHKYKGKAYEIQLSSGRKINITAGHSLFTVRNGEIKEVSGDGIKEGDLIVAPKKIKLNEKGVSINIPELISDLSEEETADIVMTISAKGRKNFFKGMLRTLRWMFGEENRRIRTFNRYLFHLEKLGLIKLLPRGYEVTDWERLKKYKQLYEKLAGSVKYNGNKREYLVMFNEIKDFISYFPQKELEEWKIGTLNGFRTNCILKVDEDFGKLLGYYVSEGYAGAQKNKTGGISYSVKLYNEDPNVLESMKNVAEKFFGKVRVDRNCVSISKKMAYLVMKCLCGALAENKRIPSVILTSPEPVRWSFLEAYFTGDGDIHPSKRFRLSTKSELLANQLVFLLNSLGISSVKIGFDSGVYRVYINEDLQFPQTSREKNTYYSNLIPKEILRDVFGKEFQKNMTFKKFKELVDSGKLNREKAKLLEFFINGDIVLDRVKSVKEKDYEGYVYDLSVEDNENFLVGFGLLYAHNSYYGYMGYPKARWYSKECAESVTAWGRHYIEMTIREIEEKFGFKVLYADSVSGESEIIIRQNGKIRFVKIKDLFSKVDYSIGEKEYCILEGVEALTLDDDGKLVWKPVPYVMRHRANKRMFRIWLTNSWYIDVTEDHSLIGYLNTSKTKTAKKIGERLKEVKPFELGKAVKSLICPNAPLKDENTKTSEIAVKFWELVGLIVGDGNWGGDSRWAEYYLGLSTGKDAEEIKQKLLEPLKTYGVISNYYPKNEKGDFNILAKSLVKFMKRHFKDEKGRRKIPEFMYELPVTYIEAFLRGLFSADGTVTIRKGVPEIRLTNIDADFLREVRKLLWIVGISNSIFAETTPNRYNGVSTGTYSKHLRIKNKWRFAERIGFLIERKQKRLLEHLKSARVKRNTIDFGFDLVHVKKVEEIPYEGYVYDIEVEETHRFFANNILVHNTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 284 - protein, 1312 aa, Pyrococcus sp.
  source 1..1312: note = GB-D
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSILPEEWVPLIKNGKVKIFRIGDFVDGLMKANQGKVKKTGDTEVLEVAGIHAFSFDRKSKKARVMAVKAVIRHRYSGNVYRIVLNSGRKITITEGHSLFVYRNGDLVEATGEDVKIGDLLAVPRSVNLPEKRERLNIVELLLNLSPEETEDIILTIPVKGRKNFFKGMLRTLRWIFGEEKRVRTASRYLRHLENLGYIRLRKIGYDIIDKEGLEKYRTLYEKLVDVVRYNGNKREYLVEFNAVRDVISLMPEEELKEWRIGTRNGFRMGTFVDIDEDFAKLLGYYVSEGSARKWKNQTGGWSYTVRLYNENDEVLDDMEHLAKKFFGKVKRGKNYVEIPKKMAYIIFESLCGTLAENKRVPEVIFTSSKGVRWAFLEGYFIGDGDVHPSKRVRLSTKSELLVNGLVLLLNSLGVSAIKLGYDSGVYRVYVNEELKFTEYRKKKNVYHSHIVPKDILKETFGKVFQKNISYKKFRELVENGKLDREKAKRIEWLLNGDIVLDRVVEIKREYYDGYVYDLSVDEDENFLAGFGFLYAHNSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 285 - protein, 775 aa, Pyrococcus furiosus
  source 1..775: note = DSM 3638
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 286 - protein, 771 aa, unidentified
  source 1..771: note = Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKF

SEQ ID NO: 287 - protein, 903 aa, unidentified
  source 1..903: note = Escherichia phage RB69
MKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDF

SEQ ID NO: 288 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLGNSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 289 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKSLKNEKNPERRRFLNVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 290 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELMQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 291 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLNKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 292 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPRFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 293 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPEHRRFLDVKQQSLKLLANSMYGYFGFPRARWYCLECAESITALGRKYHLHTIDIVPKFGFDVIYGDTDSVYLIKPNITDRERVMKDAEHFLDRINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 294 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKIPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLEAKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 295 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPHARWYCLECAESITALGRKYHLHTIDIVPKFGFDVIYGDTDSVYLIKPNITGRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 296 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLGALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 297 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITERERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 298 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPHARWYCLECAENITALGRKYHLHTIDIVPKFGFDVIYGDTDSVYLIKPNITDRERVMKDAEHFLDKINSELPEAKELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 299 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANGMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 300 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAEGITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 301 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 302 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKYGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 303 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQCRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPGAMELEFEGFYPRGIFITKKRYALIDERGKLIVKGLEAKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 304 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHHFCKKKRGLIPETLNELVQRRIEVKKGLKNEKNPERRRFLDVKQQSLKLLANSMFGYFGSPRARWYCLECAESITALGRKYHLHTIDIVPKFGFDVIYGDTDSVYLIKPNITDRERIVKDAEHFLDKINSNLPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 305 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSVYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 306 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRVTRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 307 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAASIIISHNIDPSTIDCECCPEDSYRSPTGHYFCKKKRGLIPETLNELVQRRIEVKKGLKNEKNPERRRFLDVKQQSLKLLANGMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 308 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAASIIISHNIDPSTIDCECCPEDSYRSPTGHYFCKKKRGLIPETLNELVQRRIEVKKGLKNEKNPERRRFLDVKQQSLKLLANSMYGYFGFPRARWYCLGCAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 309 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKDLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 310 - protein, 773 aa, synthetic construct; identical to SEQ ID NO: 306

SEQ ID NO: 311 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELRGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 312 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKRKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 313 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCRCCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 314 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVRNLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 315 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSRDELKGLGKQMKLGGF

SEQ ID NO: 316 - protein, 773 aa, synthetic construct
MKKTLLDADYITREEKAVVRLFYKTERGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 317 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGRRKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 318 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVERGDYDPDYYINNQVLPSVLRILEALGYSEDELKGLGKQMKLGGF

SEQ ID NO: 319 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPRKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 320 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGSPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 321 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAGRYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 322 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDLEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 323 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCRCCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 324 - protein, 773 aa, synthetic construct
MKKTLLDADYITREEKAVVRLFYKTEEGREIQEVADFRPYMYVLSEEHDLKKLQREIRELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 325 - protein, 773 aa, synthetic construct
MKKTLLDADYITREEKAVVRLFYKTEEGREIQEVADFRPYLYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 326 - protein, 773 aa, synthetic construct
MKKTLLDADYFTREEKAVVRLFYKTEEGREIQEVADFRPYMYVLPEEHDLKKLQREIKELKNITNVEIKRMIEGDREVEVLKVMVNQPRDVPNLRGLIKELEGCREVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 327 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCESFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 328 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLHRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 329 - protein, 773 aa, synthetic construct
MKKTLLDADYITREEKAVVRLFYKTEEGREIQEVADFRPYMYVLPEEHDLKKLQREIKELKNVTNVEIKRMIEGDRGVEVLKVMVNHPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 330 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPRKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 331 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVLVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 332 - protein, 773 aa, synthetic construct
MKKTLLDADYITREEKAVVRLFYKTEEGREIQEVADFRPYMYVLPEEHDLKKLLREIKELKNITNVEIKRMIEGDREVEVLKVMVNQPRDVPNLRGLIKELEGCKEVREAHIPFAERYLIDSGLISMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 333 - protein, 773 aa, synthetic construct
MKKTLLDADYITRGEKAVVRLFYKTEEGREIQEVADSRPYMYVLPEEHDLKKLQREIKELKNITNVEIKRMIEGDREVEVLKVMVNQPRDVPNLRGLIKELEGCKEVREAHIPFAERYLIDSGQIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 334 - protein, 773 aa, synthetic construct
MKKTLLDADYITREEKAVVRLFYKTEEGREIQEVADFRPYMYVLPEEHDLRKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 335 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEIREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 336 - protein, 773 aa, synthetic construct
MKKTLLDADYITREEKAVVRLFYKTEEGREIQEVADFRPYMYVLPEEHDLKKLQREIMELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 337 - protein, 773 aa, synthetic construct
MKKTLLDADYITREEKAVVRLFYKTEEGREIQEVADFRPYMYVLPEEHDLKKLQREIKELKNITNVEIKRMIKGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 338 - protein, 773 aa, synthetic construct
MKKTLLDADYITREEKAVVRLFYKTEEGREIQEVADFRPYMYVLPEEHDLKKLQREIKELENITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 339 - protein, 773 aa, synthetic construct
MKKTLLDADYITRGEKAVVRLFYKTEEGREIQEVADFRPYMYVLPEEHDLKKLQREIKELKNITNVEVKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 340 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGIDGSPLRLERRGMNLGAHVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 341 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRSLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 342 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIRKREIDIIVTYNGDNFDFPYLEERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 343 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRIIRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 344 - protein, 773 aa, synthetic construct
MKKTLLDADYITREEKAVVRLFYKTEEGREIQEVADFRPYMYVLPEEHDPKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 345 - protein, 773 aa, synthetic construct
MKKTLLDANYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 346 - protein, 773 aa, synthetic construct
MKKTLLDADYITREEKAVVRLFYKTEEGREIQEVADFRPYMYVLPEEHDLKKLQREIKELKNTTNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 347 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQVFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 348 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKGEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 349 - protein, 773 aa, synthetic construct
MKKTLLDADYITRKEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 350 - protein, 773 aa, synthetic construct; identical to SEQ ID NO: 321

SEQ ID NO: 351 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFVERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 352 - protein, 773 aa, synthetic construct
MKKTLLDADYITREEKAVVRLFYKTEEGREIQEVADFRPYMYVLPEEHDLKKLQREIKELKNITNVEIKRMIEGNREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 353 - protein, 766 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGK

SEQ ID NO: 354 - protein, 767 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELRVWASK

SEQ ID NO: 355 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDVEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 356 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKSLGKQMKLGGF

SEQ ID NO: 357 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKNISDKARVIDFVEEGDYDPDYYINNQVLPCVLRILEALGYSEDELKGLGKQMKLGGF

SEQ ID NO: 358 - protein, 773 aa, synthetic construct; identical to SEQ ID NO: 301

SEQ ID NO: 359 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQIARGMAEYITEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 360 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 361 - protein, 773 aa, synthetic construct
MKKTLLDADYITRKEKAVVRLFYKTEEGREIQEVADFRPYMYVLPEERDLKKLQREIKELKNITNVEIKRMIEGNREVEVLKVMVNQPRDVPNLRGLIKELEGCKEVREAHIPFVERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 362 - protein, 773 aa, synthetic construct; identical to SEQ ID NO: 360

SEQ ID NO: 363 - protein, 773 aa, synthetic construct; identical to SEQ ID NO: 351

SEQ ID NO: 364 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWNNPEKEKFKELIEYAMSDAESTLQIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 365 - protein, 773 aa, synthetic construct; identical to SEQ ID NO: 360

SEQ ID NO: 366 - protein, 773 aa, synthetic construct
MKKTLLDADYITRKEKAVVRLFYKTEEGREIQEVADFRPYMYVLPEERDLKKLQREIKELKNITNVEIKRMIEGNREVEVLKVMVNQPRDVPNLRGLIKELEGCKEVREAHIPFVERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 367 - protein, 773 aa, synthetic construct; identical to SEQ ID NO: 355

SEQ ID NO: 368 - protein, 773 aa, synthetic construct; identical to SEQ ID NO: 301

SEQ ID NO: 369 - protein, 773 aa, synthetic construct; identical to SEQ ID NO: 323

SEQ ID NO: 370 - protein, 773 aa, synthetic construct; identical to SEQ ID NO: 325

SEQ ID NO: 371 - protein, 773 aa, synthetic construct
MKKTLLDADYITREEKAVVRLFYKTEEGREIQEVADFRPYLYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 372 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCRCCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 373 - protein, 773 aa, synthetic construct
MKKTLLDADYITREEKAVVRLFYKTEEGREIQEVADFRPYMYVLPEERDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 374 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRDIKTGKIPLKDLAINTQITRGMAEYKTEGPHIVAAKKAMKRGLKFKQGSIVTYVVTKKGKSISDKARVIDFVEEGDYDPDYYINNQVLPSVLRILEALGYSEDELKGLGKQMKLGGF

SEQ ID NO: 375 - protein, 773 aa, synthetic construct
MKKTLLDADYITREEKAVVRLFYKTEEGREIQEVADFRPYMYVLPEERDLKKLQREIKELKNITNVEIKRMIEGNREVEVLKVMVNQPRDVPNLRGLIKELEGCKEVREAHIPFVERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 376 - protein, 773 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 377 - protein, 767 aa, synthetic construct
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGVKEVREAHIPFAERYLIDSGLIPMQDVENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVVRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQ

SEQ ID NO: 378 - protein, 773 aa, synthetic construct
MKKTLLDADYITRKEKAVVRLFYKTEEGREIQEVADFRPYMYVLPEERDLKKLQREIKELKNITNVEIKRMIEGNREVEVLKVMVNQPRDVPNLRGLIKELEGVREVREAHIPFVERYLIDSGLIPMQNVENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVVRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAIEKSILVPNRPSDRVVSERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 379 - protein, 773 aa, synthetic construct
MKKTLLDADYITRKEKAVVRLFYKTEEGREIQEVADFRPYMYVLPEERDLKKLQREIKELKNITNVEIKRMIEGNREVEVLKVMVNQPRDVPNLRGLIKELEGVKEVREAHIPFVERYLIDSGLIPMQNVENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVVRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAIEKSILVPNRPSDRVVSERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 380 - protein, 773 aa, synthetic construct
MKKTLLDADYITRKEKAVVRLFYKTEEGREIQEVADFRPYMYVLPEERDLKKLQREIKELKNITNVEIKRMIEGNREVEVLKVMVNQPRDVPNLRGLIKELEGVKEVREAHIPFVERYLIDSGLIPMQDVENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVVRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAIEKSILVPNRPSDRVVSERQRKTYIGAYVVEPKRGIHDNILLFDFRSSAGSIIISHNIDPSTIDCECCPEDSYRSPTGHYFCKKKRGLIPETLNELVQRRIEVKKGLKNEKNPERRRFLDVKQQSLKLLANSIYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 381 - protein, 773 aa, synthetic construct
MKKTLLDADFITRKEKAVVRLFYKTEEGREIQEVADFRPYMYVLPEERDLKKLQREIKELKNITNVEIKRMIEGNREVEVLKVMVNQPRDVPNLRGLIKELEGVKEVREAHIPFVERYLIDSGLIPMQDVENFDLRIAAFAMAVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSFAGSIIISHNIDPSTIDCECCPEDSYRSPTGHYFCKKKRGLIPETLNELVQRRIEVKKGLKNEKNPERRRFLDVKQQALKLLANSMYGYFGFPRARWYCLECAESITALGRKYHLHTIDIVPKFGFDVIYGDTDSVYLIKPNITDRERVMKDAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMADYKTEGPHIVAAKKAMKRGLEFKQGDIVTYVVTKKGKSISDKARVIDFVEEGDYDPDYYINNQVLPSVLRILEALGYSEDELKGLGKQMKLGGF

SEQ ID NO: 382-390 - skipped (9 entries)

SEQ ID NO: 391 - protein, 773 aa, unidentified
  source 1..773: note = Thermococci archaeon
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFDIETFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLYPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLYPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 392 - protein, 773 aa, unidentified
  source 1..773: note = Thermococci archaeon
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLYPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 393 - protein, 773 aa, unidentified
  source 1..773: note = Thermococci archaeon
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAGSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 394 - protein, 773 aa, unidentified; identical to SEQ ID NO: 393
  source 1..773: note = Thermococci archaeon

SEQ ID NO: 395 - protein, 773 aa, unidentified
  source 1..773: note = Thermococci archaeon
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDIRSSAASIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 396 - protein, 773 aa, unidentified
  source 1..773: note = Thermococci archaeon
MILDTDAITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 397 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLAPSIIVTHNVSPDTLEKECKNYDVAPIVGYRFCKDFKGFIPSILEDLIETRQKVKRKMKATIDPIEKKMLDYRQRALKILANSIYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 398 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLAPSIIVTHNVSPDTLEKECKNYDVAPIVGYRFCKDFKGFIPSILEDLIETRQKVKRKMKATIDPIEKKMLDYRQRALKILANSTYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 399 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESITAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 400 - protein, 773 aa, synthetic construct
MILDTDAITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSAAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 401 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSAAASIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 402 - protein, 773 aa, synthetic construct; identical to SEQ ID NO: 393

SEQ ID NO: 403 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 404 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSGPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 405 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSAAASIIVTHNVSPDTLEKECKNYDVAPIVGYRFCKDFKGFIPSILEDLIETRQKVKRKMKATIDPIEKKMLDYRQRSLKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 406 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAGSIIVTHNVSPDTLEKECKNYDVAPIVGYRFCKDFKGFIPSILEDLIETRQKVKRKMKATIDPIEKKMLDYRQRSLKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 407 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAPSIIVTHNVSPDTLEKECKNYDVAPIVGYRFCKDFKGFIPSILEDLIETRQKVKRKMKATIDPIEKKMLDYRQRSLKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 408 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSGPSIIVTHNVSPDTLEKECKNYDVAPIVGYRFCKDFKGFIPSILEDLIETRQKVKRKMKATIDPIEKKMLDYRQRSLKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 409 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSAAASIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYHETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 410 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAGSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYHETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 411 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYHETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 412 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSGPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYHETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 413 - protein, 773 aa, synthetic construct
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFAIATFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLAPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSSAPSIIVTHNVSPDTLEKECKNYDVAPIVGYRFCKDFKGFIPSILEDLIETRQKVKRKMKATIDPIEKKMLDYRQRSLKILANSYYGYQGYPKARWYSLECAESVTAWGRHYHETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 414 - protein, 785 aa, unidentified
  source 1..785: note = Thermoplasmata archaeon
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFDIEVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSLYPSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 415 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAISIIVTHNIDPSTLTICKEGENKAPGLDYCFPSNKIGFIPEILKNLIDIRIKIKKEIKKVKNTEISRNLDVKQKSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 416 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAISIIVTHNIDPSTLTICKEGENKAPGLDYCFPSNKIGFIPEILKNLIDIRIKIKKEIKKVKNTEISRNLDVKQQALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 417 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAISIIVTHNIDPSTLTICKEGENKAPGLDYCFPSNKIGFIPEILKNLIDIRIKIKKEIKKVKNTEISRNLDVKQQSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 418 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAASIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 419 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAASIIVTHNIDPSTLTICKEGENKAPGLDYCFPSNKIGFIPEILKNLIDIRIKIKKEIKKVKNTEISRNLDVKQKSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 420 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSAAASIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYHKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 421 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAGSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 422 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAGSIIVTHNIDPSTLTICKEGENKAPGLDYCFPSNKIGFIPEILKNLIDIRIKIKKEIKKVKNTEISRNLDVKQKSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 423 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAGSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYHKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 424 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAPSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 425 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAPSIIVTHNIDPSTLTICKEGENKAPGLDYCFPSNKIGFIPEILKNLIDIRIKIKKEIKKVKNTEISRNLDVKQKSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 426 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAPSIIVTHNIDPSTLTICKEGENKAPGLDYCFPSNKIGFIPEILKNLIDIRIKIKKEIKKVKNTEISRNLDVKQKSLKILANSFYGYMGYARARWYKLECAESVTAFARMYHKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 427 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSAPSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYHKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 428 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSGPSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 429 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSGPSIIVTHNIDPSTLTICKEGENKAPGLDYCFPSNKIGFIPEILKNLIDIRIKIKKEIKKVKNTEISRNLDVKQKSLKILANSFYGYMGYARARWYKKECAESVTAFARMYIKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 430 - protein, 785 aa, synthetic construct
MQYLLLDADYESKNGKPIIKLCLKREKTIVAYDESFLPYFYVHVKDEEEFKKSLRKFNIKEFKIEEKMIFGKPLRIFKLILPHPQDVPKIRDKIKNLPEVEEIYEYDIPFARRYLIDKGLTPMNLVEADIEERDGRYFIKKIKPAGEATLSLRVMAFAIAVYNPMGAPRPEKDPIIMISLCSNYGLRKVLTWKKPIKDYDFVEICQDERDMLSKFLEIIEKEDIDILIGYNTDNFDFPYLKSRFKKLKLKLELGRDGGEPKIKSRRGLPEARIFGRCHVDLYPIIRRSITLSSYVLENVVRELLGIEKEKIPGEKLWEFWNSGGEKLCKLIEYSMEDATTTMKLGEKYLPIYIELSRLVGQTLFSVARMTYGQMVEWTLLREAYKHGEIAPNKPRGREYIAREEESYAGGYVVEPKKGMAENIAVFDFRSSGPSIIVTHNIDPSTLTICKEGENKAPGLDYCFPSNKIGFIPEILKNLIDIRIKIKKEIKKVKNTEISRNLDVKQKALKILANSFYGYMGYARARWYKKECAESVTAFARMYHKKVMKIAEEKFGLEVIYGDTDSLFTVIPQEKRDEIVSQFLSYVNDILPGIIELEFEGFYKRGIFVTKKRYALIDEDGKITVKGLEFVRRDWAPIARKTQEKVLRALLMYASPKKALKIVKKVIDDIRNRNVKLEDLAIYTQLTKKIQSYKNIEPHVIAAQKLEKKGKKVYPGMIIGYIIVKGSKKISFRAEPIEFVSINDYDPEYYIENQIMPAVQRIMEAIGYSKDFLKFGIKQERIEKWF

SEQ ID NO: 431 - protein, 774 aa, Thermococcus sp.
  source 1..774: note = 2319x1
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLAPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 432 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLAPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 433 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSAAASIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 434 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSAAASIIVTHNVSPDTLEKEGCENYDIAPIVGYKFCKDFPGFIPSILGDLIAMRQEIKKKMKATIDPIEKKMLDYRQRSVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 435 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSAAASIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYHEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 436 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAGSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 437 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAGSIIVTHNVSPDTLEKEGCENYDIAPIVGYKFCKDFPGFIPSILGDLIAMRQEIKKKMKATIDPIEKKMLDYRQRSVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 438 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAGSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYHEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 439 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 440 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAPSIIVTHNVSPDTLEKEGCENYDIAPIVGYKFCKDFPGFIPSILGDLIAMRQEIKKKMKATIDPIEKKMLDYRQRSVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 441 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAPSIIVTHNVSPDTLEKEGCENYDIAPIVGYKFCKDFPGFIPSILGDLIAMRQEIKKKMKATIDPIEKKMLDYRQRSVKLLANSYYGYMGYPKARWYSLECAESVTAWGRHYHEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 442 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSAPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYHEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 443 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSGPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 444 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSGPSIIVTHNVSPDTLEKEGCENYDIAPIVGYKFCKDFPGFIPSILGDLIAMRQEIKKKMKATIDPIEKKMLDYRQRSVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 445 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSSGPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYHEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 446 - protein, 774 aa, Thermococcus litoralis
MILDTDYITKDGKPIIRIFKKENGEFKIELDPHFQPYIYALLKDDSAIEEIKAIKGERHGKSVRVVDAVKVKKKFLGREVEVWKLIFEHPQDVPAMRDKIKEHPAVIDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYERNELAPNKPDEEEYKRRLRTTYLGGYVKEPEKGLWENIIYLDFRSLYPSIIVTHNVSPDTLEKEGCENYDIAPIVSYRFCKDFPGFIPSILGDLIAMRQEIKKKMKATIDPVERKMLDYRQRAVKLLANSYYGYMGYPKARWYSKECAESVTAWGRHYIEMTIKEIEEKFGFKVLYADTDGFYATISGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVLEKIAKYRVPLEKLVIHEQITRDLKDYKAIGPHVAIAKRLAARGIKVKPGTIISYIVLKGSGKISDRVILLTEYDPEKHKYDPDYYIENQVLPAVLRILEAFGYRKEDLRYQSSKQTGLDAWLKR

SEQ ID NO: 447 - protein, 774 aa, synthetic construct
MILDTDYITKDGKPIIRIFKKENGEFKIELDPHFQPYIYALLKDDSAIEEIKAIKGERHGKSVRVVDAVKVKKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYERNELAPNKPDEEEYKRRLRTTYLGGYVKEPEKGLWENIIYLDFRSLAPSIIVTHNVSPDTLEKEGCENYDIAPIVSYRFCKDFPGFIPSILGDLIAMRQEIKKKMKATIDPVERKMLDYRQRAVKLLANSYYGYMGYPKARWYSKECAESVTAWGRHYIEMTIKEIEEKFGFKVLYADTDGFYATISGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVLEKIAKYRVPLEKLVIHEQITRDLKDYKAIGPHVAIAKRLAARGIKVKPGTIISYIVLKGSGKISDRVILLTEYDPEKHKYDPDYYIENQVLPAVLRILEAFGYRKEDLRYQSSKQTGLDAWLKR

SEQ ID NO: 448 - protein, 773 aa, unidentified
  source 1..773: note = archaeon BMS3Abin16
MSRDLLLDIDYKTVDEKAELRLFLRNETVFDKDFQPYIYVELEDGGAEDELSVFGSCEAVTRLYYGVEKKVYKLTVSHPGEVPKIREELRNMDSVREIYEHDILFSRRYLIDKRLVPLNYAEFTAEKGILKEIVSTDGQEENLRVMAFDIEVYNPKGAPRVSVDPVIMLSYAASTGRRGLLTYKDAEAPDYVTVVEDEKTVLRRFCEIVEEEGIDILVGYNSDQFDIPYILDRAKTLGVDFPLGRLGLQPSIRKGRGLTESVIRGRPHVDLYPVVRRNVKLSSYVLENVVLDVLGIEKEKIPAGLMWQYWDRGGADLRAFMQYSIEDVDVTLELAEKFLPLYIVLSQVVGLPIQDTSRMTAGQLVEWLLIREAFVRGELVPNKGGGKEYLARAGDTYAGGYVMEPVKGLVENIVVFDFRSLYPSIIVTHNIDPTTLRAGRDENSPPELDFHFATDEEGFIPSVLKRILDRRLDAKRRMKDATDPFEKKMLNVTQNALKIIANSFYGYMGYPRARWYKKECAESTTSYARMYTKLVMKIAEEEYGFKVVYGDTDSLFMVVPPEKRERAMEFMRDANKRLPGTVELEYDGFYPRGIFITKKRYALIDEKENITVKGLETVRRDWVKLSRDTQQEVLSILLKEGDPMKAVQIVKDRVATLKERRVSLDEITLFTQLTKNIKSYKNKGPHVRAAEKSIERGREVNPGTSIGYIIRKGPGMISDRAEPIEDAKIDDIDVDYYIENQLLPPVARILEALGYSKEHLKGGLVQNNLKKWF

SEQ ID NO: 449 - protein, 773 aa, synthetic construct
MSRDLLLDIDYKTVDEKAELRLFLRNETVFDKDFQPYIYVELEDGGAEDELSVFGSCEAVTRLYYGVEKKVYKLTVSHPGEVPKIREELRNMDSVREIYEHDILFSRRYLIDKRLVPLNYAEFTAEKGILKEIVSTDGQEENLRVMAFAIAVYNPKGAPRVSVDPVIMLSYAASTGRRGLLTYKDAEAPDYVTVVEDEKTVLRRFCEIVEEEGIDILVGYNSDQFDIPYILDRAKTLGVDFPLGRLGLQPSIRKGRGLTESVIRGRPHVDLAPVVRRNVKLSSYVLENVVLDVLGIEKEKIPAGLMWQYWDRGGADLRAFMQYSIEDVDVTLELAEKFLPLYIVLSQVVGLPIQDTSRMTAGQLVEWLLIREAFVRGELVPNKGGGKEYLARAGDTYAGGYVMEPVKGLVENIVVFDFRSLAPSIIVTHNIDPTTLRAGRDENSPPELDFHFATDEEGFIPSVLKRILDRRLDAKRRMKDATDPFEKKMLNVTQNALKIIANSFYGYMGYPRARWYKKECAESTTSYARMYTKLVMKIAEEEYGFKVVYGDTDSLFMVVPPEKRERAMEFMRDANKRLPGTVELEYDGFYPRGIFITKKRYALIDEKENITVKGLETVRRDWVKLSRDTQQEVLSILLKEGDPMKAVQIVKDRVATLKERRVSLDEITLFTQLTKNIKSYKNKGPHVRAAEKSIERGREVNPGTSIGYIIRKGPGMISDRAEPIEDAKIDDIDVDYYIENQLLPPVARILEALGYSKEHLKGGLVQNNLKKWF

SEQ ID NO: 450 - protein, 773 aa, unidentified
  source 1..773: note = archaeon BMS3Bbin16
MSRDLLLDIDYKTVDEKAELRLFLRNETVFDKDFQPYLYVELEDSGAEDELSVFGSCEAVTRLYYGVEKKVYKLTVSHPGEVPKIREELRNMDSVREIYEHDILFSRRYLIDKRLVPLNYAEFTAEKGILKEIVSTDGQEENLRVMAFDIEVYNPKGAPRVSVDPVIMLSYAASTGRRGLLTWKDADAPDYVTVVEDEKTVLRRFCEIVEEEGIDILVGYNSDQFDIPYILERAKTLGVDFPLGRLGLQPSIRKGRGLTESVIRGRPHVDLYPVVRRNVKLSSYVLENVVLDVLGIEKEKIPPGLMWQYWDRGGADLRAFMQYSIEDVDVTLELAEKFLPLYIVLSRVVGLPIHDTSRMTAGQLVEWLLIREAFVRGELVPNKGGGKEYLARAGDTYAGGYVMEPEKGLVENIVVFDFRSLYPSIIVTHNIDPTTLRKGRSEDSPPELDFHFATDEEGFIPSVLKRILDRRLDAKRRMKEATDPFEKKMLNVTQNALKIIANSFYGYMGYPRARWYKKECAESTTSYARMYTKLVMKIAEEEYGFKVVYGDTDSLFMVVPPEKMERAMEFMRDANKRLPGTVELEYDGFYPRGIFITKKRYALIDEKDNITVKGLETVRRDWVKLSRDTQQEVLSILLKEGDPMKAAQIVKDRVATLKERRVSLEDITLYTQLTKNIKSYKNKGPHVRAAQKSIDRGREVNPGTSIGYIIRKGPGMISDRAEPIEDAKIDDIDVDYYIENQLLPPVARIMEALGYSKEHLKGGLVQNNLKQWF

SEQ ID NO: 451 - protein, 773 aa, synthetic construct
MSRDLLLDIDYKTVDEKAELRLFLRNETVFDKDFQPYLYVELEDSGAEDELSVFGSCEAVTRLYYGVEKKVYKLTVSHPGEVPKIREELRNMDSVREIYEHDILFSRRYLIDKRLVPLNYAEFTAEKGILKEIVSTDGQEENLRVMAFAIAVYNPKGAPRVSVDPVIMLSYAASTGRRGLLTWKDADAPDYVTVVEDEKTVLRRFCEIVEEEGIDILVGYNSDQFDIPYILERAKTLGVDFPLGRLGLQPSIRKGRGLTESVIRGRPHVDLAPVVRRNVKLSSYVLENVVLDVLGIEKEKIPPGLMWQYWDRGGADLRAFMQYSIEDVDVTLELAEKFLPLYIVLSRVVGLPIHDTSRMTAGQLVEWLLIREAFVRGELVPNKGGGKEYLARAGDTYAGGYVMEPEKGLVENIVVFDFRSLAPSIIVTHNIDPTTLRKGRSEDSPPELDFHFATDEEGFIPSVLKRILDRRLDAKRRMKEATDPFEKKMLNVTQNALKIIANSFYGYMGYPRARWYKKECAESTTSYARMYTKLVMKIAEEEYGFKVVYGDTDSLFMVVPPEKMERAMEFMRDANKRLPGTVELEYDGFYPRGIFITKKRYALIDEKDNITVKGLETVRRDWVKLSRDTQQEVLSILLKEGDPMKAAQIVKDRVATLKERRVSLEDITLYTQLTKNIKSYKNKGPHVRAAQKSIDRGREVNPGTSIGYIIRKGPGMISDRAEPIEDAKIDDIDVDYYIENQLLPPVARIMEALGYSKEHLKGGLVQNNLKQWF

SEQ ID NO: 452 - protein, 806 aa, unidentified
  source 1..806: note = Candidatus Hadarchaeum yellowstonense
MLLDVDYAEEEEKPSIRLFVKTGSEVLVAIDPDFEEYFYVVSDHPAKASKLIEKVEVEEDGVSIRPKGVEIVKRTFLGNEVEAIKVSFYQAKDSSKLRHKIRELPGVREIYEFDIPPARRYLIDRGLTPMAGVEFDGRIEERQGIKTVILDSPPRPAQVEEPKLNIMSFDIEVYNPTGSVRPDKDPIIMISLADNNGLRKVITWKNFERSQEYVEVVGSEREMIKRFVDLVKERDVDILLGYNTDLFDLPYIRSRAKQLKVKLDLGRDGSELVVRKRRFATASKIRGRIHVDVFAMVDFLATIGSIKLIHYSLADVYRHLLGKEKPDFEFTEMVDAWEKGGDAGRKFLEYSMSDADATLEVGLELLPLFLGLTRVVGQTLFDVQRMTPGQLVEWLLVAEAHRIGELVPGRPVGEEYEERMEETFVGAYVMEPVKGLHENLVVFDFRSLYPSIIVTHNIDPSTLNCKDCKPGEREQVPGLEYYFCRRRKGFIPATLQRIIEERMKLKAELKKLVRGTKEYRALDARQWAMKIVANSFYGMLGYPRARWYSKECAESVTSFGRHYIHKTIDMAREFGLEVVYGDTDSLHCKLNGKTREEALAFLKKVNDSLPGIMELELEGFYPRGIFITKKRYAMIDDEGRMVVKGLEFVRRDWAAIAKKTQEEVLKAILRDGSPEKAAEIIRKTTRDVYEGRVNLEDLIIYTQLKMPIESYKAIGPHVVAAKRLRELGHEIEPGMMIAYVEVKGPGSISERAVPVEDFKGREYDPDYYVGHQVLPAVMRIMEVLGYREIDLKFERQRQVGLDRFMK

SEQ ID NO: 453 - protein, 806 aa, synthetic construct
MLLDVDYAEEEEKPSIRLFVKTGSEVLVAIDPDFEEYFYVVSDHPAKASKLIEKVEVEEDGVSIRPKGVEIVKRTFLGNEVEAIKVSFYQAKDSSKLRHKIRELPGVREIYEFDIPPARRYLIDRGLTPMAGVEFDGRIEERQGIKTVILDSPPRPAQVEEPKLNIMSFAIAVYNPTGSVRPDKDPIIMISLADNNGLRKVITWKNFERSQEYVEVVGSEREMIKRFVDLVKERDVDILLGYNTDLFDLPYIRSRAKQLKVKLDLGRDGSELVVRKRRFATASKIRGRIHVDVFAMVDFLATIGSIKLIHYSLADVYRHLLGKEKPDFEFTEMVDAWEKGGDAGRKFLEYSMSDADATLEVGLELLPLFLGLTRVVGQTLFDVQRMTPGQLVEWLLVAEAHRIGELVPGRPVGEEYEERMEETFVGAYVMEPVKGLHENLVVFDFRSLAPSIIVTHNIDPSTLNCKDCKPGEREQVPGLEYYFCRRRKGFIPATLQRIIEERMKLKAELKKLVRGTKEYRALDARQWAMKIVANSFYGMLGYPRARWYSKECAESVTSFGRHYIHKTIDMAREFGLEVVYGDTDSLHCKLNGKTREEALAFLKKVNDSLPGIMELELEGFYPRGIFITKKRYAMIDDEGRMVVKGLEFVRRDWAAIAKKTQEEVLKAILRDGSPEKAAEIIRKTTRDVYEGRVNLEDLIIYTQLKMPIESYKAIGPHVVAAKRLRELGHEIEPGMMIAYVEVKGPGSISERAVPVEDFKGREYDPDYYVGHQVLPAVMRIMEVLGYREIDLKFERQRQVGLDRFMK

SEQ ID NO: 454 - protein, 805 aa, unidentified
  source 1..805: note = candidate division MSBL1 archaeon SCGC-AAA261F17
MLLDSDYLKTRKPPAMRLFIKKDGGIVTVLDPHFTHYFYVESENPQKIAKAIERVEAEKYGKKVSPKSTKVVERKFLGEEKKVIKVLADSPRDITPLRKEIKDFPEVKGFYEHDIPPARRYLIEHELTSMSGVKAEGESQKGDYGEELVLTKPPESIEGADEELNILAFDIETYGPTGNPRAEKDPIVMISVSDNQGLEKILTWKDFDLNLDYVEVLDDEKSMIERFIQLVQERDADIIMGYNTDLFDFPYLTQRAEKLDIKLELGRDGSEPSTKKRRFATVTKIAGRVHADVYAMVEFLSRIGAIRLIDYTLENVYKHVIGKEKPDLEYSDIPKAWDEGGEKARELVEYSLSDAKATLELGTEILPLFTELSRTVKQSLFDVSRMTPGQLVEWLLIFNAHKINELILPRPLGREYKRRRGETYIGGYVKEPTPGLHEDLVVFDFRSLYPTIIITHNIDPATLDGERCPSEETVTAPDLEYEFCQDRKGFIPETLKGLVEGRAKLKQEMSQLDEESREYQSLYNRQWALKIIANSFYGMLGYPRARWYSKECAESVTSFGRHYIKDTIEMAKDEGFEVIYGDTDSLFAKLNGKSREDVENFLNKVNESLPGIMKLELEDYYKRGVFVTKKRYAMISEDDKIVVKGLEFVRRDWAALAKRTQEQVIEAILHDASPEKAAKIVLETTKAIKQGEVDLDDLVIHTQLKKPLDEYKARGPHVAAAERLQKLGEEVEPGMTITYIVEKGSGSISDRAIPTSDFEGRDYDPDYYVENQVLPAVMRIMEVLDYGEEDLRHEETRQVKLGKFK

SEQ ID NO: 455 - protein, 805 aa, synthetic construct
MLLDSDYLKTRKPPAMRLFIKKDGGIVTVLDPHFTHYFYVESENPQKIAKAIERVEAEKYGKKVSPKSTKVVERKFLGEEKKVIKVLADSPRDITPLRKEIKDFPEVKGFYEHDIPPARRYLIEHELTSMSGVKAEGESQKGDYGEELVLTKPPESIEGADEELNILAFAIATYGPTGNPRAEKDPIVMISVSDNQGLEKILTWKDFDLNLDYVEVLDDEKSMIERFIQLVQERDADIIMGYNTDLFDFPYLTQRAEKLDIKLELGRDGSEPSTKKRRFATVTKIAGRVHADVYAMVEFLSRIGAIRLIDYTLENVYKHVIGKEKPDLEYSDIPKAWDEGGEKARELVEYSLSDAKATLELGTEILPLFTELSRTVKQSLFDVSRMTPGQLVEWLLIFNAHKINELILPRPLGREYKRRRGETYIGGYVKEPTPGLHEDLVVFDFRSLAPTIIITHNIDPATLDGERCPSEETVTAPDLEYEFCQDRKGFIPETLKGLVEGRAKLKQEMSQLDEESREYQSLYNRQWALKIIANSFYGMLGYPRARWYSKECAESVTSFGRHYIKDTIEMAKDEGFEVIYGDTDSLFAKLNGKSREDVENFLNKVNESLPGIMKLELEDYYKRGVFVTKKRYAMISEDDKIVVKGLEFVRRDWAALAKRTQEQVIEAILHDASPEKAAKIVLETTKAIKQGEVDLDDLVIHTQLKKPLDEYKARGPHVAAAERLQKLGEEVEPGMTITYIVEKGSGSISDRAIPTSDFEGRDYDPDYYVENQVLPAVMRIMEVLDYGEEDLRHEETRQVKLGKFK

SEQ ID NO: 456 - protein, 809 aa, unidentified
  source 1..809: note = Hadesarchaea archaeon
MRGLLFDVDIAEEEERPNVRLFVKVASETVVAIDPQFEEYFYVVADHPAKTSKLIEKIELDEGGRPIRPKSVEMVRRTLLGNEVEAIRVSFHQPRDAAKLRHKIRELPGVKEIYEFDIPPARRYLIDRGLTPMAGIEFSGSIEVRDGVKTVVMDGPPKPAPVEETRLNIMSFDIEVYNPTGSVRPDKDPIIMISLADNRGLRKVITWKNFDKKPEYVEVVGSEREMIKKFVELVKERDVDILLGYNTDLFDLPYIRSRAKQLRVKLDLGRDGSELVVRKRRFATASKIRGRVHVDVFAMVDFLATIGSIRLIHYSLADVYRHYAGREKPDFEFSEMINAWERGGDAGRRFLEYSMSDADATLEVGSELLPLFLGLTRVVGQTLFDVQRMTPGQLVEWLLVAEAHRIGELVPPRPVGEEFEERAEGTFTGAYVMEPVKGLHEDLVVFDFRSLYPSIIVTHNIDPSTLNCRDCKPGEREQVPGLSYYFCKRRKGFIPAVLERVIEERTKLKAELKKIGRETREYRALDARQWAMKIVANSFYGMLGYPRARWYSKQCAESVTSFGRHYIHRTIEMAREFGLEVVYGDTDSLHCKLNGKTREEAMVFLRKVNESLPGIMELELEGFYPRGIFITKKRYAMVDDEGRMVVKGLEFVRRDWAAIAKKTQEEVLRAILRDGSPKKAAEIIRKTTRDVYEGRVNLEDLIIYTQLKMPIESYKAIGPHVVAAKRLRELGHEIEPGMMIAYIEVKGPGSISDRAVPVEDFEGKEYDPDYYVGHQILPAVMRIMEVLGYSEEDLKFEREKQIGLDRFMK

SEQ ID NO: 457 - protein, 809 aa, synthetic construct
MRGLLFDVDIAEEEERPNVRLFVKVASETVVAIDPQFEEYFYVVADHPAKTSKLIEKIELDEGGRPIRPKSVEMVRRTLLGNEVEAIRVSFHQPRDAAKLRHKIRELPGVKEIYEFDIPPARRYLIDRGLTPMAGIEFSGSIEVRDGVKTVVMDGPPKPAPVEETRLNIMSFAIAVYNPTGSVRPDKDPIIMISLADNRGLRKVITWKNFDKKPEYVEVVGSEREMIKKFVELVKERDVDILLGYNTDLFDLPYIRSRAKQLRVKLDLGRDGSELVVRKRRFATASKIRGRVHVDVFAMVDFLATIGSIRLIHYSLADVYRHYAGREKPDFEFSEMINAWERGGDAGRRFLEYSMSDADATLEVGSELLPLFLGLTRVVGQTLFDVQRMTPGQLVEWLLVAEAHRIGELVPPRPVGEEFEERAEGTFTGAYVMEPVKGLHEDLVVFDFRSLAPSIIVTHNIDPSTLNARDCKPGEREQVPGLSYYFCKRRKGFIPAVLERVIEERTKLKAELKKIGRETREYRALDARQWAMKIVANSFYGMLGYPRARWYSKQCAESVTSFGRHYIHRTIEMAREFGLEVVYGDTDSLHCKLNGKTREEAMVFLRKVNESLPGIMELELEGFYPRGIFITKKRYAMVDDEGRMVVKGLEFVRRDWAAIAKKTQEEVLRAILRDGSPKKAAEIIRKTTRDVYEGRVNLEDLIIYTQLKMPIESYKAIGPHVVAAKRLRELGHEIEPGMMIAYIEVKGPGSISDRAVPVEDFEGKEYDPDYYVGHQILPAVMRIMEVLGYSEEDLKFEREKQIGLDRFMK

SEQ ID NO: 458 - protein, 773 aa, unidentified
  source 1..773: note = Euryarchaeota archaeon
MARDLLLDIDYVTVDEKAQVRLFLKDKILFDPGFQPYFYVLAHDGAVEERLRDFGAVEAVQRRMLGREMRFFKLILSHPSEVPKIREEVRSIEGVEGIFEHDILFARRYLIDKGLTPLNYAEYRAEQGFLKGISSAGEGPESLRVMAFDIETYNPKGAPRAEKDPVIMLSLSTNTGLRRLLTYKSGEGLDFVELVEDEKALLHRFKELVNEEGVEVLVGYNSDQFDLPYLVARAKALGVELPLGQDGSQPQIRKGRGLVESVVKGRPHVDLYPIVRRNVRLSSYVLENVVKEVLGREKEKIPHDAMCGYWDRGGRELQRFMAYSMEDADVTLELAERFLPLYIELSRVVGLPLHDVARMTAGQLVEWLLIREAFARGEVVPNKGSGREYLARSEDTYAGGYVMEPVKGIVENIVVFDFRSLYPSIIVTHNIDPATLRPGRGENSPPELDYHFTTEEEGFIPSVLKRVLERRLSAKRRMKEARDPGEKRMLDISQRALKIIANSFYGYMGYPRARWYKKECAESVTSFARMYTKKVMAIAEEEYGFKVVYGDTDSLFIVVQPEEKERAMSFMEDVNRRLPGTVELEYDGFYPRGIFITKKRYALIDEKGNIVVKGLETVRRDWTRLSRDTQQKVLSVILREGDPKKAADIVKDTINRLKERRVDLEDITIYTQLTKGIGRYKNVGPHVKAAQKAIDRGREVNPGMAIGYIIKKGRGLISDRAEPVEDATIEDYDVDYYIENQVLPPVARIMEVLGYSKEHLKEEMVQGSLQRWF

SEQ ID NO: 459 - protein, 773 aa, synthetic construct
MARDLLLDIDYVTVDEKAQVRLFLKDKILFDPGFQPYFYVLAHDGAVEERLRDFGAVEAVQRRMLGREMRFFKLILSHPSEVPKIREEVRSIEGVEGIFEHDILFARRYLIDKGLTPLNYAEYRAEQGFLKGISSAGEGPESLRVMAFAIATYNPKGAPRAEKDPVIMLSLSTNTGLRRLLTYKSGEGLDFVELVEDEKALLHRFKELVNEEGVEVLVGYNSDQFDLPYLVARAKALGVELPLGQDGSQPQIRKGRGLVESVVKGRPHVDLAPIVRRNVRLSSYVLENVVKEVLGREKEKIPHDAMCGYWDRGGRELQRFMAYSMEDADVTLELAERFLPLYIELSRVVGLPLHDVARMTAGQLVEWLLIREAFARGEVVPNKGSGREYLARSEDTYAGGYVMEPVKGIVENIVVFDFRSLAPSIIVTHNIDPATLRPGRGENSPPELDYHFTTEEEGFIPSVLKRVLERRLSAKRRMKEARDPGEKRMLDISQRALKIIANSFYGYMGYPRARWYKKECAESVTSFARMYTKKVMAIAEEEYGFKVVYGDTDSLFIVVQPEEKERAMSFMEDVNRRLPGTVELEYDGFYPRGIFITKKRYALIDEKGNIVVKGLETVRRDWTRLSRDTQQKVLSVILREGDPKKAADIVKDTINRLKERRVDLEDITIYTQLTKGIGRYKNVGPHVKAAQKAIDRGREVNPGMAIGYIIKKGRGLISDRAEPVEDATIEDYDVDYYIENQVLPPVARIMEVLGYSKEHLKEEMVQGSLQRWF

SEQ ID NO: 460 - protein, 774 aa, Thermococcus sp.
  source 1..774: note = 2319x1
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCENYDIAPIVSYRFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGSGKISDRVILLTEYDPEKHKYDPDYYIENQVLPAVLRILEAFGYRKEDLRYQSSKQTGLDTWLKR

SEQ ID NO: 461 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLAPSIIVTHNVSPDTLEKEGCENYDIAPIVSYRFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGSGKISDRVILLTEYDPEKHKYDPDYYIENQVLPAVLRILEAFGYRKEDLRYQSSKQTGLDTWLKR

SEQ ID NO: 462 - protein, 1314 aa, Thermococcus sp.
  source 1..1314: note = M39
MILDTDYITKDGKPIIRIFKKENGEFKIELDRNFQPYIYALLENDEAIGEVKKITAKRGNKTVRILKAEKVQKKFLGRPIEVWKLIFEHPQDVPAIRDKIREHPKVIDIFEYDIPFAKRYLIDKGLIPMEGEEELKLLAFDIETFYHEGDQFGEGEIIMISYADENEAKVITWKKIDLPYVEVVSSEREMIKRFIQIVREKDPDVLITYNGDNFDLPYLLKRAQKLGLRLFISRDKEHPEPKIQRMGDRFAVEIKGRIHFDLFPVVRRAINLPTYTLEAVYEAVLGKKKSKLEAAEITAIWESEEGMKELAKYSMEDAKATYELGKEFFPMEAELARLIGQSVWDVSRSSTGNLVEWFLLRIAYQRNELAPNKPSDEEYQRRLKTTYIGGYVKEPERGLWGNIVYLDFKALYPSIIVTHNVSPDTLEKEGCKNYDVAPIVGYRFCKDFKGFIPSILEELIETRTKIKKEMKATIDPVKKKMLDYRQKAIKLLANSILPDEWVFVLENNHPRFLGIGELVDKMIDANPEKVKIVDGSEVLEVSDLWTISFDKSSKKAHLSKIKAVIRHRYRGKAYRIKLSSGRSIKITQGHSLFTVKGSEIKEITGDELKEGDVIIVPKRFKLDGREMFIILPRLFALLPPEETEDIILTISTRGKKNFFRGMLRALKWIFGEEEKRIRTFNRYLSHLEELGFVRVKPRGFEVIDWEKLKNYKKLYEILSQNVKYNGNKGEYFIKFNDLRDVIEAFPPEELAEWKIGTLNGFRMNSIVKLDKDFARFLGYYVSEGSARKQRNQTGGSSYTVRLYNENEEILKDMEHVSKVLFSEVKKTEKYVEISKKVAYLIVKAMCGIGAENKRIPEPIFLAPEEIRWEFLNAYFLGDGDVHPSKRLRLSTKSELLANQLVFLLNSLGISSIKLGFDSGVYRIYINEDLPFLRTGRQKNNYYSNLIPKEVLEIVFGKRFQKNMSFAKFKELVEKGELDWEKARLIEWLVEGDLVLDRVEKVEEFRYDGYVYDLSVENDENFLAGFGWLYAHNSFYGYMGYPKARWYSKECAESVTAWGRTYIEMTIKEIEEKFGFKVLYADTDGLYATIPGTNAETIKKKAKEFLKYINSKLPGLLELEYEGFYVRGFFVTKKRYALIDEEGKIITRGLEVVRRDWSEIAKETQAKVLEAILKDGSIEKAVEIVKKVVEDLANYKVPLEKLVIHEQITKSLDEYKATGPHVAVAKRLKAKGIKVRPGMIISYIILKGDKKISERAILLSEYDPKKHKYDPDYYIEHQVLPAVLRILEAFGYRKEDLRYQKTKQLGLGAWLNMRK

SEQ ID NO: 463 - protein, 776 aa, synthetic construct
MILDTDYITKDGKPIIRIFKKENGEFKIELDRNFQPYIYALLENDEAIGEVKKITAKRGNKTVRILKAEKVQKKFLGRPIEVWKLIFEHPQDVPAIRDKIREHPKVIDIFEYDIPFAKRYLIDKGLIPMEGEEELKLLAFDIETFYHEGDQFGEGEIIMISYADENEAKVITWKKIDLPYVEVVSSEREMIKRFIQIVREKDPDVLITYNGDNFDLPYLLKRAQKLGLRLFISRDKEHPEPKIQRMGDRFAVEIKGRIHFDLFPVVRRAINLPTYTLEAVYEAVLGKKKSKLEAAEITAIWESEEGMKELAKYSMEDAKATYELGKEFFPMEAELARLIGQSVWDVSRSSTGNLVEWFLLRIAYQRNELAPNKPSDEEYQRRLKTTYIGGYVKEPERGLWGNIVYLDFKALYPSIIVTHNVSPDTLEKEGCKNYDVAPIVGYRFCKDFKGFIPSILEELIETRTKIKKEMKATIDPVKKKMLDYRQKAIKLLANSFYGYMGYPKARWYSKECAESVTAWGRTYIEMTIKEIEEKFGFKVLYADTDGLYATIPGTNAETIKKKAKEFLKYINSKLPGLLELEYEGFYVRGFFVTKKRYALIDEEGKIITRGLEVVRRDWSEIAKETQAKVLEAILKDGSIEKAVEIVKKVVEDLANYKVPLEKLVIHEQITKSLDEYKATGPHVAVAKRLKAKGIKVRPGMIISYIILKGDKKISERAILLSEYDPKKHKYDPDYYIEHQVLPAVLRILEAFGYRKEDLRYQKTKQLGLGAWLNMRK

SEQ ID NO: 464 - protein, 778 aa, unidentified
  source 1..778: note = Candidatus Altiarchaeales archaeon
MKKTLLDADYITREEKAVVRLFYKTEEGREIQEVADFRPYMYVLPEEHDLKKLQREIKELKNVTNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGAKVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKNILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGSQGSYT

SEQ ID NO: 465 - protein, 773 aa, unidentified
  source 1..773: note = Thermococci archaeon
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFDIETFYHEGDEFAEGEILMISYADESGAKVITWKKIDLPYVEVVSTEREAIKRFLQVLREKDPDVLLTYNGDNFDFAYIKKRCEKLGLKFTIGREGSEPKIQRMGDRFAVEVKGRIHLDLYPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLYPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 466 - protein, 183 aa, unidentified
  source 1..183: note = Streptavidin avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 467 - protein, 160 aa, unidentified
  source 1..160: note = Streptavidin avidinii
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 468 - protein, 159 aa, unidentified
  source 1..159: note = Streptavidin lavendulae
DPSKESKAQAAVAEAGITGTWYNQLGSTFIVTANPDGSLTGTYESAVGNAESRYVLTGRYDSTPATDGSGTALGWTVAWKNNYRNAHSATTWSGQYVGGAEARINTQWLLTSGTTAANAWKSTLVGHDTFTKVKPSAASIDAAKKAGVNNGNPLDAVQQ

SEQ ID NO: 469 - protein, 128 aa, synthetic construct
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 470 - protein, 167 aa, synthetic construct
MVHHHHHHDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 471 - protein, 152 aa, Gallus gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 472 - protein, 128 aa, Gallus gallus
ARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

(19) United States
(12) Patent Application Publication
(10) Pub. No.: US 2024/0117428 A1
(43) Pub. Date: Apr. 11, 2024

(54) MULTIVALENT BINDING COMPOSITIONS WITH REACTIVE GROUPS

(71) Applicant: Element Biosciences, Inc., San Diego, CA (US)

(72) Inventors: MICHAEL PREVITE, SAN DIEGO, CA (US); MARK AMBROSO, SAN DIEGO, CA (US); TYLER LOPEZ, SAN DIEGO, CA (US); MICHAEL KLEIN, SAN DIEGO, CA (US); VIRGINIA SAADE, SAN DIEGO, CA (US); MATTHEW KELLINGER, SAN DIEGO, CA (US)

(21) Appl. No.: 18/364,085

(22) Filed: Aug. 2, 2023

Related U.S. Application Data
(63) Continuation of application No. PCT/US23/65467, filed on Apr. 6, 2023.
(60) Provisional application No. 63/328,663, filed on Apr. 7, 2022.

(51) Int. Cl. C12Q 1/6876

(52) U.S. Cl. CPC C12Q 1/6876

(57) ABSTRACT

The present disclosure provides nucleotide conjugates each configured to include a core attached to multiple nucleotide-arms, where the nucleotide-arms are modular and comprise (i) a core attachment moiety, (ii) a spacer, (iii) a linker, and (iv) a nucleotide unit. The nucleotide unit of each nucleotide-arm can bind a polymerase which is complexed with a nucleic acid template and nucleic acid primer. The nucleotide unit can bind the 3′ end of the primer at a position that is opposite a complementary nucleotide in the template strand. Under suitable conditions, the nucleotide unit of the nucleotide conjugates binds the primer strand but does not undergo polymerase-catalyzed incorporation. The binding event can be detected, and the specific base of the nucleotide unit can be identified. The nucleotide conjugates described herein are useful for nucleic acid sequencing methods, particularly for massively parallel sequencing methods employed for next gen sequencing platforms.

### CROSS-REFERENCE

This application is a continuation of International Patent Application No. PCT/US23/65467, filed Apr. 6, 2023, which claims the benefit of U.S. Provisional Application No. 63/328,663, filed Apr. 7, 2022, which is incorporated herein by reference in its entirety.

### SEQUENCE LISTING

The present application is being filed along with a Sequence Listing in electronic format. The Sequence Listing is provided as a file entitled 52933-750_301SL.XML, created on Apr. 5, 2023, which is 759,342 bytes in size. The information in the electronic format of the Sequence Listing is incorporated by reference in its entirety.

### BACKGROUND

Nucleic acid sequencing can be used to obtain information in a wide variety of biomedical contexts, including diagnostics, prognostics, biotechnology, and forensic biology. Various sequencing methods have been developed including Maxam-Gilbert sequencing and chain-termination methods, or de novo sequencing methods including shotgun sequencing and bridge PCR, or next-generation methods including polony sequencing, 454 pyrosequencing, Illumina sequencing, SOLiD sequencing, Ion Torrent semiconductor sequencing, HeliScope single molecule sequencing, SMRT® sequencing, and others. Despite advances in DNA sequencing, many challenges remain unaddressed.

### SUMMARY

Aspects disclosed herein, in some embodiments, are synthetic polypeptides, comprising: an amino acid sequence having at least 90% sequence identity to SEQ ID NO: 391, wherein the amino acid sequence comprises a D141A mutation or a D143A mutation with reference to SEQ ID NO: 391. In some embodiments, the amino acid sequence comprises the D141A mutation and the D143A mutation. In some embodiments, the amino acid sequence further comprises a Y410A mutation, a L409S mutation, a Y261A mutation, a P411G mutation, a F406I mutation, a P411A mutation, a Y7A mutation, a Y493I mutation, a Y493T mutation, a V513I mutation, a L409A mutation, an A485S mutation, a Y410G mutation, an I521H mutation, or a K507L mutation, or any combination thereof, with reference to SEQ ID No: 391. In some embodiments, the amino acid sequence further comprises: (a) the Y410A mutation, with reference to SEQ ID No: 391; (b) the L409S mutation, the Y410A mutation, and the Y261A mutation, with reference to SEQ ID No: 391; (c) the L409S mutation, the Y410A mutation, the P411G mutation, and the Y261A mutation, with reference to SEQ ID No: 391; (d) the Y261A mutation, the F406I mutation, the L409S mutation, the Y410A mutation, and the P411A mutation, with reference to SEQ ID No: 391; (e) the Y7A mutation, the Y261A mutation, and the Y410A mutation, with reference to SEQ ID No: 391; (f) the Y261A mutation, the Y410A mutation, and the Y493I mutation, with reference to SEQ ID No: 391; (g) the Y261A mutation, the Y410A mutation, and the Y493T mutation, with reference to SEQ ID No: 391; (h) the Y261A mutation, the Y410A mutation, and the V513I mutation, with reference to SEQ ID No: 391; (i) the Y7A mutation, the Y261A mutation, the L409S mutation, and the Y410A mutation, with reference to SEQ ID No: 391; (j) the Y261A mutation, the L409A mutation, the Y410A mutation, and the P411A mutation, with reference to SEQ ID No: 391; (k) the Y261A mutation, the L409S mutation, the Y410A mutation, and the P411G mutation, with reference to SEQ ID No: 391; (1) the Y261A mutation, the L409S mutation, and the Y410A mutation, with reference to SEQ ID No: 391; (m) the Y261A mutation, the L409S mutation, and the Y410G mutation, with reference to SEQ ID No: 391; (n) the Y261A mutation, the L409A mutation, the Y410A mutation, the P411A mutation, and the A485S mutation, with reference to SEQ ID No: 391; (o) the Y261A mutation, the L409S mutation, the Y410A mutation, the P411G mutation, and the A485S mutation, with reference to SEQ ID No: 391; (p) the Y261A mutation, the L409S mutation, the Y410A mutation, and the A485S mutation, with reference to SEQ ID No: 391; (q) the Y261A mutation, the L409S mutation, the Y410G mutation, and the A485S mutation, with reference to SEQ ID No: 391; (r) the Y261A mutation, the L409A mutation, the Y410A mutation, the P411A mutation, and the I521H mutation, with reference to SEQ ID No: 391; (s) the Y261A mutation, the L409S mutation, the Y410A mutation, the P411G mutation, and the I521H mutation, with reference to SEQ ID No: 391; (t) the Y261A mutation, the L409S mutation, the Y410A mutation, and the I521H mutation, with reference to SEQ ID No: 391; (u) the Y261A mutation, the L409S mutation, the Y410G mutation, and the I521H mutation, with reference to SEQ ID No: 391; or (v) the Y261A mutation, the L409S mutation, the Y410A mutation, the A485S mutation, the K507L mutation, and the I521H mutation, with reference to SEQ ID No: 391. In some embodiments, the amino acid sequence further comprises any one of SEQ ID NOs: 392-413. In some embodiments, the amino acid sequence is at least 90%, 91%, 92%, 93%, 94%, 95%, 96%, 97%, 98%, 99% or 100% identical to any one of SEQ ID NOs: 392-413. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprises: an amino acid sequence having at least 90% sequence identity to SEQ ID NO: 414, wherein the amino acid sequence comprises a D158A mutation or an E160A mutation with reference to SEQ ID NO: 414. In some embodiments, the amino acid sequence comprises the D158A mutation and the E160A mutation. In some embodiments, the amino acid sequence further comprises a L431A mutation, a Y432A mutation, a P433I mutation, an A507S mutation, a K506Q mutation, a P433A mutation, an I543H mutation, a L431S mutation, a P433G mutation, a K529L mutation, or a Y432G mutation, or any combination thereof, with reference to SEQ ID No: 414. In some embodiments, the amino acid sequence further comprises: (a) the L431A mutation, the Y432A mutation, the P433I mutation, and the A507S mutation, with reference to SEQ ID No: 414; (b) the L431A mutation, the Y432A mutation, the P433I mutation, and the K506Q mutation, with reference to SEQ ID No: 414; (c) the L431A mutation, the Y432A mutation, the P433I mutation, the K506Q mutation, and the A507S mutation, with reference to SEQ ID No: 414; (d) the L431A mutation, the Y432A mutation, and the P433A mutation, with reference to SEQ ID No: 414; (e) the L431A mutation, the Y432A mutation, the P433A mutation, and the A507S mutation, with reference to SEQ ID No: 414; (f) the L431A mutation, the Y432A mutation, the P433A mutation, and the I543H mutation, with reference to SEQ ID No: 414; (g) the L431S mutation, the Y432A mutation, and the P433G mutation, with reference to SEQ ID No: 414; (h) the L431S mutation, the Y432A mutation, the P433G mutation, and the A507S mutation, with reference to SEQ ID No: 414; (i) the L431S mutation, the Y432A mutation, the P433G mutation, and the I543H mutation, with reference to SEQ ID No: 414; (j) the L431S mutation and the Y432A mutation, with reference to SEQ ID No: 414; (l) the L431S mutation, the Y432A mutation, and the A507S mutation, with reference to SEQ ID No: 414; (m) the L431S mutation, the Y432A mutation, the A507S mutation, the K529L mutation, and the I543H mutation, with reference to SEQ ID No: 414; (n) the L431S mutation, the Y432A mutation, and the I543H mutation, with reference to SEQ ID No: 414; (o) the L431S mutation and the Y432G mutation, with reference to SEQ ID No: 414; (p) the L431S mutation, the Y432G mutation, and the A507S mutation, with reference to SEQ ID No: 414; or (q) the L431S mutation, the Y432G mutation, and the I543H mutation, with reference to SEQ ID No: 414. In some embodiments, the amino acid sequence further comprises any one of SEQ ID NOs: 415-430. In some embodiments, the amino acid sequence is at least 90%, 91%, 92%, 93%, 94%, 95%, 96%, 97%, 98%, 99% or 100% identical to any one of SEQ ID NOs: 415-430. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprising: an amino acid sequence having at least 90% sequence identity to SEQ ID NO: 431, wherein the amino acid sequence comprises a D141A mutation or an E143A mutation with reference to SEQ ID NO: 431. In some embodiments, the amino acid sequence comprises the D141A mutation and the E143A mutation. In some embodiments, the amino acid sequence further comprises a Y412A mutation, a L411A mutation, a P413A mutation, an A488S mutation, an I524H mutation, a L411S mutation, a P413G mutation, a K510L mutation, or a Y412G mutation, or any combination thereof, with reference to SEQ ID No: 431. In some embodiments, the amino acid sequence further comprises: (a) the Y412A mutation with reference to SEQ ID No: 431; (b) the L411A mutation, the Y412A mutation, and the P413A mutation, with reference to SEQ ID No: 431; (c) the L411A mutation, the Y412A mutation, the P413A mutation, and the A488S mutation, with reference to SEQ ID No: 431; (d) the L411A mutation, the Y412A mutation, the P413A mutation, and the I524H mutation, with reference to SEQ ID No: 431; (e) the L411S mutation, the Y412A mutation, and the P413G mutation, with reference to SEQ ID No: 431; (f) the L411S mutation, the Y412A mutation, the P413G mutation, and the A488S mutation, with reference to SEQ ID No: 431; (g) the L411S mutation, the Y412A mutation, the P413 G mutation, and the I524H mutation, with reference to SEQ ID No: 431; (h) the L411S mutation and the Y412A mutation, with reference to SEQ ID No: 431; (i) the L411S mutation, the Y412A mutation, and the A488S mutation, with reference to SEQ ID No: 431; (j) the L411S mutation, the Y412A mutation, the A488S mutation, the K510L mutation, and the I524H mutation, with reference to SEQ ID No: 431; (k) the L411S mutation, the Y412A mutation, and the I524H mutation, with reference to SEQ ID No: 431; (1) the L411S mutation and Y412G mutation, with reference to SEQ ID No: 431; (m) the L411S mutation, Y412G mutation, and the A488S mutation, with reference to SEQ ID No: 431; or (n) the L411S mutation, Y412G mutation, and the I524H mutation, with reference to SEQ ID No: 431. In some embodiments, the amino acid sequence further comprises any one of SEQ ID NOs: 432-445. In some embodiments, the amino acid sequence is at least 90%, 91%, 92%, 93%, 94%, 95%, 96%, 97%, 98%, 99% or 100% identical to any one of SEQ ID NOs: 432-445. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprising: an amino acid sequence having at least 90% sequence identity to SEQ ID NO: 446, wherein the amino acid sequence comprises a D141A mutation, an E143A mutation, or a Y412A mutation, with reference to SEQ ID NO: 446. In some embodiments, the amino acid sequence comprises two or more of the D141A mutation, the E143A mutation and the Y412A mutation, with reference to SEQ ID NO: 446. In some embodiments, the amino acid sequence further comprises SEQ ID NO: 447. In some embodiments, the amino acid sequence is at least 90%, 91%, 92%, 93%, 94%, 95%, 96%, 97%, 98%, 99% or 100% identical to SEQ ID NO: 447. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects, disclosed herein, in some embodiments, are synthetic polypeptides, comprising: an amino acid sequence having at least 90% sequence identity to SEQ ID NO: 448, wherein the amino acid sequence comprises a D149A mutation, an E151A mutation, a Y272A mutation, or a Y422A mutation, with reference to SEQ ID NO: 448. In some embodiments, the amino acid sequence comprises two or more of the D149A mutation, the E151A mutation, the Y272A mutation, and the Y422A mutation, with reference to SEQ ID NO: 448. In some embodiments, the amino acid sequence further comprises SEQ ID NO: 449. In some embodiments, the amino acid sequence is at least 90%, 91%, 92%, 93%, 94%, 95%, 96%, 97%, 98%, 99% or 100% identical to SEQ ID NO: 449. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects, disclosed herein, in some embodiments, are synthetic polypeptides, comprising: an amino acid sequence having at least 90% sequence identity to SEQ ID NO: 450, wherein the amino acid sequence comprises a D149A mutation, an E151A mutation, a Y272A mutation, or a Y422A mutation, with reference to SEQ ID NO: 450. In some embodiments, the amino acid sequence comprises two or more of the D149A mutation, the E151A mutation, the Y272A mutation, and the Y422A mutation, with reference to SEQ ID NO: 450. In some embodiments, the amino acid sequence further comprises SEQ ID NO: 451. In some embodiments, the amino acid sequence is at least 90%, 91%, 92%, 93%, 94%, 95%, 96%, 97%, 98%, 99% or 100% identical to SEQ ID NO: 451. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprising: an amino acid sequence having at least 90% sequence identity to SEQ ID NO: 452, wherein the amino acid sequence comprises a D170A mutation, an E172A mutation, or a Y449A mutation, with reference to SEQ ID NO: 452. In some embodiments, the amino acid sequence comprises two or more of the D170A mutation, the E172A mutation, and the Y449A mutation, with reference to SEQ ID NO: 452. In some embodiments, the amino acid sequence further comprises SEQ ID NO: 453. In some embodiments, the amino acid sequence is at least 90%, 91%, 92%, 93%, 94%, 95%, 96%, 97%, 98%, 99% or 100% identical to SEQ ID NO: 453. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprising: an amino acid sequence having at least 90% sequence identity to SEQ ID NO: 454, wherein the amino acid sequence comprises a D170A mutation, an E172A mutation, or a Y449A mutation, with reference to SEQ ID NO: 454. In some embodiments, the amino acid sequence comprises two or more of the D170A mutation, the E172A mutation, and the Y449A mutation, with reference to SEQ ID NO: 454. In some embodiments, the amino acid sequence further comprises SEQ ID NO: 455. In some embodiments, the amino acid sequence is at least 90%, 91%, 92%, 93%, 94%, 95%, 96%, 97%, 98%, 99% or 100% identical to SEQ ID NO: 455. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are synthetic polypeptides, comprising: an amino acid sequence having at least 90% sequence identity to SEQ ID NO: 456, wherein the amino acid sequence comprises a D173A mutation, an E175A mutation, a Y452A mutation, or a C468A mutation, with reference to SEQ ID NO: 456. In some embodiments, the amino acid sequence comprises two or more of the D173A mutation, the E175A mutation, the Y452A mutation and the C468A mutation, with reference to SEQ ID NO: 456. In some embodiments, the amino acid sequence further comprises SEQ ID NO: 457. In some embodiments, the amino acid sequence is at least 90%, 91%, 92%, 93%, 94%, 95%, 96%, 97%, 98%, 99% or 100% identical to SEQ ID NO: 457. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects, disclosed herein, in some embodiments, are synthetic polypeptides, comprising: an amino acid sequence having at least 90% sequence identity to SEQ ID NO: 458, wherein the amino acid sequence comprises a D149A mutation, an E151A mutation, a Y272A mutation, or a Y422A mutation, with reference to SEQ ID NO: 458. In some embodiments, the amino acid sequence comprises two or more of the D149A mutation, the E151A mutation, the Y272A mutation, and the Y422A mutation, with reference to SEQ ID NO: 458. In some embodiments, the amino acid sequence further comprises SEQ ID NO: 459. In some embodiments, the amino acid sequence is at least 90%, 91%, 92%, 93%, 94%, 95%, 96%, 97%, 98%, 99% or 100% identical to SEQ ID NO: 459. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects, disclosed herein, in some embodiments, are synthetic polypeptides, comprising: an amino acid sequence having at least 90% sequence identity to SEQ ID NO: 460, wherein the amino acid sequence comprises a D141A mutation, an E143A mutation, or a Y412A mutation, with reference to SEQ ID NO: 460. In some embodiments, the amino acid sequence comprises two or more of the D141A mutation, the E143A mutation, and the Y412A mutation, with reference to SEQ ID NO: 460. In some embodiments, the amino acid sequence further comprises SEQ ID NO: 461. In some embodiments, the amino acid sequence is at least 90%, 91%, 92%, 93%, 94%, 95%, 96%, 97%, 98%, 99% or 100% identical to SEQ ID NO: 461. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects, disclosed herein, in some embodiments are synthetic polypeptides, comprising: an amino acid sequence having at least 90% sequence identity to SEQ ID NO: 462, wherein the amino acid sequence comprises a D141A mutation, an E143A mutation, or a Y412A mutation, with reference to SEQ ID NO: 462. In some embodiments, the amino acid sequence further comprises an amino acid deletion from amino acid position I496 to amino acid position S1033, or any portion of the amino acid sequence thereof, with reference to SEQ ID NO: 462. In some embodiments, the amino acid sequence comprises two or more of the D141A mutation, the E143A mutation, and the Y412A mutation, with reference to SEQ ID NO: 462. In some embodiments, the amino acid sequence further comprises SEQ ID NO: 463. In some embodiments, the amino acid sequence is at least 90%, 91%, 92%, 93%, 94%, 95%, 96%, 97%, 98%, 99% or 100% identical to SEQ ID NO: 463. In some embodiments, the synthetic polypeptide is purified or isolated. In some embodiments, the synthetic polypeptide is a polymerizing enzyme. In some embodiments, the polymerizing enzyme comprises a DNA polymerase or polymerizing portion thereof.

Aspects disclosed herein, in some embodiments, are methods of nucleic acid analysis, the methods comprising: providing a formulation comprising: (i) a synthetic polypeptide disclosed herein; (ii) a primed nucleic acid sequence; and (ii) a nucleotide unit complementary to a nucleotide of the primed nucleic acid, wherein the formulation is provided under conditions sufficient to form a binding complex comprising the synthetic polypeptide, the nucleotide unit, and the primed nucleic acid sequence. In some embodiments, the method comprises a nucleic acid sequencing method. In some embodiments, the method comprises a sequencing by synthesis method. In some embodiments, the formulation further comprises one or more compositions, wherein a composition of the one or more compositions comprises: (a) a core; and (b) at least two nucleotide arms, wherein a nucleotide arm of the at least two nucleotide arms comprises: (i) a core attachment moiety coupled to the core; (ii) a spacer coupled to the core attachment moiety; (iii) a linker coupled to the spacer; and (iv) the nucleotide unit coupled to the linker. In some embodiments, the linker comprises:

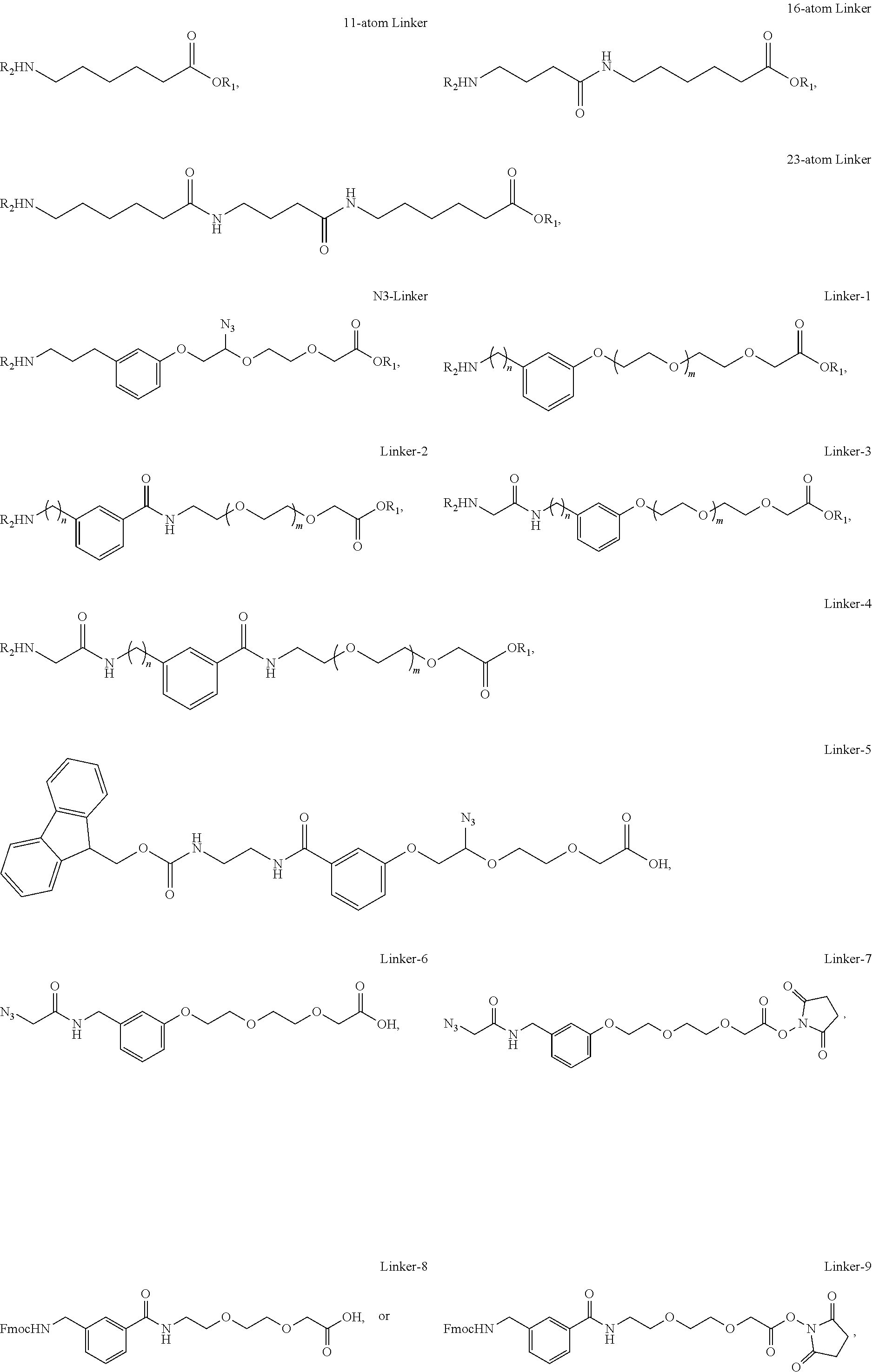

wherein n is 1 to 6 and m is 0 to 10.

Disclosed herein, in some embodiments, are kits, comprising: one or more containers comprising: the synthetic polypeptide; and a nucleotide unit, wherein the nucleotide unit is detectable; and instructions for introducing the synthetic polypeptide and the nucleotide unit to a primed nucleic acid sequence that comprises a nucleotide complementary to the nucleotide unit under conditions sufficient to form a binding complex comprising the synthetic polypeptide, the nucleotide unit, and the primed nucleic acid sequence. In some embodiments, the kit further comprises: one or more compositions, wherein a composition of the one or more compositions comprises: a core; and at least two nucleotide arms, wherein a nucleotide arm of the at least two nucleotide arms comprises: a core attachment moiety coupled to the core; a spacer coupled to the core attachment moiety; a linker coupled to the spacer; and the nucleotide unit coupled to the linker. In some embodiments, the linker comprises:

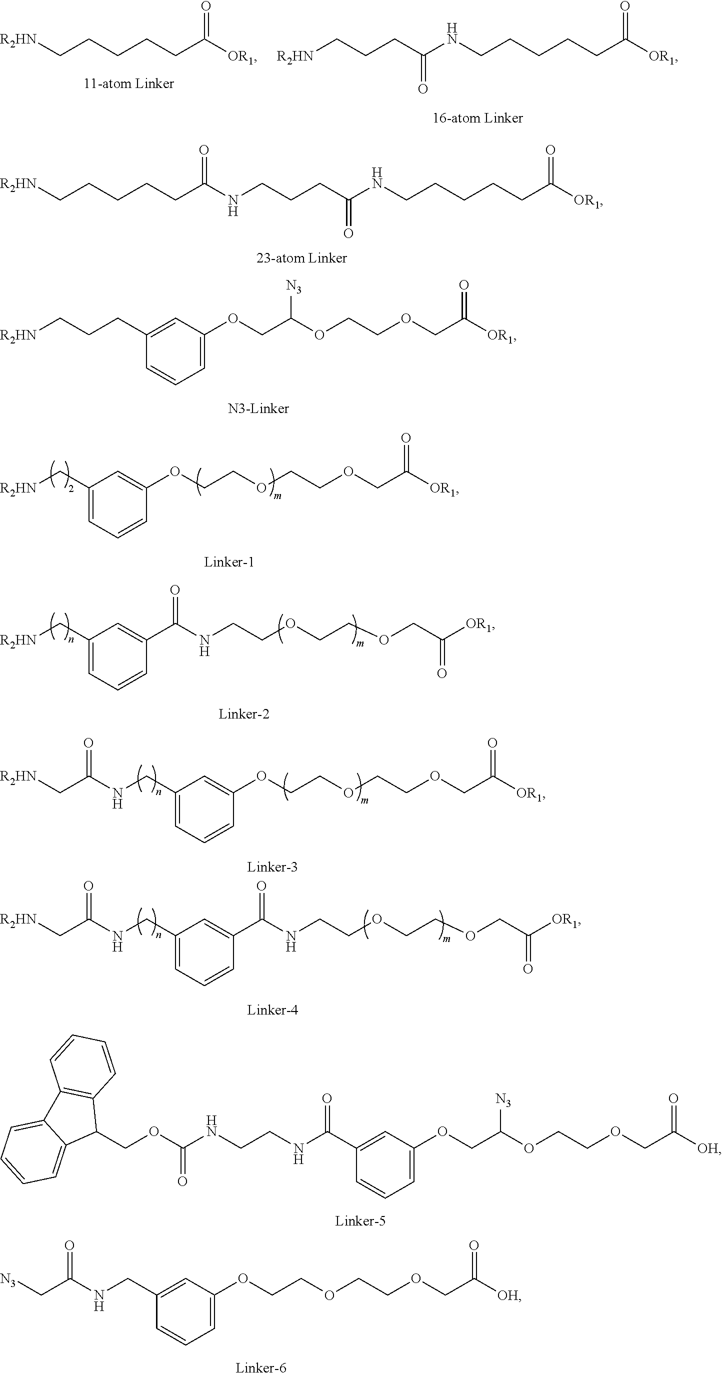

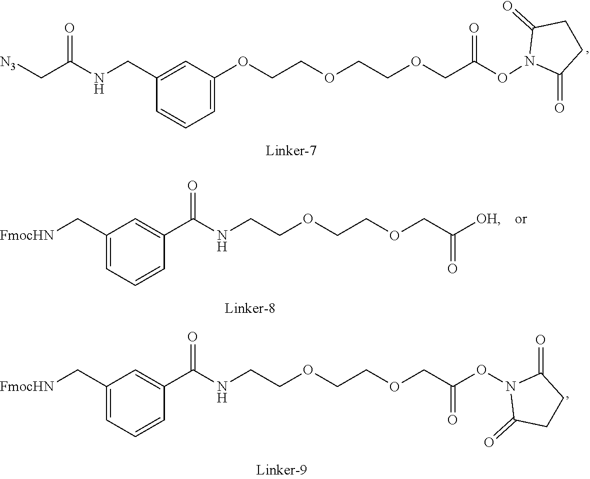

wherein n is 1 to 6 and m is 0 to 10.

Aspects disclosed herein, in some embodiments, are systems, comprising: the synthetic polypeptide; a primed nucleic acid sequence; and a nucleotide unit, wherein the nucleotide unit is detectable and complementary to a nucleotide in the primed nucleic acid sequence, wherein the system is configured to form a binding complex comprising the primed nucleic acid sequence, the synthetic polypeptide, and the nucleotide unit. In some embodiments, the system further comprises: one or more compositions, wherein a composition of the one or more compositions comprises: a core; and at least two nucleotide arms, wherein a nucleotide arm of the at least two nucleotide arms comprises: a core attachment moiety coupled to the core; a spacer coupled to the core attachment moiety; a linker coupled to the spacer; and the nucleotide unit coupled to the linker, wherein the binding complex comprises the primed nucleic acid sequence, the synthetic polypeptide and the composition. In some embodiments, the system further comprises: two or more copies of the primed nucleic acid sequence; and two or more of the synthetic polypeptide, wherein the composition is configured to form a multivalent binding complex comprising two or more of the nucleotide unit of the composition, the two or more copies of the primed nucleic acid sequence, and the two or more of the synthetic polypeptide. In some embodiments, the linker comprises:

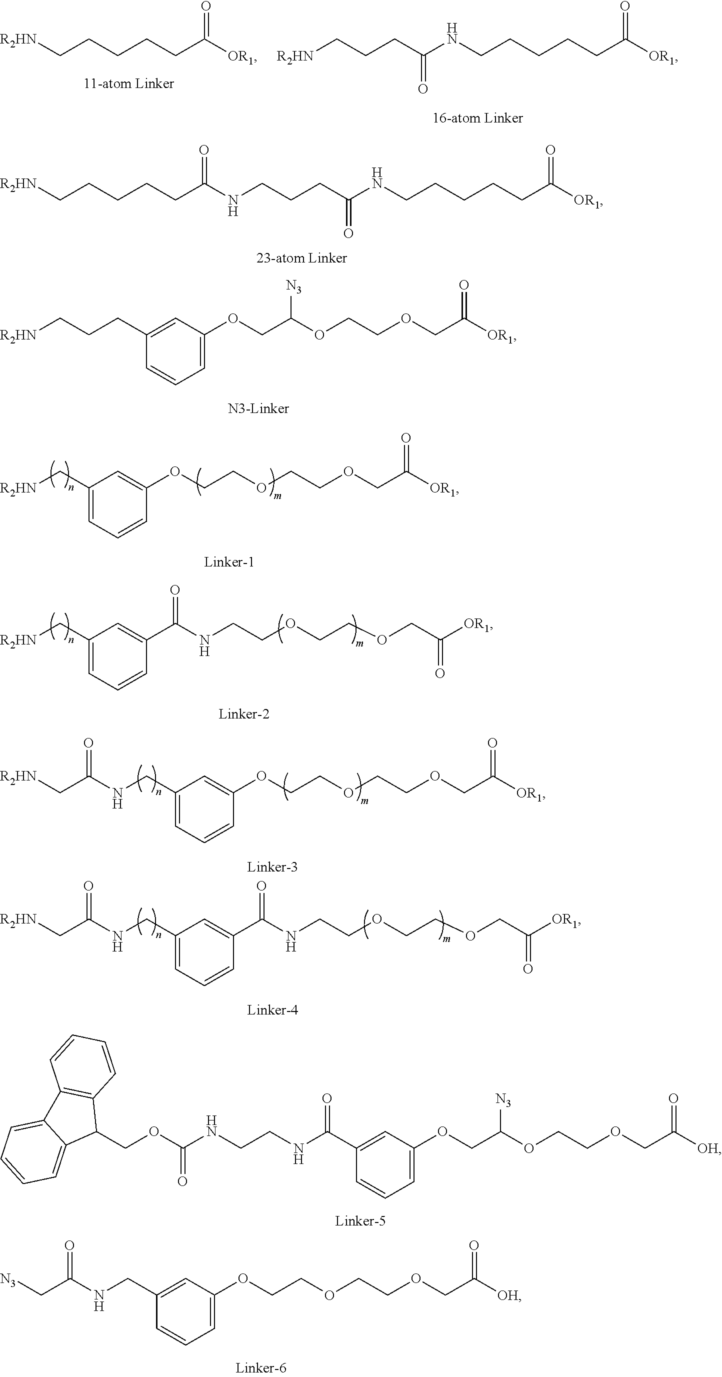

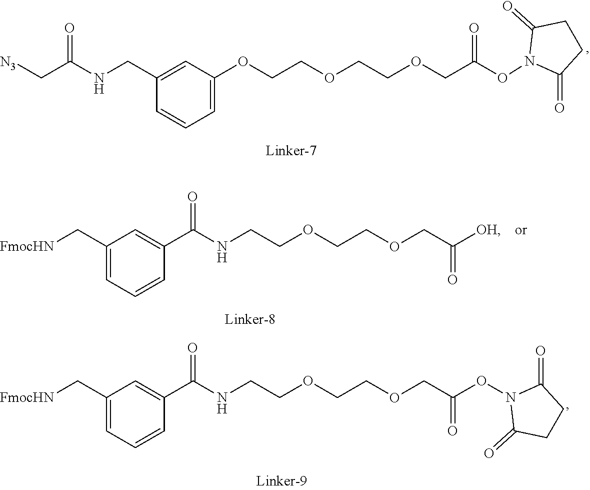

wherein n is 1 to 6 and m is 0 to 10.

Aspects disclosed herein, in some embodiments, are systems, comprising: (i) the synthetic polypeptide disclosed herein; (ii) a primed nucleic acid sequence; and (iii) a nucleotide unit complementary to a nucleotide of the primed nucleic acid, wherein the system is provided under conditions sufficient to form a binding complex comprising the synthetic polypeptide, the nucleotide unit, and the primed nucleic acid sequence.

Aspects disclosed herein, in some embodiments, are systems, comprising: (i) the synthetic polypeptide disclosed herein; (ii) a nucleotide. In some embodiments, the nucleotide comprises a blocking group. In some embodiments, the nucleotide does not comprise a blocking group. In some embodiments, the nucleotide comprises a label. In some embodiments, the nucleotide is unlabeled. In some embodiments, the blocking group is linked to the 3′ carbon of the sugar moiety of the nucleotide, and wherein the blocking group reacts with a chemical compound to remove the blocking group from the nucleotide to generate the nucleotide comprising a OH moiety on the 3′ carbon of the sugar moiety of the nucleotide. In some embodiments, the blocking group comprises an alkyl, an alkenyl, an alkynyl, an allyl, an aryl, a benzyl, an azide, an amine, an amide, a keto, an isocyanate, a phosphate, a thiol, a disulfide, a carbonate, a urea, or a silyl group. In some embodiments, (a) the alkyl, alkenyl, alkynyl, or allyl group of the blocking group reacts with tetrakis(triphenylphosphine)palladium(0) (Pd(P(C6H5)3)4) with piperidine, or 2,3-Dichloro-5,6-dicyano-1,4-benzo-quinone (DDQ); (b) the aryl or benzyl group of the blocking group reacts with H2 and Palladium on carbon (Pd/C); (c) the amine, amide, keto, isocyanate, phosphate, thiol, or disulfide group of the blocking group reacts with phosphine, or a thiol group comprising beta-mercaptoethanol, or dithiothritol (DTT); (d) the carbonate group of the blocking group reacts with potassium carbonate (K2CO3) in methanol (MeOH), triethylamine in pyridine, or Zn in acetic acid (AcOH); and (e) the urea or silyl group of the blocking group reacts with tetrabutylammonium fluoride, HF-pyridine, ammonium fluoride, or triethylamine trihydrofluoride. In some embodiments, the azide of the blocking group comprises an azide, an azido or an azidomethyl group. In some embodiments, the azide, azido, or azidomethyl group reacts with a chemical agent that comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP).

Aspects disclosed herein, in some embodiments, are compositions, comprising: (a) a core; and (b) at least two nucleotide arms, wherein a nucleotide arm of the at least two nucleotide arms comprises: (i) a core attachment moiety coupled to the core; (ii) a spacer coupled to the core attachment moiety; (iii) a linker coupled to the spacer; and (iv) a nucleotide unit coupled to the linker, wherein the linker comprises:

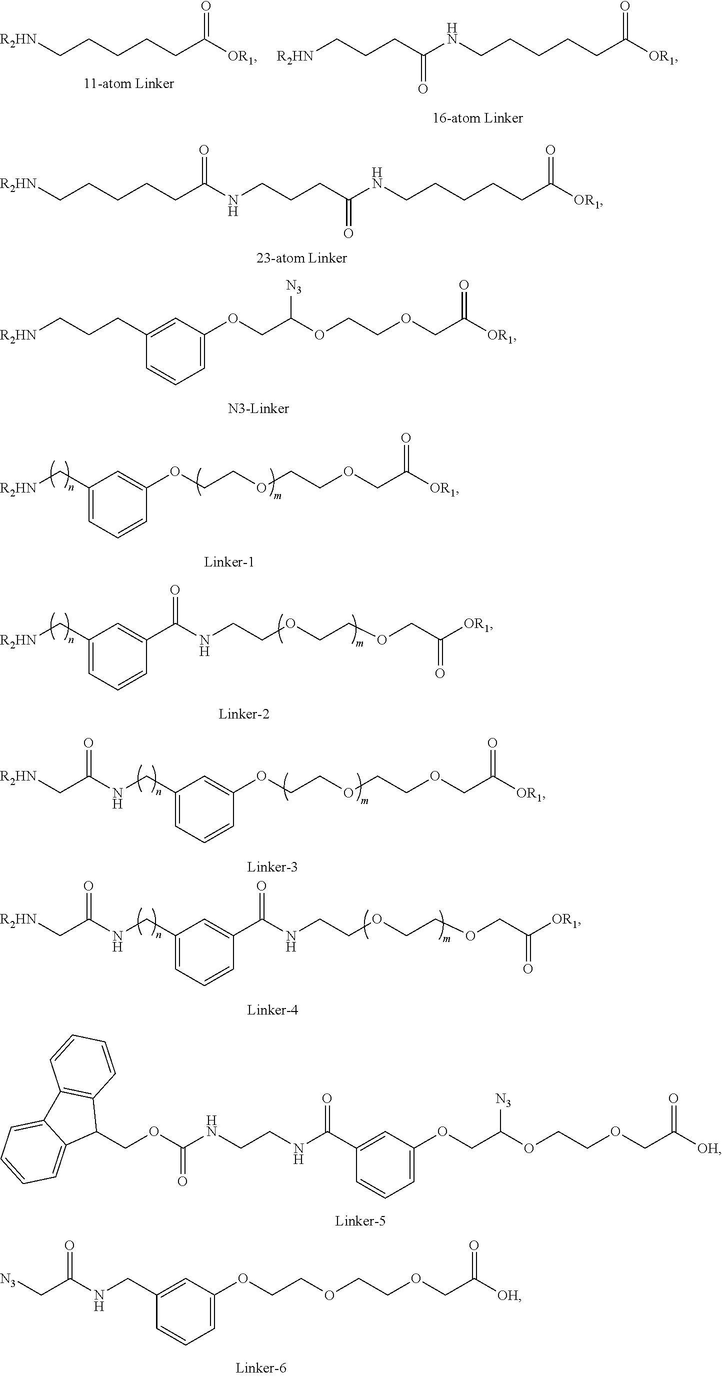

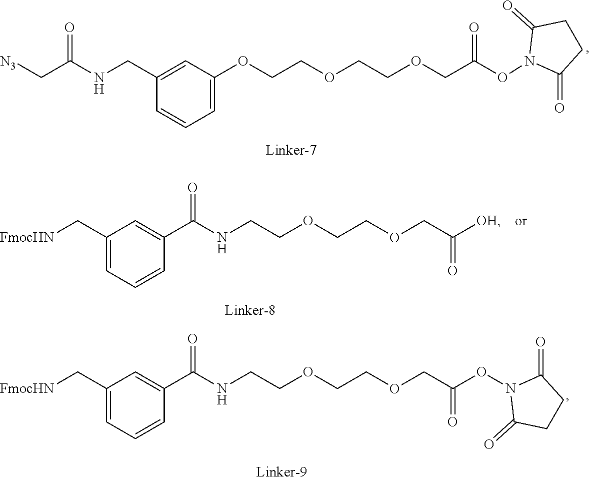

wherein n is 1 to 6 and m is 0 to 10. In some embodiments, the linker comprises:

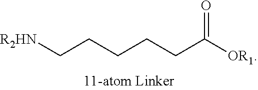

In some embodiments, the linker comprises:

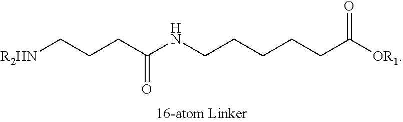

In some embodiments, the linker comprises:

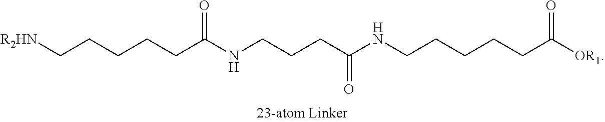

In some embodiments, the linker comprises:

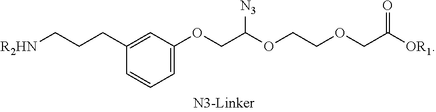

In some embodiments, the linker comprises:

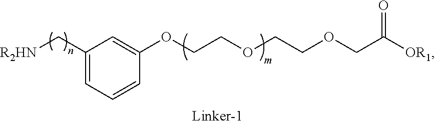

wherein n is 1 to 6 and m is 0 to 10. In some embodiments, the linker comprises:

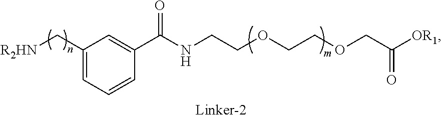

wherein n is 1 to 6 and m is 0 to 10. In some embodiments, the linker comprises:

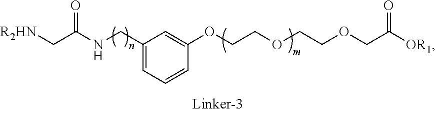

wherein n is 1 to 6 and m is 0 to 10. In some embodiments, the linker comprises:

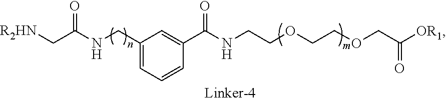

wherein n is 1 to 6 and m is 0 to 10. In some embodiments, the linker comprises:

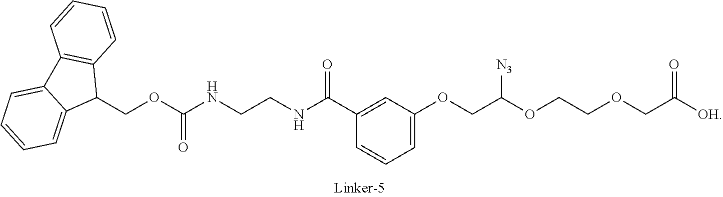 [SMILES: [N-]=[N+]=NC(COc1cccc(C(=O)NCCNC(=O)OCC2c3ccccc3-c3ccccc32)c1)OCCOCC(=O)O]

In some embodiments, the linker comprises:

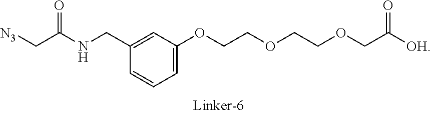 [SMILES: [N-]=[N+]=NCC(=O)NCc1cccc(OCCOCCOCC(=O)O)c1]

In some embodiments, the linker comprises:

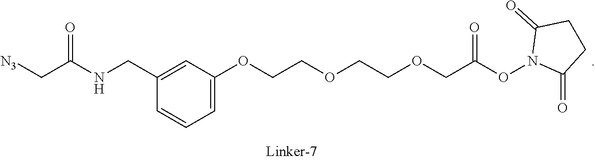 [SMILES: [N-]=[N+]=NCC(=O)NCc1cccc(OCCOCCOCC(=O)ON2C(=O)CCC2=O)c1]

In some embodiments, the linker comprises:

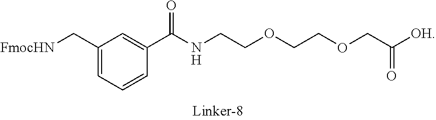

In some embodiments, the linker comprises:

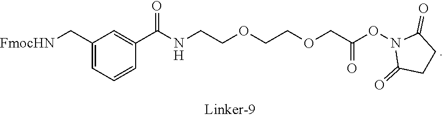

In some embodiments, the at least two nucleotide arms comprises 3 to 20 nucleotide arms. In some embodiments, the core comprises a polypeptide. In some embodiments, the polypeptide comprises streptavidin or avidin. In some embodiments, the core attachment moiety comprises biotin. In some embodiments, the composition further comprises a fluorescent label coupled to the core or the nucleotide arm. In some embodiments, the spacer comprises a structure:

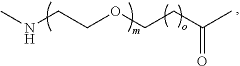

wherein m is 20 to 500 and o is 1 to 10. In some embodiments, the nucleotide arm further comprises a reactive group coupled to the nucleotide unit, wherein the reactive group is configured to react with an agent. In some embodiments, the reactive group comprises an alkyl, alkenyl, an alkynyl, an allyl, an aryl, a benzyl, an azide, an amine, an amide, a keto, an isocyanate, a phosphate, a thiol, a disulfide, a carbonate, a urea, or a silyl group. In some embodiments, (a) the alkyl, alkenyl, alkynyl or allyl group of the reactive group reacts with tetrakis(triphenylphosphine)palladium(0) (Pd(P(C₆H₅)₃)₄) with piperidine or 2,3-Dichloro-5,6-dicyano-1,4-benzoquinone (DDQ); (b) the aryl or benzyl group of the reactive group reacts with H₂ and Palladium on carbon (Pd/C); (c) the amine, amide, keto, isocyanate, phosphate, thiol, or disulfide group of the reactive group reacts with phosphine or a thiol group comprising beta-mercaptoethanol or dithiothritol (DTT); (d) the carbonate group of the reactive group reacts with potassium carbonate (K₂CO₃) in methanol (MeOH), triethylamine in pyridine, or Zn in acetic acid (AcOH); and (e) the urea or silyl group of the reactive group reacts with tetrabutylammonium fluoride, Hydrogen fluoride pyridine (HF-pyridine), ammonium fluoride, or triethylamine trihydrofluoride. In some embodiments, the azide group of the reactive group comprises an azide, an azido or an azidomethyl group. In some embodiments, the agent comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP). In some embodiments, the nucleotide unit of the at least two nucleotide arms comprises the same nucleobase type. In some embodiments, the nucleotide unit comprises a blocking group linked to the 3′ carbon of the sugar moiety of the nucleotide unit, and wherein the blocking group reacts with a chemical compound to remove the blocking group from the nucleotide unit to generate the nucleotide unit comprising a OH moiety on the 3′ carbon of the sugar moiety of the nucleotide unit. In some embodiments, the blocking group comprises an alkyl, an alkenyl, an alkynyl, an allyl, an aryl, a benzyl, an azide, an amine, an amide, a keto, an isocyanate, a phosphate, a thiol, a disulfide, a carbonate, a urea, or a silyl group. In some embodiments, (a) the alkyl, alkenyl, alkynyl, or allyl group of the blocking group reacts with tetrakis(triphenylphosphine)palladium(0) (Pd(P(C₆H₅)₃)₄) with piperidine, or 2,3-Dichloro-5,6-dicyano-1,4-benzo-quinone (DDQ); (b) the aryl or benzyl group of the blocking group reacts with H₂ and Palladium on carbon (Pd/C); (c) the amine, amide, keto, isocyanate, phosphate, thiol, or disulfide group of the blocking group reacts with phosphine, or a thiol group comprising beta-mercaptoethanol, or dithiothritol (DTT); (d) the carbonate group of the blocking group reacts with potassium carbonate (K₂CO₃) in MeOH, triethylamine in pyridine, or Zn in acetic acid (AcOH); and (e) the urea or silyl group of the blocking group reacts with tetrabutylammonium fluoride, HF-pyridine, ammonium fluoride, or triethylamine trihydrofluoride. In some embodiments, the azide of the blocking group comprises an azide, an azido or an azidomethyl group. In some embodiments, the azide, azido, or azidomethyl group reacts with a chemical agent that comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP).

Aspects disclosed herein, in some embodiments, are formulations, comprising: at least two of the composition disclosed herein, wherein the at least two of the composition comprises a first composition and a second composition, wherein the nucleotide unit of the first composition comprises a different nucleobase than the nucleotide unit of the second composition.

Aspects disclosed herein, in some embodiments, are formulations, comprising: at least two of the composition, wherein the at least two of the composition comprises a first composition and a second composition, wherein: (a) the nucleotide unit of the first composition comprises a first blocking group linked to the 3′ carbon of the sugar moiety, wherein the first blocking group reacts with a chemical compound to remove the first blocking group and generate a OH moiety on the 3′ carbon of the sugar moiety of the nucleotide unit of the first composition; and (b) the nucleotide unit of the second composition comprises a second blocking group linked to the 3′ carbon of the sugar moiety, wherein the second blocking group reacts with a chemical compound to remove the second blocking group and generate a OH moiety on the 3′ carbon of the sugar moiety of the nucleotide unit of the second composition, and wherein the nucleotide unit of the first composition differs from the nucleotide unit of the second composition,

Aspects disclosed herein, in some embodiments, are formulations, comprising at least two of the composition disclosed herein, wherein the at least two of the composition comprises a first composition and a second composition, wherein the linker of the first composition differs from the linker of the second composition.

Aspects disclosed herein, in some embodiments, are formulations, comprising: at least two of the composition disclosed herein, wherein the at least two of the composition comprises a first composition and a second composition, wherein the linker of the first composition comprises a first reactive group and the linker of the second composition comprises a second reactive group, wherein the second reactive group differs from the first reactive group.

Aspects disclosed herein, in some embodiments, are formulations, comprising: at least two of the composition disclosed herein, wherein the at least two of the composition comprises a first composition and a second composition, wherein the first composition comprise a first fluorophore and the second composition comprise a second fluorophore, and wherein the first fluorophore emits light at a wavelength that differs from the wavelength of light emitted from the second fluorophore.

Aspects disclosed herein, in some embodiments, are formulations, comprising: at least two of the composition disclosed herein, wherein the at least two of the composition comprises a first composition and a second composition, wherein the first composition comprises a fluorescent label and the second composition is unlabeled.

Aspects disclosed herein, in some embodiments, are kits for nucleic acid molecule processing, the kit comprising: (a) the composition disclosed herein, or the formulation disclosed herein; and (b) an instruction for use of the composition in a nucleotide identification reaction. In some embodiments, the kit further comprises: an agent that reacts with the reactive group in the linker of the composition. In some embodiments, the kit further comprises: an agent that reacts with the reactive group at the 3′ carbon of the sugar moiety in the nucleotide unit of the composition. In some embodiments, the kit further comprises a reagent for use in the nucleotide binding reaction. In some embodiments, the reagent comprises a cation. In some embodiments, the kit further comprises: a solution comprising a cation; one or more polymerizing enzymes; one or more primer sequences; one or more unlabeled nucleotides; or any combination of (i) to (iv).

Aspects disclosed herein, in some embodiments, are systems, comprising: (a) the composition disclosed herein; (b) two or more copies of a primed nucleic acid sequence, wherein the two or more copies of the primed nucleic acid sequence comprise a nucleotide complementary to the nucleotide unit of the composition; and (c) two or more of a polymerizing enzyme, wherein the system is configured to form a multivalent binding complex comprising the two or more primed nucleic acid sequences, the two or more of the polymerizing enzyme and the composition. In some embodiments, the multivalent binding complex is formed under conditions such that the nucleotide unit of the composition is not incorporated into the two or more copies of the primed nucleic acid sequence. In some embodiments, the two or more copies of the nucleic acid sequence and the two or more copies of the nucleic acid primer molecule are immobilized to a support under conditions sufficient to immobilize the multivalent binding complex to the support. In some embodiments, a plurality of the multivalent binding complex is immobilized on the support, wherein a density of the plurality of the multivalent binding complex immobilized on the support is 10²-10⁹ per millimeter squared (mm 2). In some embodiments, the plurality of the multivalent binding complex on the support is in fluid communication with each other and a solution of reagents under conditions sufficient that the plurality of the multivalent binding complex reacts with the solution of reagents in a massively parallel manner.

Aspects disclosed herein, in some embodiments, are methods of nucleic acid analysis, the method comprising: introducing the composition disclosed herein to a primed nucleic acid sequence under conditions sufficient to form a binding complex comprising (i) the nucleotide unit of the composition and (ii) a nucleotide in the primed nucleic acid sequence, wherein the nucleotide is complementary to the nucleotide unit of the composition.

Aspects disclosed herein, in some embodiments, are methods of nucleic acid analysis, the method comprising: introducing the composition disclosed herein to two or more copies of a primed nucleic acid sequence under conditions sufficient to form a multivalent binding complex comprising (i) two or more of the nucleotide units of the composition and (ii) two or more nucleotides in the two or more copies of the primed nucleic acid sequence, wherein the two or more nucleotides are complementary to the two or more nucleotide units of the composition.

Aspects disclosed herein, in some embodiments, are synthetic polypeptides, comprising: an amino acid sequence having at least 90% sequence identity to SEQ ID NO: 1, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 391, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 414, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 431, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 446, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 448, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 450, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 452, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 454, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 456, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 458, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 460, wherein the amino acid sequence provides a binding constant of at least 3: (i) between the amino acid sequence and a primed nucleic acid sequence; (ii) between the amino acid sequence and a nucleotide; or (iii) both (i) and (ii).

Aspects disclosed herein, in some embodiments, are synthetic polypeptides, comprising: an amino acid sequence having at least 90% sequence identity to SEQ ID NO: 462, wherein the amino acid sequence provides a binding constant of at least 3: (i) between the amino acid sequence and a primed nucleic acid sequence; (ii) between the amino acid sequence and a nucleotide; or (iii) both (i) and (ii).

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the methods and systems are set forth with particularity in the appended claims. A better understanding of the features and advantages of the present methods and systems will be obtained by reference to the following detailed description that sets forth illustrative embodiments, in which the principles of the methods and systems are utilized, and the accompanying drawings of which:

FIG. 1 is a schematic of a non-limiting example of a nucleotide conjugate comprising a generic core conjugated to a plurality of nucleotide-arms according to some embodiments herein. In some embodiments, individual nucleotide arms comprise a spacer, linker and nucleotide unit.

FIG. 2 is a schematic of a non-limiting example of a nucleotide conjugate comprising a dendrimer core conjugated to a plurality of nucleotide-arms according to some embodiments herein. In some embodiments, individual nucleotide arms comprise a spacer, linker and nucleotide unit.

FIG. 3 shows a schematic of a non-limiting example of a nucleotide conjugate comprising a core attached/bound to a plurality of nucleotide-arms, where individual nucleotide arms comprise a core attachment moiety, spacer, linker and nucleotide unit, according to some embodiments herein.

FIG. 4 is a schematic of a non-limiting example of a nucleotide-arm comprising a core attachment moiety, spacer, linker and nucleotide unit, according to some embodiments herein.

FIG. 5A shows the chemical structure of a non-limiting example of a spacer at the top and the chemical structure of non-limiting examples of linkers at the bottom, including an 11 atom Linker, 16 atom Linker, 23 atom Linker and N3 Linker, according to some embodiments herein.

FIG. 5B shows the chemical structures of non-limiting examples of linkers, including Linkers 1-6, according to some embodiments herein.

FIG. 5C shows the chemical structures of non-limiting examples of linkers, including Linkers 7-9, according to some embodiments herein.

FIG. 5D shows the chemical structures of non-limiting examples of linkers, including Linkers 10 and 11 which are joined to a nucleotide unit, according to some embodiments herein.

FIG. 5E shows the chemical structures of non-limiting examples of linkers, including Linkers 12 and 13 which are joined to a nucleotide unit according to some embodiments herein.

FIG. 5F shows the chemical structures of non-limiting examples of linkers, including Linkers 14-16 which are joined to a nucleotide unit according to some embodiments herein.

FIG. 6A show the chemical structures of non-limiting examples of nucleotide-arms comprising a spacer joined to a linker, and the linker joined to a nucleotide unit according to some embodiments herein.

FIG. 6B show the chemical structures of non-limiting examples of nucleotide-arms comprising a spacer joined to a linker, and the linker joined to a nucleotide unit according to some embodiments herein.

FIG. 7A shows the chemical structures of non-limiting examples of linkers joined to nucleotide units via a propargyl amine attachment according to some embodiments herein.

FIG. 7B shows additional chemical structures of non-limiting examples of linkers joined to nucleotide units via a propargyl amine attachment according to some embodiments herein.

FIG. 7C shows additional chemical structures of non-limiting examples of linkers joined to nucleotide units via a propargyl amine attachment according to some embodiments herein.

FIG. 7D shows additional chemical structures of non-limiting examples of linkers joined to nucleotide units via a propargyl amine attachment, according to some embodiments herein.

FIG. 7E shows additional chemical structures of non-limiting examples of linkers joined to nucleotide units via a propargyl amine attachment, according to some embodiments herein.

FIG. 7F shows additional chemical structures of non-limiting examples of linkers joined to nucleotide units via a propargyl amine attachment, according to some embodiments herein.

FIG. 7G shows additional chemical structures of non-limiting examples of linkers joined to nucleotide units via a propargyl amine attachment, according to some embodiments herein.

FIG. 8A shows the chemical structure of a non-limiting example of a biotinylated nucleotide-arm comprising a biotin moiety, spacer, linker (e.g., 11 atom Linker) and nucleotide unit, according to some embodiments herein. In this non-limiting example, the nucleotide unit is connected to the linker via a propargyl amine attachment at the 5 position of a pyrimidine base or the 7 position of a purine base, or at the 1 position of a pyrimidine base or 9 position of a purine base.

FIG. 8B shows the chemical structure of a non-limiting example of a biotinylated nucleotide-arm comprising a biotin moiety, spacer, linker (e.g., Linker 6), and nucleotide unit according to some embodiments herein. In this non-limiting example, the nucleotide unit is connected to the linker via a propargyl amine attachment at the 5 position of a pyrimidine base or the 7 position of a purine base, or at the 1 position of a pyrimidine base or 9 position of a purine base.

FIG. 9 is a bar graph showing the results of a trapping assay conducted by reacting various fluorescently-labeled nucleotide conjugates with a corresponding correct DNA template.

FIG. 10 is a bar graph showing the results of a trapping assay in which increasing concentrations of various fluorescently-labeled nucleotide conjugates were reacted with corresponding correct DNA templates.

FIG. 11 presents four graphs showing the results of a trapping assay comparing the signal intensity of fluorescently-labeled nucleotide conjugates carrying nucleotide arms comprising either an N3-Linker, Linker-6, Linker-8 or propargyl Linker. The nucleotide conjugates were labeled with CF680 or CF532 fluorophores. Two different concentrations of nucleotide conjugates were tested (20 and 80 nM). The graphs show trap time in seconds (x-axis) and P90 signal intensity (y-axis).

FIG. 12 presents four graphs showing the results of a trapping assay comparing the signal intensity of fluorescently-labeled nucleotide conjugates carrying nucleotide arms comprising either an N3-Linker, Linker-6, Linker-8 or propargyl Linker. The nucleotide conjugates were labeled with AF647 or CF570 fluorophores. Two different concentrations of nucleotide conjugates were tested (20 and 80 nM). The graphs show trap time in seconds (x-axis) and P90 signal intensity (y-axis).

FIG. 13 presents three graphs showing the results of real-time imaging trapping kinetics assays comparing signal intensity of fluorescently-labeled nucleotide conjugates carrying nucleotide arms comprising one of Linkers 6 or 10-16. Three different concentrations of the nucleotide conjugates were tested (15, 7.5 and 2.5 nM). The graphs show trap time in second (x-axis) and signal intensity (y-axis).

FIG. 14 is a graph showing the results of a binding kinetic study of fluorescently-labeled nucleotide conjugates carrying nucleotide arms comprising one of Linkers 6 or 10-16. The graph shows nucleotide conjugate concentration (x-axis, nM) and rate (y-axis). The legend shown in FIG. 14 is also applicable to FIG. 13.

FIG. 15 is a bar graph showing the binding constant (K) determined for fluorescently-labeled nucleotide conjugates carrying nucleotide arms comprising one of Linkers 6 or 10-16.

FIG. 16 shows a 2.8 Angstrom model determined from X-ray crystallography in which a sequencing polymerase was co-crystallized with a template molecule hybridized to a primer having a 3′ terminal di-deoxynucleotide, and a nucleotide conjugate comprising nucleotide arms with Linker 6 and dCTP nucleotide units. The model shows two magnesium ions (spheres) in the active site of the polymerase.

FIG. 17 is an amino acid sequence of a non-limiting example of a streptavidin subunit (SEQ ID NO:466).

FIG. 18 is an amino acid sequence of a non-limiting example of a streptavidin subunit (SEQ ID NO:467).

FIG. 19 is an amino acid sequence of a non-limiting example of a streptavidin subunit (SEQ ID NO:468).

FIG. 20 is an amino acid sequence of a non-limiting example of a streptavidin subunit (SEQ ID NO:469).

FIG. 21 is an amino acid sequence of a non-limiting example of a streptavidin subunit (SEQ ID NO:470).

FIG. 22 is an amino acid sequence of a non-limiting example of an avidin subunit (SEQ ID NO:471).

FIG. 23 is an amino acid sequence of a non-limiting example of an avidin subunit (SEQ ID NO:472).

FIG. 24 is the amino acid sequence of a wild type DNA polymerase from Candidatus altiarchaeales archaeon (GenBank accession RLI89578.1) (SEQ ID NO:1).

FIG. 25 is the amino acid sequence of a wild type DNA polymerase from Candidatus altiarchaeales archaeon (GenBank accession OYT41123.1) (SEQ ID NO:464).

FIG. 26 is the amino acid sequences of an N-terminal domain of DNA polymerase from Candidatus altiarchaeales archaeon RLI 89578.1 (SEQ ID NOS:269).

FIG. 27 is the amino acid sequences of an exonuclease domain of DNA polymerase from Candidatus altiarchaeales archaeon RLI 89578.1 (SEQ ID NOS:270).

FIG. 28 is the amino acid sequences of a palm (1) domain of DNA polymerase from Candidatus altiarchaeales archaeon RLI 89578.1 (SEQ ID NOS:271).

FIG. 29 is the amino acid sequences of a finger domain of DNA polymerase from Candidatus altiarchaeales archaeon RLI 89578.1 (SEQ ID NOS:272).

FIG. 30 is the amino acid sequences of a palm (2) domain of DNA polymerase from Candidatus altiarchaeales archaeon RLI 89578.1 (SEQ ID NOS:273).

FIG. 31 is the amino acid sequences of a thumb domain of DNA polymerase from Candidatus altiarchaeales archaeon RLI 89578.1 (SEQ ID NOS:274).

FIG. 32 is the amino acid sequence of a 9° N polymerase (SEQ ID NO:280).

FIG. 33 is the amino acid sequence of a 9° N polymerase UniProtKB—Q56366 (DPOL_THES9) (SEQ ID NO:281).

FIG. 34 is the amino acid sequence of a THERMINATOR polymerase (SEQ ID NO:282).

FIG. 35 is the amino acid sequence of a DNA polymerase from Pyrococcus abyssi (SEQ ID NO:286).

FIG. 36 is the amino acid sequence of a VENT polymerase (SEQ ID NO:283).

FIG. 37 is the amino acid sequence of a DEEP VENT polymerase (SEQ ID NO:284).

FIG. 38 is the amino acid sequence of a Pfu polymerase (SEQ ID NO:285).

FIG. 39 is the amino acid sequence of wild type DNA polymerase from Geobacillus stearothermophilus (SEQ ID NO:275).

FIG. 40 is the amino acid sequence of an RB69 polymerase (SEQ ID NO:287).

FIG. 41A is the amino acid sequence of a wild type DNA polymerase from Thermococci archaeon having a backbone sequence from RLF 89458.1 (SEQ ID NO:391).

FIG. 41B is the amino acid sequence of a wild type DNA polymerase from Thermococci archaeon having a backbone sequence from RLF 78286.1 (SEQ ID NO:465).

FIG. 42 is the amino acid sequence of a wild type DNA polymerase from Thermoplasmata archaeon having a backbone sequence from RLF 60390.1 (SEQ ID NO:414).

FIG. 43 is the amino acid sequence of a wild type DNA polymerase from Thermococcus sp. 2319x1 having a backbone sequence from WP 175059460.1 (SEQ ID NO:431).

FIG. 44 is the amino acid sequence of a wild type DNA polymerase from Thermococcus litoralis having a backbone sequence from ADK 47977.1 (SEQ ID NO:446).

FIG. 45 is the amino acid sequence of a wild type DNA polymerase from archaeon BMS3Abin16 having a backbone sequence from GBE 17769.1 (SEQ ID NO:448).

FIG. 46 is the amino acid sequence of a wild type DNA polymerase from archaeon BMS3Bbin16 having a backbone sequence from GBE 55812.1 (SEQ ID NO:450).

FIG. 47 is the amino acid sequence of a wild type DNA polymerase from Candidatus Hadarchaeum yellowstonense having a backbone sequence from KUO 42443.1 (SEQ ID NO:452).

FIG. 48 is the amino acid sequence of a wild type DNA polymerase having a backbone sequence from KXB 02540.1 (SEQ ID NO:454).

FIG. 49 is the amino acid sequence of a wild type DNA polymerase having a backbone sequence from MBC 7218772.1 (SEQ ID NO:456).

FIG. 50 is the amino acid sequence of a wild type DNA polymerase having a backbone sequence from RMF 90817.1 (SEQ ID NO:458).

FIG. 51 is the amino acid sequence of a wild type DNA polymerase having a backbone sequence from WP 058946753.1 (SEQ ID NO:460).

FIG. 52 is the amino acid sequence of a wild type DNA polymerase having a backbone sequence from WP 167886206.1 (SEQ ID NO:462).

FIG. 53 is a block diagram depicting an exemplary machine that includes a computer system 100 for nucleic acid processing.

FIG. 54 is an embodiment of an application provision system.

FIG. 55 is another embodiment of an application provision system.

### DETAILED DESCRIPTION

Disclosed herein, in some embodiments, are compositions, systems and kits for use in processing and/or analyzing a nucleic acid sequence. Also provided are methods of processing and/or analyzing a nucleic acid sequence with the compositions, systems and kits disclosed herein. In some embodiments, the inventive concepts disclosed herein utilize a nucleotide conjugate comprising a core having one or more nucleotide units coupled thereto that are complementary to a nucleotide in a nucleic acid sequence to be processed or analyzed. Such nucleotide conjugate is capable for forming a multivalent binding complex comprising the nucleotide conjugate bound to a nucleotide of a nucleic acid sequence and, optionally, a polymerizing enzyme. In some embodiments, the inventive concepts disclosed herein utilize a synthetic polymerizing enzyme having one or more mutations in an amino acid sequence conferring enhanced binding to a nucleotide or nucleotide unit of the present disclosure as compared with an otherwise identical polymerizing enzyme without the one or more mutations.

### Compositions

Disclosed herein, in some embodiments, are compositions. In embodiments, the composition may be used in any method, formulation, or system disclosed herein. In some embodiments, the composition may be used in a method of nucleic acid processing, analysis, identification, or detection disclosed herein. In some embodiments, the composition may be used with a polypeptide disclosed herein. In some embodiment, the composition may be or comprise a nucleotide-conjugate disclosed herein. In some embodiment, the composition may have the ability to form a binding complex with a primed nucleic acid sequence and a polypeptide disclosed herein. In some embodiment, the composition may have the ability to form a multivalent binding complex with two or more primed nucleic acid sequences and two or more polypeptides disclosed herein.

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise the same type of nucleotide units. For example, a nucleotide conjugate can comprise a core (e.g., streptavidin or avidin core) attached to a plurality of nucleotide arms or biotinylated nucleotide arms, where all of the attached arms have a nucleotide unit selected from a group consisting of dATP, dGTP, dCTP, dTTP and dUTP.

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise different types of nucleotide units. For example, a nucleotide conjugate can comprise a core (e.g., streptavidin or avidin core) attached to a plurality of nucleotide arms or biotinylated nucleotide arms, where at least a first attached arm can have a first nucleotide unit selected from a group consisting of dATP, dGTP, dCTP, dTTP and dUTP, and a second attached arm can have a second nucleotide unit selected from a group consisting of dATP, dGTP, dCTP, dTTP and dUTP, where the first and second nucleotide units are different.

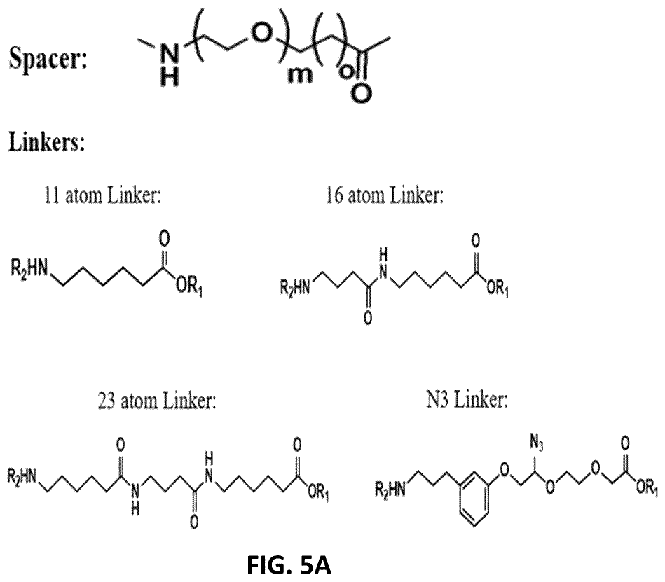

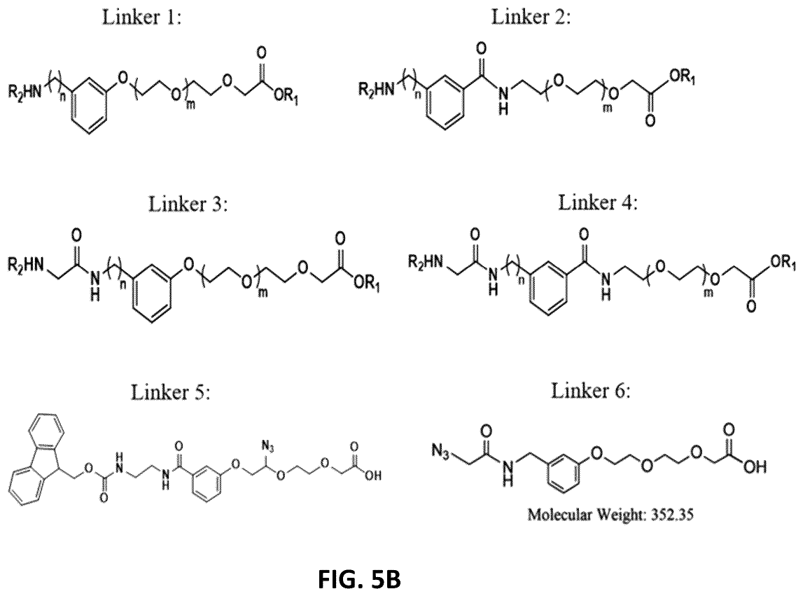

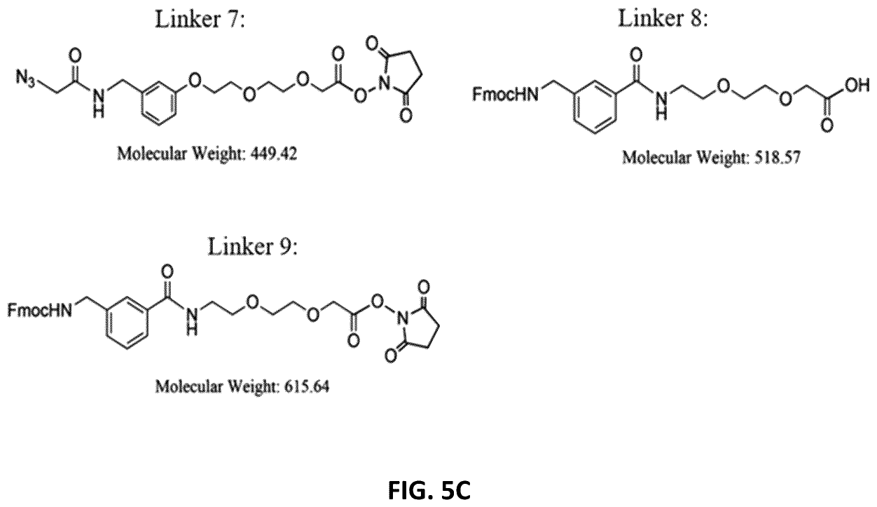

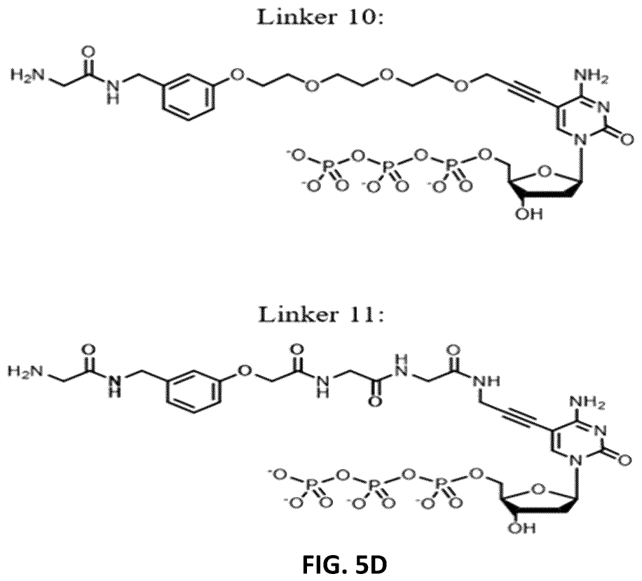

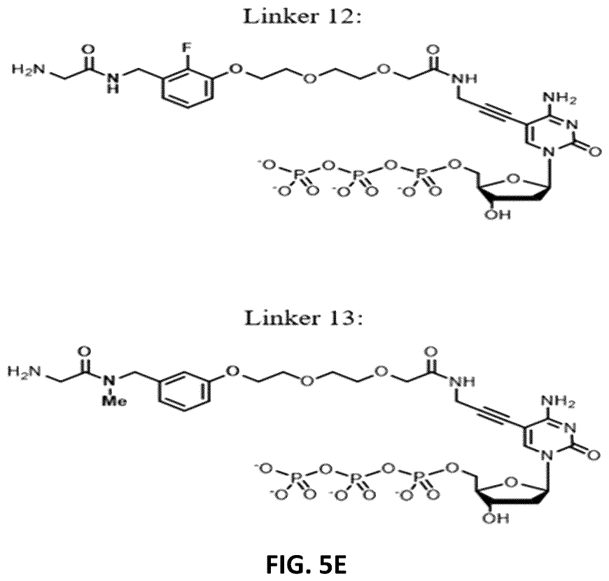

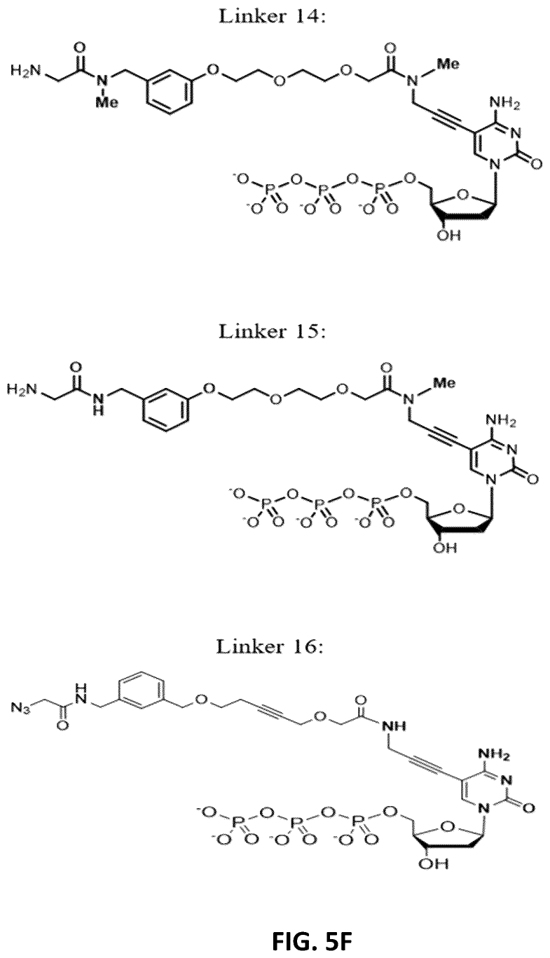

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise the same type of spacer For example, a nucleotide conjugate can comprise a core (e.g., streptavidin or avidin core) attached to a plurality of nucleotide arms or biotinylated nucleotide arms, where all of the attached arms have the same spacer. In some embodiments, the spacer can be selected from any of the spacers described herein (e.g., FIG. 5A (top)).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise different types of spacers. For example, a nucleotide conjugate can comprise a core (e.g., streptavidin or avidin core) attached to a plurality of nucleotide arms or biotinylated nucleotide arms, where at least a first attached arm can have a first type of spacer, and a second attached arm can have a second type of spacer, where the first and second spacer units are different. In some embodiments, the first and second type of linker can be selected from any of the spacers described herein (e.g., FIG. 5A (top)).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise the same type of linker. For example, a nucleotide conjugate can comprise a core (e.g., streptavidin or avidin core) attached to a plurality of nucleotide arms or biotinylated nucleotide arms, where all of the attached arms have the same linker. In some embodiments, the linker can be selected from any of the linkers described herein (e.g., FIG. 5A (bottom) and 5B-F).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise different types of linkers. For example, a nucleotide conjugate can comprise a core (e.g., streptavidin or avidin core) attached to a plurality of nucleotide arms or biotinylated nucleotide arms, where at least a first attached arm can have a first type of linker, and a second attached arm can have a second type of linker, where the first and second linker units are different. In some embodiments, the first and second type of linker can be selected from any of the linkers described herein (e.g., FIGS. 5A (bottom) and 5B-F).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise the same type of spacer and linker. For example, a nucleotide conjugate can comprise a core (e.g., streptavidin or avidin core) attached to a plurality of nucleotide arms or biotinylated nucleotide arms, where all of the attached arms have the same spacer and linker. In some embodiments, the spacer and linker can be selected from any of the spacers and linkers described herein.

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise the same type of reactive group. For example, a nucleotide conjugate can comprise a core (e.g., streptavidin or avidin core) attached to a plurality of nucleotide arms or biotinylated nucleotide arms, where all of the attached arms have the same reactive group. In some embodiments, the reactive group can comprise an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group.

In some embodiments, the reactive group in the linker can be reactive with a chemical reagent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H₂ and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in methanol (MeOH), with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride.

In some embodiments, the nucleotide-arms can have the same type of reactive group in the linker where the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise different types of reactive groups in the linkers. For example, a nucleotide conjugate can comprise a core (e.g., streptavidin or avidin core) attached to a plurality of nucleotide arms or biotinylated nucleotide arms, where at least a first attached arm can have a first type of reactive group in a first linker unit, and a second attached arm can have a second type of reactive group in a second linker unit, where the first and second reactive groups are different.

In some embodiments, the first reactive group in the first linker unit, and the second reactive group in the second linker unit, can be selected in any combination from a group consisting of an alkyl group, alkenyl group, alkynyl group, allyl group, aryl group, benzyl group, azide group, amine group, amide group, keto group, isocyanate group, phosphate group, thio group, disulfide group, carbonate group, urea group, and silyl group.

In some embodiments, the first and second reactive groups can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride.

In some embodiments, the nucleotide-arms can have the different types of reactive groups in the linkers where the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise a nucleotide unit with the same type of sugar 3′OH group. For example, a nucleotide conjugate can comprise a core (e.g., streptavidin or avidin core) attached to a plurality of nucleotide arms or biotinylated nucleotide arms, where all of the attached arms have a nucleotide unit having the same type of sugar 3′OH group.

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise a nucleotide unit with the same type of sugar 3′ blocking group (e.g., chain terminating moiety. For example, a nucleotide conjugate can comprise a core (e.g., streptavidin or avidin core) attached to a plurality of nucleotide arms or biotinylated nucleotide arms, where all of the attached arms can have a nucleotide unit having the same type of sugar 3′ blocking group. In some embodiments, the sugar 3′ blocking group can comprise an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the sugar 3′ blocking group can comprise a 3′-O-alkyl hydroxylamino group, a 3′-phosphorothioate group, a 3′-O-malonyl group, or a 3′-O-benzyl group. In some embodiments, the sugar 3′ blocking group can comprise an azide, azido or azidomethyl group.

In some embodiments, the sugar 3′ blocking group can be reactive with a chemical reagent. For example, the sugar 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The sugar 3′ blocking groups aryl and benzyl can be reactive with H2 and Palladium on carbon (Pd/C). The sugar 3′ blocking groups amine, amide, keto, isocyanate, phosphate, thiol, disulfide can be reactive with phosphine or with a thiol group including beta-mercaptoethanol or dithiothritol (DTT). The sugar 3′ blocking group carbonate can be reactive with potassium carbonate (K₂CO₃) in MeOH, with triethylamine in pyridine, or with Zn in acetic acid (AcOH). The sugar 3′ blocking groups urea and silyl can be reactive with tetrabutylammonium fluoride, pyridine-HF, with ammonium fluoride, or with triethylamine trihydrofluoride.

In some embodiments, the sugar 3′ blocking group (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise a nucleotide unit with different sugar 3′ blocking groups. For example, a nucleotide conjugate can comprise a core (e.g., streptavidin or avidin core) attached to a plurality of nucleotide arms or biotinylated nucleotide arms, where at least a first attached arm can have a first nucleotide unit having a first 3′ blocking group, and a second attached arm can have a second nucleotide unit having a second 3′ blocking group, where the first and second 3′ blocking groups are different.

In some embodiments, the first 3′ blocking group in the first nucleotide unit, and the second 3′ blocking group in the second nucleotide unit, can be selected in any combination from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the first 3′ blocking group in the first nucleotide unit, and the second 3′ blocking group in the second nucleotide unit, can be selected in any combination from a group consisting of an 3′-O-alkyl hydroxylamino group, a 3′-phosphorothioate group, a 3′-O-malonyl group, or a 3′-O-benzyl group. In some embodiments, the first 3′ blocking group in the first nucleotide unit, and the second 3′ blocking group in the second nucleotide unit, can be selected in any combination from a group consisting of an azide, azido or azidomethyl group.

In some embodiments, the first and second 3′ blocking groups can be reactive with a chemical reagent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride.

In some embodiments, the first and second 3′ blocking groups (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, methods, and kits comprising a nucleotide conjugate. In some embodiments, the nucleotide conjugate can comprise a core attached to a plurality of nucleotide-arms. In some embodiments, the plurality of nucleotide-arms can comprise a nucleotide unit with a first sugar 3′ OH blocking groups. In some embodiments, the plurality of nucleotide-arms can comprise a nucleotide unit with a second 3′ OH blocking group. In some cases, the first and second 3′ OH blocking groups can be different. For example, a nucleotide conjugate can comprise a core (e.g., streptavidin or avidin core) attached to a plurality of nucleotide arms or biotinylated nucleotide arms, where (a) at least a first arm can comprise a first nucleotide unit having a sugar moiety which includes a 3′OH group, (b) at least second arm can comprise a second nucleotide unit having a first 3′ blocking group, and (c) at least third arm can comprise a third nucleotide unit having a second blocking group, wherein the first and second 3′ blocking groups are different from each other.

In some embodiments, the first 3′ blocking group in the first nucleotide unit, and the second 3′ blocking group in the second nucleotide unit, can be selected in any combination from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the first 3′ blocking group in the first nucleotide unit, and the second 3′ blocking group in the second nucleotide unit, can be selected in any combination from a group consisting of an 3′-O-alkyl hydroxylamino group, a 3′-phosphorothioate group, a 3′-O-malonyl group, or a 3′-O-benzyl group. In some embodiments, the first 3′ blocking group in the first nucleotide unit, and the second 3′ blocking group in the second nucleotide unit, can be selected in any combination from a group consisting of an azide, azido or azidomethyl group.

In some embodiments, the first and second 3′ blocking groups can be reactive with a chemical reagent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride.

In some embodiments, the first and second 3′ blocking groups (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, methods, and kits comprising a nucleotide conjugate. In some embodiments, the nucleotide conjugate can have a core. In some embodiments, the core can be labeled with at least one detectable reporter moiety to form a labeled core. In some embodiments, a labeled core attached to two or more nucleotide-arms can comprise a labeled nucleotide conjugate. In some embodiments, a streptavidin or avidin core can be labeled with 1-6 or more reporter moieties. In some embodiments, the reporter moiety can comprise a fluorophore.

A mixture of nucleotide conjugates having different units in their nucleotide-arms, where distinction between the different nucleotide conjugates can be achieved. In some embodiments, the core of a first nucleotide conjugate can be labeled with a reporter moiety to distinguish it from a second labeled (or non-labeled) nucleotide conjugate. For example, a unit in a nucleotide-arm of the labeled first nucleotide conjugate can differ from a unit in a nucleotide-arm of a labeled second nucleotide conjugate. Any unit in the first nucleotide conjugate (e.g., spacer, linker, reactive group, nucleotide base, sugar 3′OH, 3′ blocking group, or a combination thereof) can differ from a corresponding unit in the second nucleotide conjugate, where the first and second reporter moieties correspond to the differentiating unit. In some embodiments, the first and second reporter moieties can be spectrally distinguishable from each other.

In some embodiments, the core of a first nucleotide conjugate can be labeled with a first reporter moiety that corresponds to the base (e.g., dATP, dGTP, dCTP, dTTP or dUTP) in the attached nucleotide-arms, and the core of a second nucleotide conjugate can be labeled with a second reporter moiety that corresponds to the base (e.g., dATP, dGTP, dCTP, dTTP or dUTP) in the attached nucleotide-arms, where the base in the first nucleotide conjugate and the base in the second nucleotide conjugate are different. In some embodiments, the first and second reporter moieties are spectrally distinguishable from each other. In some embodiment, detection of the first reporter moiety indicates a binding event, an incorporation event, or a combination of binding and incorporation events of the first nucleotide conjugate having the first base, and detection of the second reporter moiety indicates a binding event, an incorporation event, or a combination of binding and incorporation events of the second nucleotide conjugate having the second base. The binding event can be a nucleotide conjugate binding to a complexed polymerase. The incorporation event can be a nucleotide unit incorporating into the terminal 3′ end of an extendible primer in a complexed polymerase, where the nucleotide unit is part of a nucleotide conjugate.

### Synthetic Polypeptides

Disclosed herein, in some embodiments, are synthetic polypeptides. In some embodiments, the synthetic polypeptide is purified or isolated. In some embodiments, the synthetic polypeptide is a polymerizing enzyme. In some embodiments, the polymerizing enzyme comprises a nucleic acid polymerase or polymerizing portion thereof. In some embodiments, the polymerizing enzyme comprises a DNA polymerase or polymerizing portion thereof. In some embodiments, the polymerizing enzyme comprises an RNA polymerase or a polymerizing portion thereof. In some embodiments, the synthetic polypeptide may be used a formulation, system, or method disclosed herein. For example, the synthetic polypeptide may be used in method of nucleic acid sequence analysis, sequencing, identification, or processing, such as those disclosed herein.

The synthetic polypeptides disclosed herein comprise an amino acid sequence. In some embodiments, the amino acid sequence is greater than or equal to about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOS: 1, 391, 414, 431, 446, 448, 450, 452, 454, 456, 458, 460, and 462. In some embodiments, the amino acid sequence comprises one or more mutations. In some embodiments, the one or more mutations confers enhanced binding to a nucleotide or nucleotide unit disclosed herein, as compared with an otherwise identical synthetic polypeptide that does not have the one or more mutations. In some embodiments, binding is enhanced by at least about 5%, 10%, 15%, 20%, 25%, 30%, 35%, 40% 45%, 50%, 55%, 60%, 65%, 70%, 75%, 80%, 85%, 90%, 95%, or 100%, as compared with an otherwise identical synthetic polypeptide that does not have the one or more mutations. In some embodiments binding is enhanced by at least about 1-fold, 2-fold, 3-fold, 4-fold, 5-fold, 6-fold, 7-fold, 8-fold, 9-fold, or 10-fold, as compared with an otherwise identical synthetic polypeptide that does not have the one or more mutations. Affinity (e.g., strength of nucleotide binding by the synthetic polypeptide) may be measured by calculating the dissociation constant (K_{d}) of the binding complex formed between the nucleotide and the synthetic polypeptide using suitable techniques, such as those provided in Wu, J., de Paz, A., Zamft, B. M. et al. DNA binding strength increases the processivity and activity of a Y-Family DNA polymerase. Sci Rep 7, 4756 (2017), which is incorporated by reference herein in its entirety.

The one or more mutations can be a substitution, insertion, deletion, or chemical modification of one or more amino acids in the amino acid sequence. The amino acids disclosed herein may be referred to by the single letter or three letter code, set forth in Table 6 below.

TABLE 6
International Union of Pure and Applied
Chemistry (IUPAC) Amino Acid Codes

| Amino Acid Code: | Three letter Code: | Amino Acid: |
|---|---|---|
| A | Ala | Alanine |
| B | Asx | Aspartic acid or Asparagine |
| C | Cys | Cysteine |
| D | Asp | Aspartic acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Any amino acid |
| Y | Tyr | Tyrosine |
| Z | Glx | Glutamine or Glutamic acid |

A missense substitution of an amino acid may be indicated with an A1 #A2, wherein A1 is the amino acid at amino acid position, #, that is substituted with the amino acid, A2. For example, W26C denotes that amino acid 26 (Tryptophan, W) with reference to a base sequence is changed to a Cysteine (C). A nonsense substitution, by contrast, is denoted by A1 #X, where A1 is the amino acid at amino acid position # that is substituted with the stop codon, X. A deletion is denoted with a “del” after the amino acids flanking the deletion site. For example K29del denotes a deletion of a Lysine (K) at amino acid position 29 with reference to a base sequence. An insertion is denoted with “ins” between the amino acids flanking the insertion site followed by the amino acid(s) to be inserted. For example, K29_M30insQSK denotes an insertion of the amino acid sequence QSK between the lysine (K) at amino acid position 29 and methionine (M) at amino acid position 30, with reference to a base sequence. In some embodiments, the base sequence is any one of SEQ ID NOS: 1, 391, 414, 431, 446, 448, 450, 452, 454, 456, 458, 460, and 462.

In some embodiments, the synthetic polypeptide comprises an amino acid sequence having at least 70% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 71% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 72% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 73% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 74% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 75% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 76% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 77% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 78% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 79% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 80% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 81% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 82% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 83% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 84% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 85% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 86% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 87% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 88% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 89% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 90% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 91% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 92% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 93% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 94% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 95% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 96% sequence identity to SEQ ID NO: 1 and the amino acid sequence comprises one or more mutations listed in Table 1A with reference to SEQ ID NO: 1. In some embodiments, the synthetic polypeptide comprises comprising an amino acid sequence having at least 97% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 98% sequence identity to SEQ ID NO: 1 and the amino acid sequence comprises one or more mutations listed in Table 1A with reference to SEQ ID NO: 1. In some embodiments, the synthetic polypeptide comprises an amino acid sequence having at least 99% sequence identity to SEQ ID NO: 1 and the amino acid sequence comprises one or more mutations listed in Table 1A with reference to SEQ ID NO: 1.

Disclosed herein is a synthetic polypeptide comprising an amino acid sequence having at least 70% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 71% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 72% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 73% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 74% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 75% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 76% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 77% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 78% sequence identity to SEQ ID NO: 391 and the amino acid sequence comprises a D141A mutation or a D143A mutation with reference to SEQ ID NO: 391. Disclosed herein is a synthetic polypeptide comprising an amino acid sequence having at least 79% sequence identity to SEQ ID NO: 391 and the amino acid sequence comprises a D141A mutation or a D143A mutation with reference to SEQ ID NO: 391. Disclosed herein, in some embodiments, is a synthetic polypeptide comprising an amino acid sequence having at least 80% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 81% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 82% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 83% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 84% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 85% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 86% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 87% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 88% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 89% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 90% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 91% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 92% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 93% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 94% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 95% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 96% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 97% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 98% sequence identity to SEQ ID NO: 391 and the amino acid sequence comprises a D141A mutation or a D143A mutation with reference to SEQ ID NO: 391. In some embodiments, the synthetic polypeptide comprises an amino acid sequence having at least 99% sequence identity to SEQ ID NO: 391 and the amino acid sequence comprises a D141A mutation or a D143A mutation with reference to SEQ ID NO: 391.

In some embodiments, the amino acid sequence comprises the D141A mutation and the D143A mutation with reference to SEQ ID NO: 391. In some embodiments, the amino acid sequence further comprises a Y410A mutation, a L409S mutation, a Y261A mutation, a P411G mutation, a F406I mutation, a P411A mutation, a Y7A mutation, a Y493I mutation, a Y493T mutation, a V513I mutation, a L409A mutation, an A485S mutation, a Y410G mutation, an I521H mutation, or a K507L mutation, or any combination thereof, with reference to SEQ ID No: 391. In some embodiments, the amino acid sequence further comprises the Y410A mutation, with reference to SEQ ID No: 391. In some embodiments, the amino acid sequence further comprises the L409S mutation, the Y410A mutation, and the Y261A mutation, with reference to SEQ ID No: 391. In some embodiments, the amino acid sequence further comprises the L409S mutation, the Y410A mutation, the P411G mutation, and the Y261A mutation, with reference to SEQ ID No: 391. In some embodiments, the amino acid sequence further comprises the Y261A mutation, the F406I mutation, the L409S mutation, the Y410A mutation, and the P411A mutation, with reference to SEQ ID No: 391. In some embodiments, the amino acid sequence further comprises the Y7A mutation, the Y261A mutation, and the Y410A mutation, with reference to SEQ ID No: 391. In some embodiments, the amino acid sequence further comprises the Y261A mutation, the Y410A mutation, and the Y493I mutation, with reference to SEQ ID No: 391. In some embodiments, the amino acid sequence further comprises the Y261A mutation, the Y410A mutation, and the Y493T mutation, with reference to SEQ ID No: 391. In some embodiments, the amino acid sequence further comprises the Y261A mutation, the Y410A mutation, and the V513I mutation, with reference to SEQ ID No: 391. In some embodiments, the amino acid sequence further comprises the Y7A mutation, the Y261A mutation, the L409S mutation, and the Y410A mutation, with reference to SEQ ID No: 391. In some embodiments, the amino acid sequence further comprises the Y261A mutation, the L409A mutation, the Y410A mutation, and the P411A mutation, with reference to SEQ ID No: 391. In some embodiments, the amino acid sequence further comprises the Y261A mutation, the L409S mutation, the Y410A mutation, and the P411G mutation, with reference to SEQ ID No: 391. In some embodiments, the amino acid sequence further comprises the Y261A mutation, the L409S mutation, and the Y410A mutation, with reference to SEQ ID No: 391. In some embodiments, the amino acid sequence further comprises the Y261A mutation, the L409S mutation, and the Y410G mutation, with reference to SEQ ID No: 391. In some embodiments, the amino acid sequence further comprises the Y261A mutation, the L409A mutation, the Y410A mutation, the P411A mutation, and the A485S mutation, with reference to SEQ ID No: 391. In some embodiments, the amino acid sequence further comprises the Y261A mutation, the L409S mutation, the Y410A mutation, the P411G mutation, and the A485S mutation, with reference to SEQ ID No: 391. In some embodiments, the amino acid sequence further comprises the Y261A mutation, the L409S mutation, the Y410A mutation, and the A485S mutation, with reference to SEQ ID No: 391. In some embodiments, the amino acid sequence further comprises the Y261A mutation, the L409S mutation, the Y410G mutation, and the A485S mutation, with reference to SEQ ID No: 391. In some embodiments, the amino acid sequence further comprises the Y261A mutation, the L409A mutation, the Y410A mutation, the P411A mutation, and the I521H mutation, with reference to SEQ ID No: 391. In some embodiments, the amino acid sequence further comprises the Y261A mutation, the L409S mutation, the Y410A mutation, the P411G mutation, and the I521H mutation, with reference to SEQ ID No: 391. In some embodiments, the amino acid sequence further comprises the Y261A mutation, the L409S mutation, the Y410A mutation, and the I521H mutation, with reference to SEQ ID No: 391. In some embodiments, the amino acid sequence further comprises the Y261A mutation, the L409S mutation, the Y410G mutation, and the I521H mutation, with reference to SEQ ID No: 391. In some embodiments, the amino acid sequence further comprises the Y261A mutation, the L409S mutation, the Y410A mutation, the A485S mutation, the K507L mutation, and the I521H mutation, with reference to SEQ ID No: 391. In some embodiments, the amino acid sequence further comprises any one of SEQ ID NOs: 392-413.

Disclosed herein is a synthetic polypeptide comprising an amino acid sequence having at least 70% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 71% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 72% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 73% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 74% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 75% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 76% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 77% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 78% sequence identity to SEQ ID NO: 414 and the amino acid sequence comprises a D158A mutation or an E160A mutation with reference to SEQ ID NO: 414. Disclosed herein is a synthetic polypeptide comprising an amino acid sequence having at least 79% sequence identity to SEQ ID NO: 414 and the amino acid sequence comprises a D158A mutation or an E160A mutation with reference to SEQ ID NO: 414. Disclosed herein, in some embodiments, is a synthetic polypeptide comprising an amino acid sequence having at least 80% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 81% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 82% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 83% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 84% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 85% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 86% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 87% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 88% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 89% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 90% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 91% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 92% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 93% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 94% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 95% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 96% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 97% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 98% sequence identity to SEQ ID NO: 414 and the amino acid sequence comprises a D158A mutation or an E160A mutation with reference to SEQ ID NO: 414. In some embodiments, the synthetic polypeptide comprises an amino acid sequence having at least 99% sequence identity to SEQ ID NO: 414 and the amino acid sequence comprises a D158A mutation or an E160A mutation with reference to SEQ ID NO: 414.

In some embodiments, the amino acid sequence comprises the D158A mutation and the E160A mutation with reference to SEQ ID NO: 414. In some embodiments, the amino acid sequence further comprises a L431A mutation, a Y432A mutation, a P433I mutation, an A507S mutation, a K506Q mutation, a P433A mutation, an I543H mutation, a L431S mutation, a P433G mutation, a K529L mutation, or a Y432G mutation, or any combination thereof, with reference to SEQ ID No: 414. In some embodiments, the amino acid sequence further comprises the L431A mutation, the Y432A mutation, the P433I mutation, and the A507S mutation, with reference to SEQ ID No: 414. In some embodiments, the amino acid sequence further comprises the L431A mutation, the Y432A mutation, the P433I mutation, and the K506Q mutation, with reference to SEQ ID No: 414. In some embodiments, the amino acid sequence further comprises the L431A mutation, the Y432A mutation, the P433I mutation, the K506Q mutation, and the A507S mutation, with reference to SEQ ID No: 414. In some embodiments, the amino acid sequence further comprises the L431A mutation, the Y432A mutation, and the P433A mutation, with reference to SEQ ID No: 414. In some embodiments, the amino acid sequence further comprises the L431A mutation, the Y432A mutation, the P433A mutation, and the A507S mutation, with reference to SEQ ID No: 414. In some embodiments, the amino acid sequence further comprises the L431A mutation, the Y432A mutation, the P433A mutation, and the I543H mutation, with reference to SEQ ID No: 414. In some embodiments, the amino acid sequence further comprises the L431S mutation, the Y432A mutation, and the P433G mutation, with reference to SEQ ID No: 414. In some embodiments, the amino acid sequence further comprises the L431S mutation, the Y432A mutation, the P433G mutation, and the A507S mutation, with reference to SEQ ID No: 414. In some embodiments, the amino acid sequence further comprises the L431S mutation, the Y432A mutation, the P433G mutation, and the I543H mutation, with reference to SEQ ID No: 414. In some embodiments, the amino acid sequence further comprises the L431S mutation and the Y432A mutation, with reference to SEQ ID No: 414. In some embodiments, the amino acid sequence further comprises the L431S mutation, the Y432A mutation, and the A507S mutation, with reference to SEQ ID No: 414. In some embodiments, the amino acid sequence further comprises the L431S mutation, the Y432A mutation, the A507S mutation, the K529L mutation, and the I543H mutation, with reference to SEQ ID No: 414. In some embodiments, the amino acid sequence further comprises the L431S mutation, the Y432A mutation, and the I543H mutation, with reference to SEQ ID No: 414. In some embodiments, the amino acid sequence further comprises the L431S mutation and the Y432G mutation, with reference to SEQ ID No: 414. In some embodiments, the amino acid sequence further comprises the L431S mutation, the Y432G mutation, and the A507S mutation, with reference to SEQ ID No: 414. In some embodiments, the amino acid sequence further comprises the L431S mutation, the Y432G mutation, and the I543H mutation, with reference to SEQ ID No: 414. In some embodiments, the amino acid sequence further comprises any one of SEQ ID NOs: 415-430.

Disclosed herein is a synthetic polypeptide comprising an amino acid sequence having at least 70% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 71% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 72% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 73% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 74% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 75% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 76% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 77% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 78% sequence identity to SEQ ID NO: 431 and the amino acid sequence comprises a D141A mutation or an E143A mutation with reference to SEQ ID NO: 431. Disclosed herein is a synthetic polypeptide comprising an amino acid sequence having at least 79% sequence identity to SEQ ID NO: 431 and the amino acid sequence comprises a D141A mutation or an E143A mutation with reference to SEQ ID NO: 431. Disclosed herein, in some embodiments, is a synthetic polypeptide comprising an amino acid sequence having at least 80% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 81% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 82% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 83% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 84% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 85% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 86% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 87% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 88% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 89% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 90% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 91% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 92% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 93% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 94% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 95% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 96% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 97% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 98% sequence identity to SEQ ID NO: 431 and the amino acid sequence comprises a D141A mutation or an E143A mutation with reference to SEQ ID NO: 431. In some embodiments, the synthetic polypeptide comprises an amino acid sequence having at least 99% sequence identity to SEQ ID NO: 431 and the amino acid sequence comprises a D141A mutation or an E143A mutation with reference to SEQ ID NO: 431.

In some embodiments, the amino acid sequence comprises the D141A mutation and the E143A mutation with reference to SEQ ID NO: 431. In some embodiments, the amino acid sequence further comprises a Y412A mutation, a L411A mutation, a P413A mutation, an A488S mutation, an I524H mutation, a L411S mutation, a P413G mutation, a K510L mutation, or a Y412G mutation, or any combination thereof, with reference to SEQ ID No: 431. In some embodiments, the amino acid sequence further comprises the Y412A mutation with reference to SEQ ID No: 431. In some embodiments, the amino acid sequence further comprises the L411A mutation, the Y412A mutation, and the P413A mutation, with reference to SEQ ID No: 431. In some embodiments, the amino acid sequence further comprises the L411A mutation, the Y412A mutation, the P413A mutation, and the A488S mutation, with reference to SEQ ID No: 431. In some embodiments, the amino acid sequence further comprises the L411A mutation, the Y412A mutation, the P413A mutation, and the I524H mutation, with reference to SEQ ID No: 431. In some embodiments, the amino acid sequence further comprises the L411S mutation, the Y412A mutation, and the P413G mutation, with reference to SEQ ID No: 431. In some embodiments, the amino acid sequence further comprises the L411S mutation, the Y412A mutation, the P413G mutation, and the A488S mutation, with reference to SEQ ID No: 431. In some embodiments, the amino acid sequence further comprises the L411S mutation, the Y412A mutation, the P413G mutation, and the I524H mutation, with reference to SEQ ID No: 431. In some embodiments, the amino acid sequence further comprises the L411S mutation and the Y412A mutation, with reference to SEQ ID No: 431. In some embodiments, the amino acid sequence further comprises the L411S mutation, the Y412A mutation, and the A488S mutation, with reference to SEQ ID No: 431. In some embodiments, the amino acid sequence further comprises the L411S mutation, the Y412A mutation, the A488S mutation, the K510L mutation, and the I524H mutation, with reference to SEQ ID No: 431. In some embodiments, the amino acid sequence further comprises the L411S mutation, the Y412A mutation, and the I524H mutation, with reference to SEQ ID No: 431. In some embodiments, the amino acid sequence further comprises the L411S mutation and Y412G mutation, with reference to SEQ ID No: 431. In some embodiments, the amino acid sequence further comprises the L411S mutation, Y412G mutation, and the A488S mutation, with reference to SEQ ID No: 431. In some embodiments, the amino acid sequence further comprises the L411S mutation, Y412G mutation, and the I524H mutation, with reference to SEQ ID No: 431. In some embodiments, the amino acid sequence further comprises any one of SEQ ID NOs: 432-445.

Disclosed herein is a synthetic polypeptide comprising an amino acid sequence having at least 70% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 71% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 72% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 73% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 74% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 75% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 76% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 77% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 78% sequence identity to SEQ ID NO: 446 and the amino acid sequence comprises a D141A mutation, an E143A mutation, or a Y412A mutation, with reference to SEQ ID NO: 446. Disclosed herein is a synthetic polypeptide comprising an amino acid sequence having at least 79% sequence identity to SEQ ID NO: 446 and the amino acid sequence comprises a D141A mutation, an E143A mutation, or a Y412A mutation, with reference to SEQ ID NO: 446. Disclosed herein, in some embodiments, is a synthetic polypeptide comprising an amino acid sequence having at least 80% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 81% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 82% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 83% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 84% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 85% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 86% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 87% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 88% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 89% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 90% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 91% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 92% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 93% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 94% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 95% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 96% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 97% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 98% sequence identity to SEQ ID NO: 446 and the amino acid sequence comprises a D141A mutation, an E143A mutation, or a Y412A mutation, with reference to SEQ ID NO: 446. In some embodiments, the synthetic polypeptide comprises an amino acid sequence having at least 99% sequence identity to SEQ ID NO: 446 and the amino acid sequence comprises a D141A mutation, an E143A mutation, or a Y412A mutation, with reference to SEQ ID NO: 446. In some embodiments, the amino acid sequence comprises two or more of the D141A mutation, the E143A mutation and the Y412A mutation, with reference to SEQ ID NO: 446. In some embodiments, the amino acid sequence comprises the D141A mutation, the E143A mutation and the Y412A mutation, with reference to SEQ ID NO: 446. In some embodiments, the amino acid sequence further comprises SEQ ID NO: 447.

Disclosed herein is a synthetic polypeptide comprising an amino acid sequence having at least 70% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 71% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 72% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 73% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 74% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 75% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 76% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 77% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 78% sequence identity to SEQ ID NO: 448 and the amino acid sequence comprises a D149A mutation, an E151A mutation, a Y272A mutation, or a Y422A mutation, with reference to SEQ ID NO: 448. Disclosed herein is a synthetic polypeptide comprising an amino acid sequence having at least 79% sequence identity to SEQ ID NO: 448 and the amino acid sequence comprises a D149A mutation, an E151A mutation, a Y272A mutation, or a Y422A mutation, with reference to SEQ ID NO: 448. Disclosed herein, in some embodiments, is a synthetic polypeptide comprising an amino acid sequence having at least 80% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 81% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 82% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 83% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 84% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 85% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 86% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 87% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 88% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 89% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 90% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 91% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 92% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 93% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 94% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 95% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 96% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 97% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 98% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the synthetic polypeptide comprises an amino acid sequence having at least 99% sequence identity to SEQ ID NO: 448 and the amino acid sequence comprises a D149A mutation, an E151A mutation, a Y272A mutation, or a Y422A mutation, with reference to SEQ ID NO: 448. In some embodiments, the amino acid sequence comprises two or more of the D149A mutation, the E151A mutation, the Y272A mutation, and the Y422A mutation, with reference to SEQ ID NO: 448. In some embodiments, the amino acid sequence comprises three or more of the D149A mutation, the E151A mutation, the Y272A mutation, and the Y422A mutation, with reference to SEQ ID NO: 448. In some embodiments, the amino acid sequence comprises the D149A mutation, the E151A mutation, the Y272A mutation, and the Y422A mutation, with reference to SEQ ID NO: 448. In some embodiments, the amino acid sequence further comprises SEQ ID NO: 449.

Disclosed herein is a synthetic polypeptide comprising an amino acid sequence having at least 70% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 71% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 72% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 73% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 74% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 75% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 76% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 77% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 78% sequence identity to SEQ ID NO: 450 and the amino acid sequence comprises a D149A mutation, an E151A mutation, a Y272A mutation, or a Y422A mutation, with reference to SEQ ID NO: 450. Disclosed herein is a synthetic polypeptide comprising an amino acid sequence having at least 79% sequence identity to SEQ ID NO: 450 and the amino acid sequence comprises a D149A mutation, an E151A mutation, a Y272A mutation, or a Y422A mutation, with reference to SEQ ID NO: 450. Disclosed herein, in some embodiments, is a synthetic polypeptide comprising an amino acid sequence having at least 80% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 81% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 82% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 83% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 84% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 85% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 86% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 87% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 88% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 89% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 90% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 91% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 92% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 93% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 94% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 95% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 96% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 97% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 98% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the synthetic polypeptide comprises an amino acid sequence having at least 99% sequence identity to SEQ ID NO: 450 and the amino acid sequence comprises a D149A mutation, an E151A mutation, a Y272A mutation, or a Y422A mutation, with reference to SEQ ID NO: 450. In some embodiments, the amino acid sequence comprises two or more of the D149A mutation, the E151A mutation, the Y272A mutation, and the Y422A mutation, with reference to SEQ ID NO: 450. In some embodiments, the amino acid sequence comprises three or more of the D149A mutation, the E151A mutation, the Y272A mutation, and the Y422A mutation, with reference to SEQ ID NO: 450. In some embodiments, the amino acid sequence comprises the D149A mutation, the E151A mutation, the Y272A mutation, and the Y422A mutation, with reference to SEQ ID NO: 450. In some embodiments, the amino acid sequence further comprises SEQ ID NO: 451.

Disclosed herein is a synthetic polypeptide comprising an amino acid sequence having at least 70% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 71% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 72% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 73% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 74% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 75% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 76% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 77% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 78% sequence identity to SEQ ID NO: 452 and the amino acid sequence comprises a D170A mutation, an E172A mutation, or a Y449A mutation, with reference to SEQ ID NO: 452. Disclosed herein is a synthetic polypeptide comprising an amino acid sequence having at least 79% sequence identity to SEQ ID NO: 452 and the amino acid sequence comprises a D170A mutation, an E172A mutation, or a Y449A mutation, with reference to SEQ ID NO: 452. Disclosed herein, in some embodiments, is a synthetic polypeptide comprising an amino acid sequence having at least 80% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 81% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 82% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 83% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 84% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 85% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 86% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 87% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 88% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 89% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 90% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 91% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 92% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 93% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 94% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 95% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 96% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 97% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 98% sequence identity to SEQ ID NO: 452 and the amino acid sequence comprises a D170A mutation, an E172A mutation, or a Y449A mutation, with reference to SEQ ID NO: 452. In some embodiments, the synthetic polypeptide comprises an amino acid sequence having at least 99% sequence identity to SEQ ID NO: 452 and the amino acid sequence comprises a D170A mutation, an E172A mutation, or a Y449A mutation, with reference to SEQ ID NO: 452. In some embodiments, the amino acid sequence comprises two or more of the D170A mutation, the E172A mutation, and the Y449A mutation, with reference to SEQ ID NO: 452. In some embodiments, the amino acid sequence comprises the D170A mutation, the E172A mutation, and the Y449A mutation, with reference to SEQ ID NO: 452. In some embodiments, the amino acid sequence further comprises SEQ ID NO: 453.

Disclosed herein is a synthetic polypeptide comprising an amino acid sequence having at least 70% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 71% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 72% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 73% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 74% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 75% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 76% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 77% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 78% sequence identity to SEQ ID NO: 454 and the amino acid sequence comprises a D170A mutation, an E172A mutation, or a Y449A mutation, with reference to SEQ ID NO: 454. Disclosed herein is a synthetic polypeptide comprising an amino acid sequence having at least 79% sequence identity to SEQ ID NO: 454 and the amino acid sequence comprises a D170A mutation, an E172A mutation, or a Y449A mutation, with reference to SEQ ID NO: 454. Disclosed herein, in some embodiments, is a synthetic polypeptide comprising an amino acid sequence having at least 80% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 81% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 82% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 83% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 84% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 85% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 86% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 87% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 88% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 89% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 90% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 91% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 92% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 93% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 94% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 95% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 96% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 97% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 98% sequence identity to SEQ ID NO: 454 and the amino acid sequence comprises a D170A mutation, an E172A mutation, or a Y449A mutation, with reference to SEQ ID NO: 454. In some embodiments, the synthetic polypeptide comprises an amino acid sequence having at least 99% sequence identity to SEQ ID NO: 454 and the amino acid sequence comprises a D170A mutation, an E172A mutation, or a Y449A mutation, with reference to SEQ ID NO: 454. In some embodiments, the amino acid sequence comprises two or more of the D170A mutation, the E172A mutation, and the Y449A mutation, with reference to SEQ ID NO: 454. In some embodiments, the amino acid sequence comprises the D170A mutation, the E172A mutation, and the Y449A mutation, with reference to SEQ ID NO: 454. In some embodiments, the amino acid sequence further comprises SEQ ID NO: 455.

Disclosed herein is a synthetic polypeptide comprising an amino acid sequence having at least 70% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 71% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 72% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 73% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 74% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 75% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 76% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 77% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 78% sequence identity to SEQ ID NO: 456 and the amino acid sequence comprises a D173A mutation, an E175A mutation, a Y452A mutation, or a C468A mutation, with reference to SEQ ID NO: 456. Disclosed herein is a synthetic polypeptide comprising an amino acid sequence having at least 79% sequence identity to SEQ ID NO: 456 and the amino acid sequence comprises a D173A mutation, an E175A mutation, a Y452A mutation, or a C468A mutation, with reference to SEQ ID NO: 456. Disclosed herein, in some embodiments, is a synthetic polypeptide comprising an amino acid sequence having at least 80% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 81% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 82% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 83% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 84% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 85% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 86% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 87% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 88% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 89% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 90% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 91% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 92% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 93% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 94% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 95% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 96% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 97% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 98% sequence identity to SEQ ID NO: 456 and the amino acid sequence comprises a D173A mutation, an E175A mutation, a Y452A mutation, or a C468A mutation, with reference to SEQ ID NO: 456. In some embodiments, the synthetic polypeptide comprises an amino acid sequence having at least 99% sequence identity to SEQ ID NO: 456 and the amino acid sequence comprises a D173A mutation, an E175A mutation, a Y452A mutation, or a C468A mutation, with reference to SEQ ID NO: 456. In some embodiments, the amino acid sequence comprises two or more of the D173A mutation, the E175A mutation, the Y452A mutation and the C468A mutation, with reference to SEQ ID NO: 456. In some embodiments, the amino acid sequence comprises three or more of the D173A mutation, the E175A mutation, the Y452A mutation and the C468A mutation, with reference to SEQ ID NO: 456. In some embodiments, the amino acid sequence comprises the D173A mutation, the E175A mutation, the Y452A mutation and the C468A mutation, with reference to SEQ ID NO: 456. In some embodiments, the amino acid sequence further comprises SEQ ID NO: 457.

Disclosed herein is a synthetic polypeptide comprising an amino acid sequence having at least 70% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 71% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 72% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 73% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 74% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 75% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 76% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 77% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 78% sequence identity to SEQ ID NO: 458 and the amino acid sequence comprises a D149A mutation, an E151A mutation, a Y272A mutation, or a Y422A mutation, with reference to SEQ ID NO: 458. Disclosed herein is a synthetic polypeptide comprising an amino acid sequence having at least 79% sequence identity to SEQ ID NO: 458 and the amino acid sequence comprises a D149A mutation, an E151A mutation, a Y272A mutation, or a Y422A mutation, with reference to SEQ ID NO: 458. Disclosed herein, in some embodiments, is a synthetic polypeptide comprising an amino acid sequence having at least 80% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 81% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 82% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 83% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 84% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 85% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 86% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 87% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 88% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 89% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 90% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 91% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 92% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 93% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 94% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 95% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 96% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 97% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 98% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the synthetic polypeptide comprises an amino acid sequence having at least 99% sequence identity to SEQ ID NO: 458 and the amino acid sequence comprises a D149A mutation, an E151A mutation, a Y272A mutation, or a Y422A mutation, with reference to SEQ ID NO: 458. In some embodiments, the amino acid sequence comprises two or more of the D149A mutation, the E151A mutation, the Y272A mutation, and the Y422A mutation, with reference to SEQ ID NO: 458. In some embodiments, the amino acid sequence comprises three or more of the D149A mutation, the E151A mutation, the Y272A mutation, and the Y422A mutation, with reference to SEQ ID NO: 458. In some embodiments, the amino acid sequence comprises the D149A mutation, the E151A mutation, the Y272A mutation, and the Y422A mutation, with reference to SEQ ID NO: 458. In some embodiments, the amino acid sequence further comprises SEQ ID NO: 459.

Disclosed herein is a synthetic polypeptide comprising an amino acid sequence having at least 70% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 71% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 72% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 73% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 74% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 75% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 76% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 77% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 78% sequence identity to SEQ ID NO: 460 and the amino acid sequence comprises a D141A mutation, an E143A mutation, or a Y412A mutation, with reference to SEQ ID NO: 460. Disclosed herein is a synthetic polypeptide comprising an amino acid sequence having at least 79% sequence identity to SEQ ID NO: 460 and the amino acid sequence comprises a D141A mutation, an E143A mutation, or a Y412A mutation, with reference to SEQ ID NO: 460. Disclosed herein, in some embodiments, is a synthetic polypeptide comprising an amino acid sequence having at least 80% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 81% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 82% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 83% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 84% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 85% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 86% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 87% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 88% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 89% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 90% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 91% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 92% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 93% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 94% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 95% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 96% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 97% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 98% sequence identity to SEQ ID NO: 460 and the amino acid sequence comprises a D141A mutation, an E143A mutation, or a Y412A mutation, with reference to SEQ ID NO: 460. In some embodiments, the synthetic polypeptide comprises an amino acid sequence having at least 99% sequence identity to SEQ ID NO: 460 and the amino acid sequence comprises a D141A mutation, an E143A mutation, or a Y412A mutation, with reference to SEQ ID NO: 460. In some embodiments, the amino acid sequence comprises two or more of the D141A mutation, the E143A mutation, and the Y412A mutation, with reference to SEQ ID NO: 460. In some embodiments, the amino acid sequence comprises the D141A mutation, the E143A mutation, and the Y412A mutation, with reference to SEQ ID NO: 460. In some embodiments, the amino acid sequence further comprises SEQ ID NO: 461.

Disclosed herein is a synthetic polypeptide comprising an amino acid sequence having at least 70% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 71% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 72% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 73% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 74% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 75% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 76% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 77% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 78% sequence identity to SEQ ID NO: 462 and the amino acid sequence comprises a D141A mutation, an E143A mutation, or a Y412A mutation, with reference to SEQ ID NO: 462. Disclosed herein is a synthetic polypeptide comprising an amino acid sequence having at least 79% sequence identity to SEQ ID NO: 462 and the amino acid sequence comprises a D141A mutation, an E143A mutation, or a Y412A mutation, with reference to SEQ ID NO: 462. Disclosed herein, in some embodiments, is a synthetic polypeptide comprising an amino acid sequence having at least 80% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 81% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 82% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 83% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 84% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 85% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 86% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 87% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 88% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 89% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 90% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 91% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 92% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 93% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 94% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 95% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 96% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 97% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 98% sequence identity to SEQ ID NO: 462 and the amino acid sequence comprises a D141A mutation, an E143A mutation, or a Y412A mutation, with reference to SEQ ID NO: 462. In some embodiments, the synthetic polypeptide comprises an amino acid sequence having at least 99% sequence identity to SEQ ID NO: 462 and the amino acid sequence comprises a D141A mutation, an E143A mutation, or a Y412A mutation, with reference to SEQ ID NO: 462. In some embodiments, the amino acid sequence further comprises an amino acid deletion from amino acid position I496 to amino acid position S1033, or any portion of the amino acid sequence thereof, with reference to SEQ ID NO: 462. In some embodiments, the amino acid sequence comprises two or more of the D141A mutation, the E143A mutation, and the Y412A mutation, with reference to SEQ ID NO: 462. In some embodiments, the amino acid sequence comprises the D141A mutation, the E143A mutation, and the Y412A mutation, with reference to SEQ ID NO: 462. In some embodiments, the amino acid sequence further comprises (i) an amino acid deletion from amino acid position 1496 to amino acid position S1033, or any portion of the amino acid sequence thereof, with reference to SEQ ID NO: 462; and (ii) two or more of the D141A mutation, the E143A mutation, and the Y412A mutation, with reference to SEQ ID NO: 462. In some embodiments, the amino acid sequence further comprises (i) an amino acid deletion from amino acid position 1496 to amino acid position S1033, or any portion of the amino acid sequence thereof, with reference to SEQ ID NO: 462; and (ii) the D141A mutation, the E143A mutation, and the Y412A mutation, with reference to SEQ ID NO: 462. In some embodiments, the amino acid sequence further comprises SEQ ID NO: 463.

Disclosed herein is a synthetic polypeptide comprising an amino acid sequence having at least 70% sequence identity to any one of SEQ ID NOs: 1-472. Disclosed herein is a synthetic polypeptide comprising an amino acid sequence having at least 71% sequence identity to any one of SEQ ID NOs: 1-472. Disclosed herein is a synthetic polypeptide comprising an amino acid sequence having at least 72% sequence identity to any one of SEQ ID NOs: 1-472. Disclosed herein is a synthetic polypeptide comprising an amino acid sequence having at least 73% sequence identity to any one of SEQ ID NOs: 1-472. Disclosed herein is a synthetic polypeptide comprising an amino acid sequence having at least 74% sequence identity to any one of SEQ ID NOs: 1-472. Disclosed herein is a synthetic polypeptide comprising an amino acid sequence having at least 75% sequence identity to any one of SEQ ID NOs: 1-472. Disclosed herein is a synthetic polypeptide comprising an amino acid sequence having at least 76% sequence identity to any one of SEQ ID NOs: 1-472. Disclosed herein is a synthetic polypeptide comprising an amino acid sequence having at least 77% sequence identity to any one of SEQ ID NOs: 1-472. Disclosed herein is a synthetic polypeptide comprising an amino acid sequence having at least 78% sequence identity to any one of SEQ ID NOs: 1-472. Disclosed herein is a synthetic polypeptide comprising an amino acid sequence having at least 79% sequence identity to any one of SEQ ID NOs: 1-472. In some embodiments, the synthetic polypeptide comprises an amino acid sequence having at least 80% sequence identity to any one of SEQ ID NOs: 1-472. In some embodiments, the synthetic polypeptide comprises an amino acid sequence having at least 81% sequence identity to any one of SEQ ID NOs: 1-472. In some embodiments, the synthetic polypeptide comprises an amino acid sequence having at least 82% sequence identity to any one of SEQ ID NOs: 1-472. In some embodiments, the synthetic polypeptide comprises an amino acid sequence having at least 83% sequence identity to any one of SEQ ID NOs: 1-472. In some embodiments, the synthetic polypeptide comprises an amino acid sequence having at least 84% sequence identity to any one of SEQ ID NOs: 1-472. In some embodiments, the synthetic polypeptide comprises an amino acid sequence having at least 85% sequence identity to any one of SEQ ID NOs: 1-472. In some embodiments, the synthetic polypeptide comprises an amino acid sequence having at least 86% sequence identity to any one of SEQ ID NOs: 1-472. In some embodiments, the synthetic polypeptide comprises an amino acid sequence having at least 87% sequence identity to any one of SEQ ID NOs: 1-472. In some embodiments, the synthetic polypeptide comprises an amino acid sequence having at least 88% sequence identity to any one of SEQ ID NOs: 1-472. In some embodiments, the synthetic polypeptide comprises an amino acid sequence having at least 89% sequence identity to any one of SEQ ID NOs: 1-472. In some embodiments, the synthetic polypeptide comprises an amino acid sequence having at least 90% sequence identity to any one of SEQ ID NOs: 1-472. In some embodiments, the synthetic polypeptide comprises an amino acid sequence having at least 91% sequence identity to any one of SEQ ID NOs: 1-472. In some embodiments, the synthetic polypeptide comprises an amino acid sequence having at least 92% sequence identity to any one of SEQ ID NOs: 1-472. In some embodiments, the synthetic polypeptide comprises an amino acid sequence having at least 93% sequence identity to any one of SEQ ID NOs: 1-472. In some embodiments, the synthetic polypeptide comprises an amino acid sequence having at least 94% sequence identity to any one of SEQ ID NOs: 1-472. In some embodiments, the synthetic polypeptide comprises an amino acid sequence having at least 95% sequence identity to any one of SEQ ID NOs: 1-472. In some embodiments, the synthetic polypeptide comprises an amino acid sequence having at least 96% sequence identity to any one of SEQ ID NOs: 1-472. In some embodiments, the synthetic polypeptide comprises an amino acid sequence having at least 97% sequence identity to any one of SEQ ID NOs: 1-472. In some embodiments, the synthetic polypeptide comprises an amino acid sequence having at least 98% sequence identity to any one of SEQ ID NOs: 1-472. In some embodiments, the synthetic polypeptide comprises an amino acid sequence having at least 99% sequence identity to any one of SEQ ID NOs: 1-472. In some embodiments, the synthetic polypeptide comprises an amino acid sequence of any one of SEQ ID NOs: 1-472.

Disclosed herein is a synthetic polypeptide, comprising: an amino acid sequence having at least 70% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 71% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 72% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 73% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 74% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 75% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 76% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 77% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 78% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 79% sequence identity to any one of SEQ ID NOs: 1, 391, 414, 431, 446, 448, 450, 452, 454, 456, 458, 460, and 462. In some embodiments, the amino acid sequence provides a binding constant of at least 1, 2, 3, 4, 5, 6, 7, 8, 9, 10, 11, 12, 13, 14, 15, 16, 17, 18, 19, or 20, or any value therebetween: (i) between the amino acid sequence and a primed nucleic acid sequence; (ii) between the amino acid sequence and a nucleotide; or (iii) both (i) and (ii).

Disclosed herein is a synthetic polypeptide, comprising: an amino acid sequence having at least 80% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 81% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 82% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 83% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 84% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 85% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 86% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 87% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 88% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 89% sequence identity to any one of SEQ ID NOs: 1, 391, 414, 431, 446, 448, 450, 452, 454, 456, 458, 460, and 462. In some embodiments, the amino acid sequence provides a binding constant of at least 1, 2, 3, 4, 5, 6, 7, 8, 9, 10, 11, 12, 13, 14, 15, 16, 17, 18, 19, or 20, or any value therebetween: (i) between the amino acid sequence and a primed nucleic acid sequence; (ii) between the amino acid sequence and a nucleotide; or (iii) both (i) and (ii).

Disclosed herein is a synthetic polypeptide, comprising: an amino acid sequence having at least 90% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 91% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 92% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 93% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 94% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 95% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 96% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 97% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 98% sequence identity to any one of SEQ ID NOs: 1, 391, 414, 431, 446, 448, 450, 452, 454, 456, 458, 460, and 462. Disclosed herein is a synthetic polypeptide, comprising: an amino acid sequence having at least 99% sequence identity to any one of SEQ ID NOs: 1, 391, 414, 431, 446, 448, 450, 452, 454, 456, 458, 460, and 462. In some embodiments, the amino acid sequence provides a binding constant of at least 1, 2, 3, 4, 5, 6, 7, 8, 9, 10, 11, 12, 13, 14, 15, 16, 17, 18, 19, or 20, or any value therebetween: (i) between the amino acid sequence and a primed nucleic acid sequence; (ii) between the amino acid sequence and a nucleotide; or (iii) both (i) and (ii).

In some embodiments, the synthetic polypeptide is purified or isolated. In some embodiments, the synthetic polypeptide is a polymerizing enzyme. In some embodiments, the polymerizing enzyme comprises a nucleic acid polymerase or polymerizing portion thereof. In some embodiments, the polymerizing enzyme comprises a DNA polymerase or polymerizing portion thereof.

Disclosed herein are compositions comprising a nucleic acid sequence that encodes any of the synthetic polypeptide disclosed herein. Disclosed herein are compositions comprising a DNA sequence that encodes any of the synthetic polypeptide disclosed herein. Disclosed herein are compositions comprising a messenger RNA sequence that encodes any of the synthetic polypeptide disclosed herein. Disclosed herein are vectors comprising a nucleic acid sequence encoding any of the synthetic polypeptide disclosed herein. In some embodiments, the vector may comprise a plasmid, a viral vector, a non-viral vector, a bacterial vector, a yeast vector, a baculovirus vector, a plant vector, or a mammalian vector. In some embodiments, the vector may comprise a DNA vector. Disclosed herein are cells comprising a nucleic acid that encodes any of the synthetic polypeptide disclosed herein. Disclosed herein are cells comprising a vector comprising a nucleic acid sequence encoding any of the synthetic polypeptide disclosed herein. In some embodiments, the cell is transduced with the nucleic acid or vector disclosed herein. In some embodiments, the cell expresses any of the synthetic polypeptide disclosed herein. In some embodiments, the synthetic polypeptide is isolated or extracted from the cell.

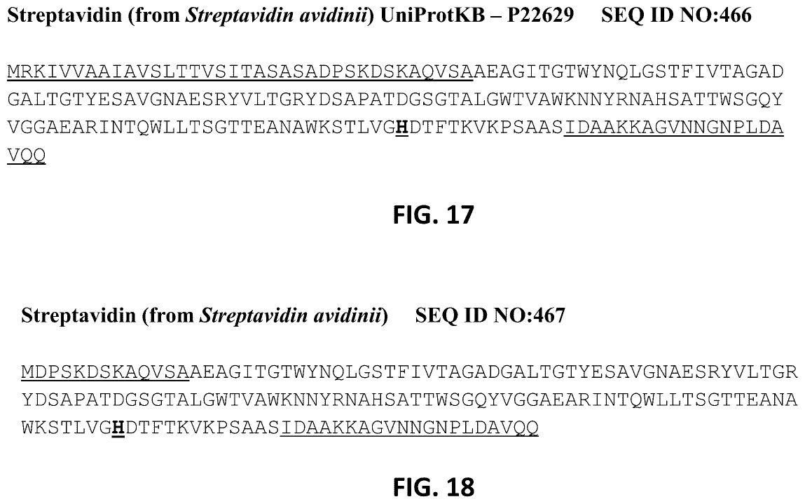

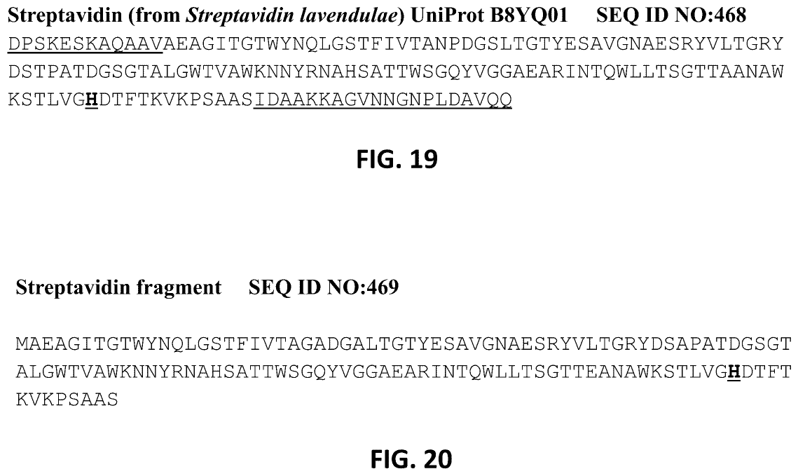

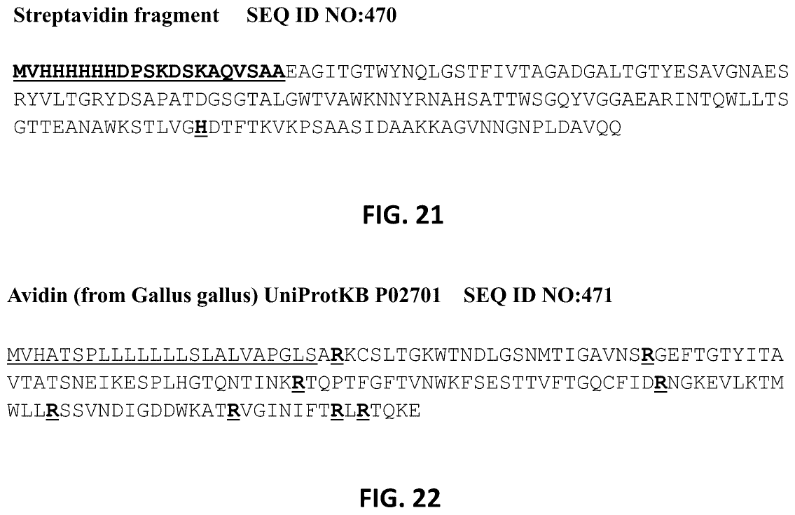

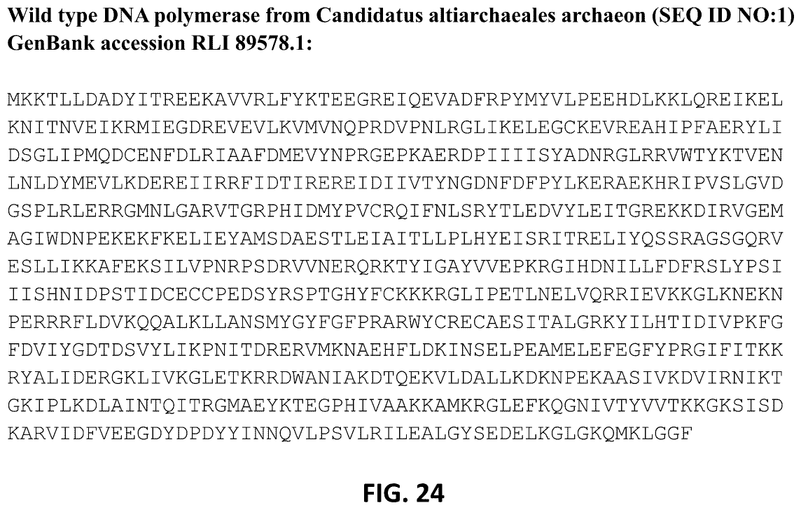

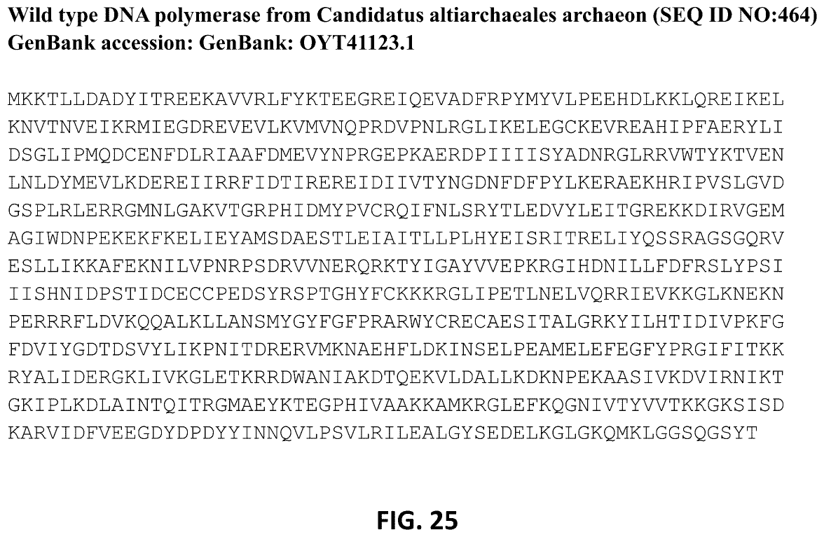

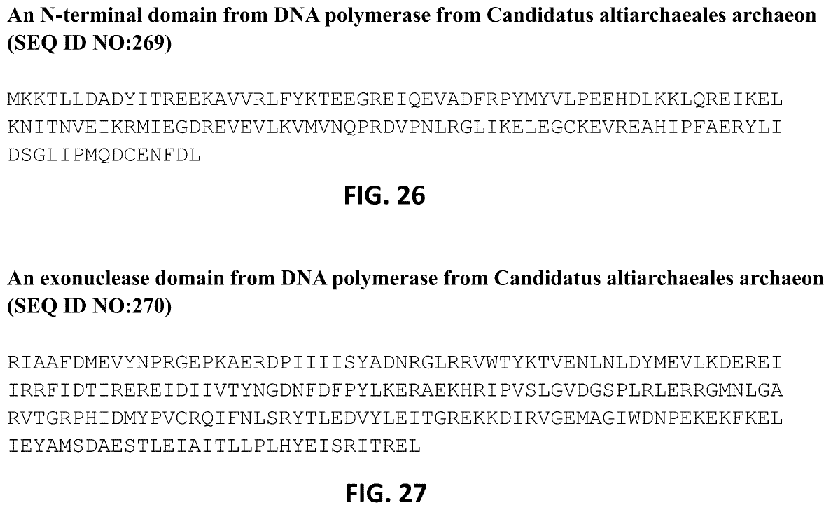

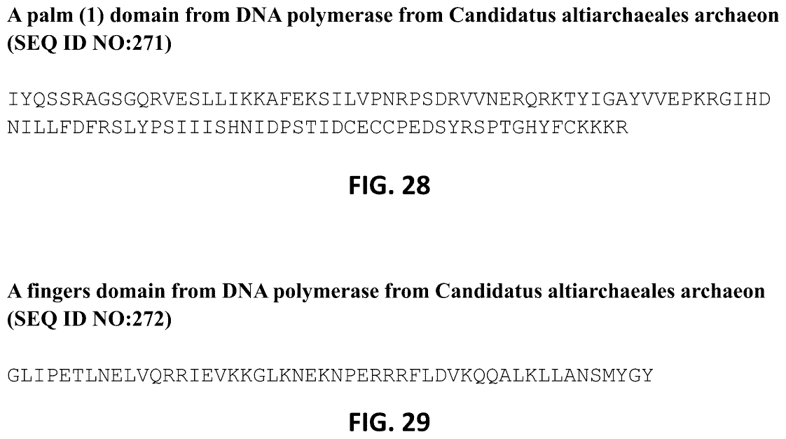

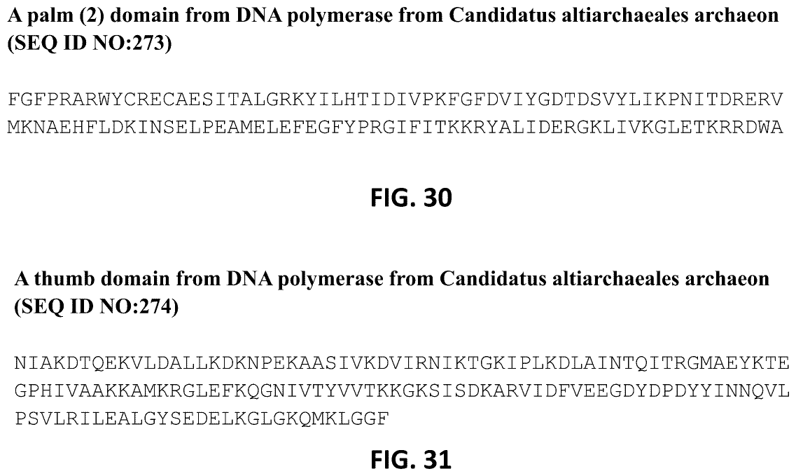

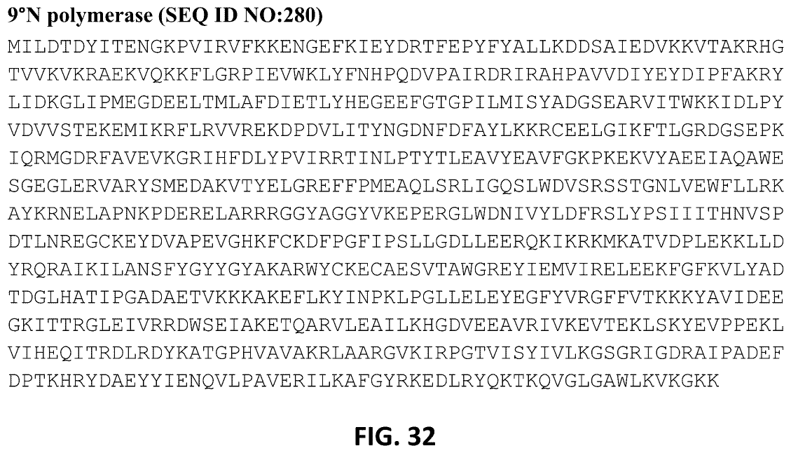

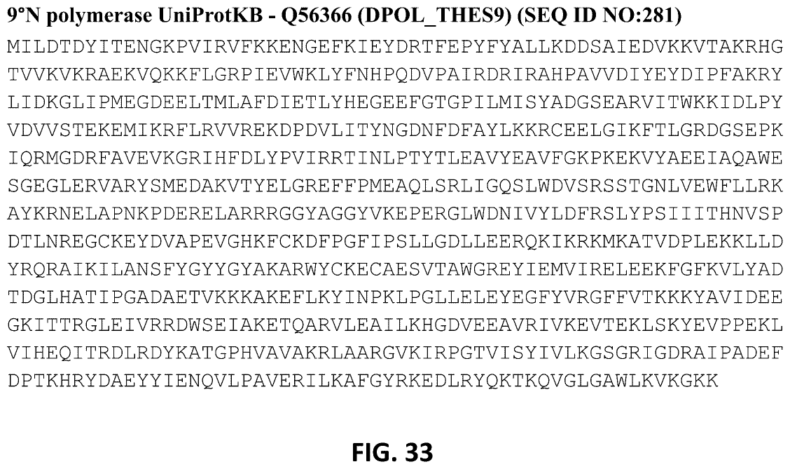

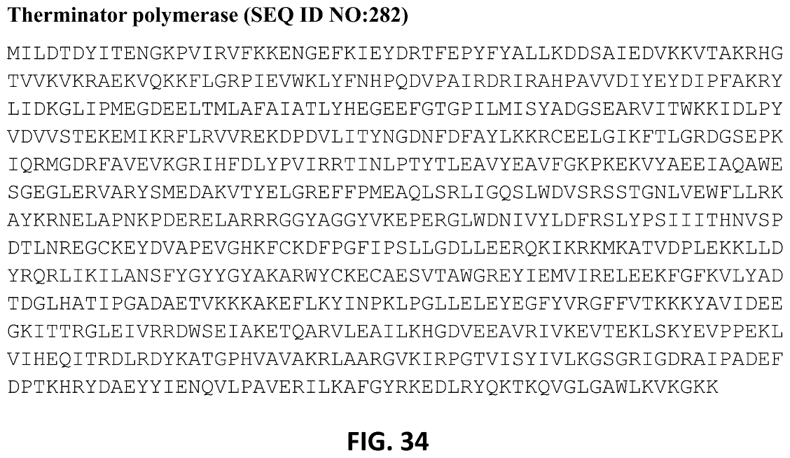

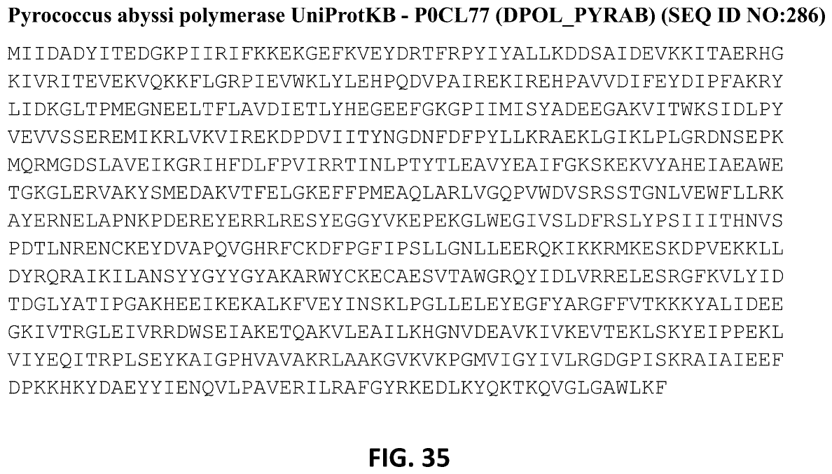

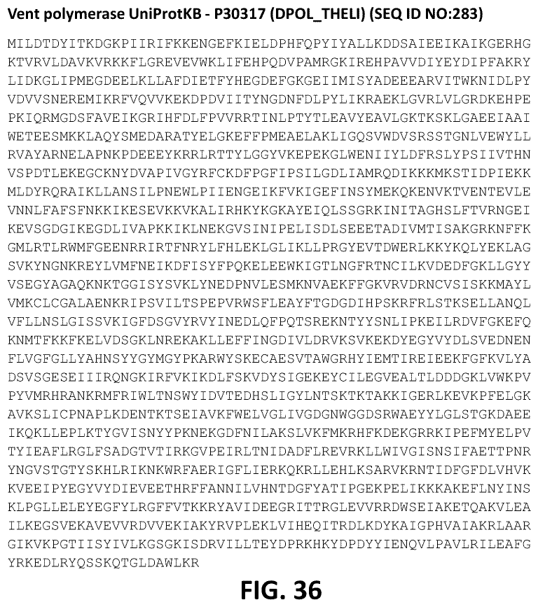

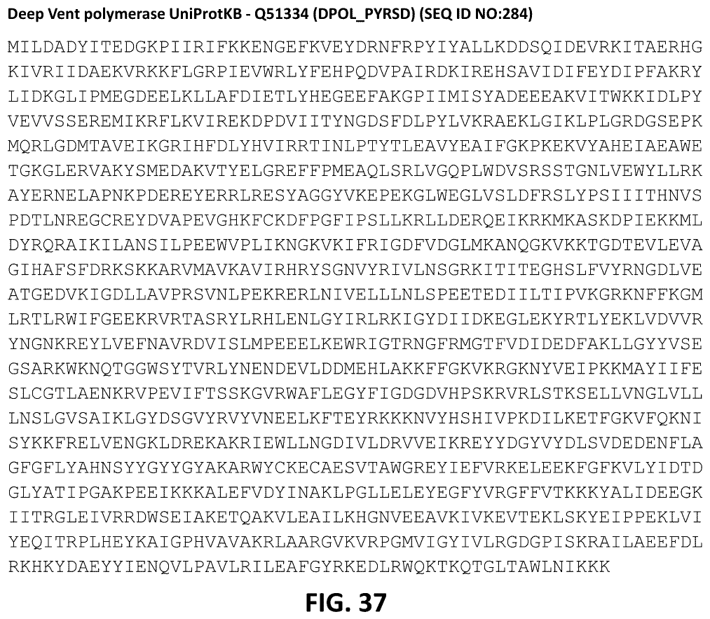

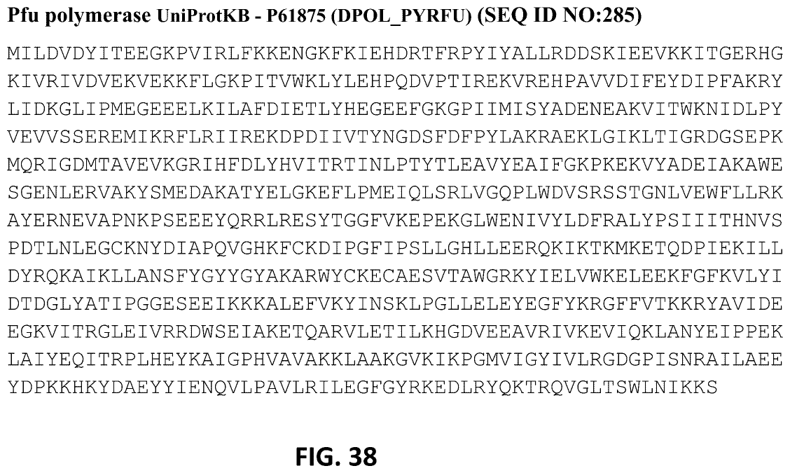

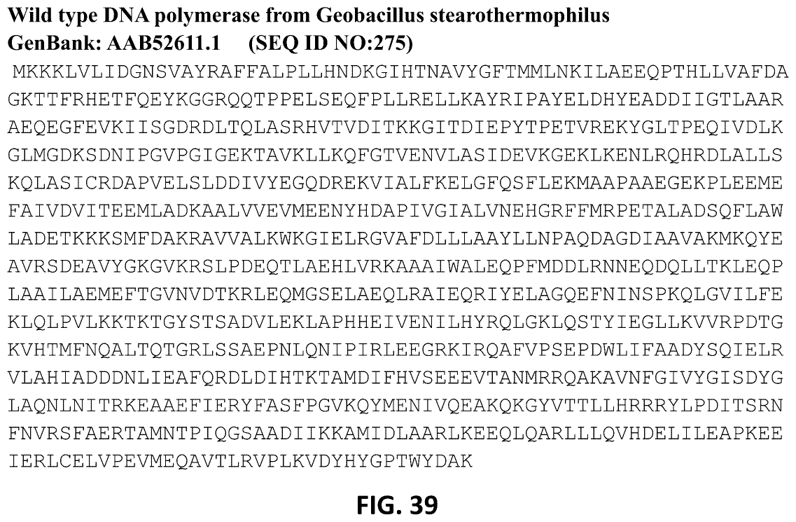

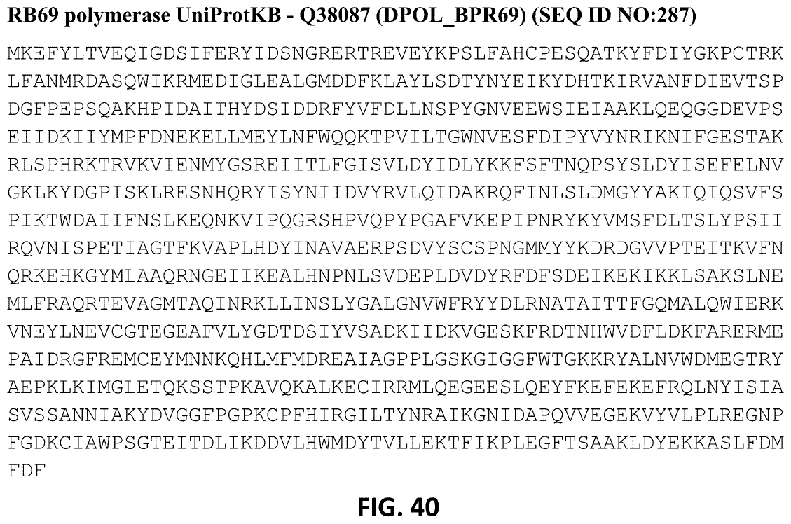

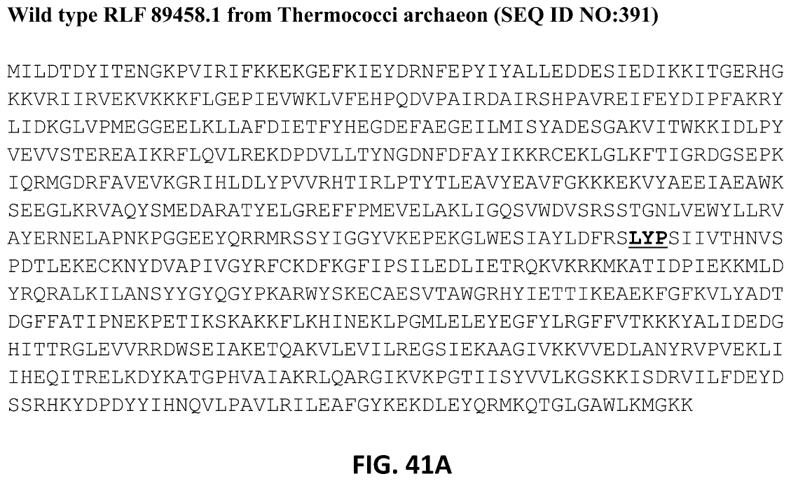

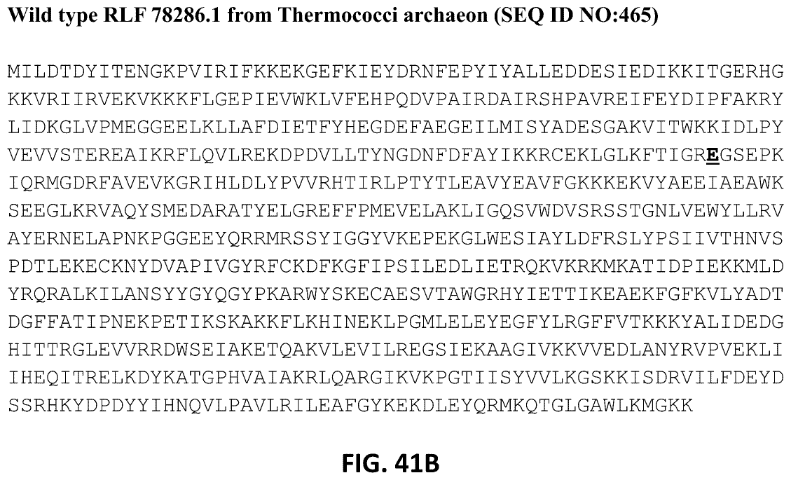

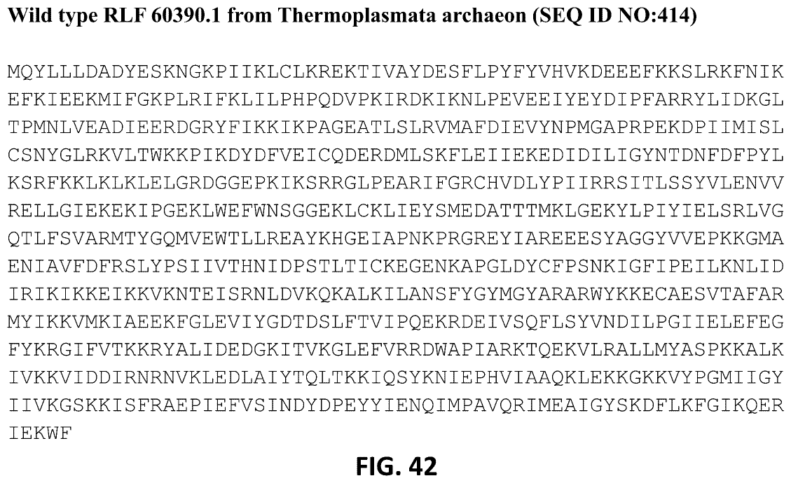

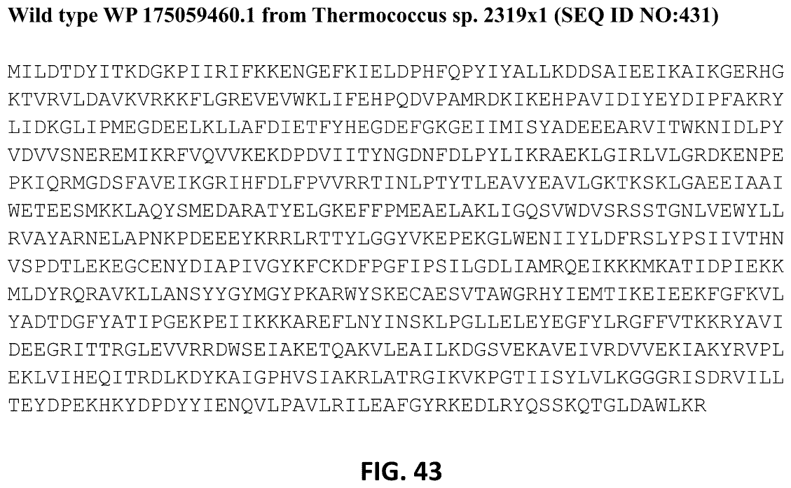

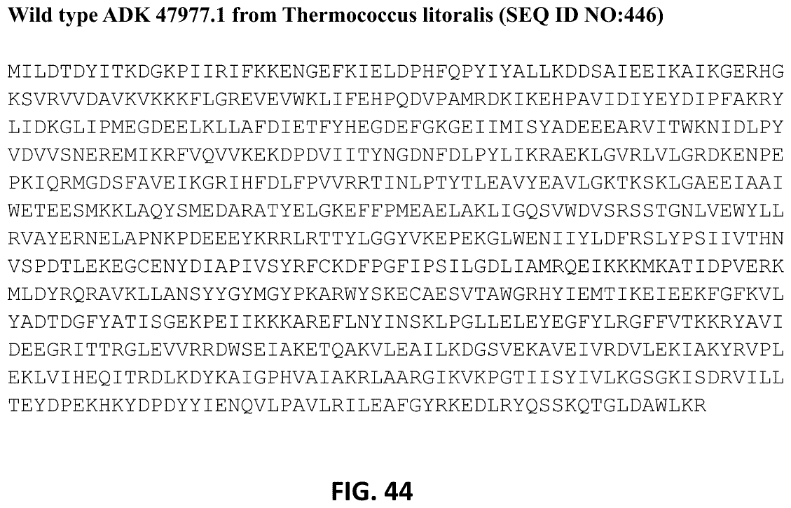

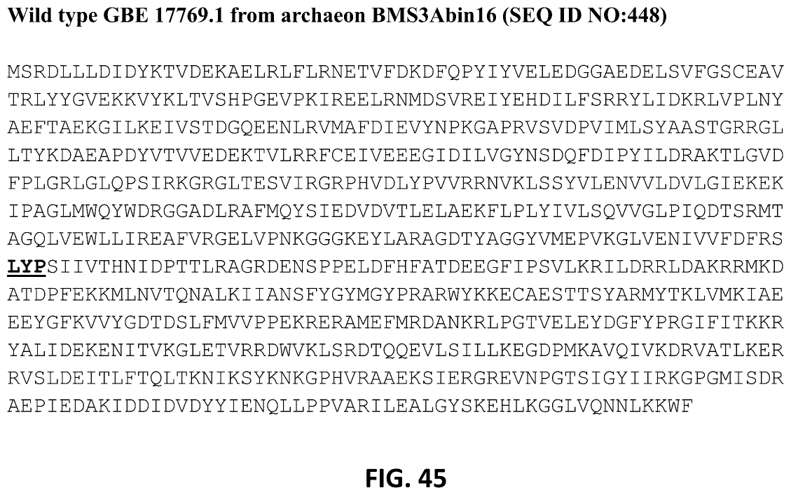

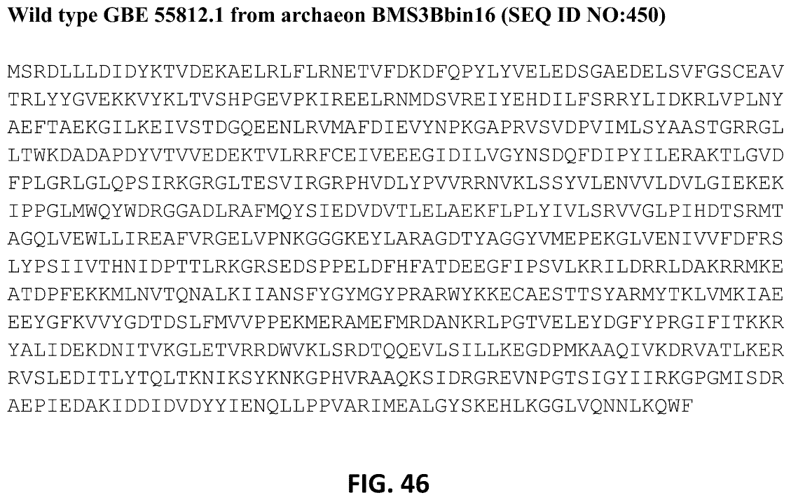

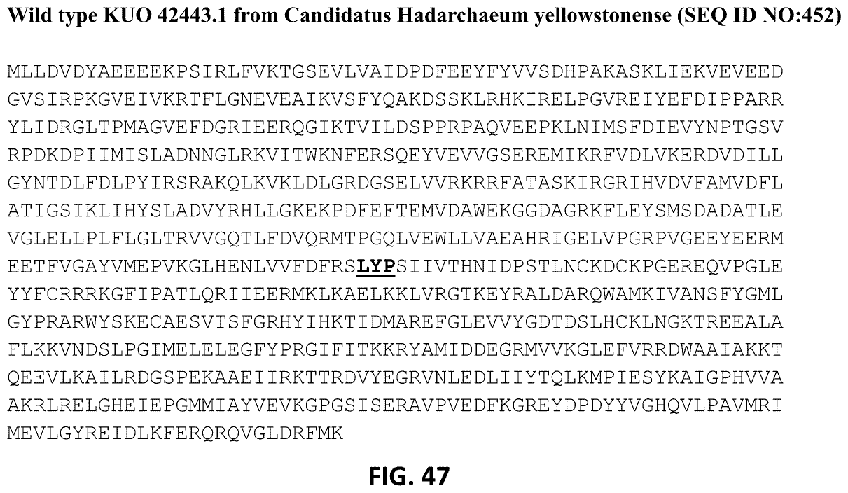

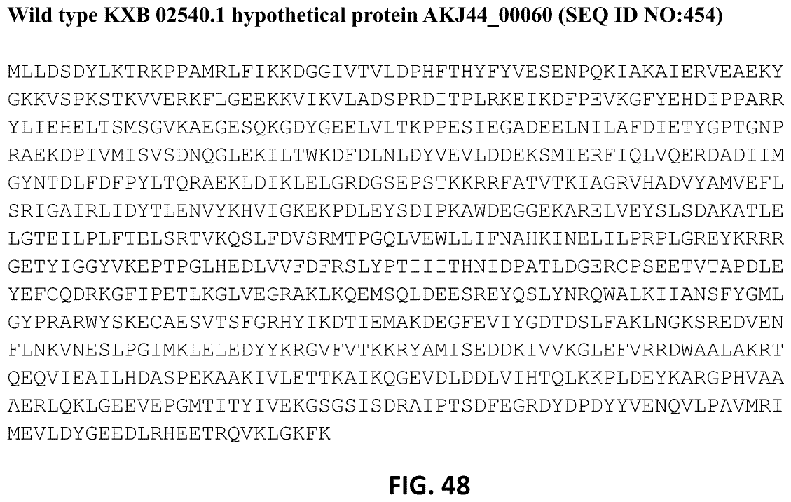

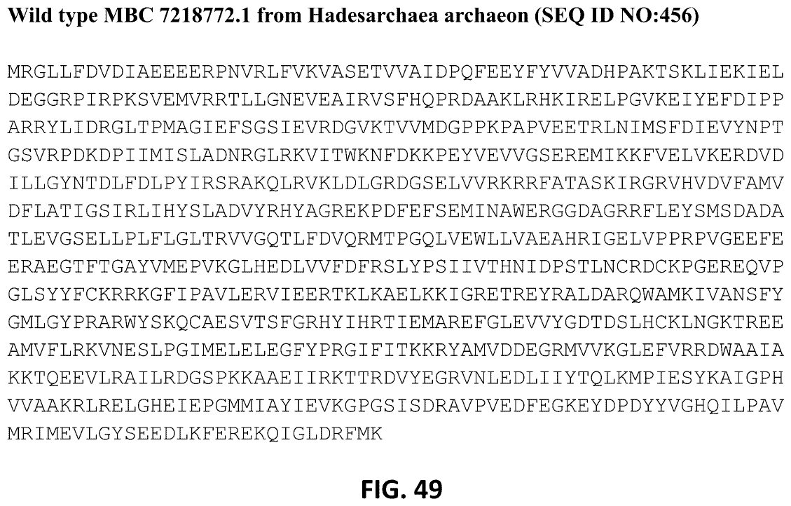

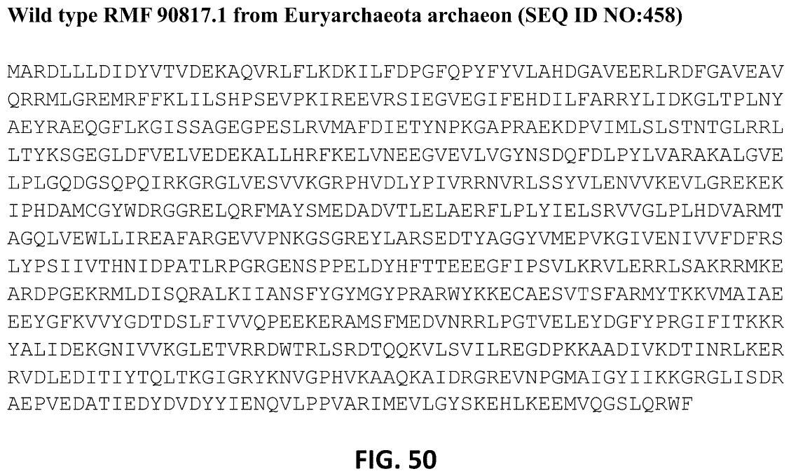

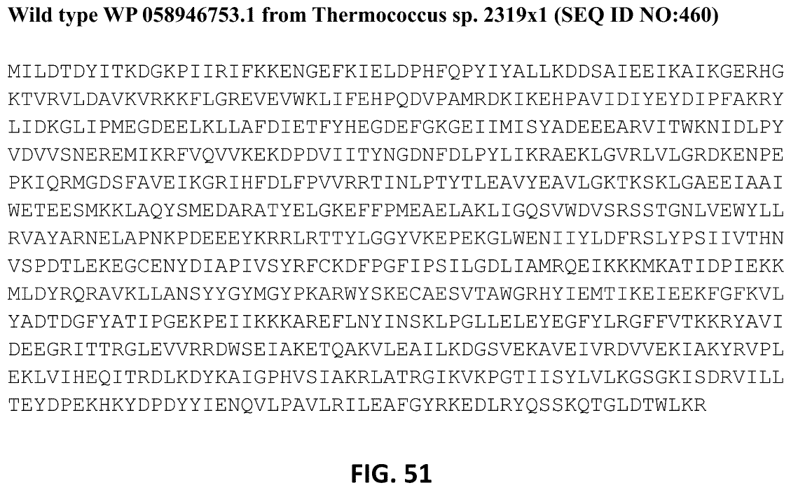

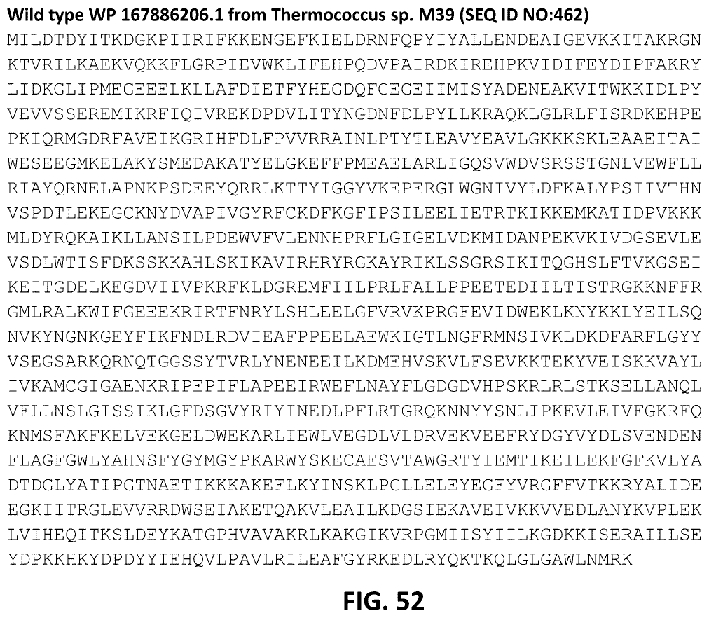

In some embodiments, the synthetic polypeptide is a polymerizing enzyme, such as a polymerase. Non-limiting polymerases of the present disclosure include: Klenow DNA polymerase, Thermus aquaticus DNA polymerase I (Taq polymerase), KlenTaq polymerase, and bacteriophage T7 DNA polymerase; human alpha, delta and epsilon DNA polymerases; bacteriophage polymerases such as T4, and phi29 bacteriophage DNA polymerases; Bacillus subtilis DNA polymerase III, and E. coli DNA polymerase III alpha and epsilon; reverse transcriptases such as HIV type M or O reverse transcriptases, avian myeloblastosis virus reverse transcriptase, or Moloney Murine Leukemia Virus (MMLV) reverse transcriptase, or telomerase. In some embodiments, the polymerase can comprise a Klenow polymerase. In some embodiments, the polymerizing enzyme is a DNA polymerizing enzyme. Non-limiting examples of DNA polymerizing enzymes include DNA polymerases derived from various archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases such as 9 degree N polymerase (e.g., SEQ ID NOS:280-281), Vent® DNA Polymerase (e.g., SEQ ID NO:283), Deep Vent® (e.g., SEQ ID NO:284), Therminator™ (e.g., SEQ ID NO:282), Pyrococcus furiosus DNA polymerase (Pfu polymerase) (e.g., SEQ ID NO:285), RB69 (e.g., SEQ ID NO:287), KOD (Sigma-Aldrich), Pfx, and Tgo polymerases. In some embodiments, the polymerizing enzyme can comprise a wild type or mutant backbone sequence of a polymerase from Candidatus Altiarchaeales archaeon (e.g., SEQ ID NOS:1-268, 288-381), from RLF 89458.1 (e.g., SEQ ID NO:391-413), from RLF 60390.1 (e.g., SEQ ID NO:414-430), from WP 175059460.1 (e.g., SEQ ID NO:431-445), from ADK 47977.1 (e.g., SEQ ID NOS:446-447), from GBE 17769.1 (e.g., SEQ ID NOS:448-449), from GBE 55812.1 (e.g., SEQ ID NOS:450-451), from KUO 42443.1 (e.g., SEQ ID NOS:452-453), from KXB 02540.1 (e.g., SEQ ID NOS:454-455), from MBC 7218772.1 (e.g., SEQ ID NOS:456-457), from RMF 90817.1 (e.g., SEQ ID NOS:458-459), from WP 058946753.1 (e.g., SEQ ID NOS:460-461), or from WP 167886206.1 (e.g., SEQ ID NOS:462-463). In some embodiments, the polymerizing enzyme can comprise an amino acid sequence having at least 80%, at least 85%, at least 90%, at least 95%, at least 99% or higher levels of identity to any of SEQ ID NOS: 1-390, 464, 391-463, or 465. The sequences of wildtype and mutated polymerases are listed and described in Tables 1A, 1B, 2, 3, 4, and 5 and FIGS. 17-52. Table 1A lists a wild-type polymerase having a backbone sequence from RLI 89578.1 (SEQ ID NO:1) and mutant variants (SEQ ID NOS: 2-268, 288-381) comprising amino acid substitutions relative to wild-type RLI 89578.1. Any of the amino acid sequences listed in Table 1A can comprise the substitutions D141A and/or E143A. Table 1B lists various polymerase backbones and their variants (SEQ ID NOS:269-287). Table 2 lists a wild-type polymerase having a backbone sequence from RLF 89458.1 (SEQ ID NO:391) and mutant variants (SEQ ID NOS:392-413) comprising amino acid substitutions relative to wild-type RLF 89458.1. Table 3 lists a wild-type polymerase having a backbone sequence from RLF 60390.1 (SEQ ID NO:414) and mutant variants (SEQ ID NOS:415-430) comprising amino acid substitutions relative to wild-type RLF 60390.1. Table 4 lists a wild-type polymerase having a backbone sequence from WP 175059460.1 (SEQ ID NO:431) and mutant variants (SEQ ID NOS:431-445) comprising amino acid substitutions relative to wild-type WP 175059460.1. Table 5 lists various wild-type and mutant polymerases having backbone sequences from ADK 47977.1 (SEQ ID NOS:446-447), GBE 17769.1 (SEQ ID NOS:448-449), GBE 55812.1 (SEQ ID NOS:450-451), KUO 42443.1 (SEQ ID NOS:452-453), KXB 02540.1 (SEQ ID NOS:454-455), MBC 7218772.1 (SEQ ID NOS:456-457), RMF 90817.1 (SEQ ID NOS:458-459), WP 058946753.1 (SEQ ID NOS:460-461), or WP 167886206.1 (SEQ ID NOS:462-463). In Tables 1A, 1B, 2, 3, 4, and 5, an underscore (“_”) indicates a space between two or more amino acid substitutions.

TABLE 1A
Mutants of Candidatus Altiarchaeales Archaeon Polymerase Backbone

| SEQ | | SEQ | |
|---|---|---|---|
| ID NO: | Mutations relative to SEQ ID NO: 1 | ID NO: | Mutations relative to SEQ ID NO: 1 |
| 1 | Wild Type; no mutation | 22 | L416A |
| 2 | Y502V | 23 | I529V |
| 3 | Y502S | 24 | I529S |
| 4 | Y502Q | 25 | I529H |
| 5 | Y502P | 26 | I529G |
| 6 | Y502H | 27 | I529A |
| 7 | Y502G | 28 | Y417V_P418V |
| 8 | Y502F | 29 | Y417V_P418S |
| 9 | Y502A | 30 | Y417V_P418A |
| 10 | S415V | 31 | Y417T_P418K |
| 11 | S415G | 32 | Y417S_P418S |
| 12 | S415A | 33 | Y417S_P418G |
| 13 | R468V | 34 | Y417S_P418A |
| 14 | R468S | 35 | Y417G_P418V |
| 15 | R468K | 36 | Y417G_P418S |
| 16 | R468H | 37 | Y417G_P418G |
| 17 | R468G | 38 | Y417G_P418C |
| 18 | R468A | 39 | Y417G_P418A |
| 19 | R414S | 40 | Y417A_P418V |
| 20 | L416V | 41 | Y417A_P418S |
| 21 | L416S | 42 | Y417A_P418G |
| 43 | Y417A_P418A | 64 | S415A_L416S |
| 44 | S415V_Y417S | 65 | L416V_P418S |
| 45 | S415V_Y417A | 66 | L416V_P418G |
| 46 | S415V_P418V | 67 | L416S_Y417G |
| 47 | S415V_P418S | 68 | L416S_Y417A |
| 48 | S415V_P418G | 69 | L416S_P418V |
| 49 | S415V_L416V | 70 | L416S_P418G |
| 50 | S415V_L416S | 71 | L416G_Y417G |
| 51 | S415G_Y417V | 72 | L416G_Y417A |
| 52 | S415G_Y417S | 73 | L416G_P418G |
| 53 | S415G_P418V | 74 | L416G_P418A |
| 54 | S415G_P418S | 75 | L416A_Y417S |
| 55 | S415G_P418G | 76 | L416A_P418S |
| 56 | S415G_P418A | 77 | L416A_P418G |
| 57 | S415G_L416V | 78 | L416A_P418A |
| 58 | S415G_L416S | 79 | Y417V_P418V_Y502S |
| 59 | S415G_L416G | 80 | Y417V_P418G_Y502G |
| 60 | S415G_L416A | 81 | Y417V_P418A_Y502R |
| 61 | S415A_Y417G | 82 | Y417S_P418V_Y502R |
| 62 | S415A_Y417A | 83 | Y417S_P418G_Y502S |
| 63 | S415A_P418G | 84 | Y417G_P418G_Y502V |
| 85 | Y417G_P418A_Y502N | 106 | L416A_Y417A_P418G |
| 86 | Y417G_P418A_Y502G | 107 | L416A_Y417A_P418I |
| 87 | Y417A_P418S_Y502R | 108 | L416A_Y417S_P418A |
| 88 | G403A_H405S_D406H | 109 | L416A_Y417S_P418G |
| 89 | A493V_K495V_N499S | 110 | L416A_Y417S_P418S |
| 90 | A493V_K495V_N499G | 111 | L416G_Y417G_P418G |
| 91 | A493V_K495V_N499A | 112 | L416I_Y417A_P418G |
| 92 | A493V_K495S_N499V | 113 | L416I_Y417A_P418S |
| 93 | A493V_K495S_N499S | 114 | L416I_Y417G_P418A |
| 94 | A493V_K495S_N499G | 115 | L416I_Y417I_P418V |
| 95 | A493V_K495Q_N499V | 116 | L416S_Y417A_P418G |
| 96 | A493V_K495Q_N499G | 117 | L416S_Y417G_P418A |
| 97 | A493V_K495Q_N499A | 118 | L416T_Y417A_P418A |
| 98 | A493V_K495G_N499V | 119 | L416T_Y417G_P418A |
| 99 | A493V_K495G_N499S | 120 | L416V_Y417A_P418A |
| 100 | A493V_K495G_N499G | 121 | L416V_Y417G_P418G |
| 101 | A493V_K495G_N499A | 122 | L416I_Y417S_P418S |
| 102 | A493V_K495A_N499G | 123 | L416V_Y417V_P418G |
| 103 | A493V_K495A_N499A | 124 | H405P_A493V_K495G_N499A |
| 104 | A493V_K495S_N499G | 125 | A493V_K495S_N499A_Y502T |
| 105 | L416A_Y417A_P418A | 126 | A493V_K495Q_N499G_F507S |
| 127 | A493V_K495G_N499G_M501I | 148 | L416T_Y417G_P418A_I529H |
| 128 | L416A_Y417A_P418A_I529L | 149 | L416V_Y417A_P418A_I529H |
| 129 | L416A_Y417A_P418A_I529H | 150 | L416V_Y417A_P418G_I529S |
| 130 | L416A_Y417A_P418S_I529H | 151 | L416V_Y417A_P418G_I529T |
| 131 | L416A_Y417G_P418A_I529H | 152 | L416V_Y417A_P418G_I529H |
| 132 | L416A_Y417G_P418G_I529H | 153 | L416V_Y417A_P418S_I529H |
| 133 | L416A_Y417G_P418S_I529H | 154 | L416V_Y417G_P418A_I529H |
| 134 | L416G_Y417T_P418S_I529H | 155 | L416V_Y417G_P418G_I529H |
| 135 | L416I_Y417A_P418A_I529L | 156 | L416V_Y417G_P418S_I529H |
| 136 | L416I_Y417A_P418G_I529F | 157 | L416V_Y417T_P418S_I529H |
| 137 | L416I_Y417A_P418S_I529H | 158 | Y502I |
| 138 | L416I_Y417G_P418A_I529L | 159 | L416A_Y417G_P418A |
| 139 | L416I_Y417S_P418G_I529H | 160 | L416I_Y417A_P418A |
| 140 | L416L_Y417Y_P418P_I529H | 161 | L416V_Y417A_P418G |
| 141 | L416S_Y417A_P418G_I529H | 162 | L416A_Y417A_P418S |
| 142 | L416S_Y417A_P418G_I529F | 163 | L416I_Y417S_P418G |
| 143 | L416S_Y417A_P418T_I529H | 164 | L416V_Y417A_P418S |
| 144 | L416S_Y417G_P418G_I529H | 165 | L416A_Y417I_P418T |
| 145 | L416S_Y417G_P418V_I529H | 166 | L416S_Y417G_P418V |
| 146 | L416T_Y417A_P418A_I529H | 167 | L416S_Y417G_P418G |
| 147 | L416T_Y417A_P418G_I529H | 168 | L416V_Y417G_P418A |
| 169 | L416A_Y417A_P418V | 300 | L416F_Y417A_P418G_A493S_I529H_{—} |
| | | | R515L_N567D_S520G |
| 170 | L416V_Y417T_P418I | 301 | L416F_Y417A_P418G_A493S_I529H_{—} |
| | | | R515L_N567D_M501I |
| 171 | L416V_Y417V_P418A | 302 | L416F_Y417A_P418G_A493S_I529H_{—} |
| | | | R515L_N567D_F539Y |
| 172 | S415V_Y417A_A493V_K495G_N499S | 303 | L416F_Y417A_P418G_A493S_I529H_{—} |
| | | | R515L_N567D_R467C_E581G_T618A |
| 173 | H405P_A493V_K495V_N499A | 304 | L416F_Y417A_P418G_A493S_I529H_{—} |
| | | | R515L_N567D_Y448H_F507S_Y502F_{—} |
| | | | V564I_M565V_E578N |
| 174 | A493V_K495S_N499G_F507S | 305 | L416F_Y417A_P418G_A493S_I529H_{—} |
| | | | R515L_N567D_M501V |
| 175 | L416S_Y417A_P418G_I529H_R608K | 306 | L416F_Y417A_P418G_A493S_I529H_{—} |
| | | | R515L_N567D_I343V |
| 176 | L416S_Y417A_P418G_I529H_K473A | 307 | L416F_Y417A_P418A_A493S_I529H_{—} |
| | | | R515L_N567D_S500G |
| 177 | L416S_Y417A_P418G_I529H_L496A | 308 | L416F_Y417A_P418A_A493S_I529H_{—} |
| | | | R515L_N567D_E516G |
| 178 | L416S_Y417A_P418G_I529H_V489I | 309 | L416F_Y417A_P418G_A493S_I529H_{—} |
| | | | R515L_N567D_G474D |
| 179 | L416S_Y417A_P418G_I529H_R515L | 310 | L416F_Y417A_P418G_A493S_I529H_{—} |
| | | | R515L_N567D_I343V |
| 180 | L416S_Y417A_P418G_I529H_A493S | 311 | L416S_Y417A_P418G_I529H_A493S_{—} |
| | | | R515L_N567D_E102R |
| 181 | L416S_Y417A_P418G_I529H_D622T | 312 | L416S_Y417A_P418G_I529H_A493S_{—} |
| | | | R515L_N567D_E310R |
| 182 | L416S_Y417A_P418G_I529H_Q492R | 313 | L416S_Y417A_P418G_I529H_A493S_{—} |
| | | | R515L_N567D_E434R |
| 183 | L416S_Y417A_P418G_I529H_Q673I | 314 | L416S_Y417A_P418G_I529H_A493S_{—} |
| | | | R515L_N567D_E179R |
| 184 | L416S_Y417A_P418G_I529H_A493S_D622T | 315 | L416S_Y417A_P418G_I529H_A493S_{—} |
| | | | R515L_N567D_E758R |
| 185 | L416S_Y417A_P418G_I529H_A493S_Q492R | 316 | L416S_Y417A_P418G_I529H_A493S_{—} |
| | | | R515L_N567D_E27R |
| 186 | L416S_Y417A_P418G_I529H_K473A_L496A | 317 | L416S_Y417A_P418G_I529H_A493S_{—} |
| | | | R515L_N567D_E291R |
| 187 | L416S_Y417A_P418G_I529H_K473A_Q492R | 318 | L416S_Y417A_P418G_I529H_A493S_{—} |
| | | | R515L_N567D_E730R |
| 188 | L416S_Y417A_P418G_I529H_K473A_R515L | 319 | L416S_Y417A_P418G_I529H_A493S_{—} |
| | | | R515L_N567D_E308R |
| 189 | L416S_Y417A_P418G_I529H_L496A_A493S_Q492R | 320 | L416F_Y417A_P418P_A493S_I529H_{—} |
| | | | R515L_N567D_F507S |
| 190 | L416S_Y417A_P418G_I529H_L496A_A493S_V489I | 321 | L416F_Y417A_P418G_A493S_I529H_{—} |
| | | | R515L_N567D_E116G |
| 191 | L416S_Y417A_P418G_I529H_L496A_R515L | 322 | L416F_Y417A_P418G_A493S_I529H_{—} |
| | | | R515L_N567D_M142L |
| 192 | L416S_Y417A_P418G_I529H_L496A_V489I | 323 | L416F_Y417A_P418G_I529H_A493S_{—} |
| | | | R515L_N567D_E434R |
| 193 | L416S_Y417A_P418G_I529H_Q492R_P335Q | 324 | L416F_Y417A_P418G_A493S_I529H_{—} |
| | | | R515L_N567D_P45S_K58R |
| 194 | L416S_Y417A_P418G_I529H_Q492R_Q673I | 325 | L416F_Y417A_P418G_A493S_I529H_{—} |
| | | | R515L_N567D_M41L |
| 195 | L416S_Y417A_P418G_I529H_R515L_Q492R | 326 | L416F_Y417A_P418G_A493S_I529H_{—} |
| | | | R515L_N567D_I11F_K105R |
| 196 | L416S_Y417A_P418G_I529H_R515L_Q673I | 327 | L416F_Y417A_P418G_A493S_I529H_{—} |
| | | | R515L_N567D_N132S |
| 197 | L416S_Y417A_P418G_I529H_R608K_A493S | 328 | L416F_Y417A_P418G_A493S_I529H_{—} |
| | | | R515L_N567D_R170H |
| 198 | L416S_Y417A_P418G_I529H_R608K_L496A | 329 | L416F_Y417A_P418G_A493S_I529H_{—} |
| | | | R515L_N567D_I63V_E77G_Q87H |
| 199 | L416S_Y417A_P418G_I529H_R608K_Q492R | 330 | L416F_Y417A_P418G_A493S_I529H_{—} |
| | | | R515L_N567D_E308R |
| 200 | L416S_Y417A_P418G_I529H_V4891_A493S | 331 | L416F_Y417A_P418G_A493S_I529H_{—} |
| | | | R515L_N567D_M84L |
| 201 | L416S_Y417A_P418G_I529H_V489I_Q492R | 332 | L416F_Y417A_P418G_A493S_I529H_{—} |
| | | | R515L_N567D_Q54L_P126S |
| 202 | L416S_Y417A_P418G_I529H_V489I_R515L | 333 | L416F_Y417A_P418G_A493S_I529H_{—} |
| | | | R515L_N567D_L124Q_F37S_E14G |
| 203 | L416S_Y417A_P418G_I529H_A493S_R515L | 334 | L416F_Y417A_P418G_A493S_I529H_{—} |
| | | | R515L_N567D_K51R |
| 204 | L416S_Y417A_P418G_A493S_I529H_K610E | 335 | L416F_Y417A_P418G_A493S_I529H_{—} |
| | | | R515L_N567D_V107I |
| 205 | L416S_Y417A_P418G_I529H_A493S_L496G | 336 | L416F_Y417A_P418G_A493S_I529H_{—} |
| | | | R515L_N567D_K58M |
| 206 | L416S_Y417A_P418G_I529H_A493S_R515L_L611S | 337 | L416F_Y417A_P418G_A493S_I529H_{—} |
| | | | R515L_N567D_E73K |
| 207 | L416S_Y417A_P418G_I529H_A493S_L496S_D439S | 338 | L416F_Y417A_P418G_A493S_I529H_{—} |
| | | | R515L_N567D_K61E |
| 208 | L416S_Y417A_P418G_I529H_A493S_R515W | 339 | L416F_Y417A_P418G_A493S_I529H_{—} |
| | | | R515L_N567D_E14G_I68V |
| 209 | L416S_Y417A_P418G_I529H_A493S_L496A | 340 | L416F_Y417A_P418G_A493S_I529H_{—} |
| | | | R515L_N567D_V239I_R256H |
| 210 | L416S_Y417A_P418G_I529H_A493S_Q492C_L496R | 341 | L416F_Y417A_P418G_A493S_I529H_{—} |
| | | | R515L_N567D_G96S |
| 211 | L416S_Y417A_P418G_I529H_A493S_R723H | 342 | L416F_Y417A_P418G_A493S_I529H_{—} |
| | | | R515L_N567D_E205K_K225E |
| 212 | L416S_Y417A_P418G_I529H_A493S_D653G_A669D_S717G_I750V | 343 | L416F_Y417A_P418G_A493S_I529H_{—} |
| | | | R515L_N567D_T344I |
| 213 | L416S_Y417A_P418G_I529H_A493S_L496S | 344 | L416F_Y417A_P418G_A493S_I529H_{—} |
| | | | R515L_N567D_L50P |
| 214 | L416S_Y417A_P418G_I529H_A493S_R515Y | 345 | L416F_Y417A_P418G_A493S_I529H_{—} |
| | | | R515L_N567D_D9N |
| 215 | L416S_Y417A_P418G_I529H_A493S_Q492G_L496A | 346 | L416F_Y417A_P418G_A493S_I529H_{—} |
| | | | R515L_N567D_I63T |
| 216 | L416S_Y417A_P418G_I529H_A493S_S443N_Q492F | 347 | L416F_Y417A_P418G_A493S_I529H_{—} |
| | | | R515L_N567D_I272V |
| 217 | L416S_Y417A_P418G_I529H_A493S_R697G | 348 | L416F_Y417A_P418G_A493S_I529H_{—} |
| | | | R515L_N567D_D191G |
| 218 | L416S_Y417A_P418G_I529H_A493S_L496H | 349 | L416F_Y417A_P418G_A493S_I529H_{—} |
| | | | R515L_N567D_E14K |
| 219 | L416S_Y417A_P418G_I529H_A493S_Q492A_L496S | 350 | L416F_Y417A_P418G_A493S_I529H_{—} |
| | | | R515L_N567D_E116G |
| 220 | L416S_Y417A_P418G_I529H_A493S_L494V_L496N | 351 | L416F_Y417A_P418G_A493S_I529H_{—} |
| | | | R515L_N567D_A115V |
| 221 | L416S_Y417A_P418G_I529H_A493S_V651M | 352 | L416F_Y417A_P418G_A493S_I529H_{—} |
| | | | R515L_N567D_D75N |
| 222 | L416S_Y417A_P418G_A493S_I529H_E569G | 353 | L416F_Y417A_P418G_A493S_I529H_{—} |
| | | | R515L_N567D_Q767* |
| 223 | L416S_Y417A_P418G_I529H_A493S_R515L_S717G | 354 | L416F_Y417A_P418G_A493S_I529H_{—} |
| | | | R515L_N567D_K762R_G763V_L764W_{—} |
| | | | G765A_K766S_Q767K_M768* |
| 224 | L416S_Y417A_P418G_I529H_A493S_S717G | 355 | L416F_Y417A_P418G_A493S_I529H_{—} |
| | | | R515L_N567D_A568V |
| 225 | L416S_Y417A_P418G_I529H_A493S_R515L_N567D | 356 | L416F_Y417A_P418G_A493S_I529H_{—} |
| | | | R515L_N567D_M501I_G763S |
| 226 | L416S_Y417A_P418G_I529H_A493S_L496R | 357 | L416F_Y417A_P418G_A493S_I529H_{—} |
| | | | R515L_N567D_M501I_S717N_S746C |
| 227 | L416S_Y417A_P418G_I529H_A493S_Q492G | 358 | L416F_Y417A_P418G_A493S_I529H_{—} |
| | | | R515L_N567D_M501I |
| 228 | L416S_Y417A_P418G_I529H_A493S_R515L_S577I | 359 | L416F_Y417A_P418G_A493S_I529H_{—} |
| | | | R515L_N567D_M501I_T675A_{—} |
| | | | K682I_N705D |
| 229 | L416S_Y417A_P418G_A493S_I529H_K58M | 360 | L416F_Y417A_P418G_A493S_I529H_{—} |
| | | | R515L_N567D_M501I_N705D |
| 230 | L416S_Y417A_P418G_I529H_A493S_R515P | 361 | L416F_Y417A_P418G_A493S_I529H_{—} |
| | | | R515L_N567D_E14K_H48R_D75N_{—} |
| | | | A115V_M501I_N705D |
| 231 | L416S_Y417A_P418G_I529H_A493S_Q492G_L496I | 362 | L416F_Y417A_P418G_A493S_I529H_{—} |
| | | | R515L_N567D_M501I_N705D |
| 232 | L416S_Y417A_P418G_I529H_A493S_L496N | 363 | L416F_Y417A_P418G_A493S_I529H_{—} |
| | | | R515L_N567D_A115V |
| 233 | L416S_Y417A_P418G_I529H_A493S_R515F | 364 | L416F_Y417A_P418G_A493S_I529H_{—} |
| | | | R515L_N567D_D305N_E328Q |
| 234 | L416S_Y417A_P418G_I529H_A493S_D381Y_Q492R_L496M | 365 | L416F_Y417A_P418G_A493S_I529H_{—} |
| | | | R515L_N567D_M501I_N705D |
| 235 | L416S_Y417A_P418G_I529H_A493S_S443N | 366 | L416F_Y417A_P418G_A493S_I529H_{—} |
| | | | R515L_N567D_E14K_H48R_D75N_{—} |
| | | | A115V_M501I |
| 236 | L416S_Y417A_P418G_I529H_A493S_E370D | 367 | L416F_Y417A_P418G_A493S_I529H_{—} |
| | | | R515L_N567D_A568V |
| 237 | L416S_Y417A_P418G_I529H_A493S_E760G | 368 | L416F_Y417A_P418G_A493S_I529H_{—} |
| | | | R515L_N567D_M501I |
| 238 | L416S_Y417A_P418G_I529H_A493S_Q492T_L496S | 369 | L416F_Y417A_P418G_A493S_I529H_{—} |
| | | | R515L_N567D_E434R |
| 239 | L416S_Y417A_P418G_I529H_A493S_L496C | 370 | L416F_Y417A_P418G_A493S_I529H_{—} |
| | | | R515L_N567D_M41L |
| 240 | L416S_Y417A_P418G_I529H_A493S_L496I | 371 | L416F_Y417A_P418G_A493S_I529H_{—} |
| | | | R515L_N567D_M501I_M41L |
| 241 | L416S_Y417A_P418G_I529H_A493S_L496Y | 372 | L416F_Y417A_P418G_A493S_I529H_{—} |
| | | | R515L_N567D_M501I_E434R |
| 242 | L416S_Y417A_P418G_I529H_A493S_G355S_S440N_R515L | 373 | L416F_Y417A_P418G_A493S_I529H_{—} |
| | | | R515L_N567D_H48R |
| 243 | L416S_Y417A_P418G_I529H_A493S_Q492L_L496M | 374 | L416F_Y417A_P418G_A493S_M501I_{—} |
| | | | R515L_I529H_N567D_N657D_E700K_{—} |
| | | | N705S |
| 244 | L416F_Y417A_P418G_A493S_I529H_R515L_N567D | 375 | H48R_D75N_A115V_L416F_Y417A_{—} |
| | | | P418G_A493S_M501I_R515L_I529H_{—} |
| | | | N567D_N705D |
| 245 | L416Y_Y417A_P418G_A493S_I529H_R515L_N567D | 376 | D141A_E143A_L416F_Y417A_P418G_{—} |
| | | | A493S_R515L_I529H_N567D |
| 246 | L416S_Y417T_P418G_I529H_A493S_R515L_N567D | 377 | D141A_E143A_C104V_C130V_C269V_{—} |
| | | | L416F_Y417A_P418G_A493S_R515L_{—} |
| | | | 1529H_N567D_M768* |
| 247 | L416M_Y417A_P418G_A493S_I529H_R515L_N567D | 378 | E14K_H48R_D75N_C104V_K105R_{—} |
| | | | A115V_D129N_C130V_D141A_E143A_{—} |
| | | | C269V_F369I_N385S_L416F_Y417A_{—} |
| | | | P418G_A493S_M501I_R515L_I529H_{—} |
| | | | N567D_N705D |
| 248 | L416A_Y417A_P418G_I529H_A493S_R515L_N567D | 379 | E14K_H48R_D75N_C104V_A115V_{—} |
| | | | D129N_C130V_D141A_E143A_C269V_{—} |
| | | | F369I_N385S_L416F_Y417A_P418G_{—} |
| | | | A493S_M501I_R515L_I529H_N567D_{—} |
| | | | N705D |
| 249 | L416S_Y417A_P418G_I529H_A493S_R515L_N567D_C104S | 380 | E14K_H48R_D75N_C104V_A115V_{—} |
| | | | C130V_D141A_E143A_C269V_F369I_{—} |
| | | | N385S_L416S_Y417A_P418G_A493S_{—} |
| | | | M501I_R515L_I529H_N567D_N705D |
| 250 | L416S_Y417A_P418G_I529H_A493S_R515L_N567D_C514S | 381 | Y10F_E14K_H48R_D75N_C104V_{—} |
| | | | A115V_C130V_D141A_E143A_L416F_{—} |
| | | | Y417A_P418G_R515L_I529H_N567D_{—} |
| | | | E680D_N705D |
| 251 | L416S_Y417A_P418G_I529H_A493S_R515L_N567D_C130S | 288 | L416F_Y417A_P418G_A493S_I529H_{—} |
| | | | R515L_N567D_A498G |
| 252 | L416S_Y417A_P418G_I529H_A493S_R515L_N567D_C130R | 289 | L416F_Y417A_P418G_A493S_I529H_{—} |
| | | | R515L_N567D_D488N_G474S |
| 253 | L416S_Y417A_P418G_I529H_A493S_R515L_N567D_C450S | 290 | L416F_Y417A_P418G_A493S_I529H_{—} |
| | | | R515L_N567D_V465M |
| 254 | L416S_Y417A_P418G_I529H_A493S_R515L_N567D_C517S | 291 | L416F_Y417A_P418G_A493S_I529H_{—} |
| | | | R515L_N567D_D573N |
| 255 | L416S_Y417A_P418G_I529H_A493S_R515L_N567D_C269S | 292 | L416F_Y417A_P418G_A493S_I529H_{—} |
| | | | R515L_N567D_K538R |
| 256 | D141A_E143A_L416S_Y417A_P418G_I529H | 293 | L416F_Y417A_P418G_A493S_I529H_{—} |
| | | | R515L_N567D_R483H_K574R |
| 257 | D141A_E143A_L416I_Y417A_P418A_I529L | 294 | L416F_Y417A_P418G_A493S_I529H_{—} |
| | | | R515L_N567D_N480I_T618A |
| 258 | D141A_E143A_L416A_Y417A_P418S_I529H | 295 | L416F_Y417A_P418G_A493S_I529H_{—} |
| | | | R515L_N567D_R509H_D560G |
| 259 | D141A_E143A_L416V_Y417A_P418A_I529H | 296 | L416F_Y417A_P418G_A493S_I529H_{—} |
| | | | R515L_N567D_D636G |
| 260 | D141A_E143A_L416A_Y417A_P418A_I529H | 297 | L416F_Y417A_P418G_A493S_I529H_{—} |
| | | | R515L_N567D_D560E |
| 261 | D141A_E143A_L416V_Y417A_P418G_I529T | 298 | L416F_Y417A_P418G_A493S_I529H_{—} |
| | | | R515L_N567D_R509H_S520N_M583K |
| 262 | D141A_E143A_L416G_Y417T_P418S_I529H | 299 | L416F_Y417A_P418G_A493S_I529H_{—} |
| | | | R515L_N567D_S500G |
| 263 | D141A_E143A_L416V_Y417A_P418G_I529H | 266 | D141A_E143A_L416A_Y417A_P418A |
| 264 | D141A_E143A_L416I_Y417A_P418S_I529H | 267 | D141A_E143A_L416A_Y417S |
| 265 | D141A_E143A_L416A_Y417G_P418A_I529H | 268 | D141A_E143A |

*= An amino acid deletion starts from the star (*) labeled codon to the stop codon.

TABLE 1B
Polymerase of Various Backbone Sequences

| SEQ ID NO: | Description |
|---|---|
| 269 | N-terminal domain from SEQ ID NO: 1 |
| 270 | 3′ to 5 exonuclease domain from SEQ ID NO: 1 |
| 271 | Palm (1) domain from SEQ ID NO: 1 |
| 272 | Fingers domain from SEQ ID NO: 1 |
| 273 | Palm (2) domain from SEQ ID NO: 1 |
| 274 | Thumb domain from SEQ ID NO: 1 |
| 275 | Geobacillus (WT) |
| 276 | Geobacillus (Q656S) |
| 277 | Geobacillus (Q656A) |
| 278 | Geobacillus (Q656A_I657A_E658A) |
| 279 | Geobacillus (Q656S_I657A_E658A) |
| 280 | 9° N polymerase |
| 281 | 9° N polymerase (UniProtKB - Q56366) |
| 282 | Therminator ® polymerase |
| 283 | Vent ® polymerase |
| 284 | Deep Vent ® polymerase |
| 285 | Pfu polymerase |
| 286 | Pyrococcus abyssi polymerase |
| 287 | RB69 polymerase |

TABLE 2
Mutants of RLF 89458.1 Polymerase backbone

| SEQ ID | Mutations based on | SEQ ID | Mutations based on |
|---|---|---|---|
| NO: | SEQ ID NO: 391 | NO: | SEQ ID NO: 391 |
| 391 | Wild type RLF 89458.1 | 405 | D141A_D143A_Y261A_L409A_Y410A_P411A_A485S |
| 392 | Y410A_D141A_D143A | 406 | D141A_D143A_Y261A_L409S_Y410A_P411G_A485S |
| 393 | D141A_D143A_L409S_Y410A_Y261A | 407 | D141A_D143A_Y261A_L409S_Y410A_A485S |
| 394 | D141A_D143A_L409S_Y410A_P411G_Y261A | 408 | D141A_D143A_Y261A_L409S_Y410G_A485S |
| 395 | D141A_D143A_Y261A_F406I_L409S_Y410A_P411A | 409 | D141A_D143A_Y261A_L409A_Y410A_P411A_I521H |
| 396 | Y7A_D141A_D143A_Y261A_Y410A | 410 | D141A_D143A_Y261A_L409S_Y410A_P411G_I521H |
| 397 | D141A_D143A_Y261A_Y410A_Y493I | 411 | D141A_D143A_Y261A_L409S_Y410A_I521H |
| 398 | D141A_D143A_Y261A_Y410A_Y493T | 412 | D141A_D143A_Y261A_L409S_Y410G_I521H |
| 399 | D141A_D143A_Y261A_Y410A_V513I | 413 | D141A_D143A_Y261A_L409S_Y410A_A485S_K507L_I521H |
| 400 | Y7A_D141A_D143A_Y261A_L409S_Y410A | 403 | D141A_D143A_Y261A_L409S_Y410A |
| 401 | D141A_D143A_Y261A_L409A_Y410A_P411A | 404 | D141A_D143A_Y261A_L409S_Y410G |
| 402 | D141A_D143A_Y261A_L409S_Y410A_P411G | | |

TABLE 3
Mutants of RLF 60390.1 Polymerase Backbone

| SEQ ID NO: | Mutations based on SEQ ID NO: 414 |
|---|---|
| 414 | Wild type RLF 60390.1 |
| 415 | D158A_E160A_L431A_Y432A_P433I_A507S |
| 416 | D158A_E160A_L431A_Y432A_P433I_K506Q |
| 417 | D158A_E160A_L431A_Y432A_P433I_K506Q_A507S |
| 418 | D158A_E160A_L431A_Y432A_P433A |
| 419 | D158A_E160A_L431A_Y432A_P433A_A507S |
| 420 | D158A_E160A_L431A_Y432A_P433A_I543H |
| 421 | D158A_E160A_L431S_Y432A_P433G |
| 422 | D158A_E160A_L431S_Y432A_P433G_A507S |
| 423 | D158A_E160A_L431S_Y432A_P433G_I543H |
| 424 | D158A_E160A_L431S_Y432A |
| 425 | D158A_E160A_L431S_Y432A_A507S |
| 426 | D158A_E160A_L431S_Y432A_A507S_K529L_I543H |
| 427 | D158A_E160A_L431S_Y432A_I543H |
| 428 | D158A_E160A_L431S_Y432G |
| 429 | D158A_E160A_L431S_Y432G_A507S |
| 430 | D158A_E160A_L431S_Y432G_I543H |

TABLE 4
Mutants of WP 175059460.1 Polymerase Backbone

| SEQ ID NO: | Mutations based on SEQ ID NO: 431 |
|---|---|
| 431 | Wild type WP 175059460.1 |
| 432 | D141A_E143A_Y412A |
| 433 | D141A_E143A_L411A_Y412A_P413A |
| 434 | D141A_E143A_L411A_Y412A_P413A_A488S |
| 435 | D141A_E143A_L411A_Y412A_P413A_I524H |
| 436 | D141A_E143A_L411S_Y412A_P413G |
| 437 | D141A_E143A_L411S_Y412A_P413G_A488S |
| 438 | D141A_E143A_L411S_Y412A_P413G_I524H |
| 439 | D141A_E143A_L411S_Y412A |
| 440 | D141A_E143A_L411S_Y412A_A488S |
| 441 | D141A_E143A_L411S_Y412A_A488S_K510L_I524H |
| 442 | D141A_E143A_L411S_Y412A_I524H |
| 443 | D141A_E143A_L411S_Y412G |
| 444 | D141A_E143A_L411S_Y412G_A488S |
| 445 | D141A_E143A_L411S_Y412G_I524H |

TABLE 5
Mutants of Other Polymerase Backbones

| SEQ ID NO: | Description or Mutations |
|---|---|
| 446 | Wild type ADK47977.1 |
| 447 | Mutant ADK47977.1 |
| | D141A_E143A_Y412A |
| 448 | Wild type GBE17769.1 |
| 449 | Mutant GBE17769.1 |
| | D149A_E151A_Y272A_Y422A |
| 450 | Wild type GBE55812.1 |
| 451 | Mutant GBE55812.1 |
| | D149A_E151A_Y272A_Y422A |
| 452 | Wild type KUO42443.1 |
| 453 | Mutant KUO42443.1 |
| | D170A_E172A_Y449A |
| 454 | Wild type KXB02540.1 |
| 455 | Mutant KXB02540.1 |
| | D170A_E172A_Y449A |
| 456 | Wild type MBC7218772.1 |
| 457 | Mutant MBC7218772.1 |
| | D173A_E175A_Y452A_C468A |
| 458 | Wild type RMF90817.1 |
| 459 | Mutant RMF90817.1 |
| | D149A_E151A_Y272A_Y422A |
| 460 | Wild type WP058946753.1 |
| 461 | Mutant WP058946753.1 |
| | D141A_E143A_Y412A |
| 462 | Wild type WP167886206.1 |
| 463 | Mutant WP167886206.1 |
| | D141A_E143A_Y412A and [deletion: I496 - S1033] |

### Nucleotide Conjugates

Disclosed herein, in some embodiments, are compositions comprising a nucleotide conjugate. The nucleotide conjugates of the present disclosure may be useful for forming a binding complex disclosed herein, such as in a trapping reaction, a nucleotide binding reaction or a nucleic acid sequencing reaction disclosed herein. In some embodiments, the nucleotide conjugate may comprise a nucleotide and a moiety. In some embodiments, the moiety may be coupled, bound, attached, or linked to the nucleotide through a covalent or noncovalent bond. In some embodiments, the moiety may comprise a chemical or a biological moiety. In some embodiments, the nucleotide conjugate may comprise a multivalent nucleotide conjugate. In some embodiments, the multivalent nucleotide conjugate may have the ability to form a binding complex. In some embodiments, the multivalent nucleotide conjugate may form a multivalent binding complex. In some embodiments, the nucleotide conjugate may comprise a core, a core attachment moiety, a spacer, a linker, a nucleotide unit, or any combination thereof. In some embodiments, the core may comprise a single molecule, a monomer, a single molecular unit, or a polymer. In some embodiments, the polymer may comprise a polypeptide, a protein, a peptide, or a derivative thereof. In some embodiments, the polymer may comprise a polypeptide comprising a modification. In some embodiments, the modification of the polypeptide may comprise any chemical, biological, or physical modification that may enable the polypeptide to form a nucleotide conjugate disclosed herein. In some embodiments, the modification may comprise a modification with polyethylene glycol (PEG), or PEGylation. In some embodiments, the polypeptide comprising a modification may include, but not limited to, those disclosed in the following references each of which is incorporated herein by reference by its entirety: Zuma L K, Gasa N L, Makhoba X H, Pooe O J. Protein PEGylation: Navigating Recombinant Protein Stability, Aggregation, and Bioactivity. Biomed Research International. (2022) Jul. 25; 2022: 8929715. doi: 10.1155/2022/8929715. PMID: 35924267; PMCID: PMC9343206; Suk J S, Xu Q, Kim N, Hanes J, Ensign L M. PEGylation as a strategy for improving nanoparticle-based drug and gene delivery. Advanced Drug Delivery Reviews. (2016) Apr. 1; 99(Pt A):28-51. doi: 10.1016/j.addr.2015.09.012. Epub 2015 Oct. 9. PMID: 26456916; PMCID: PMC4798869.

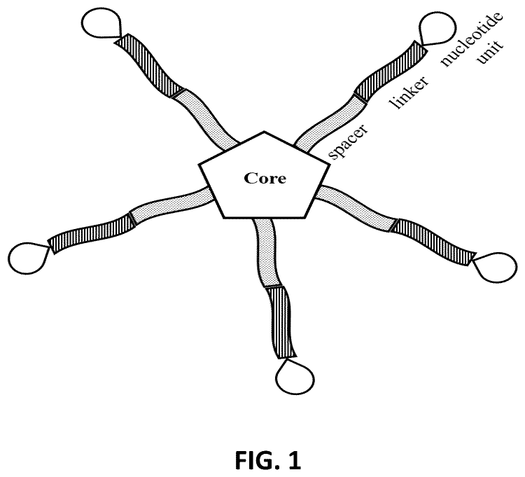

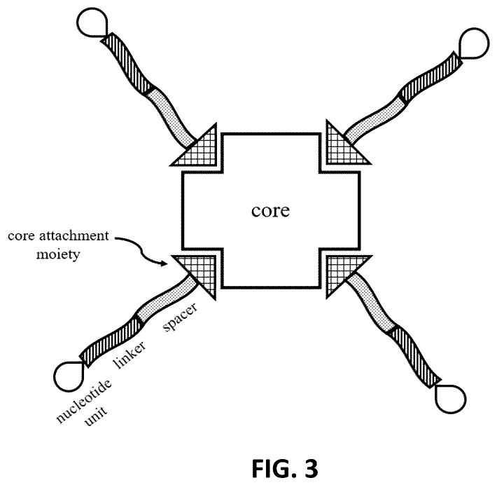

In some embodiments, a nucleotide conjugate comprises a core coupled to multiple “nucleotide-arms” such as, for example, as shown in FIGS. 1-3. In some embodiments, the nucleotide-arms can be modular. In some embodiments, the nucleotide-arms can comprise a core attachment moiety. In some embodiments, the nucleotide-arms can comprise a spacer. In some embodiments, the nucleotide-arms can comprise a linker. In some embodiments, the nucleotide-arms can comprise a nucleotide unit. In some embodiments, the nucleotide-arms can comprise a core attachment moiety, a spacer, a linker, and a nucleotide unit. Thus, a nucleotide conjugate can comprise multiple nucleotide units (e.g., see FIGS. 1-3). Alternatively, a nucleotide conjugate can comprise a single nucleotide unit.

In some embodiments, the nucleotide conjugates of the present disclosure provide an increase in binding of a nucleotide unit to a polymerizing enzyme, or to a complexed polymerizing enzyme, at least by increasing the effective concentration of the nucleotide unit with the enzyme. Such increase is observed due to the increase in the concentration of the nucleotides in solution, or by increasing the amount of the nucleotides in proximity to the relevant binding or incorporation site of the polymerizing enzyme. The increase can also be achieved by physically restricting a number of nucleotides into a limited volume resulting in a local increase in nucleotide concentration. The nucleotide unit of a nucleotide-arm (e.g., attached to a core) can bind to a polymerizing enzyme binding site with a higher apparent avidity than may be observed with unconjugated, untethered, or otherwise unrestricted individual nucleotide. One method of effecting such restriction can be by providing a nucleotide conjugate in which multiple nucleotide units are tethered to a core. In some embodiments, the core can be a particle such as a polymer, a branched polymer, a dendrimer, a micelle, a liposome, a microparticle, a nanoparticle, a quantum dot, or other suitable particle.

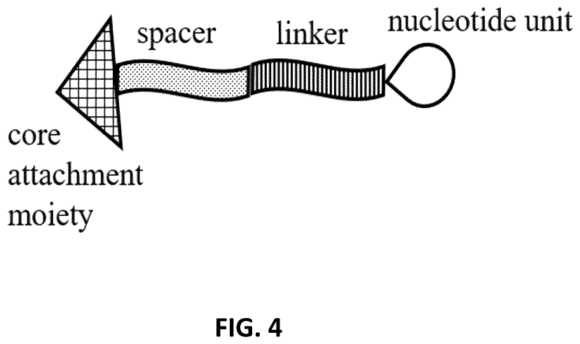

The present disclosure provides various embodiments of nucleotide conjugates each configured to include a core coupled (e.g., attached) to multiple nucleotide-arms. In some embodiments, the nucleotide-arms can comprise a core attachment moiety. In some embodiments, the nucleotide-arms can comprise a spacer. In some embodiments, the nucleotide-arms can comprise a linker. In some embodiments, the nucleotide-arms can comprise a nucleotide unit. In some embodiments, the nucleotide-arms can comprise a core attachment moiety, a spacer, a linker, and a nucleotide unit. Individual nucleotide-arms can comprise (i) a core attachment moiety, (ii) a spacer, (iii) a linker, and (iv) a nucleotide unit (e.g., see FIG. 4). In some embodiments, the nucleotide-arm can comprise a nucleoside unit instead of a nucleotide unit. The nucleotide-arm can include a biotin. In some embodiments, a nucleotide conjugate can comprise a plurality of copies of the same type of nucleotide attached to the core via multiple nucleotide-arms (e.g., see FIGS. 1, 2 and 3). In a nucleotide conjugate, the tethered nucleotide is called a nucleotide unit. In some embodiments, multiple copies of the nucleotide units may be covalently bound to or noncovalently bound to the core. The nucleotide-arm is designed so that the nucleotide units of the nucleotide-arm are capable of interacting with one or more polymerizing enzyme enzymes in a manner similar to a free nucleotide. The nucleotide unit of each nucleotide-arm can bind a polymerizing enzyme which is complexed with a nucleic acid template and nucleic acid primer (e.g., nucleotide association). The nucleotide unit can also dissociate from the complexed polymerizing enzyme and either re-bind the same complexed polymerizing enzyme or bind a different complexed polymerizing enzyme that is proximal to the nucleotide conjugate. Since a nucleotide conjugate can comprise multiple nucleotide-arms, the nucleotide units of a single nucleotide conjugate can bind multiple complexed polymerizing enzymes at the same time. The level of valency of the nucleotide units of a given nucleotide conjugate may correspond to the number of nucleotide arms linked to a core. In some embodiments, a single nucleotide conjugate can essentially simultaneously bind multiple complexed polymerizing enzymes which are bound to the same template molecule (e.g., a concatemer). The nucleotide conjugates can effectively increase the local concentration of nucleotides which can enhance signals in a nucleotide binding reaction.

A complexed polymerizing enzyme can comprise a synthetic polypeptide disclosed herein bound to a nucleic acid duplex, where the duplex can comprise a nucleic acid template molecule hybridized to a nucleic acid primer, where the primer can comprise an extendible or non-extendible terminal 3′ end. The template molecule can be a single-stranded or double-stranded linear or circularized nucleic acid molecule. The template molecule can be a clonally amplified nucleic acid molecule. The template molecule can be a nucleic acid concatemer. In some embodiments, the concatemers can comprise two or more tandem copies of a sequence of interest. The template and primer can be wholly or partially complementary along the hybridized region.

Alternatively, a complexed polymerizing enzyme can comprise a polymerizing enzyme bound to a self-priming nucleic acid template molecule, where the self-priming portion can comprise an extendible or non-extendible terminal 3′ end. The template molecule can include single-stranded or double-stranded regions. The template molecule can be a clonally amplified nucleic acid molecule. The template molecule can be a nucleic acid concatemer, for example comprising two or more tandem copies of a sequence of interest. The template and self-priming portion can be wholly or partially complementary along the hybridized region.

A single nucleotide conjugate can bind multiple complexed polymerizing enzymes which are bound to the same template molecule (e.g., a concatemer having repeats of the same target nucleic acid sequence) thereby forming a multivalent binding complex. For example, (i) a first binding complex can comprise a first nucleic acid primer, a first polymerizing enzyme, and a first nucleotide conjugate bound to a first portion of a concatemer template molecule thereby forming a first binding complex, wherein a first nucleotide unit of the first nucleotide conjugate can be bound to the first polymerizing enzyme, and (ii) a second binding complex can comprise a second nucleic acid primer, a second polymerizing enzyme, and the same first nucleotide conjugate can be bound to a second portion of the same concatemer template molecule thereby forming a second binding complex, wherein a second nucleotide unit of the first nucleotide conjugate can be bound to the second polymerizing enzyme, wherein the first and second binding complexes which include the same nucleotide conjugate can form the multivalent binding complex. Thus, in some embodiments, a multivalent binding complex of the instant disclosure comprises at least two copies of a target nucleic acid sequence bound to a single nucleotide conjugate disclosed herein. In some embodiments, the nucleotide units of the nucleotide conjugate are complementary to a nucleotide of the at least two copies of the target nucleic acid sequence. In some embodiments, the multivalent binding complex comprises one or more polymerizing enzymes (e.g., synthetic polypeptides) disclosed herein.

A nucleotide unit of a nucleotide conjugate can bind to a complexed polymerizing enzyme (e.g., a synthetic polypeptide disclosed herein), by binding the terminal 3′ end of the primer (or a nascent extended primer) or the self-priming portion, without undergoing polymerizing enzyme-catalyzed incorporation. The nucleotide unit which is bound to the complexed polymerizing enzyme can form a binding complex. The binding complex can be stable, where the nucleotide unit exhibits a low dissociation rate, and has a persistence time which is indicative of the stability of the binding complex and strength of the binding interactions. A condition that is suitable for binding a nucleotide unit to the 3′ end of the primer at a position that is opposite a complementary nucleotide in the template strand, where the nucleotide unit does not undergo polymerizing enzyme-catalyzed incorporation, can include the presence of at least one non-catalytic divalent cation comprising strontium, barium, calcium, or a combination thereof.

In some embodiments, the nucleotide conjugate can comprise at least one nucleotide unit having a sugar moiety bearing a 3′OH group or a 3′ blocking group. The nucleotide unit having the sugar 3′OH group or the 3′ blocking group can bind the complexed polymerizing enzyme and interrogate a complementary nucleotide in the template strand, and the nucleotide unit does not undergo nucleotide incorporation. A condition that is suitable for binding a nucleotide unit to the complexed polymerizing enzyme, where the nucleotide unit binds the 3′ end of the primer at a position that is opposite a complementary nucleotide in the template strand and interrogates the complementary nucleotide in the template strand, and where the nucleotide unit does not undergo polymerizing enzyme-catalyzed incorporation, can include the presence of at least one non-catalytic divalent cation comprising strontium, barium, calcium, or a combination thereof.

Alternatively, a nucleotide unit can bind to a complexed polymerizing enzyme, by binding the terminal 3′ end of the primer (or nascent extended primer) or the self-priming portion, and undergo polymerizing enzyme-catalyzed incorporation into the 3′ end of an extendible primer or the self-priming portion, resulting in primer extension. When the nucleotide unit includes a hydroxyl group at the 3′ sugar position, then a subsequent nucleotide can be incorporated into the nascent extended primer. When the nucleotide unit includes blocking group at the 3′sugar position, then a subsequent nucleotide can be blocked from being incorporated into the nascent extended primer strand. A condition that is suitable for binding a nucleotide unit to the 3′ end of the primer at a position that is opposite a complementary nucleotide in the template strand, where the nucleotide unit undergoes polymerizing enzyme-catalyzed incorporation, can include the presence of at least one catalytic divalent cation comprising magnesium, manganese, or a combination of magnesium and manganese.

In some embodiments, the nucleotide conjugate can comprise at least one nucleotide unit having a sugar moiety having a 3′OH group or a 3′ blocking group. The nucleotide unit having the sugar 3′OH group can bind the complexed polymerizing enzyme and interrogate a complementary nucleotide in the template strand, and the nucleotide unit can undergo polymerizing enzyme-catalyzed nucleotide incorporation. The nucleotide unit having a sugar 3′ blocking group can bind the complexed polymerizing enzyme, and can interrogate the complementary nucleotide in the template strand, and the nucleotide unit can undergo polymerizing enzyme-catalyzed nucleotide incorporation but the 3′ blocking group can inhibit/prevent incorporation of a subsequent nucleotide (or the next nucleotide unit of a nucleotide conjugate). The 3′ blocking group can be removed to facilitate incorporation of a subsequent nucleotide. A condition that is suitable for binding a nucleotide unit to the complexed polymerizing enzyme, where the nucleotide unit binds the 3′ end of the primer at a position that is opposite a complementary nucleotide in the template strand and interrogates the complementary nucleotide in the template strand, and where the nucleotide unit undergoes polymerizing enzyme-catalyzed incorporation, can include the presence of at least one catalytic divalent cation comprising magnesium, manganese, or a combination of magnesium and manganese.

In some embodiments, the linker unit of a nucleotide-arm can contribute to binding a polymerizing enzyme, to stabilizing the polymerizing enzyme-nucleotide ternary complex, or a combination of binding to a polymerizing enzyme and stabilizing the polymerizing enzyme-nucleotide ternary complex. We have found that both the linker and the nucleotide may be recognized and discriminated by the DNA polymerizing enzyme. We discovered that proximal portions of the linker unit may play a critical role in facilitating the polymerizing enzyme-nucleotide binding interaction. Optimization of the linker region can provide improved nucleotide conjugates useful for particular applications, such as nucleic acid sequencing.

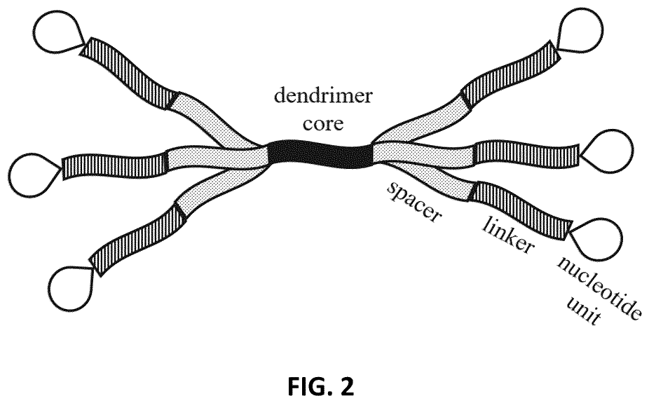

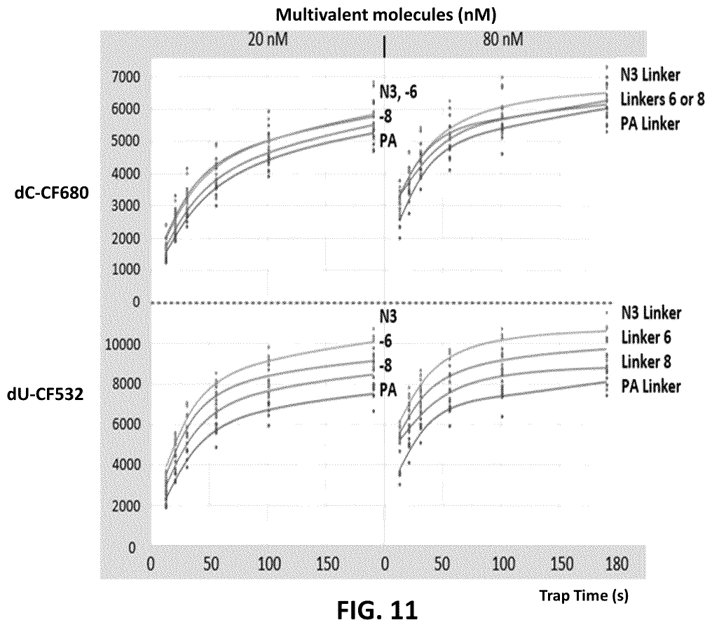

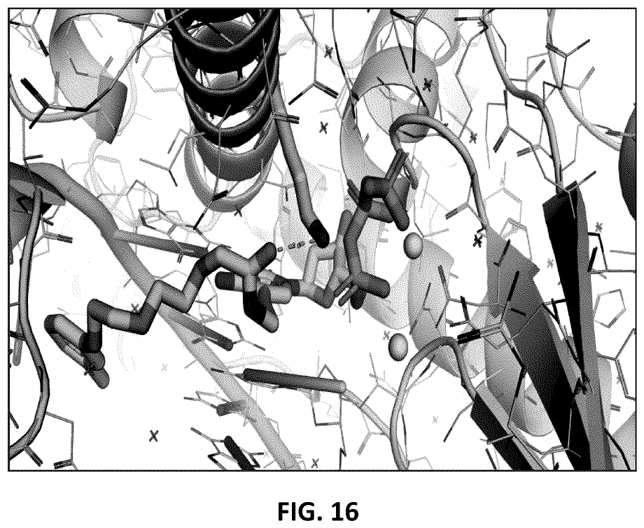

Crystal structures of a sequencing polymerizing enzyme co-crystallized with a primed template molecule and nucleotide conjugate show that a carbonyl oxygen of the nucleotide arm interacts with a lysine residue of the polymerizing enzyme (e.g., see FIG. 16 and Example 8). In the nucleotide-arm, the carbonyl oxygen is proximal to the nucleotide unit. The polymerizing enzyme used in Example 8 has a backbone sequence based on the amino acid sequence of SEQ ID NO:1 and the lysine residue is located at position 495. Additionally, binding studies comparing different types of nucleotide conjugates having nucleotide arms carrying different linkers shows varying levels of binding. Data from the binding studies suggests that the presence of an aromatic moiety (e.g., an aryl group) on the linker can improve binding of the nucleotide unit to the polymerizing enzyme (e.g., see FIGS. 11 and 2, and Example 5).

The nucleotide conjugates can be labeled with a detectable reporter moiety. In some embodiments, the core of the nucleotide conjugate can be labeled with a detectable reporter moiety (e.g., fluorophore) in a manner that permits distinction between different nucleotide conjugates carrying a different type of nucleotide units. For example, the core unit of a first nucleotide conjugate can be labeled with a first fluorophore, where the first nucleotide conjugate can comprise multiple nucleotide-arms with the same type of nucleotide units, for example dGTP nucleotide units. The core unit of a second nucleotide conjugate can be labeled with a second fluorophore (which differs from the first fluorophore), where the second nucleotide conjugate can comprise multiple nucleotide-arms with the same type of nucleotide units, for example dATP nucleotide units. Mixtures of labeled nucleotide conjugates can include any combination of two or more sub-populations of nucleotide conjugates where each sub-population includes nucleotide conjugates having one type of a nucleotide unit and a reporter-labeled core that corresponds to the particular type of nucleotide unit. Mixtures of labeled and non-labeled nucleotide conjugates can include any combination of at least one sub-population of nucleotide conjugates comprising a plurality of nucleotide conjugates having one type of a nucleotide unit and a reporter-labeled core that corresponds to the particular type of nucleotide unit, and at least one sub-population of nucleotide conjugates comprising a plurality of nucleotide conjugates having a different type of a nucleotide unit and a non-labeled core where the non-labeled core corresponds to the different type of nucleotide unit. A single population, or mixtures of different sub-populations of labeled nucleotide conjugates, can be used for nucleotide binding assays, nucleotide incorporation assays, nucleic acid sequencing methods, or a combination thereof. The nucleotide conjugates can be useful for massively parallel nucleic acid sequencing.

Disclosed herein are formulations comprising a first nucleotide conjugate and a second nucleotide conjugate. In some embodiments, the formulation comprises a first nucleotide conjugate, a second nucleotide conjugate, and a third nucleotide conjugate. In some embodiments, the formulation comprises a first nucleotide conjugate, a second nucleotide conjugate, a third nucleotide conjugate, and a fourth nucleotide conjugate. In some embodiments, the first nucleotide conjugate comprises a first label. In some embodiments, the second nucleotide conjugate comprises a second label. In some embodiments, the third nucleotide conjugate comprises a third label. In some embodiments, the fourth nucleotide conjugate comprises a fourth label. In some embodiments, the first label comprises a first fluorophore. In some embodiments, the second label comprises a second fluorophore. In some embodiments, the third label comprises a third fluorophore. In some embodiments, the fourth label comprises a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other. Mixtures of different sub-populations of labeled nucleotide conjugates may comprise a first sub-population of nucleotide conjugates comprising the first fluorophore and a second sub-population of nucleotide conjugates comprising the second fluorophore

In some embodiments, at least one nucleotide unit of a nucleotide conjugate can be labeled with a detectable reporter moiety (e.g., fluorophore) in a manner that permits distinction between different nucleotide conjugates carrying a different type of nucleotide units. For example, the base of a nucleotide unit of a first nucleotide conjugate can be labeled with a first fluorophore, where the first nucleotide conjugate can comprise multiple nucleotide-arms with the same type of nucleotide units, for example dGTP nucleotide units. The base of a nucleotide unit of a second nucleotide conjugate can be labeled with a second fluorophore (which differs from the first fluorophore), where the second nucleotide conjugate can comprise multiple nucleotide-arms with the same type of nucleotide units, for example dATP nucleotide units. Mixtures of labeled nucleotide conjugates can include any combination of two or more sub-populations of nucleotide conjugates where each sub-population includes nucleotide conjugates having one type of a nucleotide unit and a reporter-labeled nucleobase that corresponds to the particular type of nucleotide unit. Mixtures of labeled and non-labeled nucleotide conjugates can include any combination of at least one sub-population of nucleotide conjugates comprising a plurality of nucleotide conjugates having one type of a nucleotide unit and a reporter-labeled nucleobase that corresponds to the particular type of nucleotide unit, and at least one sub-population of nucleotide conjugates comprising a plurality of nucleotide conjugates having a different type of a nucleotide unit and a non-labeled nucleobase where the non-labeled nucleobase corresponds to the different type of nucleotide unit. A single population, or mixtures of different sub-populations of labeled nucleotide conjugates, can be used for nucleotide binding assays, nucleotide incorporation assays, nucleic acid sequencing methods, or a combination thereof. The nucleotide conjugates can be useful for massively parallel nucleic acid sequencing.

The nucleotide conjugates can be used to localize detectable signals to active regions of biochemical interactions, such as sites of protein-nucleic acid interactions, nucleic acid hybridization reactions, or enzymatic reactions, such as polymerizing enzyme-mediated reactions. For example, the nucleotide conjugates described herein can be utilized to identify sites of nucleobase binding or incorporation during polymerizing enzyme-catalyzed reactions and to provide base discrimination for sequencing and array-based applications. The increased binding or incorporation between the template nucleic acid and the nucleotide unit, when the nucleotide unit is complementary to the “N” base in the template nucleic acid, can provide enhanced signal that greatly improve base call accuracy and shorten imaging time.

In addition, labeled nucleotide conjugates can form multivalent binding complexes which increase base call signals from a given polony containing multiple copies of the template nucleic acid strands (e.g., concatemers). Sequencing workflows that include generating polonies having clonally-amplified copies of a template strand can have the advantage that signals can be amplified due to the presence of multiple simultaneous sequencing reactions within a defined region, each providing its own signal. The presence of multiple signals within a defined area can also reduce the impact of any erroneously-advanced or skipped cycle(s), due to the fact that the signal from a large number of correct base calls can overwhelm the signal from a smaller number of advanced or skipped incorrect base calls, therefore providing methods for reducing pre-phasing or phasing errors, improving read length in sequencing reactions, or a combination of reducing phasing errors and improving read length in sequences reactions.

The nucleotide conjugates and their use disclosed herein can lead to one or more of: (i) stronger signal for better base-calling accuracy compared to nucleic acid amplification and sequencing methodologies; (ii) allow greater discrimination of sequence-specific signal from background signals; (iii) reduced requirements for the amount of starting material, (iv) increased sequencing rate and shortened sequencing time; (v) reducing phasing errors, and (vi) improving read length in sequencing reactions.

The present disclosure provides a nucleotide conjugate comprising a core attached to at least one nucleotide-arm. In some embodiments, the at least one nucleotide-arm can comprise a core attachment moiety. In some embodiments, the at least one nucleotide-arm can comprise a spacer. In some embodiments, the at least one nucleotide-arm can comprise a linker. In some embodiments, the at least one nucleotide-arm can comprise a nucleotide unit. In some embodiments, the at least one nucleotide-arm can comprise a core attachment moiety, a spacer, a linker, and a nucleotide unit. In some embodiments, the core can comprise a bead, particle or nanoparticle. In some embodiments, the core can comprise an alkyl, alkenyl, or alkynyl core such as may be present in a branched polymer or dendrimer. In some embodiments the core can comprise a moiety that mediates conjugation of the core to the nucleotide-arm. In some embodiments, the core can be attached to a plurality of nucleotide-arms. In some cases, the core can be attached to between about 1 to about 50 nucleotide arms. In some cases, the core is attached to between about 2 to about 20 nucleotide-arms. In some cases, the core is attached to between about 2 to about 4 nucleotide-arms. In some cases, the core is attached to between about 4 to about 10 nucleotide-arms. In some cases, the core is attached to between about 10 to about 15 nucleotide-arms. In some cases, the core is attached to between about 15 to about 20 nucleotide-arms. FIGS. 1, 2 and 3 show the general architecture of nucleotide conjugates.

The present disclosure provides a nucleotide conjugate comprising a core attached to at least one biotinylated nucleotide-arm. In some embodiments, the at least one biotinylated nucleotide-arm can comprise a core attachment moiety. In some embodiments, the at least one biotinylated nucleotide-arm can comprise a spacer. In some embodiments, the at least one biotinylated nucleotide-arm can comprise a linker. In some embodiments, the at least one biotinylated nucleotide-arm can comprise a nucleotide unit. In some embodiments, the at least one biotinylated nucleotide-arm can comprise a core attachment moiety, a spacer, a linker, and a nucleotide unit. In some embodiments the core can comprise a streptavidin-type or avidin-type moiety, and the biotin unit of the biotinylated nucleotide-arm can mediate conjugation of the core to the biotinylated nucleotide-arm (FIG. 2). A streptavidin-type or avidin-type core can be a tetrameric biotin-binding protein that can bind one, two, three or up to four biotinylated nucleotide-arms.

In some embodiments, the nucleotide conjugate comprises a core. In some embodiments, the core is a particle. In some embodiments, the particle is a nanoparticle or a microparticle. In some embodiments, the material of the particle comprises a polymer or a metal. In some embodiments, the polymer is synthetic. In some embodiments, the polymer is natural. In some embodiments, the polymer comprises a plastic or a protein. In some embodiments, the protein comprises streptavidin, or avidin, or derivatives thereof, analogs thereof, and other non-native forms thereof that can bind to at least one biotin moiety. In some embodiments, the plastic is or comprises polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), or polyethylene glycol (PEG), or any combination thereof. In some embodiments, the core may comprise polyethylene glycol (PEG), polypropylene glycol (PPG), polyvinyl acetate (PVA), polylactic acid (PLA), or polyglycolic acid (PGA), poly lactic-co-glycolic acid (PLGA), a chlorinated or fluorinated derivative thereof, or combinations thereof.

In some embodiments, the core may comprise avidin, streptavidin, or the like, or a derivative thereof a branched polymer; a dendrimer; a cross linked polymer particle such as an agarose, polyacrylamide, acrylate, methacrylate, cyanoacrylate, methyl methacrylate particle; a glass particle; a ceramic particle; a metal particle; a quantum dot; a liposome; an emulsion particle, or any other suitable particle (e.g, nanoparticles, microparticles, or the like). In some embodiments, the core can comprise a streptavidin-type or avidin-type moiety, including streptavidin or avidin protein, as well as any derivatives, analogs, and other non-native forms of streptavidin or avidin that can bind to at least one biotin moiety. The streptavidin or avidin moiety can comprise native or recombinant forms, as well as mutant versions and derivatized molecules. Mutant versions of streptavidin and avidin can comprise any one or any combination of two or more of amino acid insertions, deletions, substitutions, or truncations. Mutant versions can also include fusion polypeptides.

The nucleotide conjugates can be configured using a streptavidin or avidin core having a high affinity for the biotin moiety on a biotinylated nucleotide-arm to reduce dissociation of the nucleotide-arms from the core. A mixture of nucleotide conjugates can be prepared, where the mixture contains two or more sub-populations of nucleotide conjugates and each sub-population contains nucleotide conjugates having one type of nucleotide units (e.g., dATP, dGTP, dCTP, dTTP or dUTP). Nucleotide conjugates that are configured to have high affinity between the core and nucleotide-arms can reduce undesirable dissociation of nucleotide-arms from the core, and exchange of nucleotide arms between different cores. Exchange of nucleotide arms during a sequencing reaction can lead to incorrect based calling and reduced sequencing accuracy. In some embodiments, nucleotide conjugates having increased stability (e.g., reduced dissociation of biotinylated nucleotide-arms) can comprise a dye labeled streptavidin, where the streptavidin subunits carry a Lys121Arg mutation which can exhibit reduced dissociation of a biotinylated nucleotide-arm from the streptavidin core.

The streptavidin moiety can comprise full-length or truncated forms having a high affinity for binding biotin. For example, the streptavidin moiety can exhibit a dissociation constant (K_{d}) of about 10⁻¹⁴ mol/L, or about 10⁻¹⁵ mol/L. In some embodiments, the streptavidin moiety can comprise a polypeptide having the backbone sequence any of SEQ ID NOs:466-470. The streptavidin moiety can comprise an amino acid sequence having at least 80%, at least 85%, at least 90%, at least 95%, at least 99% or higher levels of identity to any of SEQ ID NOS:466-470. The streptavidin moiety can comprise the core portion of a streptavidin protein which is truncated at the N-terminal, C-terminal end, or a combination of the N-terminal and the C-terminal of a streptavidin protein having an amino acid sequence of any of SEQ ID NOS:466-470. For example, the streptavidin moiety can lack the N-terminal portion of any of SEQ ID NOS:466-468, 470-471 (e.g., the underlined N-terminal portions in FIGS. 17-19, 21-22). The streptavidin moiety can lack the C-terminal portion of any of SEQ ID NOS:466-468 (e.g., the underlined C-terminal portions in FIGS. 17-19). The streptavidin moiety can comprise a core portion comprising the amino acid sequence of SEQ ID NO:469 or 470.

In some embodiments, the streptavidin moiety can comprise any amino acid substitution mutation at a site that can be labeled with a dye. For example, the dye-labeling site can comprise lysine at position 121 (e.g., see SEQ ID NO:469) which may overlap with a biotin binding site. In some embodiments, a dye attached to streptavidin at Lys121 may block or inhibit biotin binding to the dye-labeled streptavidin. A nucleotide conjugate comprising a dye labeled streptavidin carrying lysine at position 121 may exhibit dissociation of a biotinylated nucleotide-arm from the streptavidin core. A nucleotide conjugate having increased stability can comprise a dye labeled streptavidin (e.g., SEQ ID NO:469) carrying a Lys121Arg mutation which can exhibit reduced dissociation of a biotinylated nucleotide-arm from the streptavidin core.

In some embodiments, the streptavidin moiety can comprise any amino acid substitution that increases the affinity for binding biotin (e.g., increases the K_{d} to about 10⁻¹⁶ mol/L), improves retention of biotin at temperatures up to about 60° C., or about 65° C., or about 70° C. or about 80° C., or a combination of increases the affinity for binding biotin and improves retention of biotin. Amino acid substitutions can comprise His151Lys or His 151Asp of SEQ ID NO:466; His128Lys or His128Asp of SEQ ID NO:467; His127Lys or His127Asp of SEQ ID NO:468; His116Lys or His116Asp of SEQ ID NO:469; or His135Lys or His135Asp of SEQ ID NO:470. The histidine residue that can be substituted with lysine or aspartic acid are bolded and underlined in FIGS. 17-21.

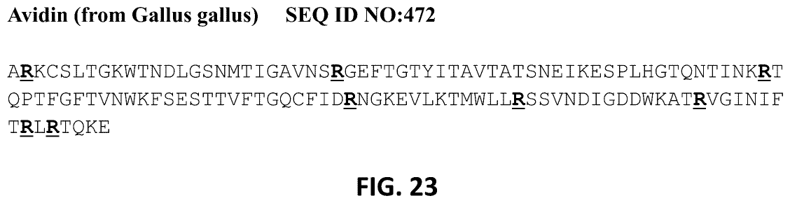

The avidin moiety can comprise full-length or truncated forms having a high affinity for binding biotin. For example, the avidin moiety can exhibit a dissociation constant (K_{d}) of about 10⁻¹⁴ mol/L, or about 10⁻¹⁵ mol/L. In some embodiments, the avidin moiety can comprise a polypeptide having the backbone sequence SEQ ID NOS:471 or 472. The avidin moiety can comprise an amino acid sequence having at least 80%, at least 85%, at least 90%, at least 95%, at least 99% or higher levels of identity to SEQ ID NOS:471 or 472. The avidin moiety can comprise the core portion of an avidin protein which is truncated at the N-terminal, the C-terminal, or a combination of the N-terminal and C-terminal ends of an avidin protein having an amino acid sequence of SEQ ID NO:471. For example, the avidin moiety can lack the N-terminal portion of SEQ ID NO:471 (e.g., the underlined N-terminal portions in FIG. 22). In some embodiments, the avidin can comprise substitutions of any one or any combination of the eight arginine residues (e.g., underlined and bolded in FIG. 22 or 23). Amino acid substitutions can comprise replacing five of the eight arginine residues with a neutral amino acid at positions 26, 50, 83, 111, 124, 138, 146, 148, or a combination thereof of SEQ ID NO:471; or replacing five of the eight arginine residues with a neutral amino acid at positions 2, 26, 59, 87, 100, 114, 122, 124, or a combination thereof of SEQ ID NO:472.

The avidin can comprise partially de-glycosylated forms and non-glycosylated forms. The avidin moiety can include derivatized forms, for example, N-acyl avidins, e.g., N-acetyl, N-phthalyl and N-succinyl avidin, and the commercially-available products including ExtrAvidin®, CaptAvidin™ (selective nitration of tyrosine residues at the four biotin-binding sites to generate avidin that reversibly binds biotin), NeutrAvidin™ (having chemically de-glycosylated and include modified arginine residues), and Neutralite Avidin™ (five of the eight arginine residues are replaced with neutral amino acids, two of the lysine residues are replaced with glutamic acid, and Asp17 is replaced with isoleucine). Amino acids having neutral non-polar side chains include alanine, glycine, isoleucine, leucine, methionine, phenylalanine, proline tryptophan and valine. Amino acids having neutral polar side chains include asparagine, cysteine, glutamine, serine, threonine, and tyrosine.

In some embodiments, the core can be labeled with a detectable moiety. Non-limiting examples of detectable moieties include fluorescent, bioluminescent, chemiluminescent, radiological detectable moieties. In some embodiments, the nucleotide conjugate may be unlabeled. The core can be streptavidin or avidin which are homo-tetramers. Each subunit in the homo-tetramer can include at least one lysine residue which can be conjugated to a fluorophore. A labeling reaction can employ N-hydroxysuccinimide (NHS) ester-conjugated fluorophores. The maximum number of fluorophores that can be attached to a streptavidin or avidin subunit can be dictated by the number of lysine residues in the subunit. The amino acid sequences of various streptavidin and avid subunits are provided in FIGS. 17-23. For example, streptavidin subunits can include 4 lysines (e.g., SEQ ID NO:469), 8 lysines (e.g., SEQ ID NOS:467, 468, 470) or 9 lysines (e.g., SEQ ID NO:466). Avidin subunits can include 9 lysines (e.g., SEQ ID NOS: 471 and 472).

When preparing labeled streptavidin or avidin cores, the labeling reaction can be optimized to achieve a predetermined degree of labeling (sometimes abbreviated as DoL). The degree of labeling can be expressed as a molar ratio in the form of label/protein. Dye-core conjugates with a lower degree of labeling will exhibit weaker fluorescent intensities. Dye-core conjugates with very high degree of labeling (e.g., DoL>6) may exhibit reduced fluorescence due to self-quenching from the conjugated fluorophore. In some embodiments, the predetermined degree of labeling for streptavidin or avidin cores may depend upon the dye. Fluorescent dyes include but are not limited to: CF647, CF680, CF570 and CF532 dyes from Biotium; AF647, AF680, AF568 and AF532 from Thermo Fisher Scientific; IFluor 647, IFluor 680, IFlour 568 and IFlour 532 from AATBio; DY648P1, DY679P1, DY585 and DY530 from Dyomics; and AFDy 647, IRFlour 680LT, AFDye 568 and AFDye 532 from Fluoroprobes. The predetermined degree of labeling can be about 1-10, or about 3-8, or about 3.5-7, or about 1.6-4.

Red fluorophores are brighter (higher intensity) than green dyes, which can cause color bleeding when imaging both red-labeled and green-labeled nucleotide conjugates on the same support (e.g., flow cell). The degree of labeling of a sub-population of nucleotide conjugates can be increased or decreased to achieve improved signal balance from a mixture of labeled nucleotide conjugates. For example, the degree of labeling of a sub-population of nucleotide conjugates labeled with a red fluorophore can be decreased compared to the degree of labeling of a sub-population of nucleotide conjugates labeled with a green fluorophore. In some embodiments, the degree of labeling of a sub-population of nucleotide conjugates labeled with a red fluorophore can be about 1-3, or about 2-3, or about 3-6. In some embodiments, the degree of labeling of a sub-population of nucleotide conjugates labeled with a green fluorophore can be about 4-7.

Solution fluorescence measurements can be used to determine the relative brightness of the labeled streptavidin or avidin cores. Alternatively, the degree of labeling can be determined by employing a functional assay (e.g., a flow cell trap assay) in which clonally-amplified template molecules immobilized on a flow cell are contacted with primers, polymerizing enzymes and fluorescently-labeled nucleotide conjugates, under a condition suitable for binding the nucleotide conjugates to complexed polymerizing enzymes without incorporating the nucleotide units into the primer, and signal intensity can be detected.

In some embodiments, the nucleotide conjugate comprises at least two nucleotide arms. In some embodiments, a nucleotide arm of the at least two nucleotide arms comprises: a core attachment moiety coupled to the core. In some embodiments, a nucleotide arm of the at least two nucleotide arms comprises a spacer coupled to the core attachment moiety. In some embodiments, a nucleotide arm of the at least two nucleotide arms comprises a linker coupled to the spacer. In some embodiments, a nucleotide arm of the at least two nucleotide arms comprises a nucleotide unit coupled to the linker. In some embodiments, the nucleotide conjugate comprises: (a) a core; and (b) at least two nucleotide arms. In some embodiments, a nucleotide arm of the at least two nucleotide arms comprises: (i) a core attachment moiety coupled to the core; (ii) a spacer coupled to the core attachment moiety; (iii) a linker coupled to the spacer; and (iv) a nucleotide unit coupled to the linker. In some embodiments, the linker comprises:

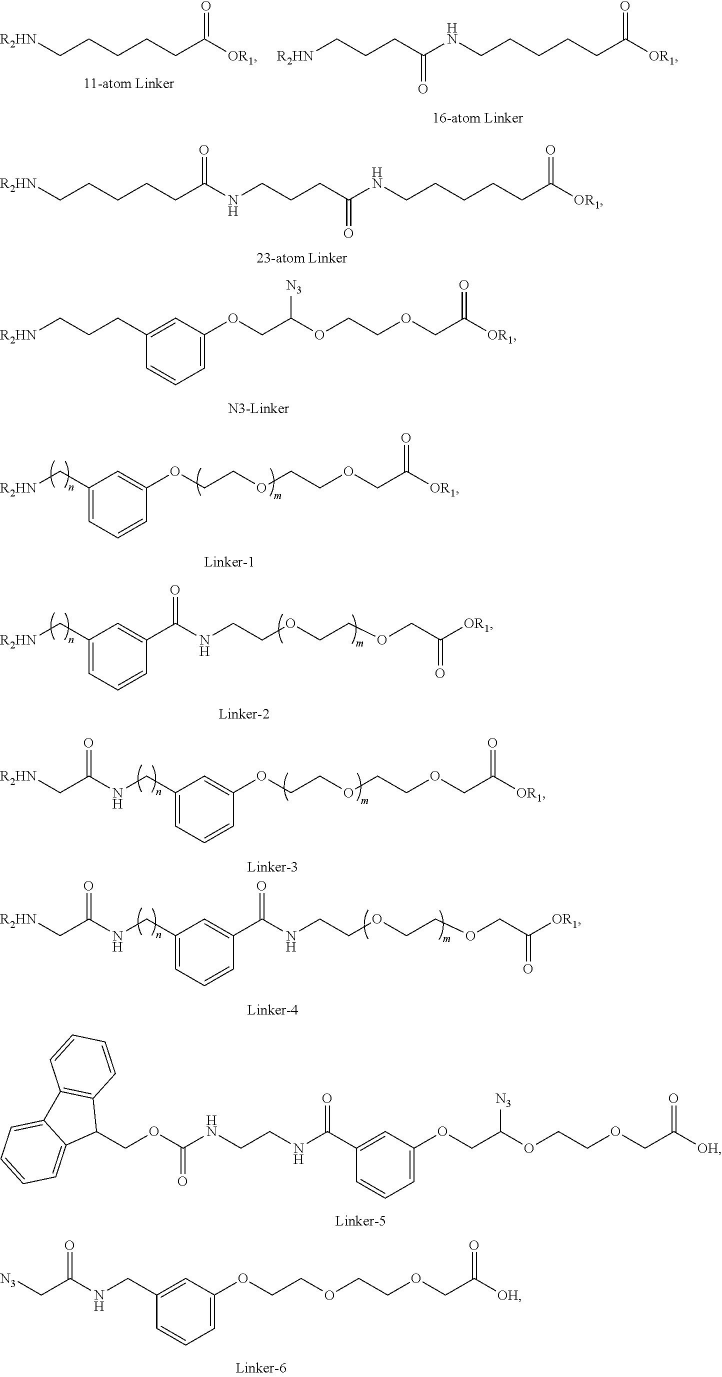

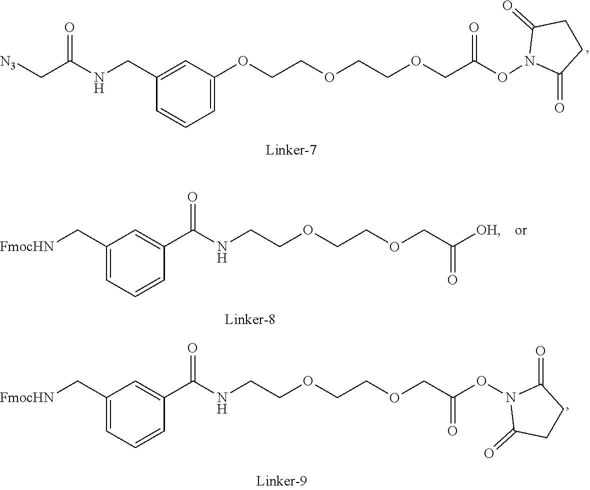

and n is 1 to 6 and m is Oto 10.

In some embodiments, the linker comprises

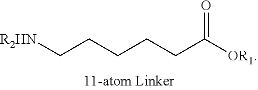

In some embodiments, the linker comprises

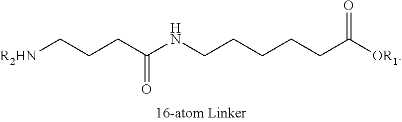

In some embodiments, the linker comprises

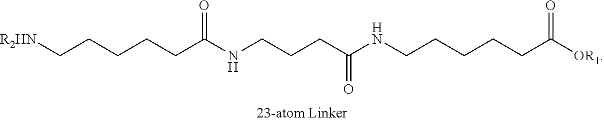

In some embodiments, the linker comprises

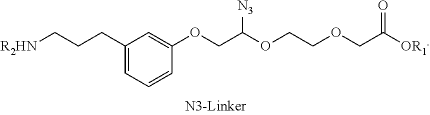

In some embodiments, the linker comprises

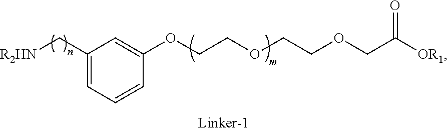

wherein n is 1 to 6 and m is 0 to 10.

In some embodiments, the linker comprises

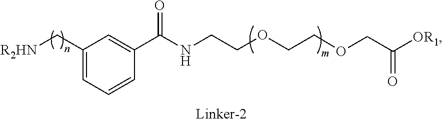

wherein n is 1 to 6 and m is 0 to 10.

In some embodiments, the linker comprises

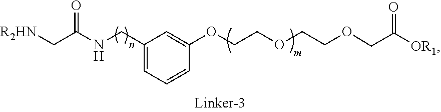

wherein n is 1 to 6 and m is 0 to 10.

In some embodiments, the linker comprises

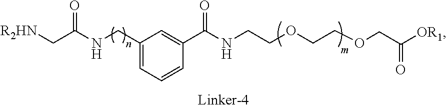

wherein n is 1 to 6 and m is 0 to 10.

In some embodiments, the linker comprises

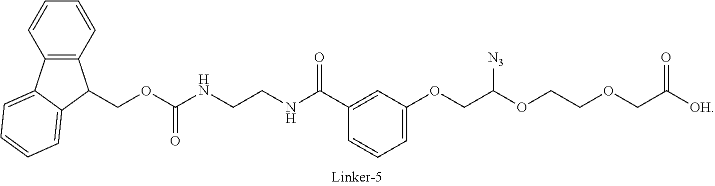 [SMILES: [N-]=[N+]=NC(COc1cccc(C(=O)NCCNC(=O)OCC2c3ccccc3-c3ccccc32)c1)OCCOCC(=O)O]

In some embodiments, the linker comprises

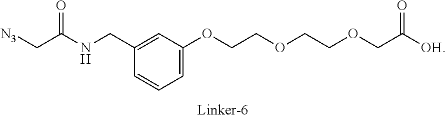 [SMILES: [N-]=[N+]=NCC(=O)NCc1cccc(OCCOCCOCC(=O)O)c1]

In some embodiments, the linker comprises

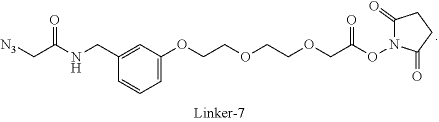 [SMILES: [N-]=[N+]=NCC(=O)NCc1cccc(OCCOCCOCC(=O)ON2C(=O)CCC2=O)c1]

In some embodiments, the linker comprises

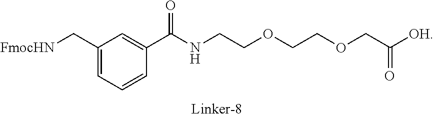

In some embodiments, the linker comprises

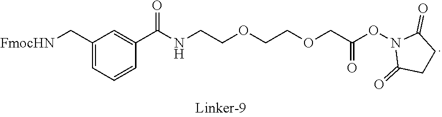

In some embodiments, the at least two nucleotide arms comprises 3 to 20 nucleotide arms, for example, 3, 4, 5, 6, 7, 8, 9, 10, 11, 12, 13, 14, 15, 16, 17, 18, 19, or 20 nucleotide arms. In some embodiments, the at least two nucleotide arms comprises from 4 to 19, 5 to 18, 6 to 17, 7 to 16, 8 to 15, 9 to 14, 10 to 13, or 12 nucleotide arms. In some embodiments, the core comprises a polypeptide. In some embodiments, the polypeptide comprises streptavidin or avidin. In some embodiments, the polypeptide comprises streptavidin. In some embodiments, the polypeptide comprises avidin. In some embodiments, the core attachment moiety comprises biotin.

In some embodiments, the nucleotide conjugate comprises a label, such as a detectable label. In some embodiments, the label is coupled to the core or the nucleotide arm. In some embodiments, the label is coupled to the core. In some embodiments, the label is coupled to the nucleotide arm. In some embodiments, the label is or comprises a fluorescent label. In some embodiments, the composition further comprises a fluorescent label coupled to the core. In some embodiments, the composition further comprises a fluorescent label coupled to the nucleotide arm.

In some embodiments, the spacer comprises a structure:

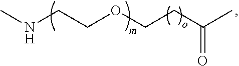

wherein m is 20 to 500 and o is 1 to 10.

In some embodiments, the nucleotide arm further comprises a reactive group. In some embodiments, the reactive group is coupled to the nucleotide unit. In some embodiments, the reactive group is configured to react with an agent. In some embodiments, the nucleotide arm further comprises a reactive group coupled to the nucleotide unit and the reactive group is configured to react with an agent. In some embodiments, the reactive group comprises an alkyl, alkenyl, an alkynyl, an allyl, an aryl, a benzyl, an azide, an amine, an amide, a keto, an isocyanate, a phosphate, a thiol, a disulfide, a carbonate, a urea, or a silyl group. In some embodiments, the alkyl, alkenyl, alkynyl or allyl group of the reactive group reacts with tetrakis(triphenylphosphine)palladium(0) (Pd(P(C₆H₅)₃)₄) with piperidine or 2,3-Dichloro-5,6-dicyano-1,4-benzoquinone (DDQ). In some embodiments, the aryl or benzyl group of the reactive group reacts with H₂ and Palladium on carbon (Pd/C). In some embodiments, the amine, amide, keto, isocyanate, phosphate, thiol, or disulfide group of the reactive group reacts with phosphine or a thiol group comprising beta-mercaptoethanol or dithiothritol (DTT). In some embodiments, the carbonate group of the reactive group reacts with potassium carbonate (K₂CO₃) in methanol (MeOH), triethylamine in pyridine, or Zn in acetic acid (AcOH). In some embodiments, the urea or silyl group of the reactive group reacts with tetrabutylammonium fluoride, Hydrogen fluoride pyridine (HF-pyridine), ammonium fluoride, or triethylamine trihydrofluoride. In some embodiments, the azide group of the reactive group comprises an azide, an azido or an azidomethyl group. In some embodiments, the agent comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP). In some embodiments, the nucleotide unit of the at least two nucleotide arms comprises the same nucleobase type.

In some embodiments, the nucleotide unit comprises a blocking group. In some embodiments, the blocking group is linked to the 3′ carbon of the sugar moiety of the nucleotide unit. In some embodiments, the blocking group reacts with a chemical compound to remove the blocking group from the nucleotide unit. In some embodiments, the blocking group reacts with a chemical compound to remove the blocking group from the nucleotide unit to generate the nucleotide unit comprising a OH moiety. In some embodiments, the nucleotide unit comprises a blocking group linked to the 3′ carbon of the sugar moiety of the nucleotide unit and the blocking group reacts with a chemical compound to remove the blocking group from the nucleotide unit to generate the nucleotide unit comprising a OH moiety on the 3′ carbon of the sugar moiety of the nucleotide unit. In some embodiments, the blocking group reacts with a chemical compound to remove the blocking group from the nucleotide unit to generate the nucleotide unit comprising a OH moiety on the 3′ carbon of the sugar moiety of the nucleotide unit. In some embodiments, the blocking group comprises an alkyl, an alkenyl, an alkynyl, an allyl, an aryl, a benzyl, an azide, an amine, an amide, a keto, an isocyanate, a phosphate, a thiol, a disulfide, a carbonate, a urea, or a silyl group.

In some embodiments, the alkyl, alkenyl, alkynyl, or allyl group of the blocking group reacts with tetrakis(triphenylphosphine)palladium(0) (Pd(P(C₆H₅)₃)₄) with piperidine, or 2,3-Dichloro-5,6-dicyano-1,4-benzo-quinone (DDQ). In some embodiments, the aryl or benzyl group of the blocking group reacts with H₂ and Palladium on carbon (Pd/C). In some embodiments, the amine, amide, keto, isocyanate, phosphate, thiol, or disulfide group of the blocking group reacts with phosphine, or a thiol group comprising beta-mercaptoethanol, or dithiothritol (DTT). In some embodiments, the carbonate group of the blocking group reacts with potassium carbonate (K₂CO₃) in methanol (MeOH), triethylamine in pyridine, or Zn in acetic acid (AcOH). In some embodiments, the urea or silyl group of the blocking group reacts with tetrabutylammonium fluoride, HF-pyridine, ammonium fluoride, or triethylamine trihydrofluoride.

In some embodiments, the azide of the blocking group comprises an azide, an azido or an azidomethyl group. In some embodiments, the azide, azido, or azidomethyl group reacts with a chemical agent. In some embodiments, the chemical agent comprises a phosphine compound. In some embodiments, the azide, azido, or azidomethyl group reacts with a chemical agent that comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP).

### Nucleotide-Arms

The present disclosure provides nucleotide conjugates comprising one or more nucleotide arms. In some embodiments, the “nucleotide-arm” can be modular. In some embodiments, the nucleotide-arms can comprise a core attachment moiety. In some embodiments, the nucleotide-arms can comprise a spacer. In some embodiments, the nucleotide-arms can comprise a linker. In some embodiments, the nucleotide-arms can comprise a nucleotide unit. In some embodiments, the nucleotide-arms can comprise a core attachment moiety, a spacer, a linker, and a nucleotide unit. In some embodiments, the nucleotide-arm can be a nucleoside instead of a nucleotide. In some embodiments, the nucleotide or nucleoside can comprise an analogue thereof.

In some embodiments, the nucleotide-arm may be attached to a core. In some embodiments, two or more nucleotide-arms can be attached to a core to form a nucleotide conjugate. The nucleotide conjugate can comprise multiple nucleotide-arms (e.g., FIGS. 1-3) where individual nucleotide-arms include a nucleotide unit that can bind a different complexed polymerase to form an multivalent binding complex.

### Spacers

In some embodiments, the compositions (e.g., nucleotide conjugate) comprises a spacer. In some embodiments, the nucleotide arm can comprise a spacer. In some embodiments, the spacer may be coupled to any of the other components of the nucleotide arm, including but not limited to, a core, a nucleotide unit, a linker, or any other component disclosed herein.

The spacer can physically separate the nucleotide unit or nucleoside unit from the core. A spacer is shown in FIG. 5A. The spacer can have any length, for example the value of m can be 1 or at least 2, at least 5, at least 10, at least 20, at least 25, at least 50, at least 75, at least 100, at least 200, at least 500, at least 750, at least 1000, or 2000 or more. In some embodiments, the value of m can be about 20-500, or about 100-110 (e.g., 5,000 g/mol PEG). In some embodiments, in the spacer shown in FIG. 5A, the value of o can bel-50. In some embodiments, the value of o can be about 1-10, or the value of o is about 4. The spacer can be a linear or branched molecule.

In some embodiments, the spacer can comprise polyethylene glycol, polypropylene glycol, polyvinyl acetate, polylactic acid, or polyglycolic acid.

In some embodiments, the spacer can comprise polyethylene glycol (PEG) having a molecular weight of about 100-200 Da, 200-300 Da, 300-400 Da, 400-500 Da, 1K Da, 2K Da, 3K Da, 4K Da, 5K Da, 10K Da, 15 K Da, 20K Da, 30K Da, 40K Da, 50K Da, or larger molecular weight PEG.

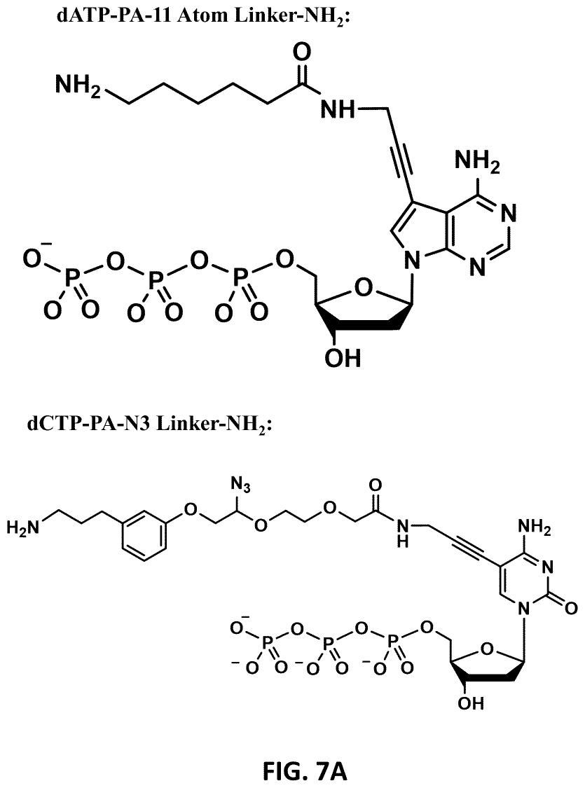

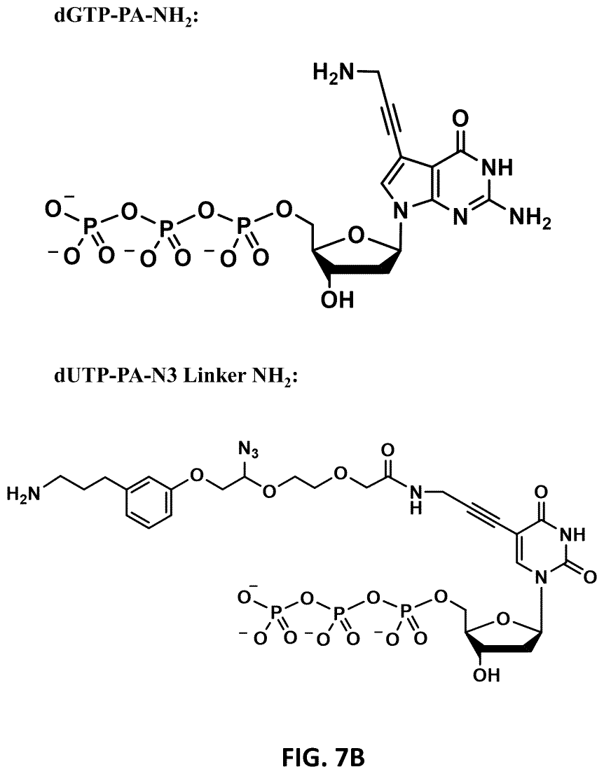

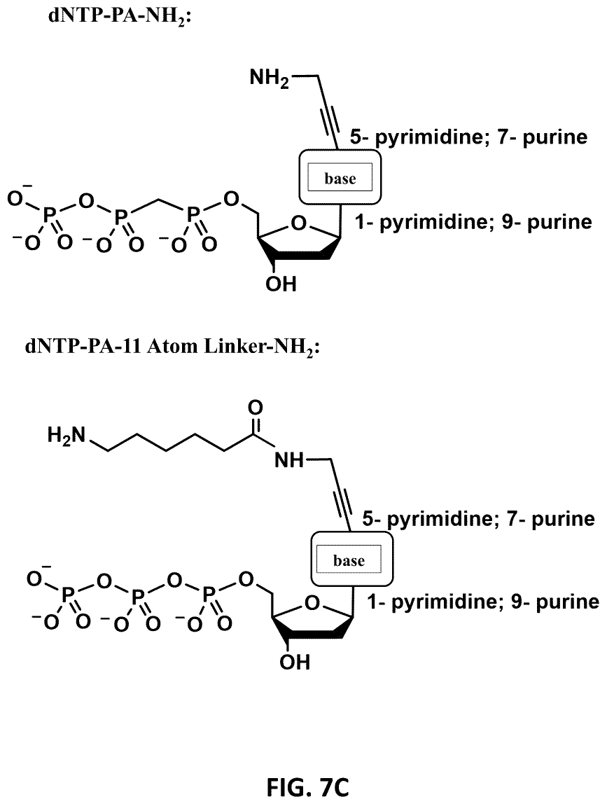

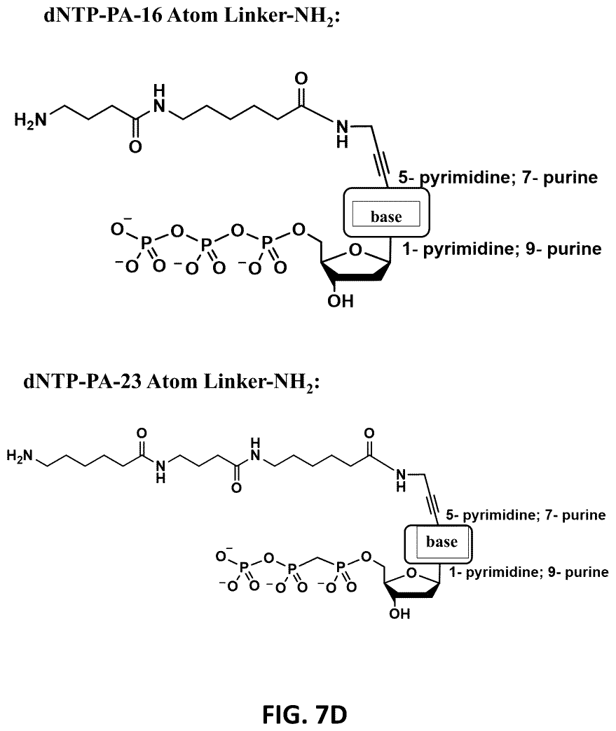

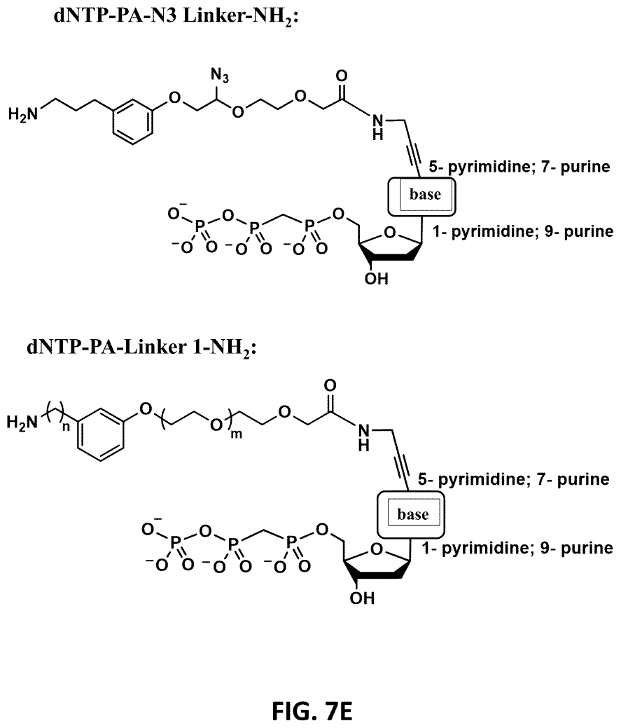

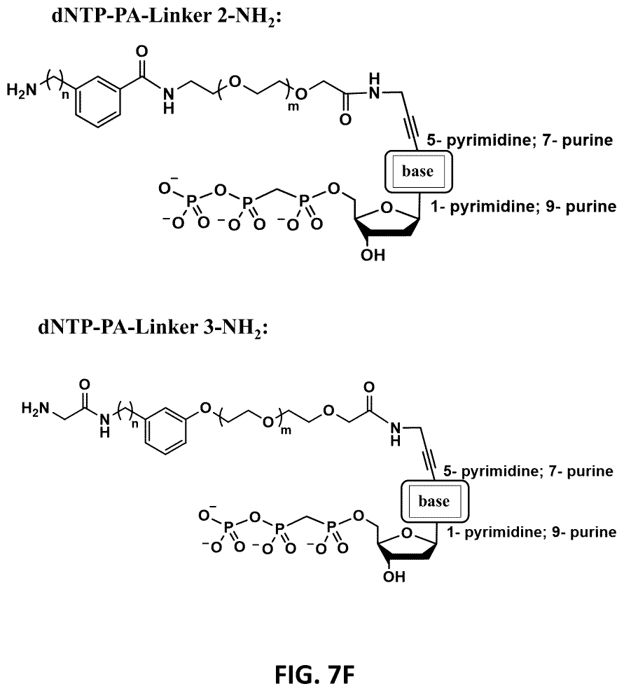

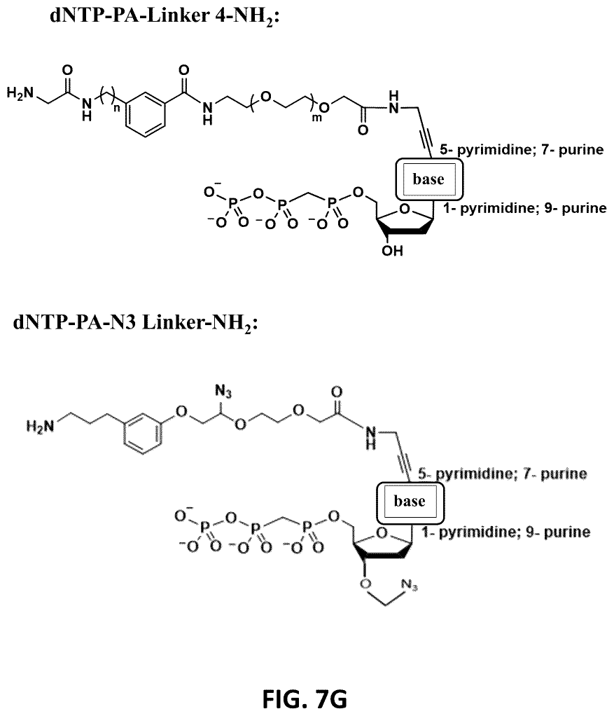

In some embodiments, the spacer unit of a nucleotide-arm can be attached to a biotin moiety, thereby forming a biotinylated nucleotide-arm. In some embodiments, the biotinylated nucleotide-arm can comprise a core attachment moiety. In some embodiments, the biotinylated nucleotide-arm can comprise a spacer. In some embodiments, the biotinylated nucleotide-arm can comprise a linker. In some embodiments, the biotinylated nucleotide-arm can comprise a nucleotide unit. In some embodiments, the biotinylated nucleotide-arm can comprise a core attachment moiety, a spacer, a linker, and a nucleotide unit. In some embodiments, the biotinylated nucleotide-arm can comprise (i) biotin, (ii) spacer, (iii) linker, and (iv) nucleotide (or nucleoside) (e.g., see FIGS. 7A-D).

### Linkers

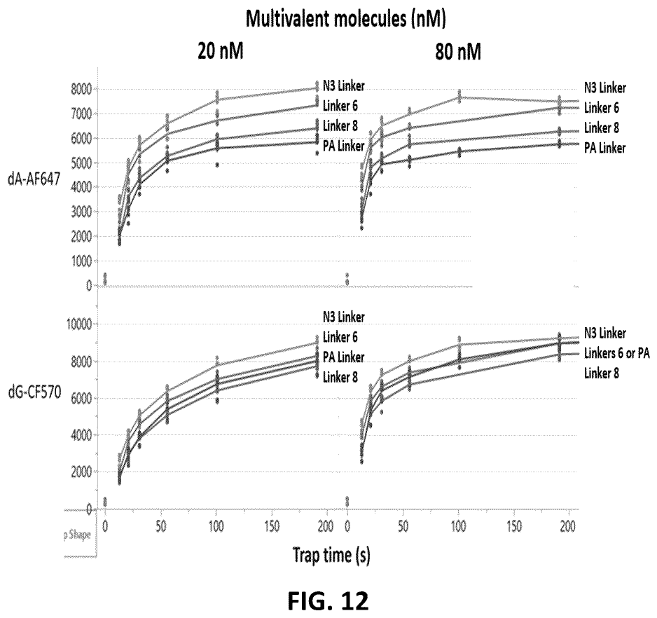

In some embodiments, the nucleotide arm may comprise a linker. In some embodiments, the linker may be coupled to any of the other components of the nucleotide arm, including but not limited to, a core, a nucleotide unit, a spacer, or any other component disclosed herein. In some embodiments, the nucleotide arm can comprise a linker having any one or any combination of two or more moieties including an amide, carbonyl oxygen, an aromatic moiety, a polyether moiety, or a combination thereof. The aromatic moiety can comprise a six-carbon ring structure such as a benzene ring. The polyether moiety can be polyethylene glycol, polypropylene glycol, polyvinyl acetate, polylactic acid, or polyglycolic acid. The linker can comprise a linear or branched molecule. The linker can include or lack a reactive moiety (e.g., a cleavable moiety). The linker portion of a nucleotide-arm can interact with a polymerase. The type of moiety and location of the moiety in the linker can be selected to optimize interaction with a polymerase binding pocket. For example, the amide moiety in a linker can interact with a polymerase via hydrogen bonding. The aromatic moiety in a linker can interact with the polymerase via hydrophobic interaction. A nucleotide-arm can comprise Linker-6 which includes a carbonyl oxygen proximal to the nucleotide unit and an aryl moiety. Crystallography data indicates that the carbonyl oxygen interacts with a lysine residue in the polymerase binding pocket. Trapping assays indicate that linkers carrying an aromatic moiety can exhibit improved binding to polymerases when compared with linkers that lack an aromatic moiety (e.g., see FIGS. 11 and 12, and Example 5).

In some embodiments, the nucleotide arm can comprise a linker which can comprise any of the linker structures shown in FIGS. 5A-F. In some embodiments, the R1 of a linker can comprise any group, for example a nucleotide, nucleoside, or analog thereof. In some embodiments, the R2 of a linker can comprise any group, for example a spacer (e.g., see the top of FIG. 5A). In some embodiments, in the linker, the value of m can be 0-10. In some embodiments, in the linker, the value of n can be 1-6.

In some embodiments, the linker can comprise an aliphatic chain having 2-8 units. In some embodiments, the linker can comprise an oligo ethylene glycol moiety having 2-8 units. In some embodiments, the linker can comprise an aromatic group. In some embodiments, the linker can comprise an aromatic group and an oligo ethylene glycol moiety having 2-8 units. In some embodiments, the linker can comprise an aliphatic chain having 2-6 subunits. In some embodiments, the linker can comprise an oligo ethylene glycol chain having 2-6 subunits. In some embodiments, the aromatic group can comprise an aryl group (e.g., see N3 Linker and Linkers 1-7 in FIGS. 5A-C). In some embodiments, the aromatic group can comprise a six-carbon ring group. In some embodiments, the aromatic group can comprise a meta-aminomethylbenzoic acid (also called 3-aminomethylbenzoic acid) group (mAMBA) (e.g., Linkers 8 and 9 of FIG. 5C). In some embodiments, the linker can comprise a fluorenylmethoxycarbonyl protecting group (Fmoc) which can be removed when joining the linker to a spacer. In some embodiments, the linker can comprise an NHS ester group (N-Hydroxysuccinimide) which can be removed when joining the linker to a nucleotide unit.

### Nucleotide Arms: Spacer-Linker-Nucleotide

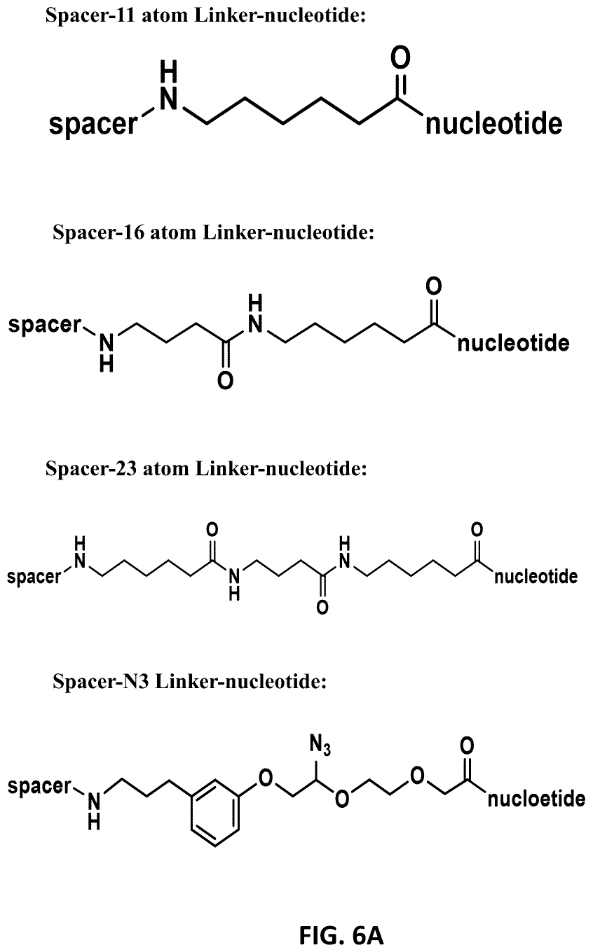

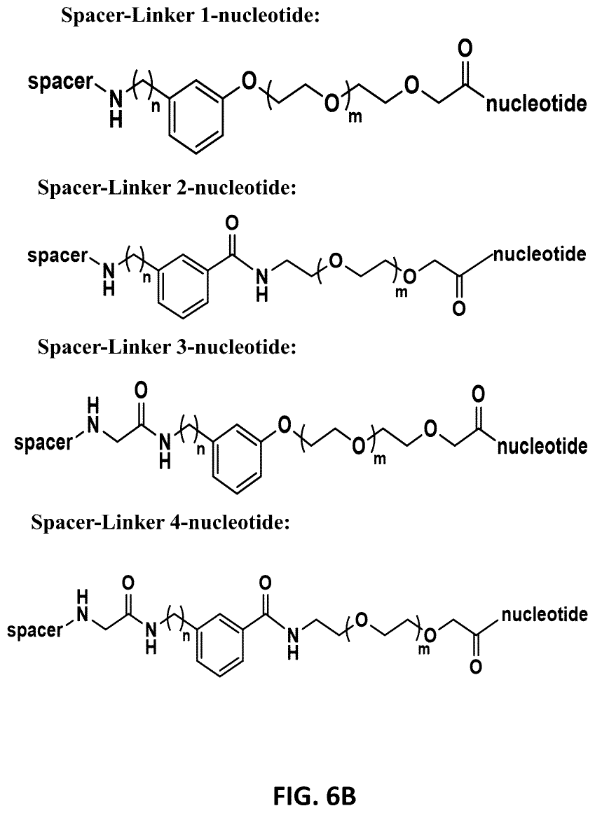

In some embodiments, a nucleotide-arm can comprise a spacer joined to any of the linkers shown in FIGS. 5A-F, and the linker can be joined to any nucleotide or nucleoside. Nucleotide-arms are shown in FIGS. 6A and 6B.

In some embodiments, the nucleotide-arm can comprise a spacer having the structure shown in FIG. 5A (top). The spacer can have any length, for example the value of m is 1 or at least 2, at least 5, at least 10, at least 20, at least 25, at least 50, at least 75, at least 100, at least 200, at least 500, at least 750, at least 1000, or 2000 or more. In some embodiments, the value of m can be about 20-500, or about 100-110 (e.g., 5,000 g/mol PEG). In some embodiments, in the spacer shown in FIG. 5A, the value of o can be 1-50. In some embodiments, the value of o can be about 1-10, or the value of o can be about 4. In some embodiments, the spacer can comprise polyethylene glycol, polypropylene glycol, polyvinyl acetate, polylactic acid, or polyglycolic acid.

In some embodiments, the nucleotide-arm can comprise any type of nucleotide or nucleoside joined to the linker. Nucleotides can include but are not limited to dATP, dGTP, dCTP, dTTP or dUTP. The nucleotide unit can be joined to the linker via a propargyl amine attachment at the 5 position of a pyrimidine base or the 7 position of a purine base, or at the 1 position of a pyrimidine base or 9 position of a purine base.

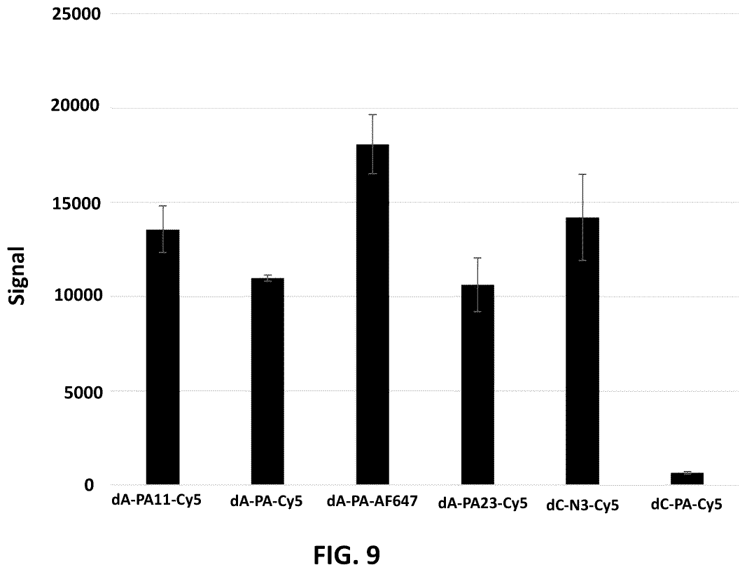

In some embodiments, certain combinations of linkers and nucleotides can exhibit improved nucleotide discrimination by a polymerase as exhibited by brighter fluorescent signals in nucleotide trapping assays (e.g., see FIGS. 9, 10 and 11, and Example 4). It is noted that the “N3” linker is photolabile. The “N3” moiety may be useful in decreasing residual fluorescent signals seen in cycling assays.

### Linker-Nucleotide Unit

In some embodiments, any of the linkers described herein can be joined to a nucleotide to generate a nucleotide-linker molecule. Non-limiting examples of nucleotide-linker configurations are shown in FIGS. 7A-G. For example, the nucleotide unit can be joined to the linker via a propargyl amine attachment at the 5 position of a pyrimidine base or the 7 position of a purine base, or at the 1 position of a pyrimidine base or 9 position of a purine base.

### Biotinylated Nucleotide Arms

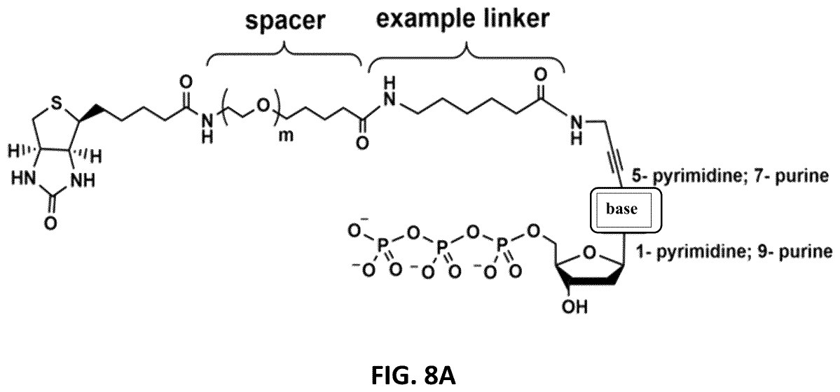

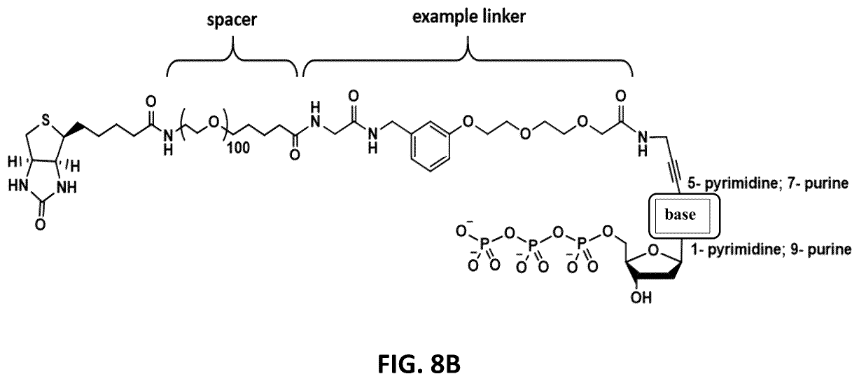

In some embodiments, the nucleotide-arm can be a biotinylated nucleotide-arm. In some embodiments, the biotinylated nucleotide-arm comprises (i) biotin, (ii) spacer, (iii) linker, and (iv) nucleotide (or nucleoside). Non-limiting examples of biotinylated nucleotide-arms are shown in FIGS. 8A and 8B.

In some embodiments, the nucleotide conjugates can comprise a core attached to a plurality of biotinylated nucleotide-arms. In some embodiments, a biotinylated nucleotide-arm can comprise a core attachment moiety. In some embodiments, a biotinylated can comprise a spacer. In some embodiments, a biotinylated can comprise a linker. In some embodiments, a biotinylated can comprise a nucleotide unit. In some embodiments, a biotinylated can comprise a core attachment moiety, a spacer, a linker, and a nucleotide unit. In some embodiments, an individual biotinylated nucleotide-arms comprise (i) a biotin moiety, (ii) a spacer, (iii) a linker, and (iv) a nucleotide unit or a nucleoside unit.

### Nucleotide Units

In some embodiments, any of the nucleotide-arms and biotinylated nucleotide-arms described herein can comprise a nucleotide unit. In some embodiments, the nucleotide unit can bind a polymerase which is complexed with a nucleic acid template and nucleic acid primer (e.g., nucleotide association). The nucleotide unit can also dissociate from the complexed polymerase and either re-bind the same complexed polymerase or bind a different complexed polymerase that is proximal to the nucleotide conjugate.

The nucleotide-arm and biotinylated nucleotide-arm can comprise a nucleotide unit, where the nucleotide unit can comprise a heterocyclic base, a sugar and at least one phosphate group. In some embodiments, the nucleotide can comprise 1, 2, 3, 4, 5, 6, 7, 8, 9 or 10 phosphate groups.

The nucleotide-arm and biotinylated nucleotide-arm can comprise a nucleotide unit (or nucleoside unit) which includes a heterocyclic base comprising a purine or pyrimidine base, or analogs thereof.

In some embodiments, the 5 position of the pyrimidine base can be joined to the linker, the 7 position of the purine base can be joined to the linker, the 1 position of the pyrimidine base can be joined to the linker, or the 9 position of the purine base can be joined to a linker.

In some embodiments, the nucleotide-arm and biotinylated nucleotide-arm can comprise a nucleotide unit which is a propargyl-amine (PA) modified nucleotide, where the 5-position of the pyrimidine base or 7-position of the purine base can be joined to the linker via a propargyl-amine group, or where the 1 position of a pyrimidine base or 9 position of a purine base can be joined to the linker via a propargyl-amine group.

In some embodiments, the nucleotide-arm and biotinylated nucleotide-arm can comprise a nucleotide unit having a ribose or deoxyribose sugar moiety. In some embodiments, the nucleotide unit can be selected from a group consisting of adenosine triphosphate (ATP), adenosine diphosphate (ADP), adenosine monophosphate (AMP), deoxyadenosine triphosphate (dATP), deoxyadenosine diphosphate (dADP), and deoxyadenosine monophosphate (dAMP), thymidine triphosphate (TTP), thymidine diphosphate (TDP), thymidine monophosphate (TMP), deoxythymidine triphosphate (dTTP), deoxythymidine diphosphate (dTDP), deoxythymidine monophosphate (dTMP), uridine triphosphate (UTP), uridine diphosphate (UDP), uridine monophosphate (UMP), deoxyuridine triphosphate (dUTP), deoxyuridine diphosphate (dUDP), deoxyuridine monophosphate (dUMP), cytidine triphosphate (CTP), cytidine diphosphate (CDP), cytidine monophosphate (CMP), deoxycytidine triphosphate (dCTP), deoxycytidine diphosphate (dCDP), deoxycytidine monophosphate (dCMP), guanosine triphosphate (GTP), guanosine diphosphate (GDP), guanosine monophosphate (GMP), deoxyguanosine triphosphate (dGTP), deoxyguanosine diphosphate (dGDP), and deoxyguanosine monophosphate (dGMP).

In some embodiments, the nucleotide-arm and biotinylated nucleotide-arm can comprise a nucleotide unit (or nucleoside unit) having a sugar moiety which comprises a ribose, deoxyribose, or analog thereof. In some embodiments, the sugar moiety can comprise a 3′OH group. In some embodiments, a nucleotide unit having a sugar 3′OH group can bind a complexed polymerase which includes a polymerase bound to a nucleic acid template which can be hybridized to a nucleic acid primer (or a self-priming template/primer nucleic acid molecule). For example, the nucleotide unit can bind the polymerase, and can bind the terminal 3′ end of the primer at a position that is opposite a complementary nucleotide in the template strand. A nucleotide unit having a sugar 3′OH group can undergo nucleotide incorporation in a polymerase-catalyzed reaction. The sugar 3′OH group on an incorporated nucleotide unit can mediate polymerase-catalyzed incorporation of a subsequent nucleotide, for example in a primer extension reaction.

In some embodiments, the nucleotide-arm and biotinylated nucleotide-arm can comprise a nucleotide unit (or nucleoside unit) having a sugar moiety with a 3′OH group substituted with a blocking group. In some embodiments, a nucleotide unit having a 3′blocking group can bind a complexed polymerase which includes a polymerase bound to a nucleic acid template which is hybridized to a nucleic acid primer (or a self-priming template/primer nucleic acid molecule). For example, the nucleotide unit can bind the polymerase, and can bind the terminal 3′ end of the primer at a position that is opposite a complementary nucleotide in the template strand. A nucleotide unit having a 3′ blocking group can undergo nucleotide incorporation in a polymerase-catalyzed reaction. The 3′ blocking group on an incorporated nucleotide unit can inhibit/block a polymerase-catalyzed incorporation of a subsequent nucleotide, for example in a primer extension reaction.

### Modified Nucleotide Arms

In some embodiments, any of the nucleotide-arms and biotinylated nucleotide-arms described herein further can comprise a linker having a reactive group at any position along the linker. For example, the azide moiety in the N3 Linker (FIG. 5A) can be replaced with a reactive group. In some embodiments, any of the nucleotide-arms and biotinylated nucleotide-arms described herein can comprise a linker having a reactive group which is selected from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group.

In some embodiments, the reactive group in the linker can be reactive with a chemical reagent. For example, the reactive groups alkyl, alkenyl, alkynyl and allyl are reactive with tetrakis(triphenylphosphine)palladium(0)(Pd(P(C6H5)3)4) with piperidine, or with 2,3-Dichloro-5,6-dicyano-1,4-benzo-quinone (DDQ). The reactive groups aryl and benzyl are reactive with H2 and Palladium on carbon (Pd/C). The reactive groups amine, amide, keto, isocyanate, phosphate, thiol, disulfide are reactive with phosphine or with a thiol group including beta-mercaptoethanol or dithiothritol (DTT). The reactive group carbonate is reactive with potassium carbonate (K₂CO₃) in MeOH, with triethylamine in pyridine, or with Zn in acetic acid (AcOH). The reactive groups urea and silyl are reactive with tetrabutylammonium fluoride, pyridine-HF, with ammonium fluoride, or with triethylamine trihydrofluoride.

In some embodiments, the reactive group in the linker can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the nucleotide-arms and biotinylated nucleotide-arms described herein further can comprise a nucleotide unit having a sugar moiety with a 3′OH group substituted with a chain terminating moiety (blocking group), where the sugar 3′ blocking group is selected from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group.

In some embodiments, the sugar 3′ blocking group can comprise a 3′-O-azidomethyl group, 3′-O-methyl group, 3′-O-alkyl hydroxylamino group, a 3′-phosphorothioate group, a 3′-0-malonyl group, or a 3′-O-benzyl group.

In some embodiments, the nucleotide-arm and biotinylated nucleotide-arm can comprise a nucleotide unit having a sugar 3′ blocking group that is reactive with a chemical reagent. For example, the sugar 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The blocking groups aryl and benzyl can be reactive with Palladium on carbon (Pd/C). The blocking groups amine, amide, keto, isocyanate, phosphate, thiol, disulfide can be reactive with phosphine or with a thiol group including beta-mercaptoethanol or dithiothritol (DTT). The blocking group carbonate can be reactive with potassium carbonate (K₂CO₃) in MeOH, with triethylamine in pyridine, or with Zn in acetic acid (AcOH). The blocking groups urea and silyl can be reactive with tetrabutylammonium fluoride, pyridine-HF, with ammonium fluoride, or with triethylamine trihydrofluoride.

In some embodiments, the nucleotide-arm and biotinylated nucleotide-arm can comprise a nucleotide unit having a sugar 3′ blocking group comprising an azide, azido or azidomethyl group.

In some embodiments, the azide, azido or azidomethyl 3′ blocking group can be reactive with a chemical reagent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, the nucleotide-arm and biotinylated nucleotide-arm can comprise a nucleotide unit having a chain of one, two or three phosphorus atoms where the chain is attached to the 5′ carbon of the sugar moiety via an ester or phosphoramide linkage. In some embodiments, the nucleotide unit can be an analog having a phosphorus chain in which the phosphorus atoms are linked together with intervening O, S, NH, methylene or ethylene. In some embodiments, the phosphorus atoms in the chain can include substituted side groups including O, S or BH₃. In some embodiments, the chain can include phosphate groups substituted with analogs including phosphoramidate, phosphorothioate, phosphordithioate, and O-methylphosphoroamidite groups.

### Nucleotide Conjugate Combinations

The present disclosure provides compositions, systems, and kits comprising a plurality of nucleotide conjugates which can comprise a mixture of at least two sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be labeled with a second reporter moiety that corresponds to a second nucleotide unit on the nucleotide-arms. In some cases, the first and second reporter moieties can differ from each other. In some embodiments, the plurality of nucleotide conjugates can further comprise at least a third sub-population of nucleotide conjugates which is labeled with a third reporter moiety, wherein the first, second and third reporter moieties can differ from each other. In some embodiments, the plurality of nucleotide conjugates can further comprises at least a fourth sub-population of nucleotide conjugates which is labeled with a fourth reporter moiety, wherein the first, second, third and fourth reporter moieties can differ from each other. In some embodiments, additional sub-populations (e.g., fifth, sixth, seventh, eighth, ninth, tenth or more) of labeled nucleotide conjugates can be added into the mixture. In some embodiments, the reporter moiety can be a fluorophore. In some embodiments, a first sub-population of nucleotide conjugates can be labeled with a first fluorophore and a second fluorophore of nucleotide conjugates can be labeled with a second fluorophore. In some cases, the first fluorophore and the second fluorophore can be different.

The present disclosure provides compositions, systems, and kits comprising a plurality of nucleotide conjugates which can comprises a mixture of at least two sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be non-labeled (e.g., a dark nucleotide conjugate).

The present disclosure provides compositions, systems, and kits comprising a plurality of nucleotide conjugates which comprises a mixture of at least three sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be labeled with a second reporter moiety that corresponds to a second nucleotide unit on the nucleotide-arms. In some embodiments, at least a third sub-population of nucleotide conjugates can be non-labeled (e.g., a dark nucleotide conjugate). In some embodiments, the first and second reporter moieties can differ from each other.

The present disclosure provides compositions, systems, and kits comprising a plurality of nucleotide conjugates which comprises a mixture of at least four sub-populations of nucleotide conjugates labeled with different reporter moieties. In some embodiments, the mixture of nucleotide conjugates can have at least a first sub-population of nucleotide conjugates can be labeled with a first reporter moiety that corresponds to a first nucleotide unit on the nucleotide-arms. In some embodiments, the mixture of nucleotide conjugates can have at least a second sub-population of nucleotide conjugates can be labeled with a second reporter moiety that corresponds to a second nucleotide unit on the nucleotide-arms. In some embodiments, the mixture of nucleotide conjugates can have at least a third sub-population of nucleotide conjugates is labeled with a third reporter moiety. In some embodiments, the mixture of nucleotide conjugates can have at least a fourth sub-population of nucleotide conjugates can be non-labeled (e.g., a dark nucleotide conjugate). In some cases, the first, second and third reporter moieties can differ from each other.

Disclosed herein are formulations comprising a first nucleotide conjugate and a second nucleotide conjugate. In some embodiments, the formulation comprises a first nucleotide conjugate, a second nucleotide conjugate, and a third nucleotide conjugate. In some embodiments, the formulation comprises a first nucleotide conjugate, a second nucleotide conjugate, a third nucleotide conjugate, and a fourth nucleotide conjugate. In some embodiments, the first nucleotide conjugate comprises a first label. In some embodiments, the second nucleotide conjugate comprises a second label. In some embodiments, the third nucleotide conjugate comprises a third label. In some embodiments, the fourth nucleotide conjugate comprises a fourth label. In some embodiments, the first label comprises a first fluorophore. In some embodiments, the second label comprises a second fluorophore. In some embodiments, the third label comprises a third fluorophore. In some embodiments, the fourth label comprises a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other. Mixtures of different sub-populations of labeled nucleotide conjugates may comprise a first sub-population of nucleotide conjugates comprising the first fluorophore and a second sub-population of nucleotide conjugates comprising the second fluorophore

An embodiment comprises: a mixture of four different types of nucleotide conjugates comprising (1) a first sub-population of nucleotide conjugates each comprising a dATP nucleotide unit and a core labeled with a first type of fluorophore, (2) a second sub-population of nucleotide conjugates each comprising a dGTP nucleotide unit and a core labeled with a second type of fluorophore, (3) a third sub-population of nucleotide conjugates each comprising a dCTP nucleotide unit and a core labeled with a third type of fluorophore, and (4) a fourth sub-population of nucleotide conjugates each comprising a dTTP nucleotide unit and a core labeled with a fourth type of fluorophore, where the first, second, third and fourth fluorophores can be spectrally distinguishable. In some embodiments, any one of the sub-populations of nucleotide conjugates can be non-labeled for use as “dark” nucleotide conjugates.

The present disclosure provides compositions, systems, and kits comprising a plurality (e.g., a population) of nucleotide conjugates, wherein individual nucleotide conjugates in the plurality can comprise a core bound to at least one nucleotide-arm. In some embodiments, individual nucleotide conjugates in the plurality can comprise a core bound to 2-5 nucleotide-arms. In some embodiments, individual nucleotide conjugates in the plurality can comprise a streptavidin or avidin core bound to 2-5 biotinylated nucleotide-arms.

The present disclosure provides compositions, systems, methods, and kits comprising a plurality (e.g., a population) of nucleotide conjugates, wherein individual nucleotide conjugates in the plurality can comprise a core bound to at least one nucleotide-arm having one type of nucleotide unit comprising dATP, dGTP, dCTP, dTTP or dUTP. In some embodiments, individual nucleotide conjugates in the plurality can comprise a core bound to 2-5 nucleotide-arms, where the nucleotide-arms have one type of nucleotide unit comprising dATP, dGTP, dCTP, dTTP or dUTP. In some embodiments, individual nucleotide conjugates in the plurality can comprise a core bound to 2-5 biotinylated nucleotide-arms, where the biotinylated nucleotide-arms have one type of nucleotide unit comprising dATP, dGTP, dCTP, dTTP or dUTP.

The present disclosure provides compositions, systems, and kits comprising a plurality of nucleotide conjugates comprising a mixture (sub-populations) of two or more different types of nucleotide conjugates. In some embodiments, the plurality of nucleotide conjugates can have at least a first nucleotide conjugate in the plurality. In some cases, the at least the first nucleotide conjugate can comprise a core bound to at least one nucleotide-arm having a first type of nucleotide selected from a group consisting of dATP, dGTP, dCTP, dTTP or dUTP. In some embodiments, the plurality of nucleotide conjugates can have at least a second nucleotide conjugate. In some embodiments, the plurality of nucleotide conjugates can comprise at least a first nucleotide conjugate in the plurality and at least a second nucleotide conjugate. In some cases, the at least second nucleotide conjugate can comprise a core bound to at least one nucleotide-arm having a second type of nucleotide that differs from the first nucleotide in the first nucleotide conjugate. In some embodiments, the first nucleotide conjugate can comprise a core bound to 2-5 biotinylated nucleotide arms, where the biotinylated-arms can have a first type of nucleotide selected from a group consisting of dATP, dGTP, dCTP, dTTP or dUTP. In some embodiments, the second nucleotide conjugate can comprise a core bound to 2-5 biotinylated nucleotide arms, where the biotinylated-arms can have a second type of nucleotide selected from a group consisting of dATP, dGTP, dCTP, dTTP or dUTP, where the first and second type of nucleotides are different. In some embodiments, the mixture can comprise two, three, four, five, or more different types of nucleotide conjugates having nucleotides selected in any combination from a group consisting of dATP, dGTP, dCTP, dTTP or dUTP.

The present disclosure provides compositions, systems, and kits comprising a plurality (e.g., a population) of nucleotide conjugates, wherein individual nucleotide conjugates in the plurality can comprise a core bound to at least one nucleotide-arm. In some embodiments, the at least one nucleotide arm that are bound to a core can have the same spacer. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides compositions, systems, and kits comprising a plurality (e.g., a population) of nucleotide conjugates, wherein individual nucleotide conjugates in the plurality can comprise a core bound to at least one nucleotide-arm. In some embodiments, the at least one nucleotide-arm that are bound to a core can have the same linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides compositions, systems, and kits comprising a plurality (e.g., a population) of nucleotide conjugates, wherein individual nucleotide conjugates in the plurality can comprise a core bound to at least one nucleotide-arm. In some embodiments, all of the nucleotide arms that are bound to a core can have the same spacer and linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides compositions, systems, and kits comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first type of spacer. In some embodiments, the plurality of nucleotide conjugates can comprise at least a second nucleotide conjugate can comprise a core bound to at least one nucleotide-arm having a second type of spacer. In some embodiments, the plurality of nucleotide conjugates can comprise a mixture of the at least the first nucleotide conjugate and the at least the second nucleotide conjugate. In some cases, the second type of spacer in the second nucleotide conjugate can differ from the first spacer in the first nucleotide conjugate. In some embodiments, the first nucleotide conjugate can comprise a core bound to 2-5 biotinylated nucleotide arms, where the biotinylated-arms can have a first type of spacer. In some embodiments, the second nucleotide conjugate can comprise a core bound to 2-5 biotinylated nucleotide arms, where the biotinylated-arms can have a second type of spacer, where the first and second type of spacers are different.

The present disclosure provides compositions, systems, and kits comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality comprises a core bound to at least one nucleotide-arm having a first type of linker. In some embodiments, the plurality of nucleotide conjugates can comprise at least a second nucleotide conjugate comprises a core bound to at least one nucleotide-arm having a second type of linker. In some embodiments, the plurality of nucleotide conjugates can comprise a mixture of the at least the first nucleotide conjugate and the at least the second nucleotide conjugate. In some cases, the second type of linker in the second nucleotide conjugate can differ from the first linker in the first nucleotide conjugate. In some embodiments, the first nucleotide conjugate can comprise a core bound to 2-5 biotinylated nucleotide arms, where the biotinylated-arms can have a first type of linker. In some embodiments, the second nucleotide conjugate can comprise a core bound to 2-5 biotinylated nucleotide arms, where the biotinylated-arms can have a second type of linker, where the first and second type of spacers are different.

The present disclosure provides compositions, systems and kits comprising a plurality (e.g., a population) of nucleotide conjugates, wherein individual nucleotide conjugates in the plurality can comprise a core bound to at least one nucleotide-arm. In some embodiments, all of the nucleotide arms that are bound to a core can have the same reactive group in the linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the reactive group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a reactive group that can be reactive with a chemical agent. For example, the reactive groups alkyl, alkenyl, alkynyl and allyl are reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems and kits comprising a plurality of nucleotide conjugates comprising a mixture (sub-populations) of two or more different types of nucleotide conjugates. In some embodiments, the plurality of nucleotide conjugates can have at least a first nucleotide conjugate (a first subpopulation) in the plurality. In some embodiments, the at least the first subpopulation can comprise a core bound to at least one nucleotide-arm having a first type of reactive group in the linker. In some embodiments, the plurality of nucleotide conjugates can have at least a second nucleotide conjugate (a second subpopulation) comprises a core bound to at least one nucleotide-arm having a second type of reactive group in the linker. In some cases, the first reactive group in the first type of linker in the first sub-population differ from the second reactive group in the second type of linker in the second sub-population. In some embodiments, the first nucleotide conjugate can comprise a core bound to 2-5 biotinylated nucleotide arms, where the biotinylated-arms can have a first type of reactive group in the linker. In some embodiments, the second nucleotide conjugate can comprise a core bound to 2-5 biotinylated nucleotide arms, where the biotinylated-arms can have a second type of reactive group in the linker, where the first reactive group differs from the second reactive group.

In some embodiments, the first and second reactive group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second reactive group that can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the first or second reactive can be selected, in any combination, from a group consisting of an azide, azido or azidomethyl group. In some embodiments, the azide, azido or azidomethyl reactive group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, and kits comprising a plurality (e.g., a population) of nucleotide conjugates, wherein individual nucleotide conjugates in the plurality can comprise a core bound to at least one nucleotide-arm, wherein all of the nucleotide arms that are bound to a core can have a nucleotide unit having the same sugar 3′OH group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides compositions, systems, methods, and kits comprising a plurality (e.g., a population) of nucleotide conjugates, wherein individual nucleotide conjugates in the plurality can comprise a core bound to at least one nucleotide-arm, wherein all of the nucleotide arms that are bound to a core can have a nucleotide unit having the sugar 3′ OH group substituted with the same 3′ blocking group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the sugar 3′ blocking group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the 3′ blocking group can comprise a 3′-O-alkyl hydroxylamino group, a 3′-phosphorothioate group, a 3′-O-malonyl group, or a 3′-O-benzyl group. In some embodiments, the 3′ blocking group can comprise an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems and kits comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first nucleotide unit with a first type of sugar 3′ OH blocking group (chain terminating moiety). In some embodiments, the plurality of nucleotide conjugates can comprise at least a second nucleotide conjugate comprises a core bound to at least one nucleotide-arm having a second nucleotide unit having a second type of sugar 3′ blocking group (chain terminating moiety). In some embodiments, the plurality can comprise the first nucleotide conjugate and the second nucleotide conjugate. In some cases, the first 3′ blocking group can differs from the second 3′ blocking group. In some embodiments, the first nucleotide conjugate can comprise a core bound to 2-5 biotinylated nucleotide arms, where the biotinylated-arms can have a first type of 3′ blocking group. In some embodiments, the second nucleotide conjugate can comprise a core bound to 2-5 biotinylated nucleotide arms, where the biotinylated-arms can have a second type of 3′ blocking group, where the first 3′ blocking group differs from the second 3′ blocking group.

In some embodiments, the first and second 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first or second 3′ blocking group can be selected, in any combination, from a group consisting of an azide, azido or azidomethyl group. In some embodiments, the azide, azido or azidomethyl 3′ blocking group is reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, and kits comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first nucleotide unit with a sugar 3′ OH group. In some embodiments, the plurality of nucleotide conjugates can comprise at least a second nucleotide conjugate comprises a core bound to at least one nucleotide-arm having a second nucleotide unit having a first type of sugar 3′ blocking group. In some embodiments, the plurality of nucleotide conjugates can comprise the first nucleotide conjugate and the second nucleotide conjugate. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group.

In some embodiments, the first 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first 3′ blocking group can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides compositions, systems, and kits comprising a plurality of nucleotide conjugates comprising a mixture (sub-populations) of three or more different types of nucleotide conjugates. In some embodiments, the plurality of nucleotide conjugates can comprise at least a first nucleotide conjugate. In some embodiments, the at least the first nucleotide conjugate can comprise a core bound to at least one nucleotide-arm having a first nucleotide unit with a sugar 3′ OH group. In some embodiments, the plurality of nucleotide conjugates can comprise at least a second nucleotide conjugate. In some embodiments, the at least the second nucleotide conjugate can comprise a core bound to at least one nucleotide-arm having a second nucleotide unit having a first type of sugar 3′ blocking group. In some embodiments, the plurality of nucleotide conjugates can comprise at least a third nucleotide conjugate. In some embodiments, the at least third nucleotide conjugate can comprise a core bound to at least one nucleotide-arm having a third nucleotide unit having a second type of sugar 3′ blocking group. In some cases, the first and second 3′ blocking groups are different. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group. In some embodiments, the third nucleotide conjugate can comprise a core bound to 2-5 biotinylated nucleotide arms, where the biotinylated-arms can have a second type of 3′ blocking group.

In some embodiments, the first and second 3′ blocking groups can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking groups can be selected, in any combination, from a group consisting of a 3′-O-alkyl hydroxylamino group, a 3′-phosphorothioate group, a 3′-O-malonyl group, and a 3′-O-benzyl group. In some embodiments, the first and second 3′ blocking groups can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

### Formulations

Disclosed herein, in some embodiments, are formulations comprising any one of the compositions disclosed herein. Formulations of the present disclosure can comprise two or more compositions disclosed herein, such as, for example two types of nucleotide conjugates, or a combination of a nucleotide conjugate and a synthetic polypeptide disclosed herein. In some embodiments, the formulation further comprises one or more of a buffer, a solvent, diluent, target nucleic acid (e.g., DNA, RNA), nucleotides (e.g., dNTPs, rNTPs, etc.), a nucleic acid primer sequence (e.g., DNA primer, or a polymerizing enzyme (e.g., DNA polymerase disclose herein). In some embodiments, the nucleotides are labeled. In some embodiments, the nucleotides are unlabeled.

Disclosed herein, in some embodiments, are formulations comprising at least two of the compositions disclosed herein. In some embodiments, the at least two of the composition comprises a first composition and a second composition. In some embodiments, the nucleotide unit of the first composition comprises a different nucleobase than the nucleotide unit of the second composition. In some embodiments, the nucleotide unit of the first composition comprises a first blocking group. In some embodiments, the first blocking group is linked to the 3′ carbon of the sugar moiety. In some embodiments, the first blocking group reacts with a chemical compound to remove the first blocking group. In some embodiments, the first blocking group reacts with a chemical compound to remove the first blocking group and generate a OH moiety on the 3′ carbon of the sugar moiety of the nucleotide unit of the first composition. In some embodiments, the nucleotide unit of the second composition comprises a second blocking group. In some embodiments, the second blocking group is linked to the 3′ carbon of the sugar moiety. In some embodiments, the second blocking group reacts with a chemical compound to remove the second blocking group. In some embodiments, the second blocking group reacts with a chemical compound to remove the second blocking group and generate a OH moiety on the 3′ carbon of the sugar moiety of the nucleotide unit of the second composition. In some embodiments, the nucleotide unit of the first composition differs from the nucleotide unit of the second composition. In some embodiments, the nucleotide unit of the first composition comprises a first blocking group linked to the 3′ carbon of the sugar moiety and the first blocking group reacts with a chemical compound to remove the first blocking group and generate a OH moiety on the 3′ carbon of the sugar moiety of the nucleotide unit of the first composition. In some embodiments, the nucleotide unit of the second composition comprises a second blocking group linked to the 3′ carbon of the sugar moiety and the second blocking group reacts with a chemical compound to remove the second blocking group and generate a OH moiety on the 3′ carbon of the sugar moiety of the nucleotide unit of the second composition. In some embodiments, the nucleotide unit of the first composition differs from the nucleotide unit of the second composition. In some embodiments, the linker of the first composition differs from the linker of the second composition. In some embodiments, the linker of the first composition comprises a first reactive group and the linker of the second composition comprises a second reactive group. In some embodiments, the second reactive group differs from the first reactive group. In some embodiments, the first composition comprise a first fluorophore and the second composition comprise a second fluorophore. In some embodiments, the first fluorophore emits light at a wavelength that differs from the wavelength of light emitted from the second fluorophore. In some embodiments, the first composition comprises a fluorescent label and the second composition is unlabeled.

The present disclosure provides formulations comprising separate batches (sub-populations) of labeled nucleotide conjugates. In some embodiments, the separate batches of labeled nucleotide conjugates can be prepared using a different reporter moiety for each batch. In some embodiments, the different reporter moiety reporter moiety can correspond to a particular base in the nucleotide arms. A particular batch can be distinguishable from other batches based on the reporter moiety attached to the core. Two, three, four, five or more separate batches (sub-populations) can be mixed together to form a plurality of labeled nucleotide conjugates comprising two or more sub-populations of spectrally distinguishable nucleotide conjugates. In some embodiments, at least one batch of nucleotide conjugates in the mixture can be non-labeled (e.g., dark nucleotide conjugates).

The present disclosure provides formulations comprising a plurality of nucleotide conjugates which can comprise a mixture of at least two sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be labeled with a second reporter moiety that corresponds to a second nucleotide unit on the nucleotide-arms. In some cases, the first and second reporter moieties can differ from each other. In some embodiments, the plurality of nucleotide conjugates can further comprise at least a third sub-population of nucleotide conjugates which is labeled with a third reporter moiety, wherein the first, second and third reporter moieties can differ from each other. In some embodiments, the plurality of nucleotide conjugates can further comprises at least a fourth sub-population of nucleotide conjugates which is labeled with a fourth reporter moiety, wherein the first, second, third and fourth reporter moieties can differ from each other. In some embodiments, additional sub-populations (e.g., fifth, sixth, seventh, eighth, ninth, tenth or more) of labeled nucleotide conjugates can be added into the mixture. In some embodiments, the reporter moiety can be a fluorophore. In some embodiments, a first sub-population of nucleotide conjugates can be labeled with a first fluorophore and a second fluorophore of nucleotide conjugates can be labeled with a second fluorophore. In some cases, the first fluorophore and the second fluorophore can be different.

The present disclosure provides formulations comprising a plurality of nucleotide conjugates which can comprises a mixture of at least two sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be non-labeled (e.g., a dark nucleotide conjugate).

The present disclosure provides compositions, systems, methods, and kits comprising a plurality of nucleotide conjugates which comprises a mixture of at least three sub-populations of nucleotide conjugates labeled with different reporter moieties. In some embodiments, at least a first sub-population of nucleotide conjugates can be labeled with a first reporter moiety that corresponds to a first nucleotide unit on the nucleotide-arms. In some embodiments, at least a second sub-population of nucleotide conjugates can be labeled with a second reporter moiety that corresponds to a second nucleotide unit on the nucleotide-arms. In some embodiments, at least a third sub-population of nucleotide conjugates can be non-labeled (e.g., a dark nucleotide conjugate). In some embodiments, the first and second reporter moieties can differ from each other.

The present disclosure provides formulations comprising a plurality of nucleotide conjugates which comprises a mixture of at least four sub-populations of nucleotide conjugates labeled with different reporter moieties. In some embodiments, the mixture of nucleotide conjugates can have at least a first sub-population of nucleotide conjugates can be labeled with a first reporter moiety that corresponds to a first nucleotide unit on the nucleotide-arms. In some embodiments, the mixture of nucleotide conjugates can have at least a second sub-population of nucleotide conjugates can be labeled with a second reporter moiety that corresponds to a second nucleotide unit on the nucleotide-arms. In some embodiments, the mixture of nucleotide conjugates can have at least a third sub-population of nucleotide conjugates is labeled with a third reporter moiety. In some embodiments, the mixture of nucleotide conjugates can have at least a fourth sub-population of nucleotide conjugates can be non-labeled (e.g., a dark nucleotide conjugate). In some cases, the first, second and third reporter moieties can differ from each other.

An embodiment comprises: a mixture of four different types of nucleotide conjugates comprising (1) a first sub-population of nucleotide conjugates each comprising a dATP nucleotide unit and a core labeled with a first type of fluorophore, (2) a second sub-population of nucleotide conjugates each comprising a dGTP nucleotide unit and a core labeled with a second type of fluorophore, (3) a third sub-population of nucleotide conjugates each comprising a dCTP nucleotide unit and a core labeled with a third type of fluorophore, and (4) a fourth sub-population of nucleotide conjugates each comprising a dTTP nucleotide unit and a core labeled with a fourth type of fluorophore, where the first, second, third and fourth fluorophores can be spectrally distinguishable. In some embodiments, any one of the sub-populations of nucleotide conjugates can be non-labeled for use as “dark” nucleotide conjugates.

Disclosed herein are formulations comprising a first nucleotide conjugate and a second nucleotide conjugate. In some embodiments, the formulation comprises a first nucleotide conjugate, a second nucleotide conjugate, and a third nucleotide conjugate. In some embodiments, the formulation comprises a first nucleotide conjugate, a second nucleotide conjugate, a third nucleotide conjugate, and a fourth nucleotide conjugate. In some embodiments, the first nucleotide conjugate comprises a first label. In some embodiments, the second nucleotide conjugate comprises a second label. In some embodiments, the third nucleotide conjugate comprises a third label. In some embodiments, the fourth nucleotide conjugate comprises a fourth label. In some embodiments, the first label comprises a first fluorophore. In some embodiments, the second label comprises a second fluorophore. In some embodiments, the third label comprises a third fluorophore. In some embodiments, the fourth label comprises a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other. Mixtures of different sub-populations of labeled nucleotide conjugates may comprise a first sub-population of nucleotide conjugates comprising the first fluorophore and a second sub-population of nucleotide conjugates comprising the second fluorophore

The present disclosure provides formulations comprising a plurality (e.g., a population) of nucleotide conjugates, wherein individual nucleotide conjugates in the plurality can comprise a core bound to at least one nucleotide-arm. In some embodiments, individual nucleotide conjugates in the plurality can comprise a core bound to 2-5 nucleotide-arms. In some embodiments, individual nucleotide conjugates in the plurality can comprise a streptavidin or avidin core bound to 2-5 biotinylated nucleotide-arms.

The present disclosure provides formulations comprising a plurality (e.g., a population) of nucleotide conjugates, wherein individual nucleotide conjugates in the plurality can comprise a core bound to at least one nucleotide-arm having one type of nucleotide unit comprising dATP, dGTP, dCTP, dTTP or dUTP. In some embodiments, individual nucleotide conjugates in the plurality can comprise a core bound to 2-5 nucleotide-arms, where the nucleotide-arms have one type of nucleotide unit comprising dATP, dGTP, dCTP, dTTP or dUTP. In some embodiments, individual nucleotide conjugates in the plurality can comprise a core bound to 2-5 biotinylated nucleotide-arms, where the biotinylated nucleotide-arms have one type of nucleotide unit comprising dATP, dGTP, dCTP, dTTP or dUTP.

The present disclosure provides formulations comprising a plurality of nucleotide conjugates comprising a mixture (sub-populations) of two or more different types of nucleotide conjugates. In some embodiments, the plurality of nucleotide conjugates can have at least a first nucleotide conjugate in the plurality. In some cases, the at least the first nucleotide conjugate can comprise a core bound to at least one nucleotide-arm having a first type of nucleotide selected from a group consisting of dATP, dGTP, dCTP, dTTP or dUTP. In some embodiments, the plurality of nucleotide conjugates can have at least a second nucleotide conjugate. In some embodiments, the plurality of nucleotide conjugates can comprise at least a first nucleotide conjugate in the plurality and at least a second nucleotide conjugate. In some cases, the at least second nucleotide conjugate can comprise a core bound to at least one nucleotide-arm having a second type of nucleotide that differs from the first nucleotide in the first nucleotide conjugate. In some embodiments, the first nucleotide conjugate can comprise a core bound to 2-5 biotinylated nucleotide arms, where the biotinylated-arms can have a first type of nucleotide selected from a group consisting of dATP, dGTP, dCTP, dTTP or dUTP. In some embodiments, the second nucleotide conjugate can comprise a core bound to 2-5 biotinylated nucleotide arms, where the biotinylated-arms can have a second type of nucleotide selected from a group consisting of dATP, dGTP, dCTP, dTTP or dUTP, where the first and second type of nucleotides are different. In some embodiments, the mixture can comprise two, three, four, five, or more different types of nucleotide conjugates having nucleotides selected in any combination from a group consisting of dATP, dGTP, dCTP, dTTP or dUTP.

The present disclosure provides formulations comprising a plurality (e.g., a population) of nucleotide conjugates, wherein individual nucleotide conjugates in the plurality can comprise a core bound to at least one nucleotide-arm. In some embodiments, the at least one nucleotide arm that are bound to a core can have the same spacer. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides formulations comprising a plurality (e.g., a population) of nucleotide conjugates, wherein individual nucleotide conjugates in the plurality can comprise a core bound to at least one nucleotide-arm. In some embodiments, the at least one nucleotide-arm that are bound to a core can have the same linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides formulations comprising a plurality (e.g., a population) of nucleotide conjugates, wherein individual nucleotide conjugates in the plurality can comprise a core bound to at least one nucleotide-arm. In some embodiments, all of the nucleotide arms that are bound to a core can have the same spacer and linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides formulations comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first type of spacer. In some embodiments, the plurality of nucleotide conjugates can comprise at least a second nucleotide conjugate can comprise a core bound to at least one nucleotide-arm having a second type of spacer. In some embodiments, the plurality of nucleotide conjugates can comprise a mixture of the at least the first nucleotide conjugate and the at least the second nucleotide conjugate. In some cases, the second type of spacer in the second nucleotide conjugate can differ from the first spacer in the first nucleotide conjugate. In some embodiments, the first nucleotide conjugate can comprise a core bound to 2-5 biotinylated nucleotide arms, where the biotinylated-arms can have a first type of spacer. In some embodiments, the second nucleotide conjugate can comprise a core bound to 2-5 biotinylated nucleotide arms, where the biotinylated-arms can have a second type of spacer, where the first and second type of spacers are different.

The present disclosure provides formulations comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality comprises a core bound to at least one nucleotide-arm having a first type of linker. In some embodiments, the plurality of nucleotide conjugates can comprise at least a second nucleotide conjugate comprises a core bound to at least one nucleotide-arm having a second type of linker. In some embodiments, the plurality of nucleotide conjugates can comprise a mixture of the at least the first nucleotide conjugate and the at least the second nucleotide conjugate. In some cases, the second type of linker in the second nucleotide conjugate can differ from the first linker in the first nucleotide conjugate. In some embodiments, the first nucleotide conjugate can comprise a core bound to 2-5 biotinylated nucleotide arms, where the biotinylated-arms can have a first type of linker. In some embodiments, the second nucleotide conjugate can comprise a core bound to 2-5 biotinylated nucleotide arms, where the biotinylated-arms can have a second type of linker, where the first and second type of spacers are different.

The present disclosure provides formulations comprising a plurality (e.g., a population) of nucleotide conjugates, wherein individual nucleotide conjugates in the plurality can comprise a core bound to at least one nucleotide-arm. In some embodiments, all of the nucleotide arms that are bound to a core can have the same reactive group in the linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the reactive group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a reactive group that can be reactive with a chemical agent. For example, the reactive groups alkyl, alkenyl, alkynyl and allyl are reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides formulations comprising a plurality of nucleotide conjugates comprising a mixture (sub-populations) of two or more different types of nucleotide conjugates. In some embodiments, the plurality of nucleotide conjugates can have at least a first nucleotide conjugate (a first subpopulation) in the plurality. In some embodiments, the at least the first subpopulation can comprise a core bound to at least one nucleotide-arm having a first type of reactive group in the linker. In some embodiments, the plurality of nucleotide conjugates can have at least a second nucleotide conjugate (a second subpopulation) comprises a core bound to at least one nucleotide-arm having a second type of reactive group in the linker. In some cases, the first reactive group in the first type of linker in the first sub-population differ from the second reactive group in the second type of linker in the second sub-population. In some embodiments, the first nucleotide conjugate can comprise a core bound to 2-5 biotinylated nucleotide arms, where the biotinylated-arms can have a first type of reactive group in the linker. In some embodiments, the second nucleotide conjugate can comprise a core bound to 2-5 biotinylated nucleotide arms, where the biotinylated-arms can have a second type of reactive group in the linker, where the first reactive group differs from the second reactive group.

In some embodiments, the first and second reactive group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second reactive group that can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the first or second reactive can be selected, in any combination, from a group consisting of an azide, azido or azidomethyl group. In some embodiments, the azide, azido or azidomethyl reactive group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides formulations comprising a plurality (e.g., a population) of nucleotide conjugates, wherein individual nucleotide conjugates in the plurality can comprise a core bound to at least one nucleotide-arm, wherein all of the nucleotide arms that are bound to a core can have a nucleotide unit having the same sugar 3′OH group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

The present disclosure provides formulations comprising a plurality (e.g., a population) of nucleotide conjugates, wherein individual nucleotide conjugates in the plurality can comprise a core bound to at least one nucleotide-arm, wherein all of the nucleotide arms that are bound to a core can have a nucleotide unit having the sugar 3′ OH group substituted with the same 3′ blocking group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the sugar 3′ blocking group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the 3′ blocking group can comprise a 3′-O-alkyl hydroxylamino group, a 3′-phosphorothioate group, a 3′-O-malonyl group, or a 3′-O-benzyl group. In some embodiments, the 3′ blocking group can comprise an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides formulations comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first nucleotide unit with a first type of sugar 3′ OH blocking group (chain terminating moiety). In some embodiments, the plurality of nucleotide conjugates can comprise at least a second nucleotide conjugate comprises a core bound to at least one nucleotide-arm having a second nucleotide unit having a second type of sugar 3′ blocking group (chain terminating moiety). In some embodiments, the plurality can comprise the first nucleotide conjugate and the second nucleotide conjugate. In some cases, the first 3′ blocking group can differs from the second 3′ blocking group. In some embodiments, the first nucleotide conjugate can comprise a core bound to 2-5 biotinylated nucleotide arms, where the biotinylated-arms can have a first type of 3′ blocking group. In some embodiments, the second nucleotide conjugate can comprise a core bound to 2-5 biotinylated nucleotide arms, where the biotinylated-arms can have a second type of 3′ blocking group, where the first 3′ blocking group differs from the second 3′ blocking group.

In some embodiments, the first and second 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first or second 3′ blocking group can be selected, in any combination, from a group consisting of an azide, azido or azidomethyl group. In some embodiments, the azide, azido or azidomethyl 3′ blocking group is reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides formulations comprising a plurality of nucleotide conjugates comprising a mixture (sub-populations) of two or more different types of nucleotide conjugates. In some embodiments, the plurality of nucleotide conjugates can comprise at least a first nucleotide conjugate in the plurality can comprise a core bound to at least one nucleotide-arm having a first nucleotide unit with a sugar 3′ OH group. In some embodiments, the plurality of nucleotide conjugates can comprise at least a second nucleotide conjugate comprises a core bound to at least one nucleotide-arm having a second nucleotide unit having a first type of sugar 3′ blocking group. In some embodiments, the plurality of nucleotide conjugates can comprise the first nucleotide conjugate and the second nucleotide conjugate. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group.

In some embodiments, the first 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first 3′ blocking group can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

The present disclosure provides formulations comprising a plurality of nucleotide conjugates comprising a mixture (sub-populations) of three or more different types of nucleotide conjugates. In some embodiments, the plurality of nucleotide conjugates can comprise at least a first nucleotide conjugate. In some embodiments, the at least the first nucleotide conjugate can comprise a core bound to at least one nucleotide-arm having a first nucleotide unit with a sugar 3′ OH group. In some embodiments, the plurality of nucleotide conjugates can comprise at least a second nucleotide conjugate. In some embodiments, the at least the second nucleotide conjugate can comprise a core bound to at least one nucleotide-arm having a second nucleotide unit having a first type of sugar 3′ blocking group. In some embodiments, the plurality of nucleotide conjugates can comprise at least a third nucleotide conjugate. In some embodiments, the at least third nucleotide conjugate can comprise a core bound to at least one nucleotide-arm having a third nucleotide unit having a second type of sugar 3′ blocking group. In some cases, the first and second 3′ blocking groups are different. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group. In some embodiments, the third nucleotide conjugate can comprise a core bound to 2-5 biotinylated nucleotide arms, where the biotinylated-arms can have a second type of 3′ blocking group.

In some embodiments, the first and second 3′ blocking groups can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking groups can be selected, in any combination, from a group consisting of a 3′-O-alkyl hydroxylamino group, a 3′-phosphorothioate group, a 3′-O-malonyl group, and a 3′-O-benzyl group. In some embodiments, the first and second 3′ blocking groups can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

### Systems

Disclosed herein, in some embodiments, are systems comprising at least one of the compositions described herein (e.g., any or any combination of two or more of the compositions described herein) and a second component. In some embodiments, the second component comprises a second composition disclosed herein, such as at least one synthetic polypeptide disclosed herein. In some embodiments, the system further comprises at least one of a buffer, a solvent, diluent, target nucleic acid (e.g., DNA, RNA), nucleotides (e.g., dNTPs, rNTPs, etc.), a nucleic acid primer sequence (e.g., DNA primer, or a polymerizing enzyme (e.g., DNA polymerase disclose herein). In some embodiments, the nucleotides are labeled. In some embodiments, the nucleotides are unlabeled. In some embodiments, the target nucleic acid is a concatemer comprising multiple repeats of a target nucleic acid sequence. In some embodiments, the system comprises a computer systems having one or more system modules configured for performing any of the disclosed methods for nucleic acid processing, sequencing, detection and/or analysis.

In some embodiments, the systems comprise a binding complex formed by a nucleotide conjugate, one or more polymerizing enzymes (e.g., synthetic polypeptide) and/or a target nucleic acid sequence. In some embodiments, the binding complex comprises a multivalent binding complex comprising two or more copies of the target nucleic acid sequence bound to two or more nucleotide units of a nucleotide conjugate disclosed herein, and two or more polymerizing enzymes (e.g., synthetic polypeptides) disclosed herein. In some embodiments, the binding complex can comprise a synthetic polypeptide bound to a nucleic acid template molecule which is hybridized to a nucleic acid primer, and the composition can be bound to the nucleic acid primer. In some embodiments, the multivalent binding complex can comprise two or more of the synthetic polypeptide bound to two or more the nucleic acid template molecule which are hybridized to two or more of the nucleic acid primer, and the composition can be bound to the nucleic acid primer. In some embodiments, the systems can comprise a plurality of the binding complexes. In some embodiments, the system can further comprise at least one nucleic acid template molecule, at least one nucleic acid primer molecule, or a combination of at least one nucleic acid template molecule and at least one nucleic acid primer molecule. In some embodiments, the composition may not be bound to the synthetic polypeptide. In some embodiments, the composition may not be bound to the template molecule. In some embodiments, the composition may not be bound to the primer. In some embodiments, the composition may be bound to the synthetic polypeptide, the template molecule, the primer, or a combination thereof. In some embodiments, the system can comprise at least one synthetic polypeptide where individual synthetic polypeptide can be bound to a nucleic acid template molecule which is hybridized to a nucleic acid primer molecule to form a complexed synthetic polypeptide, and the complexed synthetic polypeptide can further comprise a nucleotide conjugate. In some embodiments, in the complexed synthetic polypeptide, the nucleic acid template and primer can be replaced with a nucleic acid template that includes a primer sequence to form a self-priming template nucleic acid molecule. In some embodiments, the complexed synthetic polypeptide can include a composition which is not bound to the synthetic polypeptide, the template or the primer molecule. In some embodiments, the complexed synthetic polypeptide can include a composition having a nucleotide unit which is bound to the 3′ terminal end of the primer at a position that is opposite a complementary nucleotide in the template strand.

Disclosed herein is a system comprising a synthetic polypeptide disclosed herein. In some embodiments, the system comprises a primed nucleic acid sequence; and a nucleotide unit. In some embodiments, the nucleotide unit is detectable. In some embodiments, the nucleotide unit is complementary to a nucleotide in the primed nucleic acid sequence. In some embodiments, the system is configured to form a binding complex. In some embodiments, the binding complex comprises the primed nucleic acid sequence, the synthetic polypeptide, and the nucleotide unit. In some embodiments, the system further comprises: one or more compositions. In some embodiments, a composition of the one or more compositions comprises: a core; and at least two nucleotide arms. In some embodiments, a nucleotide arm of the at least two nucleotide arms comprises: a core attachment moiety coupled to the core; a spacer coupled to the core attachment moiety; a linker coupled to the spacer; and the nucleotide unit coupled to the linker. In some embodiments, the binding complex comprises the primed nucleic acid sequence, the synthetic polypeptide and the composition. In some embodiments, the system further comprises: two or more copies of the primed nucleic acid sequence; and two or more of the synthetic polypeptide. In some embodiments, the composition is configured to form a multivalent binding complex. In some embodiments, multivalent binding complex comprises two or more of the nucleotide unit of the composition, the two or more copies of the primed nucleic acid sequence, and the two or more of the synthetic polypeptide. In some embodiments, the linker comprises:

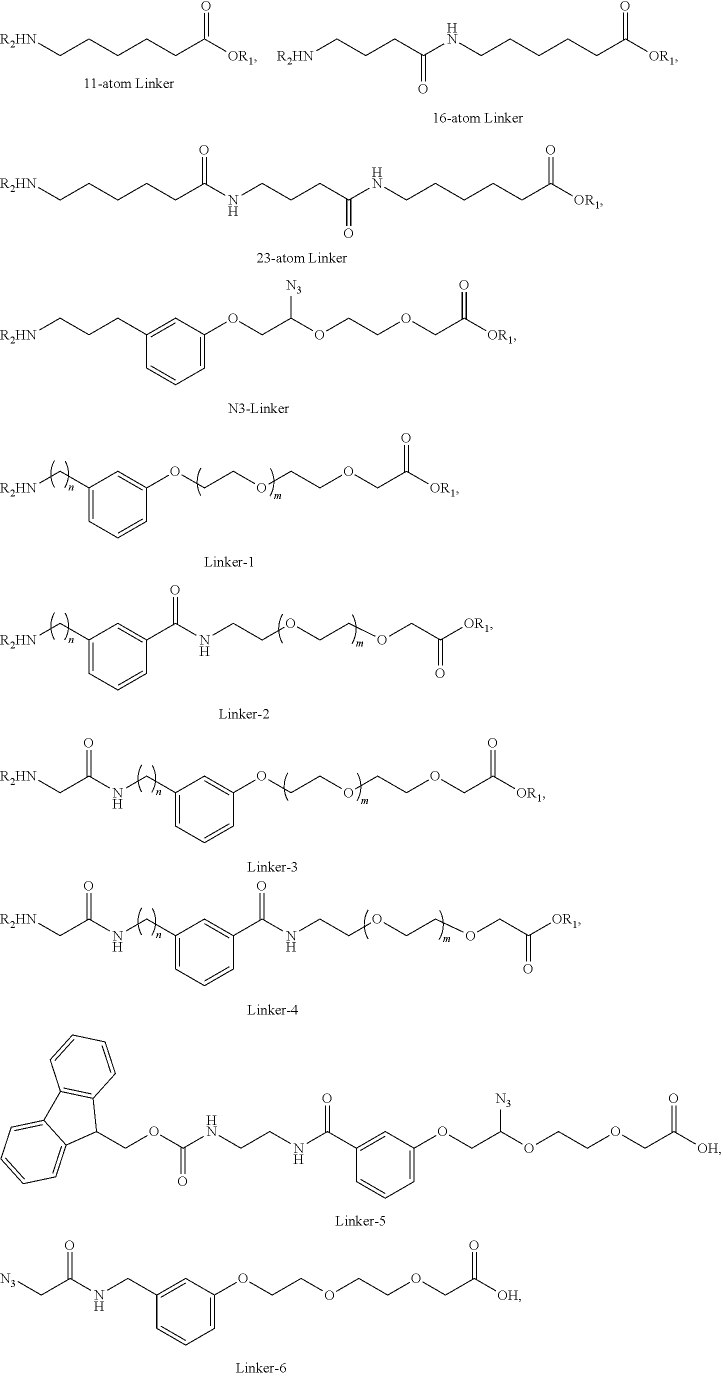

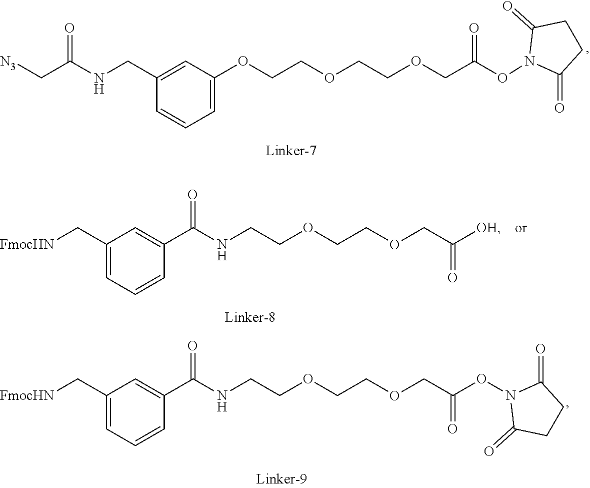

and n is 1 to 6 and m is 0 to 10.

Disclosed herein is a system comprising a composition disclosed herein. In some embodiments, the system further comprises two or more copies of a primed nucleic acid sequence. In some embodiments, the two or more copies of the primed nucleic acid sequence comprise a nucleotide complementary to the nucleotide unit of the composition. In some embodiments, the system further comprises two or more of a polymerizing enzyme. Disclosed herein is a system comprising: (i) a composition disclosed herein; (ii) two or more copies of a primed nucleic acid sequence and the two or more copies of the primed nucleic acid sequence comprise a nucleotide complementary to the nucleotide unit of the composition; and (iii) two or more of a polymerizing enzyme. In some embodiments, the system is configured to form a multivalent binding complex. In some embodiments, the multivalent binding complex comprises the two or more primed nucleic acid sequences, the two or more of the polymerizing enzyme and the composition. In some embodiments, the system is configured to form a multivalent binding complex comprising the two or more primed nucleic acid sequences, the two or more of the polymerizing enzyme and the composition. In some embodiments, the multivalent binding complex is formed under conditions such that the nucleotide unit of the composition is not incorporated into the two or more copies of the primed nucleic acid sequence. In some embodiments, the two or more copies of the nucleic acid sequence and the two or more copies of the nucleic acid primer molecule are immobilized to a support under conditions sufficient to immobilize the multivalent binding complex to the support. In some embodiments, a plurality of the multivalent binding complex is immobilized on the support. In some embodiments, a density of the plurality of the multivalent binding complex immobilized on the support is 10²-10⁹ per millimeter squared (mm²). In some embodiments, the plurality of the multivalent binding complex on the support is in fluid communication with each other. In some embodiments, the plurality of the multivalent binding complex on the support is in fluid communication with a solution of reagents under conditions sufficient that the plurality of the multivalent binding complex reacts with the solution of reagents in a massively parallel manner. In some embodiments, the plurality of the multivalent binding complex on the support is in fluid communication with each other and a solution of reagents under conditions sufficient that the plurality of the multivalent binding complex reacts with the solution of reagents in a massively parallel manner.

In some embodiments, the composition may comprise at least two of the composition disclosed herein. In some embodiments, the at least two of the composition comprises a first composition and a second composition. In some embodiments, the at least two of the composition comprises a first composition, a second composition, and a third composition. In some embodiments, the at least two of the composition comprises a first composition, a second composition, a third composition, and a fourth composition. In some embodiments, the first composition comprises a first fluorophore. In some embodiments, the second composition comprise a second fluorophore. In some embodiments, the third composition comprise a third fluorophore. In some embodiments, the fourth composition comprise a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other.

Disclosed herein, in another aspect, are systems comprising a synthetic polypeptide disclosed herein. In some embodiments, the system further comprises a primed nucleic acid sequence. In some embodiments, the system further comprises a nucleotide unit complementary to a nucleotide of the primed nucleic acid. In some embodiments, the system is provided under conditions sufficient to form a binding complex comprising the synthetic polypeptide, the nucleotide unit, and the primed nucleic acid sequence. Disclosed herein, in another aspect, are systems comprising: (i) a synthetic polypeptide disclosed herein; (ii) a primed nucleic acid sequence; and (iii) a nucleotide unit complementary to a nucleotide of the primed nucleic acid and the system is provided under conditions sufficient to form a binding complex comprising the synthetic polypeptide, the nucleotide unit, and the primed nucleic acid sequence.

Disclosed herein, in another aspect, are systems comprising a synthetic polypeptide disclosed herein. In some embodiments, the system further comprises a nucleotide. In some embodiments, the system comprises a synthetic polypeptide disclosed herein and a nucleotide. In some embodiments, the nucleotide comprises a blocking group. In some embodiments, the nucleotide does not comprise a blocking group. In some embodiments, the nucleotide comprises a label. In some embodiments, the nucleotide is unlabeled. In some embodiments, the blocking group is linked to the 3′ carbon of the sugar moiety of the nucleotide. In some embodiments, the blocking group reacts with a chemical compound to remove the blocking group from the nucleotide to generate the nucleotide comprising a OH moiety on the 3′ carbon of the sugar moiety of the nucleotide. In some embodiments, the blocking group comprises an alkyl, an alkenyl, an alkynyl, an allyl, an aryl, a benzyl, an azide, an amine, an amide, a keto, an isocyanate, a phosphate, a thiol, a disulfide, a carbonate, a urea, or a silyl group. In some embodiments, the alkyl, alkenyl, alkynyl, or allyl group of the blocking group reacts with tetrakis(triphenylphosphine)palladium(0) (Pd(P(C6H5)3)4) with piperidine, or 2,3-Dichloro-5,6-dicyano-1,4-benzo-quinone (DDQ). In some embodiments, the aryl or benzyl group of the blocking group reacts with H2 and Palladium on carbon (Pd/C). In some embodiments, the amine, amide, keto, isocyanate, phosphate, thiol, or disulfide group of the blocking group reacts with phosphine, or a thiol group comprising beta-mercaptoethanol, or dithiothritol (DTT). In some embodiments, the carbonate group of the blocking group reacts with potassium carbonate (K₂CO3) in methanol (MeOH), triethylamine in pyridine, or Zn in acetic acid (AcOH). In some embodiments, the urea or silyl group of the blocking group reacts with tetrabutylammonium fluoride, HF-pyridine, ammonium fluoride, or triethylamine trihydrofluoride. In some embodiments, the azide of the blocking group comprises an azide, an azido or an azidomethyl group. In some embodiments, the azide, azido, or azidomethyl group reacts with a chemical agent that comprises a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP).

In any of the systems or methods described above, the nucleotide conjugate(s) can be labeled with a detectable reporter moiety, non-labeled, or a mixture of labeled and non-labeled forms. In some embodiments, at least one of the polymerases can be labeled with a detectable reporter moiety. In some embodiments, the nucleic acid template can comprise a linear or circular molecule. In some embodiments, the template molecule can be labeled with a detectable reporter moiety. In some embodiments, the nucleic acid template can be a clonally-amplified template molecule. A clonally-amplified template molecule can include a concatemer molecule. The template and primer can be wholly or partially complementary along the hybridized region. The primer can comprise an extendible 3′ terminal end or a non-extendible 3′ terminal end. In some embodiments, the template, the primer, or a combination of the template and the primer can be immobilized to a support. In some embodiments, the polymerase can be immobilized to a support.

The present disclosure provides a system or method comprising at least one binding complex. In some embodiments, the binding complex can comprise a polymerase bound to a nucleic acid template molecule. In some cases, the polymerase bound to the nucleic acid template molecule can be hybridized to a primer. In some cases, the polymerase bound to the nucleic template molecule can be hybridized to a nucleotide conjugate. In some cases, the polymerase bound to the nucleic acid template molecule can be hybridized to the primer and the nucleotide conjugate. In some cases, a first nucleotide unit of the nucleotide conjugate can be bound to the 3′ terminal end of the primer at a position that is opposite a first complementary nucleotide in the template molecule. In some embodiments, the template molecule, primer molecule, polymerase, or a combination thereof can be immobilized to a support or immobilized to a coating on the support.

The present disclosure provides a system or method comprising at least two binding complexes located on the same template molecule. In some embodiment, a first binding complex can comprise a first polymerase bound to a first nucleic acid template molecule. In some cases, the first polymerase bound to the first nucleic acid template molecule can be hybridized to a first primer. In some cases, the first polymerase bound to the first nucleic acid template molecule can be hybridized to a first nucleotide conjugate. In some cases, the first polymerase bound to the first nucleic acid template molecule can be hybridized to the first primer and the first nucleotide conjugate. In some cases, a first nucleotide unit of the first nucleotide conjugate can be bound to the 3′ terminal end of the first primer at a position that is opposite a first complementary nucleotide in the first template molecule. In some embodiments, a second binding complex can comprise a second polymerase bound to the first nucleic acid template molecule. In some cases, the second polymerase bound to the first nucleic acid template molecule can be hybridized to a second primer. In some cases, the second polymerase bound to the first nucleic acid template molecule can be hybridized to a second nucleotide conjugate. In some cases, the second polymerase bound to the first nucleic acid template molecule can be hybridized to the second primer and the second nucleotide conjugate. In some cases, a second nucleotide unit of the second nucleotide conjugate can be bound to the 3′ terminal end of the second primer at a position that is opposite a second complementary nucleotide in the second template molecule. In some embodiments, the system can further comprise 1, 2, 3, 4, 5, 6, 7, 8, 9, 10 or more binding complexes on the same template molecule. In some embodiments, the template, the primer, or a combination of the template and the primer can be immobilized to a support or to a coating on the support. In some embodiments, the polymerase can be immobilized to the support or to a coating on the support.

In some embodiments, the system or method can comprise at least one multivalent binding complex which includes at least two binding complexes on the same template molecule. In some embodiments, the first multivalent binding complex can comprise a first and a second binding complex and a nucleotide conjugate. In some cases, the first binding complex can comprise a first nucleic acid primer, a first polymerase, and a first nucleotide conjugate bound to a first portion of a concatemer template molecule. In some cases, a first nucleotide unit of the nucleotide conjugate can be bound to the first polymerase. In some cases, the second binding complex can comprise a second nucleic acid primer, a second polymerase, and the first nucleotide conjugate bound to a second portion of the same concatemer template molecule. In some cases, a second nucleotide unit of the nucleotide conjugate can be bound to the second polymerase, wherein the first and second binding complexes which include the same nucleotide conjugate forms the first multivalent binding complex. In some embodiments, the first multivalent binding complex can further comprise a third binding complex. In some cases, the third binding complex can comprise a third nucleic acid primer, a third polymerase, and the first nucleotide conjugate bound to a third portion of the concatemer template molecule. In some cases, a third nucleotide unit of the nucleotide conjugate can be bound to the third polymerase. In some embodiments, the first multivalent binding complex can further comprise a fourth binding complex. In some cases, the fourth binding complex can comprise a fourth nucleic acid primer, a fourth polymerase, and the first nucleotide conjugate bound to a fourth portion of the concatemer template molecule. In some cases, a fourth nucleotide unit of the nucleotide conjugate can be bound to the fourth polymerase. The concatemer template molecule can comprise tandem repeat sequences of a sequence of interest and at least one universal sequencing primer binding site. The first, second, third and fourth nucleic acid primers can bind to the sequencing primer binding sites along the concatemer template molecule. The level of valency of the nucleotide units of a given nucleotide conjugate corresponds to the number of nucleotide arms linked to a core. For example, a nucleotide conjugate comprising a streptavidin or avidin core can have a valency of 1, 2, 3 or 4. In some embodiments, a single nucleotide conjugate can essentially simultaneously bind multiple complexed polymerases which are bound to the same template molecule (e.g., a concatemer).

The present disclosure provides a system or method comprising at least two binding complexes located on different template molecules. In some embodiments, a first binding complex can comprise a first polymerase bound to a first nucleic acid template molecule which is hybridized to a first primer. and a first nucleotide conjugate. In some cases, a first nucleotide unit of the nucleotide conjugate can be bound to the 3′ terminal end of the first primer at a position that is opposite a first complementary nucleotide in the first template molecule. In some embodiments, the second binding complex can comprise a second polymerase bound to a second nucleic acid template molecule which is hybridized to a second primer, and a second nucleotide conjugate. In some cases, a second nucleotide unit of the nucleotide conjugate can be bound to the 3′ terminal end of the second primer at a position that is opposite a second complementary nucleotide in the second template molecule. In some embodiments, the system can further comprise 1, 2, 3, 4, 5, 6, 7, 8, 9, 10 or more binding complexes where individual binding complexes can be located on different template molecules. In some embodiments, the template, the primer, or a combination of the template and the primer can be immobilized to a support or to a coating on the support. In some embodiments, the polymerase can be immobilized to the support or to a coating on the support.

In some embodiments, the system can comprise at least one multivalent binding complex. In some embodiments, an multivalent binding complex can comprise at least two binding complexes on different clonally-amplified template molecules described herein which can be localized in close proximity of each other. In some embodiments, the clonally-amplified template molecules can comprise a plurality of linear template molecules that can be generated via bridge amplification and can be immobilized to the same location or feature on a support. In some embodiments, the first multivalent binding complex can comprise a first and a second binding complex and a nucleotide conjugate. In some cases, the first binding complex comprises a first nucleic acid primer, a first polymerase, and a first nucleotide conjugate bound to a first portion of a first clonally amplified template molecule. In some cases, a first nucleotide unit of the nucleotide conjugate can be bound to the first polymerase. In some embodiments, the second binding complex can comprise a second nucleic acid primer, a second polymerase, and the first nucleotide conjugate bound to a second clonally-amplified template molecule. In some cases, a second nucleotide unit of the nucleotide conjugate can be bound to the second polymerase, wherein the first and second binding complexes which include the same nucleotide conjugate forms an multivalent binding complex. In some embodiments, the first clonally-amplified template molecule and the second clonally-amplified template molecule can be generated via bridge amplification. In some cases, the first clonally-amplified template molecule and the second clonally-amplified template molecule can be generated via bridge amplification. In some cases, the first clonally-amplified template molecule and the second clonally-amplified template molecule can be generated immobilized to the same location or feature on a support. The first multivalent binding complex can further comprise a third binding complex. In some embodiments, the third binding complex can comprise a third nucleic acid primer, a third polymerase, and the first nucleotide conjugate bound to a third template molecule. In some cases, a third nucleotide unit of the nucleotide conjugate can be bound to the third polymerase. The first multivalent binding complex can further comprise a fourth binding complex. In some embodiments, the fourth binding complex can comprise a fourth nucleic acid primer, a fourth polymerase, and the first nucleotide conjugate bound to a fourth template molecule. In some cases, a fourth nucleotide unit of the nucleotide conjugate is bound to the fourth polymerase. The linear template molecules can comprise a sequence of interest and at least one universal sequencing primer binding site. The first, second, third and fourth nucleic acid primers can bind to the sequencing primer binding sites on the first, second, third and fourth template molecules, respectively. The level of valency of the nucleotide units of a given nucleotide conjugate corresponds to the number of nucleotide arms linked to a core. For example, a nucleotide conjugate comprising a streptavidin or avidin core can have a valency of 1, 2, 3 or 4. In some embodiments, a single nucleotide conjugate can essentially simultaneously bind multiple complexed polymerases which are bound to different clonally amplified template molecules.

In some embodiments, the system or method can further comprise a reagent suitable to facilitate nucleic acid interactions, such as template-primer hybridization, self-hybridization, secondary or tertiary structure formation, nucleobase pairing, surface association, peptide association, protein binding, or the like. In some embodiments, the reagent can comprise cations including sodium, magnesium, strontium, barium, potassium, manganese, calcium, lithium, nickel, cobalt, or other cations.

In some embodiments, the system or method can further comprise a reagent suitable for binding a nucleotide unit of a nucleotide conjugate to the complexed polymerase and inhibit polymerase-catalyzed incorporation of the nucleotide unit. In some embodiments, the nucleotide unit of the nucleotide conjugate can be bound to the 3′ terminal end of a primer at a position that is opposite a complementary nucleotide in the template strand. In some embodiments, the reagent can comprise at least one non-catalytic cation including strontium, barium, calcium, or a combination thereof.

In some embodiments, the system or method can further comprise a reagent suitable for binding a nucleotide unit of a nucleotide conjugate to the complexed polymerase and promote polymerase-catalyzed incorporation of the nucleotide unit. In some embodiments, the nucleotide unit of the nucleotide conjugate can be bound to the 3′ terminal end of a primer at a position that is opposite a complementary nucleotide in the template strand. In some embodiments, the reagent can comprise at least one catalytic cation including magnesium, manganese, or a combination or magnesium and manganese.

In some embodiments, the system or method can further comprise a reagent which can include salts, ions or additives. Additives include, but are not limited to, betaine, spermidine, detergents such as Triton X-100, Tween 20, SDS, or NP-40, ethylene glycol, polyethylene glycol, dextran, polyvinyl alcohol, vinyl alcohol, methylcellulose, heparin, heparan sulfate, glycerol, sucrose, 1,2-propanediol, DMSO, N,N,N-trimethylglycine, ethanol, ethoxyethanol, propylene glycol, polypropylene glycol, block copolymers such as the Pluronic (r) series polymers, arginine, histidine, imidazole, or any combination thereof, or any substance referred to as a DNA “relaxer” (e.g., a compound, that alters the persistence length of DNA, altering the number of within-polymer junctions or crossings, or altering the conformational dynamics of a DNA molecule such that the accessibility of sites within the strand to DNA binding or incorporation moieties is increased). In some embodiments, the reagent can include sucrose, trehalose, glycerol, or a combination thereof.

### Solid Supports

Systems disclosed herein can comprise a solid support (referred to herein as “support”). The solid support can be used to conduct a binding reaction, a nucleotide incorporation reaction, or a combination of a binding reaction and a nucleotide incorporation reaction that employs at least one nucleotide conjugate. The methods and systems can further comprise any one or any combination of a nucleic acid template, nucleic acid primer, polymerase, or a combination thereof. In some embodiments, the nucleic acid template, nucleic acid primer, polymerase, or a combination thereof can be immobilized to the support. In some embodiments, a plurality of binding complexes can be immobilized on the support. In some embodiments, the nucleic acid template molecule can be a concatemer nucleic acid template molecule. In some embodiments, the nucleic acid template molecule can be a clonally-amplified template molecule. In some embodiments, the methods and systems can comprise at least one binding complex immobilized to a support, where the binding complex comprises a polymerase bound to a nucleic acid template molecule which is hybridized to a primer, and a nucleotide conjugate. In some embodiments, a nucleotide unit of the nucleotide conjugate can be bound to the 3′ terminal end of the primer at a position that is opposite a complementary nucleotide in the template molecule. In some embodiments, any of the nucleic acid template, nucleic acid primer, polymerase, or a combination thereof can be immobilized to the support. In some embodiments, the composition can comprise a plurality of binding complexes immobilized to the support. In some embodiments, about 10²-10¹⁵ binding complexes can be immobilized per mm² on the support. In some embodiments, about 10²-10⁹ binding complexes can immobilized per mm² on the support. In some embodiments, the plurality of binding complexes can immobilized to pre-determined sites (e.g., locations) on the support. In some embodiments, the plurality of binding complexes can immobilized to random sites (e.g., locations) on the support. In some embodiments, the plurality of immobilized binding complexes can in fluid communication with each other to permit flowing a solution of reagents (e.g., enzymes including polymerases, nucleotide conjugates, nucleotides, divalent cations, or a combination thereof, and the like) onto the support so that the plurality of immobilized binding complexes on the support can be reacted with the solution of reagents in a massively parallel manner.

In some embodiments, the support can be solid, semi-solid, or a combination of both. In some embodiments, the support can be porous, semi-porous, non-porous, or any combination of porosity. In some embodiments, the surface of the support can be coated with one or more compounds to produce a passivated layer on the support. In some embodiments, the passivated layer can form a porous or semi-porous layer. In some embodiments, the nucleic acid primer or template, or the polymerase, can be attached to the passivated layer to immobilize the primer, template, polymerase, or a combination thereof to the support. In some embodiments, the support can comprise a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached primer molecules that may be used for immobilizing a plurality of nucleic acid template molecules to the support by hybridizing the template molecules to the immobilized primer molecules. In some embodiments, the glass or polymer support can comprise at least one hydrophilic polymer coating layer, and a plurality of surface capture primer molecules attached to the at least one hydrophilic polymer coating layer. In some embodiments, the at least one hydrophilic polymer coating layer can comprise PEG. In some cases, the at least one hydrophilic polymer layer can comprise a branched hydrophilic polymer having at least 4, 8, 16 or 32 branches. In some embodiments, the support further can comprise at least one layer of a plurality of primer molecules. In some embodiments, the surface of the support can be coated with a first layer comprising a monolayer of polymer molecules tethered to a surface of the substrate; a second layer comprising polymer molecules tethered to the polymer molecules of the first layer; and a third layer comprising polymer molecules tethered to the polymer molecules of the second layer, wherein at least one layer comprises branched polymer molecules. In some embodiments, the third layer can further comprise primer molecules tethered to the polymer molecules of the third layer. In some embodiments, the primer molecules tethered to the polymer molecules of the third layer can be distributed at a plurality of depths throughout the third layer. In some embodiments, the surface further can comprise a fourth layer comprising branched polymer molecules tethered to the polymer molecules of the third layer, and a fifth layer comprising polymer molecules tethered to the branched polymer molecules of the fourth layer. In some embodiments, the polymer molecules of the fifth layer can further comprise primer molecules tethered to the polymer molecules of the fifth layer. In some embodiments, the primer molecules tethered to the polymer molecules of the fifth layer can be distributed at a plurality of depths throughout the fifth layer.

In some embodiments, the at least one hydrophilic polymer coating layer, can comprise a molecule selected from the group consisting of poly(ethylene glycol) (PEG, also referred to as polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the primer density. Primers with different lengths of linker between the hybridization region and the surface attachment functional group can also be applied to control surface density. Example of suitable linkers can include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of a predetermined concentration.

In some embodiments, the hydrophilic polymer can be a cross linked polymer. In some embodiments, the cross-linked polymer can include one type of polymer cross linked with another type of polymer. Examples of the crossed-linked polymer can include poly(ethylene glycol) cross-linked with another polymer selected from polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or other hydrophilic polymers. In some embodiments, the cross-linked polymer can be a poly(ethylene glycol) cross-linked with polyacrylamide. As a result of the surface passivation disclosed herein, fluorophores, proteins, nucleic acids, and other biomolecules may not “stick” to the substrates, that is, they can exhibit low nonspecific binding (NSB).

In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating can comprise a poly(N-(5-azidoacet-amidylpentyl)acrylamide-co-acrylamide (PAZAM).

Silane chemistries can constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding support surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (e.g., comprising a free amino functional group), maleimide-PEG silane, biotin-PEG silane, and the like.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter, the primer sequences, or a combination of the tethered adapter and the primer sequences may range in length from about 10 nucleotides to about 100 nucleotides. In some embodiments, the tethered adapter, the primer sequences, or a combination of the tethered adapter and the primer sequences may be at least 10, at least 20, at least 30, at least 40, at least 50, at least 60, at least 70, at least 80, at least 90, or at least 100 nucleotides in length. In some embodiments, the tethered adapter, the primer sequences, or a combination of the tethered adapter and the primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter, the primer sequences, or a combination of the tethered adapter and the primer sequences may range from about 20 nucleotides to about 80 nucleotides. The length of the tethered adapter, the primer sequences, or a combination of the tethered adapter and the primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². The surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the plurality of primer molecules can be present on the support at a surface density of at least 500 molecules/mm², at least 1,000 molecules/mm², at least 5,000 molecules/mm², at least 10,000 molecules/mm², at least 20,000 molecules/mm², at least 50,000 molecules/mm², at least 100,000 molecules/mm², or at least 500,000 molecules/mm².

The present disclosure provides a plurality (e.g., two or more) of nucleic acid templates immobilized to a support. In some embodiments, the immobilized plurality of nucleic acid templates can have the same sequence or have different sequences. In some embodiments, individual nucleic acid template molecules in the plurality of nucleic acid templates can be immobilized to a different site on the support. In some embodiments, two or more individual nucleic acid template molecules in the plurality of nucleic acid templates can be immobilized to a site on the support. In some embodiments, the support can comprise a plurality of sites arranged in an array. In some embodiments, the sites on the support can be arranged in one dimension in a row or a column, or arranged in two dimensions in rows and columns. In some embodiments, the plurality of sites can be arranged on the support in a random or organized fashion, or a combination of both. In some embodiments, the plurality of sites can be arranged in any pattern, including rectilinear or hexagonal patterns. In some embodiments, the support can comprise at least 10² sites, least 10³ sites, least 10⁴ sites, least 10⁵ sites, least 10⁶ sites, least 10⁷ sites, least 10⁸ sites, least 10⁹ sites, least 10¹⁰ sites, or more than 10¹⁰ sites. In some embodiments, a plurality of sites on the support (e.g., 10²-10¹⁰ sites or more) can be immobilized with nucleic acid templates to form a nucleic acid template array. In some embodiments, the nucleic acid templates can be immobilized at a plurality of sites, for example immobilized at 10²-10¹⁰ sites or more. In some cases, the immobilized nucleic acid templates can be clonally-amplified to generate immobilized nucleic acid polonies at the plurality of sites. In some embodiment, the plurality of nucleic acid polonies immobilized on the support can in fluid communication with each other to permit flowing a solution of a plurality of nucleotide conjugates onto the support so that the plurality of nucleic acid polonies immobilized on the support can be essentially simultaneously reacted with the plurality of nucleotide conjugates in a massively parallel manner. In some embodiments, the fluid communication of the plurality of immobilized nucleic acid polonies can be used to conduct nucleotide binding assays, conduct nucleotide incorporation assays (e.g., primer extension or sequencing), or conduct a combination of nucleotide binding assays and nucleotide incorporation assays essentially simultaneously on the plurality of nucleic acid polonies, and optionally to conduct detection and imaging for massively parallel sequencing. In some embodiments, the term “immobilized” and related terms can refer to nucleic acid molecules or enzymes that are attached to a support through covalent bond or non-covalent interaction.

In some embodiments, one or more nucleic acid templates can be immobilized on the support, for example immobilized at the sites on the support. In some embodiments, the one or more nucleic acid templates can be clonally-amplified. In some embodiments, the one or more nucleic acid templates can be clonally-amplified off the support and then deposited onto the support and immobilized on the support. In some embodiments, the clonal amplification reaction of the one or more nucleic acid templates can be conducted on the support resulting in immobilization on the support. In some embodiments, the one or more nucleic acid templates can be clonally-amplified (e.g., in solution or on the support) using a nucleic acid amplification reaction, including any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, or single-stranded binding (SSB) protein-dependent amplification.

In some embodiments, the support can comprise a low non-specific binding surface which exhibits reduced non-specific binding of proteins, nucleic acids, and other components of the nucleic acid hybridization formulation(s), components of the nucleic acid amplification formulation(s), or a combination of components of the nucleic acid hybridization and nucleic acid amplification formulation(s) used for solid-phase nucleic acid amplification. The degree of non-specific binding exhibited by a given support surface may be assessed either qualitatively or quantitatively. For example, in some embodiments, exposure of the surface to fluorescent dyes (e.g., cyanines such as Cy3, or Cy5, etc., fluoresceins, coumarins, rhodamines, etc. or other dyes disclosed herein), fluorescently-labeled nucleotides, fluorescently-labeled oligonucleotides (e.g., primers), fluorescently-labeled proteins (e.g. polymerases), or a combination thereof under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, fluorescently-labeled proteins (e.g. polymerases), or a combination thereof under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under conditions where fluorescence signal is linearly related (or related in a predictable manner) to the number of fluorophores on the support surface (e.g., under conditions where signal saturation, self-quenching, or a combination of signal saturation and self-quenching of the fluorophore is not an issue) and suitable calibration standards are used. In some embodiments, other techniques such as radioisotope labeling and counting methods may be used for quantitative assessment of the degree to which non-specific binding is exhibited by the different support surface formulations of the present disclosure.

In some embodiments, the surfaces disclosed herein can exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein can exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide (primer), etc., under a standardized set of incubation and rinse conditions, followed by detection of the amount of label remaining on the surface and comparison of the signal resulting therefrom to an appropriate calibration standard. In some embodiments, the nucleotide conjugate may be labeled. In some embodiments, the label may comprise a fluorescent label. In some embodiments, the label may comprise a radioisotope. In some embodiments, the label may comprise any other detectable label. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc., or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². A given support surface of the present disclosure may exhibit non-specific binding falling anywhere within this range, for example, of less than 86 molecules per μm². For example, some modified surfaces disclosed herein may exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein may exhibit nonspecific binding of Cy3 dye molecules of less than 0.25 molecules per μm². In independent nonspecific binding assays, 1 μM labeled Cy3 SA (Thermo Fisher), 1 μM Cy5 SA dye (Thermo Fisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP—Cy3 (Jena Biosciences) were incubated on the low binding substrates at 37° C. for 15 minutes in a 384 well plate format. Each well was rinsed 2-3× with 50 μl deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, PA) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, New York), e.g., 405, 488, 532, or 633 nm dichroic reflectors/beamsplitters, and band pass filters were chosen as 532 LP or 645 LP concordant with the appropriate excitation wavelength. Some modified surfaces disclosed herein may exhibit nonspecific binding of dye molecules of less than 0.25 molecules per μm².

In some embodiments, the surfaces disclosed herein may exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein may exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled primers attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases, the contact angle may be no more than 40 degrees, or no more than 45 degrees. A given hydrophilic, low-binding support surface of the present disclosure may exhibit a water contact angle having a value of anywhere within this range.

In some embodiments, the hydrophilic surfaces disclosed herein may facilitate reduced wash times for bioassays, often due to reduced nonspecific binding of biomolecules to the low-binding surfaces. In some embodiments, an adequate wash may be performed in less than 60, 50, 40, 30, 20, 15, 10, or less than 10 seconds. For example, in some embodiments an adequate wash may be performed in less than 30 seconds.

Some low-binding surfaces of the present disclosure may exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents, elevated temperatures, or a combination thereof (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes, changes in temperature, or a combination thereof (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces may exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) can exhibit contrast-to-noise ratios (CNRs) of at least 10, 20, 30, 40, 50, 60, 70, 80, 90, 100, 110, 120, 130, 140, 150, 160, 170, 180, 190, 200, 210, 220, 230, 240, 250, or greater than 250. In some embodiments, fluorescence images of low background surfaces when used in nucleic acid hybridization or amplification applications to create polonies of hybridized or clonally-amplified nucleic acid molecules (e.g., that have been directly or indirectly labeled with a fluorophore) can exhibit contrast-to-noise ratios (CNRs) of greater than 250, 240, 230, 220, 210, 200, 190, 180, 170, 160, 150, 140, 130, 120, 110, 100, 90, 80, 70, 60, 50, 40, 30, 20 10, or less than 10.

In some instances, the performance of nucleic acid hybridization, amplification, sequencing reactions, or a combination thereof using the disclosed low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is taken to be the signal measured for the interstitial regions surrounding a particular feature (diffraction limited spot, DLS) in a specified region of interest (ROI). While signal-to-noise ratio (SNR) can be considered to be a benchmark of overall signal quality, it can be shown that improved CNR can provide a significant advantage over SNR as a benchmark for signal quality in applications that require rapid image capture (e.g., sequencing applications for which cycle times can be minimized), as shown in the example below.

In most ensemble-based sequencing approaches, the background term can be measured as the signal associated with ‘interstitial’ regions (e.g., regions between immobilized polonies or template molecules). In addition to “interstitial” background (Bᵢₙₜₑᵣ) “intrastitial” background (Bᵢₙₜᵣₐ) exists within the region occupied by a polony or template molecule. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In next generation sequencing (NGS) applications, this background signal in the current field-of-view (FOV) is averaged over time and subtracted. The signal arising from individual DNA colonies (e.g., (S)—Bᵢₙₜₑᵣ in the FOV) yields a discernable feature that can be classified. In some instances, the intrastitial background (Bᵢₙₜᵣₐ) can contribute a confounding fluorescence signal that is not specific to the target of interest, but is present in the same ROI thus making it far more difficult to average and subtract.

Nucleic acid sequencing methods employing one or more fluorophores can be conducted on the surfaces or system described herein. The fluorophore can be any type of fluorophore. In some embodiments, the fluorophore can be Cyanine dye-3 (Cy3), and wherein a fluorescence image of the surface can be acquired using an Olympus IX83 inverted fluorescence microscope equipped with 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2) under non-signal saturating conditions while the surface is immersed in a buffer following the binding or incorporation of a first Cy3-labeled nucleotide exhibits a contrast-to-noise (CNR) ratio of at least 20.

Imaging and signal processing from a specified field-of-view (FOV) to calculate the CNR may be conducted, where CNR=(Signal−Background)/(Noise), and where Background=(Bᵢₙₜᵣₐₛₜᵢₜᵢₐₗ+Bᵢₙₜₑᵣₛₜᵢₜᵢₐₗ). For the following examples of the calculation of CNR for clonally-amplified clusters of nucleic acid sequences on the low-binding supports of the present disclosure, an image analysis program was used to find representative foreground bright spots (“clusters”). Spots are defined as a small, connected region of image pixels that exhibit a light intensity above a certain intensity threshold. Connected regions that comprise a total pixel count that falls within a specified range are counted as spots or clusters. Regions that are too big or too small in terms of the number of pixels are disregarded. Once a number of spots or clusters have been identified, the average spot or foreground intensity and other signal statistics are calculated, for example, the maximum intensity, the average intensity, the interpolated maximum intensity, or a combination thereof may be calculated. The median or average value of all spot intensities is used to represent spot foreground intensity. A representative estimate of the background region intensity may be determined using one of several different methods. One method is to divide images into multiple small “tiles” which each include, e.g., 25×25 pixels. Within each tiled region, a certain percentage of the brightest pixels (e.g., 25%) are discarded, and intensity statistics are calculated for the remaining pixels. Another method for determining background intensity is to select a region of at least 500 pixels, or larger, which is free of any foreground “spots” and then to calculate intensity statistics. For either of these methods, a representative background intensity (median or average value) and standard deviation are then calculated. The standard deviation of the intensity in the selected regions is used as the representative background variation. Contrast-to-noise ratio (CNR) is then calculated as (foreground intensity−background intensity)/(background standard deviation).

### Computer Systems for Nucleic Acid Processing

System modules: Disclosed herein is a system configured for performing any of the disclosed methods for nucleic acid processing, sequencing, detection and/or analysis. In some embodiments, the disclosed systems may comprise one or more of the synthetic polypeptides, compositions, formulations, or kits described herein.

In some embodiments, the system may further comprise a fluid flow controller and/or fluid dispensing system configured to sequentially and iteratively contact template nucleic acid molecules hybridized to nucleic acid molecules (e.g., adapters or primers) tethered to a solid support with the disclosed binding complex or multivalent binding complex and/or reagents. In some embodiments, the contacting may be performed within one or more flow cells.

In some embodiments, the system may further comprise an imaging module, where the imaging module comprises, e.g., one or more light sources, one or more optical components (e.g., lenses, mirrors, prisms, optical filters, colored glass filters, narrowband interference filters, broadband interference filters, dichroic reflectors, diffraction gratings, apertures, optical fibers, or optical waveguides and the like), and one or more image sensors (e.g., charge-coupled device (CCD) sensors or cameras, complementary metal-oxide-semiconductor (CMOS) image sensors or cameras, or negative-channel metal-oxide semiconductor (NMOS) image sensors or cameras) for imaging and detection of binding of the disclosed binding complex or multivalent binding complex to target (or template) nucleic acid molecules tethered to a solid support or the interior of a flow cell.

Processors and computer systems: One or more processors may be employed to implement the systems for nucleic acid processing, sequencing, detection and/or analysis methods disclosed herein. The one or more processors may comprise a hardware processor such as a central processing unit (CPU), a graphic processing unit (GPU), a general-purpose processing unit, or computing platform. The one or more processors may be comprised of any of a variety of suitable integrated circuits (e.g., application specific integrated circuits (ASICs) designed specifically for implementing deep learning network architectures, or field-programmable gate arrays (FPGAs) to accelerate compute time, etc., and/or to facilitate deployment), microprocessors, emerging next-generation microprocessor designs (e.g., memristor-based processors), logic devices and the like. Although the disclosure is described with reference to a processor, other types of integrated circuits and logic devices may also be applicable. The processor may have any suitable data operation capability. For example, the processor may perform 512 bit, 256 bit, 128 bit, 64 bit, 32 bit, or 16 bit data operations. The one or more processors may be single core or multi core processors, or a plurality of processors configured for parallel processing.

The one or more processors or computers used to implement the disclosed methods may be part of a larger computer system and/or may be operatively coupled to a computer network (a “network”) with the aid of a communication interface to facilitate transmission of and sharing of data. The network may be a local area network, an intranet and/or extranet, an intranet and/or extranet that is in communication with the Internet, or the Internet. The network in some cases is a telecommunication and/or data network. The network may include one or more computer servers, which in some cases enables distributed computing, such as cloud computing. The network, in some cases with the aid of the computer system, may implement a peer-to-peer network, which may enable devices coupled to the computer system to behave as a client or a server.

The computer system may also include memory or memory locations (e.g., random-access memory, read-only memory, flash memory, Intel® Optane™ technology), electronic storage units (e.g., hard disks), communication interfaces (e.g., network adapters) for communicating with one or more other systems, and peripheral devices, such as cache, other memory, data storage and/or electronic display adapters. The memory, storage units, interfaces and peripheral devices may be in communication with the one or more processors, e.g., a CPU, through a communication bus, e.g., as is found on a motherboard. The storage unit(s) may be data storage unit(s) (or data repositories) for storing data.

The one or more processors, e.g., a CPU, execute a sequence of machine-readable instructions, which are embodied in a program (or software). The instructions are stored in a memory location. The instructions are directed to the CPU, which subsequently program or otherwise configure the CPU to implement the methods of the present disclosure. Examples of operations performed by the CPU include fetch, decode, execute, and write back. The CPU may be part of a circuit, such as an integrated circuit. One or more other components of the system may be included in the circuit. In some cases, the circuit is an application specific integrated circuit (ASIC).

The storage unit stores files, such as drivers, libraries and saved programs. The storage unit stores user data, e.g., user-specified preferences and user-specified programs. The computer system in some cases may include one or more additional data storage units that are external to the computer system, such as located on a remote server that is in communication with the computer system through an intranet or the Internet.

Some aspects of the methods and systems provided herein may be implemented by way of machine (e.g., processor) executable code stored in an electronic storage location of the computer system, such as, for example, in the memory or electronic storage unit. The machine-executable or machine-readable code may be provided in the form of software. During use, the code is executed by the one or more processors. In some cases, the code is retrieved from the storage unit and stored in the memory for ready access by the one or more processors. In some situations, the electronic storage unit is precluded, and machine-executable instructions are stored in memory. The code may be pre-compiled and configured for use with a machine having one or more processors adapted to execute the code or may be compiled at run time. The code may be supplied in a programming language that is selected to enable the code to execute in a pre-compiled or as-compiled fashion.

Various aspects of the technology may be thought of as “products” or “articles of manufacture”, e.g., “computer program or software products”, often in the form of machine- (or processor-) executable code and/or associated data that is stored in a type of machine readable medium, where the executable code comprises a plurality of instructions for controlling a computer or computer system in performing one or more of the methods disclosed herein. Machine-executable code may be stored in an optical storage unit comprising an optically readable medium such as an optical disc, CD-ROM, DVD, or Blu-Ray disc. Machine-executable code may be stored in an electronic storage unit, such as memory (e.g., read-only memory, random-access memory, flash memory) or on a hard disk. “Storage” type media include any or all of the tangible memory of the computers, processors or the like, or associated modules thereof, such as various semiconductor memory chips, optical drives, tape drives, disk drives and the like, which may provide non-transitory storage at any time for the software that encodes the methods and algorithms disclosed herein.

All or a portion of the software code may at times be communicated via the Internet or various other telecommunication networks. Such communications, for example, enable loading of the software from one computer or processor into another, for example, from a management server or host computer into the computer platform of an application server. Thus, other types of media that are used to convey the software encoded instructions include optical, electrical and electromagnetic waves, such as those used across physical interfaces between local devices, through wired and optical landline networks, and over various atmospheric links. The physical elements that carry such waves, such as wired or wireless links, optical links, or the like, are also considered media that convey the software encoded instructions for performing the methods disclosed herein. As used herein, unless restricted to non-transitory, tangible “storage” media, terms such as computer or machine “readable medium” refer to any medium that participates in providing instructions to a processor for execution.

The computer system often includes, or may be in communication with, an electronic display for providing, for example, images captured by a machine vision system. The display is often also capable of providing a user interface (UI). Examples of UI's include but are not limited to graphical user interfaces (GUIs), web-based user interfaces, and the like.

System control software: In some instances, the disclosed systems may comprise a computer (or processor) and computer-readable media that includes code for providing a user interface as well as manual, semi-automated, or fully-automated control of all system functions, e.g. control of a fluid flow controller and/or fluid dispensing system (or sub-system), a temperature control system (or sub-system), an imaging system (or sub-system), etc. In some instances, the system computer or processor may be an integrated component of the instrument system (e.g. a microprocessor or mother board embedded within the instrument). In some instances, the system computer or processor may be a stand-alone module, for example, a personal computer or laptop computer. Examples of fluid flow control functions that may be provided by the instrument control software include, but are not limited to, volumetric fluid flow rates, fluid flow velocities, the timing and duration for sample and reagent additions, rinse steps, and the like. Examples of temperature control functions that may be provided by the instrument control software include, but are not limited to, specifying temperature set point(s) and control of the timing, duration, and ramp rates for temperature changes. Examples of imaging system control functions that may be provided by the instrument control software include, but are not limited to, autofocus capability, control of illumination or excitation light exposure times and intensities, control of image acquisition rate, exposure time, data storage options, and the like.

Image processing software: In some instances of the disclosed systems, the system may further comprise computer-readable media that includes code for providing image processing and analysis capability. Examples of image processing and analysis capability that may be provided by the software include, but are not limited to, manual, semi-automated, or fully-automated image exposure adjustment (e.g. white balance, contrast adjustment, signal-averaging and other noise reduction capability, etc.), manual, semi-automated, or fully-automated edge detection and object identification (e.g., for identifying clusters of amplified template nucleic acid molecules on a substrate surface), manual, semi-automated, or fully-automated signal intensity measurements and/or thresholding in one or more detection channels (e.g., one or more fluorescence emission channels), manual, semi-automated, or fully-automated statistical analysis (e.g., for comparison of signal intensities to a reference value for base-calling purposes).

In some instances, the system software may provide integrated real-time image analysis and instrument control, so that sample loading, reagent addition, rinse, and/or imaging/base-calling steps may be prolonged, modified, or repeated as necessary until, e.g., optimal base-calling results are achieved. Any of a variety of image processing and analysis algorithms known to those of skill in the art may be used to implement real-time or post-processing image analysis capability. Examples include, but are not limited to, the Canny edge detection method, the Canny-Deriche edge detection method, first-order gradient edge detection methods (e.g. the Sobel operator), second order differential edge detection methods, phase congruency (phase coherence) edge detection methods, other image segmentation algorithms (e.g. intensity thresholding, intensity clustering methods, intensity histogram-based methods, etc.), feature and pattern recognition algorithms (e.g. the generalized Hough transform for detecting arbitrary shapes, the circular Hough transform, etc.), and mathematical analysis algorithms (e.g. Fourier transform, fast Fourier transform, wavelet analysis, auto-correlation, etc.), or combinations thereof.

In some instances, the system control and image processing/analysis software may be written as separate software modules. In some instances, the system control and image processing/analysis software may be incorporated into an integrated software package.

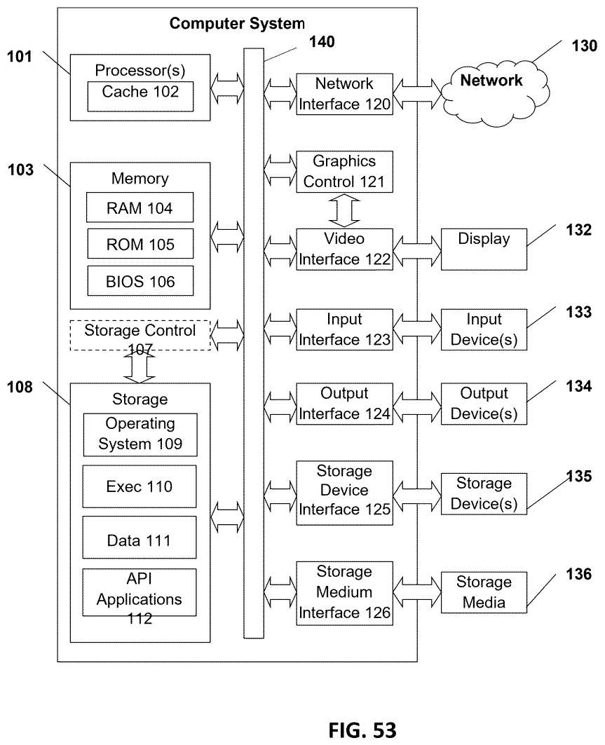

Referring to FIG. 53, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 53 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 53 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOSED Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® Mt, Sony® PS4®. Microsoft® Xbox 3600, Microsoft Xbox One, Nintendo® WHO, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

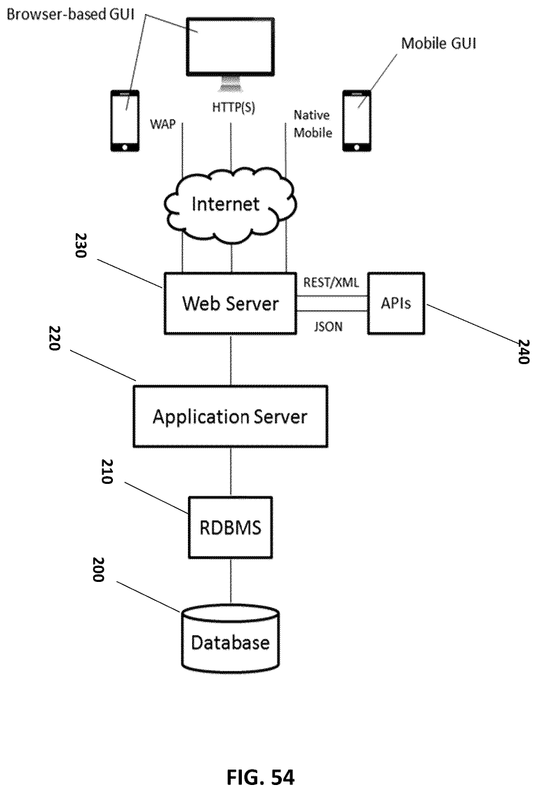

Referring to FIG. 54, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

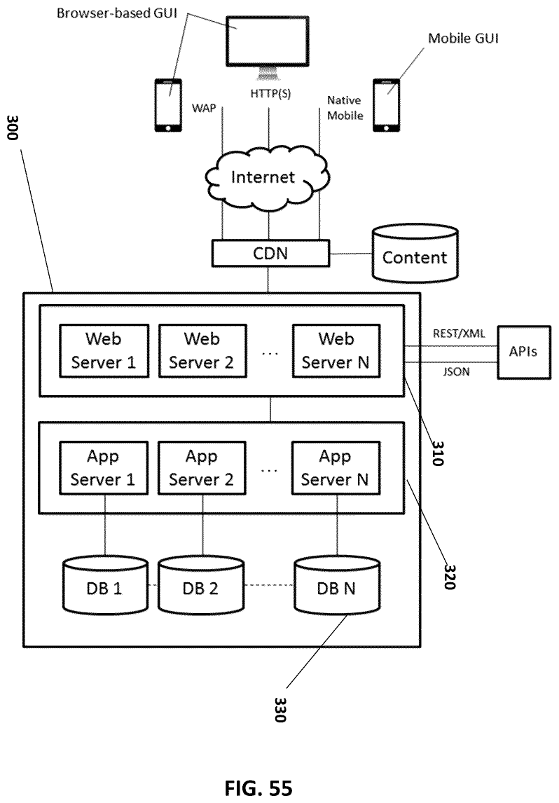

Referring to FIG. 55, in a particular embodiment, an application provision system alternatively has a distributed, cloud-based architecture 300 and comprises elastically load balanced, auto-scaling web server resources 310 and application server resources 320 as well synchronously replicated databases 330.

Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

Databases

In some embodiments, the platforms, systems, media, and methods disclosed herein include one or more databases, or use of the same. In view of the disclosure provided herein, those of skill in the art will recognize that many databases are suitable for storage and retrieval of [INSERT] information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Kits

Disclosed herein, in some embodiments, are kits comprising one or more compositions disclosed herein. In some embodiments, a kit comprises one or more containers comprising: a synthetic polypeptide disclosed herein. In some embodiments, the kit comprises a nucleotide unit. In some embodiments, the nucleotide unit is detectable. In some embodiments, the kit comprises instructions for introducing the synthetic polypeptide and the nucleotide unit to a primed nucleic acid sequence. In some embodiments, the primed nucleic acid sequence comprises a nucleotide complementary to the nucleotide unit under conditions sufficient to form a binding complex. In some embodiments, the binding complex comprises the synthetic polypeptide, the nucleotide unit, and the primed nucleic acid sequence. In some embodiments, the kit further comprises a composition, such as a nucleotide conjugate or a nucleotide conjugate disclosed herein. In some embodiments, the composition comprises a core and at least two nucleotide arms. In some embodiments, a nucleotide arm of the at least two nucleotide arms comprises: a core attachment moiety coupled to the core; a spacer coupled to the core attachment moiety; a linker coupled to the spacer; and the nucleotide unit coupled to the linker. In some embodiments, the linker comprises:

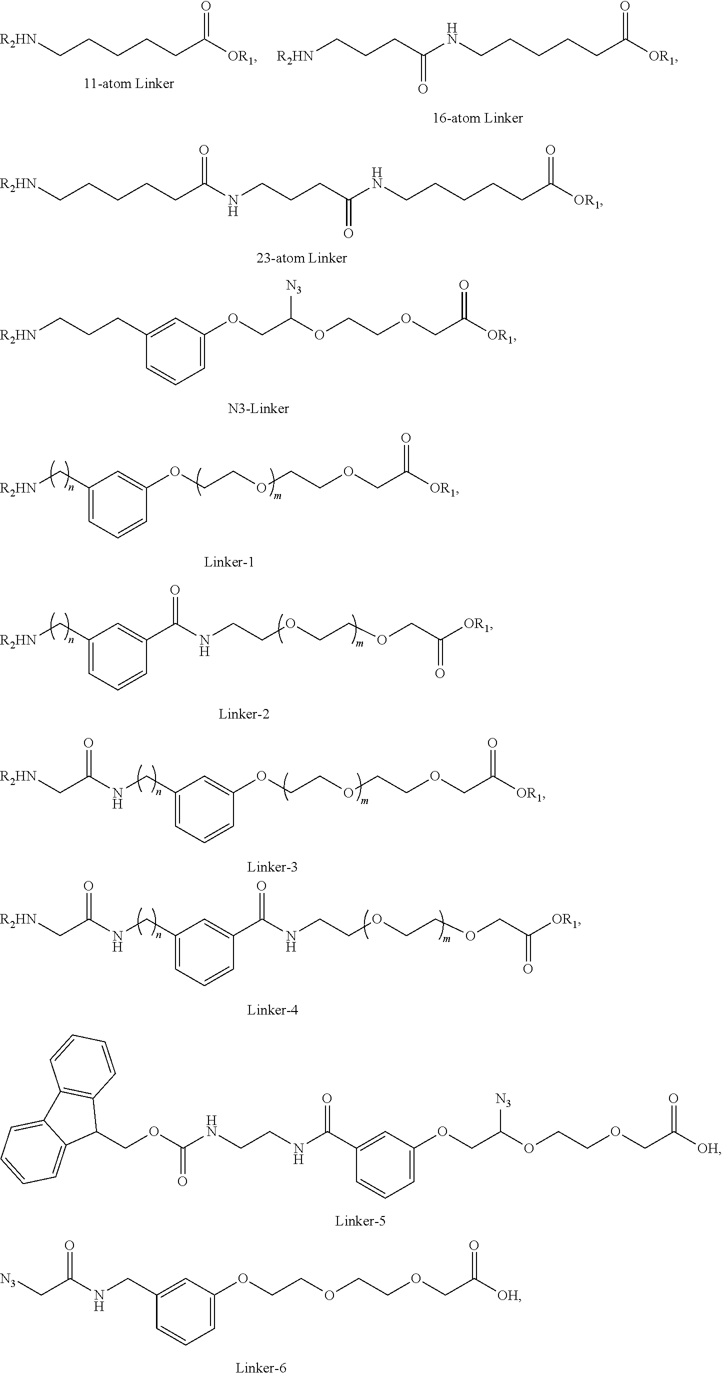

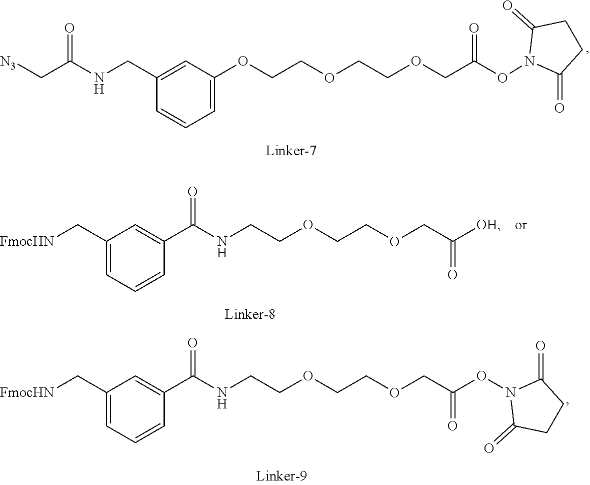

and n is 1 to 6 and m is 0 to 10.

Disclosed herein, in some embodiments, are kits for nucleic acid molecule processing. In some embodiments, the kit comprises a composition disclosed herein, or a formulation disclosed herein. In some embodiments, the kit comprises an instruction for use of the composition in a nucleotide identification reaction. In some embodiments, the instructions for use of the composition or the formulation comprise introducing the nucleotide conjugate and/or the synthetic polypeptide to a nucleic acid sequence (e.g., primed nucleic acid sequence) under conditions sufficient to form a binding complex between a nucleotide of the nucleic acid sequence and a nucleotide unit of the nucleotide conjugate or the synthetic polypeptide or a combination thereof. In some embodiments, instructions further comprise use of the composition for performing a nucleotide binding, nucleotide incorporation, or a nucleotide identification reaction therewith.

In some embodiments, the composition may comprise at least two of the composition disclosed herein. In some embodiments, the at least two of the composition comprises a first composition and a second composition. In some embodiments, the at least two of the composition comprises a first composition, a second composition, and a third composition. In some embodiments, the at least two of the composition comprises a first composition, a second composition, a third composition, and a fourth composition. In some embodiments, the first composition comprises a first fluorophore. In some embodiments, the second composition comprise a second fluorophore. In some embodiments, the third composition comprise a third fluorophore. In some embodiments, the fourth composition comprise a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other.

In some embodiments, the kit further comprises: an agent that reacts with the reactive group in the linker of the composition. In some embodiments, the kit further comprises: an agent that reacts with the reactive group at the 3′ carbon of the sugar moiety in the nucleotide unit of the composition. In some embodiments, the kit further comprises a reagent for use in the nucleotide binding reaction. In some embodiments, the reagent comprises a cation.

In some embodiments, the kit further comprises: (i) a solution comprising a cation; (ii) one or more polymerizing enzymes; (iii) one or more primer sequences; (iv) one or more unlabeled nucleotides; or any combination of (i) to (iv).

The present disclosure provides a kit comprising any one or any combination of two or more of any of the nucleotide conjugates described herein. The kit can comprise for example a plurality of one type of a nucleotide conjugate, a mixture of different types (sub-populations) of the nucleotide conjugates, or a combination of a plurality of one type of a nucleotide conjugate and a mixture of different types of the nucleotide conjugates. The nucleotide conjugates in the kit can be labeled (e.g., fluorescently labeled), non-labeled, or a mixture of labeled and non-labeled forms. The nucleotide conjugates in the kit can include wild type or mutant forms of a streptavidin or avidin core. The nucleotide conjugates in the kit can include core moieties that are labeled with the same type of detectable reporter moiety (e.g., fluorophore) or different types of detectable reporter moieties (e.g., different fluorophores). The nucleotide conjugates in the kit can include nucleotide arms having the same type of spacer or different types of spacers. The nucleotide conjugates in the kit can include nucleotide arms having the same type of linker or different types of linkers. The nucleotide conjugates in the kit can include nucleotide arms comprising linkers with the same type of reactive group or different types of reactive groups. The nucleotide conjugates in the kit can include nucleotide arms having the same type of nucleotide units or different types of nucleotide units. The nucleotide conjugates in the kit can include nucleotide arms having nucleotide units having the same type of reactive groups at the sugar 3′ position or different types of reactive groups.

The kit can further include one or more chemical agents that react with a reactive group in the linker of the nucleotide conjugates. The kit can further include one or more chemical agents that react with a reactive group at the sugar 3′ group in the nucleotide unit of the nucleotide conjugates.

In some embodiments, the kit can further comprise at least one reagent suitable for use in conducting a nucleotide unit binding reaction, a nucleotide unit incorporation reaction, or a combination of a nucleotide unit binding reaction and a nucleotide unit incorporation reaction. In some embodiments, the reagent can comprise cations including any one or any combination of two or more of sodium, magnesium, strontium, barium, potassium, manganese, calcium, lithium, nickel, cobalt, or any combination thereof or other cations suitable for conducting a nucleotide unit binding reaction, a nucleotide incorporation reaction, or a combination of a nucleotide unit binding reaction and a nucleotide unit incorporation reaction. For example, the kit can comprise a reagent comprising a non-catalytic divalent cation including strontium, barium, calcium, or a combination thereof. The kits comprise a reagent comprising a catalytic divalent cation including magnesium, manganese, or a combination of magnesium and manganese.

The kits can comprise one or more containers that contain any one or any combination of two or more of any of the nucleotide conjugates described herein. In some embodiments, the kit can further comprise one or more containers that contain at least one cation, at least one polymerase, primers, a plurality of nucleotides, or a combination thereof. The cation, polymerase and/or nucleotides can be combined in any combination and can be contained in a single container, or can be contained in separate containers, or any combination thereof.

The kit can include instructions for use of the kit for conducting a nucleotide binding reaction, a nucleotide incorporation reaction, a nucleic acid sequencing reaction, or a combination thereof using nucleotide conjugates.

### Methods

Disclosed herein, in some embodiments, are methods of processing and/or analyzing a nucleic acid sequence utilizing one or more compositions, systems, kits or formulations disclosed herein. In some embodiments, the methods are computer-implemented using a computer system disclosed herein. In some embodiments, the method comprises a nucleic acid sequencing method. For example, the sequencing methods may include, but not limited to, any of those disclosed in the following references each of which is incorporated herein by reference in its entirety: Slatko B E, Gardner A F, Ausubel F M. Overview of Next-Generation Sequencing Technologies. Current Protocols in Molecular Biology (2018) April; 122(1):e59. doi: 10.1002/cpmb.59. PMID: 29851291; PMCID: PMC6020069; McCombie W R, McPherson J D, Mardis E R. Next-Generation Sequencing Technologies. Cold Spring Harbor Perspectives in Medicine. (2019) Nov. 1; 9(11):a036798. In some embodiments, the method comprises a sequencing by synthesis method. In some embodiments, the nucleic acid sequencing method comprises a sequencing method based on zero-mode waveguides.

Disclosed herein, in some embodiments, are methods of nucleic acid processing and/or analysis. In some embodiments, the method comprises: providing a formulation comprising a synthetic polypeptide disclosed herein. In some embodiments, the formulation comprises a primed nucleic acid sequence. In some embodiments, the formulation comprises a nucleotide unit complementary to a nucleotide of the primed nucleic acid. In some embodiments, the formulation is provided under conditions sufficient to form a binding complex. In some embodiments, the method comprises: providing a formulation comprising: (i) a synthetic polypeptide disclosed herein; (ii) a primed nucleic acid sequence; and (iii) a nucleotide unit complementary to a nucleotide of the primed nucleic acid and the formulation is provided under conditions sufficient to form a binding complex comprising the synthetic polypeptide, the nucleotide unit, and the primed nucleic acid sequence. In some embodiments, the method comprises a nucleic acid sequencing method. For example, the sequencing methods may include, but not limited to, any of those disclosed in the following references each of which is incorporated herein by reference in its entirety: Slatko B E, Gardner A F, Ausubel F M. Overview of Next-Generation Sequencing Technologies. Current Protocols in Molecular Biology (2018) April; 122(1):e59. doi: 10.1002/cpmb.59. PMID: 29851291; PMCID: PMC6020069; McCombie W R, McPherson J D, Mardis E R. Next-Generation Sequencing Technologies. Cold Spring Harbor Perspectives in Medicine. (2019) Nov. 1; 9(11):a036798. In some embodiments, the method comprises a sequencing by synthesis method. In some embodiments, the nucleic acid sequencing method comprises a sequencing method based on zero-mode waveguides. In some embodiments, the method comprises performing sequencing by binding. In some embodiments, the primed nucleic acid sequence and the nucleotide unit are comprised in a formulation. In some embodiments, the formulation further comprises one or more compositions disclosed herein, such as a nucleotide conjugate. In some embodiments, the nucleotide conjugate comprises (a) a core; and (b) at least two nucleotide arms. In some embodiments, a nucleotide arm of the at least two nucleotide arms comprises: (i) a core attachment moiety coupled to the core; (ii) a spacer coupled to the core attachment moiety; (iii) a linker coupled to the spacer; and (iv) the nucleotide unit coupled to the linker. In some embodiments, the linker comprises:

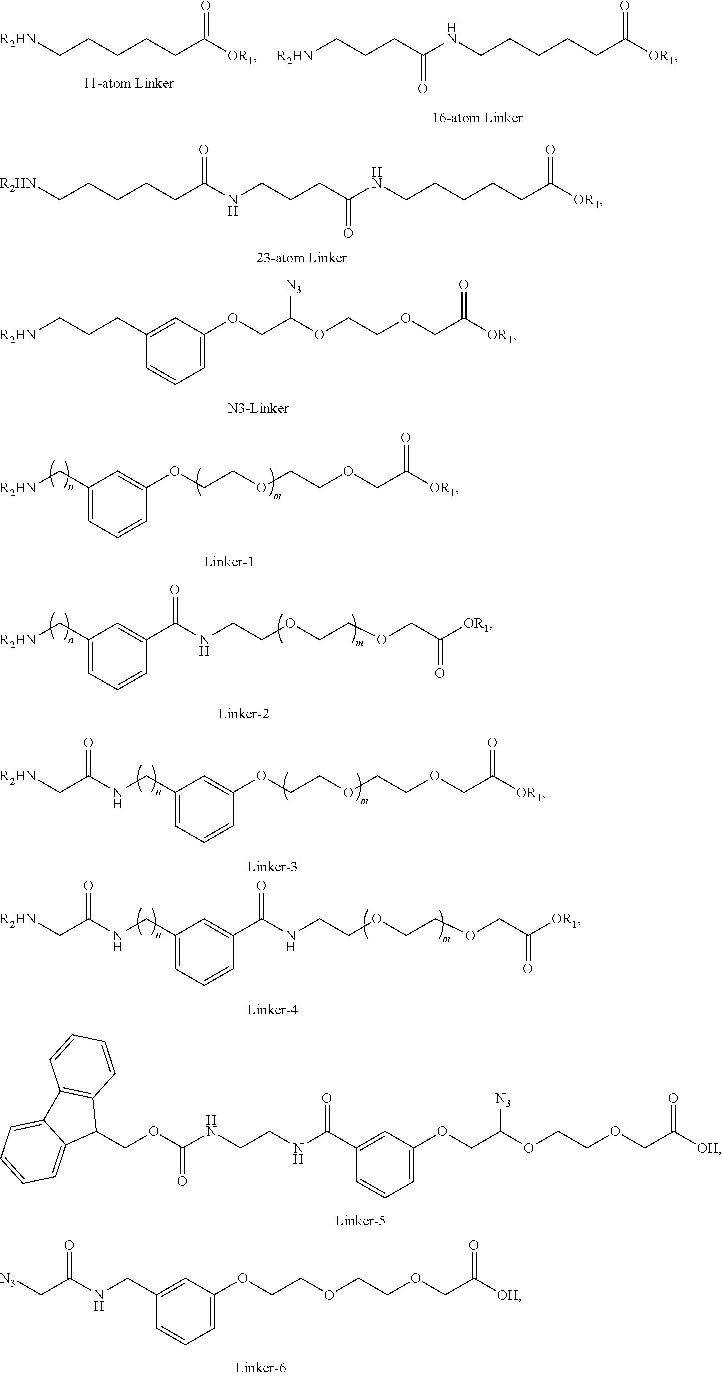

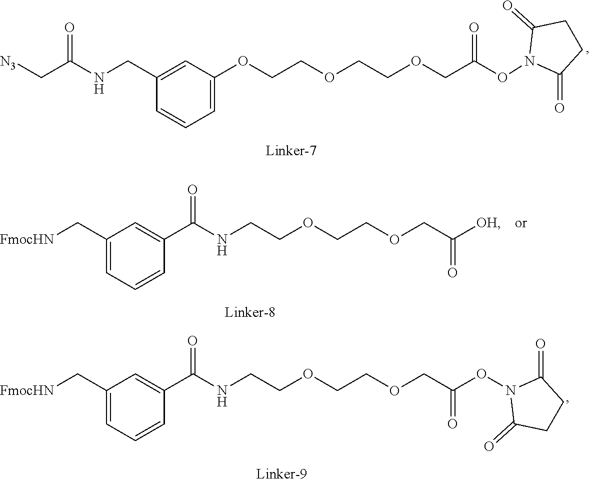

and n is 1 to 6 and m is 0 to 10.

Disclosed herein, in some embodiments, are methods of nucleic acid processing and/or analysis. In some embodiments, the method comprises introducing a composition disclosed herein to a primed nucleic acid sequence under conditions sufficient to form a binding complex. In some embodiments, the binding complex comprises the nucleotide unit of the composition. In some embodiments, the binding complex comprises a nucleotide in the primed nucleic acid sequence. In some embodiments, the nucleotide is complementary to the nucleotide unit of the composition. In some embodiments, the method comprises: introducing a composition disclosed herein to a primed nucleic acid sequence under conditions sufficient to form a binding complex comprising (i) the nucleotide unit of the composition and (ii) a nucleotide in the primed nucleic acid sequence, and the nucleotide is complementary to the nucleotide unit of the composition. In some embodiments, the method comprises a nucleic acid sequencing method. For example, the sequencing methods may include, but not limited to, any of those disclosed in the following references each of which is incorporated herein by reference in its entirety: Slatko B E, Gardner A F, Ausubel F M. Overview of Next-Generation Sequencing Technologies. Current Protocols in Molecular Biology (2018) April; 122(1):e59.doi: 10.1002/cpmb.59.PMID: 29851291; PMCID: PMC6020069; McCombie W R, McPherson J D, Mardis E R. Next-Generation Sequencing Technologies. Cold Spring Harbor Perspectives in Medicine. (2019) Nov. 1; 9(11):a036798. In some embodiments, the method comprises a sequencing by synthesis method. In some embodiments, the nucleic acid sequencing method comprises a sequencing method based on zero-mode waveguides.

In some embodiments, the method comprises: introducing a composition disclosed herein to two or more copies of a primed nucleic acid sequence under conditions sufficient to form a multivalent binding complex. In some embodiments, the multivalent binding complex comprises two or more of the nucleotide units of the composition. In some embodiments, the multivalent binding complex comprises two or more nucleotides in the two or more copies of the primed nucleic acid sequence. In some embodiments, the two or more nucleotides are complementary to the two or more nucleotide units of the composition. In some embodiments, the method comprises: introducing a composition disclosed herein to two or more copies of a primed nucleic acid sequence under conditions sufficient to form a multivalent binding complex comprising (i) two or more of the nucleotide units of the composition and (ii) two or more nucleotides in the two or more copies of the primed nucleic acid sequence, and the two or more nucleotides are complementary to the two or more nucleotide units of the composition. In some embodiments, the method comprises a nucleic acid sequencing method. For example, the sequencing methods may include, but not limited to, any of those disclosed in the following references each of which is incorporated herein by reference in its entirety: Slatko B E, Gardner A F, Ausubel F M. Overview of Next-Generation Sequencing Technologies. Current Protocols in Molecular Biology (2018) April; 122(1):e59.doi: 10.1002/cpmb.59.PMID: 29851291; PMCID: PMC6020069; McCombie W R, McPherson J D, Mardis E R. Next-Generation Sequencing Technologies. Cold Spring Harbor Perspectives in Medicine. (2019) Nov. 1; 9(11):a036798. In some embodiments, the method comprises a sequencing by synthesis method. In some embodiments, the nucleic acid sequencing method comprises a sequencing method based on zero-mode waveguides.

In some embodiments, the composition may comprise at least two of the composition disclosed herein. In some embodiments, the at least two of the composition comprises a first composition and a second composition. In some embodiments, the at least two of the composition comprises a first composition, a second composition, and a third composition. In some embodiments, the at least two of the composition comprises a first composition, a second composition, a third composition, and a fourth composition. In some embodiments, the first composition comprises a first fluorophore. In some embodiments, the second composition comprise a second fluorophore. In some embodiments, the third composition comprise a third fluorophore. In some embodiments, the fourth composition comprise a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, the first wavelength is different from the second wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other.

A nucleotide conjugate can comprise a core attached to multiple nucleotide-arms. In some embodiments, the nucleotide units of the nucleotide-arms can interact with one or more polymerase enzymes in a manner similar to free nucleotides binding (association) and un-binding (dissociation) to a polymerase. For example, the nucleotide unit of the nucleotide conjugates can bind to a complexed polymerase which comprises the polymerase bound to a nucleic acid template which is hybridized a nucleic acid primer (or the polymerase can be bound to a self-priming template/primer nucleic acid molecule). In a nucleotide conjugate, at least two of the nucleotide arms can have nucleotide units that can bind a different complexed polymerase. In some embodiments, any component of the complexed polymerase can be immobilized to a support. For example, the template, the primer, or a combination of the template and the primer (or the self-priming nucleic acid) can be immobilized to a support, or the polymerase can be immobilized to a support.

In some embodiments, methods for nucleotide binding can employ one or more nucleotide conjugates, where each molecule can comprise at least one nucleotide-arm having a nucleotide unit that can bind a complexed polymerase. The nucleotide unit can bind the 3′ terminal end of the primer at a position that is opposite a complementary nucleotide in the template strand. If the core is labeled with a reporter moiety (e.g., a fluorophore), the presence of the bound nucleotide can be detected, and the identity of the bound nucleotide can be determined based on detection of the reporter moiety. In some embodiments, the nucleotide binding reaction can be conducted using a plurality of labeled nucleotide conjugates comprising a plurality of one type of a nucleotide conjugate or a mixture of different types of nucleotide conjugates.

The present disclosure provides methods for conducting nucleotide unit binding reactions using any of the nucleotide conjugates described herein. The present disclosure also provides methods for forming binding complexes, forming multivalent binding complexes, and methods for conducting nucleic acid sequencing reactions using any of the nucleotide conjugates.

Formation of Binding Complexes with Nucleotide Conjugates

The present disclosure provides methods for forming a plurality of binding complexes, the method comprising: (a) contacting a plurality of polymerases with (i) a plurality of nucleic acid template molecules and (ii) a plurality of nucleic acid primers to form a plurality of complexed polymerases; and (b) contacting the plurality of complexed polymerases with a plurality of nucleotide conjugates of the present disclosure to form a plurality of binding complexes. In some embodiments, the method further comprises (c): detecting the nucleotide conjugates that are bound to the complexed polymerases. In some embodiments, the method further comprises (d): identifying the complementary nucleotide unit of the nucleotide conjugates that are bound to the complexed polymerases. In some embodiments, the template molecule, primer molecule, polymerase, or a combination thereof can be immobilized to a support or immobilized to a coating on the support.

In some embodiments, the methods for forming a plurality of binding complexes, the method comprising: (a) contacting a plurality of polymerases with (i) a plurality of nucleic acid template molecules and (ii) a plurality of nucleic acid primers to form a plurality of complexed polymerases wherein individual complexed polymerases comprise a polymerase bound to a nucleic acid template molecule which is hybridized to a nucleic acid primer; and (b) contacting the plurality of complexed polymerases with a plurality of nucleotide conjugates, wherein individual nucleotide conjugates in the plurality comprise a core attached to multiple nucleotide arms and each nucleotide arm is attached to a nucleotide (e.g., a nucleotide unit). In some embodiments, the nucleic acid template molecule can comprise a concatemer template molecule. In some cases, the concatemer template molecule can comprise two or more tandem copies of a sequences of interest. In some cases, the concatemer template molecule can comprise at least one universal adaptor sequence. In some cases, the concatemer template molecule can comprise two or more tandem copies of a sequence of interest and at least one universal adaptor sequence. In some embodiments, the nucleic acid template molecule can comprise a clonally-amplified template molecule. In some cases, the clonally-amplified template molecule can comprise one-copy of a sequence of interest. In some cases, the clonally-amplified template molecule can comprise at least one universal adaptor sequence. In some cases, the clonally-amplified template molecule can comprise one copy of a sequence of interest and at least one universal adaptor sequence. In some cases, the clonally-amplified template molecule can be generated via bridge amplification. In some embodiments, the binding of the complementary nucleotide unit of the nucleotide conjugates to the complexed polymerases can form a plurality of binding complexes. In some embodiments, the contacting in (b) can be conducted under a condition suitable for binding a complementary nucleotide unit of at least one of the nucleotide conjugates to at least one of the complexed polymerases. In some embodiments, the nucleotide unit of a nucleotide conjugate can be bound to the 3′ end of the primer at a position that is opposite a complementary nucleotide in the template molecule. In some embodiments, the condition can be suitable for inhibiting incorporation of the complementary nucleotide units into the primers of the plurality of multivalent-complexed polymerases. In some embodiments, the contacting in (b) can be conducted under a condition suitable for binding a nucleotide of at least one of the nucleotide conjugates to at least one of the complexed polymerases but the bound nucleotide does not incorporate into the 3′ end of the nucleic acid primer. In some embodiments, the contacting of (b) can be conducted in the presence of at least one non-catalytic divalent cation that inhibits polymerase-catalyzed nucleotide incorporation into the nucleic acid primer, where the non-catalytic cation comprises strontium, barium, calcium, or a combination thereof. In some embodiments, at least one of the nucleotide conjugates in the plurality of nucleotide conjugates can be labeled with a detectable reporter moiety. In some embodiments, the detectable reporter moiety can comprise a fluorophore. In some embodiments, the plurality of nucleotide conjugates can comprise at least one nucleotide conjugate having multiple nucleotide arms attached with a nucleotide analog (e.g., nucleotide analog unit), where the nucleotide analog can include a chain terminating moiety at the sugar 3′ position. In some embodiments, the plurality of nucleotide conjugates can comprise at least one nucleotide conjugate comprising multiple nucleotide arms each attached with a nucleotide unit that lacks a chain terminating moiety. In some embodiments, the template molecule, primer molecule, polymerase, or a combination thereof can be immobilized to a support or immobilized to a coating on the support.

In some embodiments, the method for forming a plurality of binding complexes can further comprise: (c): detecting the nucleotide conjugate which is bound to the complexed polymerase. In some embodiments, the detecting can include detecting the nucleotide conjugates that are bound to the complexed polymerases, where the complementary nucleotide units of the nucleotide conjugates are bound to the primers but incorporation of the complementary nucleotide units is inhibited. In some embodiments, at least one of the nucleotide conjugates in the plurality can be labeled with a detectable reporter moiety (e.g., fluorophore) to permit detection. In some embodiments, the labeled nucleotide conjugates can comprise a fluorophore attached to the core, the base of the nucleotide unit, or a combination of the core and the base of the nucleotide unit of the nucleotide conjugates. In some embodiments, the detecting can comprise detecting a fluorescent image of the nucleotide conjugate which is bound to the complexed polymerase.

In some embodiments, the method for forming a plurality of binding complexes can further comprise: (d) identifying the complementary nucleotide unit of the nucleotide conjugate which is bound to the complexed polymerase. In some embodiments, the identifying the complementary nucleotide unit of the nucleotide conjugate can be used to determine the sequence of the nucleic acid template. In some embodiments, the nucleotide conjugates can be labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of complexed polymerases. In some embodiments, the core of the nucleotide conjugate can be labeled with a fluorophore, and wherein the fluorophore which is attached to a given core of the nucleotide conjugate corresponds to the nucleotide base (e.g., adenine, guanine, cytosine, thymine or uracil) of the nucleotide arm. In some embodiments, at least one of the nucleotide arms of the nucleotide conjugate can comprise a nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments the detecting of (c) and the identifying of (d) can be used to determine the sequence of the nucleic acid template molecules.

In some embodiments, in the methods for forming a binding complex, the nucleotide unit of the nucleotide conjugate can be bound to the complexed polymerase (e.g., without dissociation) and has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds. The method for forming the binding complex can be conducted at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing.

The present disclosure provides methods for forming at least two binding complexes on the same template molecule to form an multivalent binding complex, the method comprising: (a) binding a first nucleic acid primer, a first polymerase, and a first nucleotide conjugate to a first portion of a concatemer template molecule thereby forming a first binding complex, wherein a first nucleotide unit of the first nucleotide conjugate binds to the first polymerase; and (b) binding a second nucleic acid primer, a second polymerase, and the first nucleotide conjugate to a second portion of the same concatemer template molecule thereby forming a second binding complex, wherein a second nucleotide unit of the first nucleotide conjugate binds to the second polymerase, wherein the first and second binding complexes which include the same nucleotide conjugate forms a first multivalent binding complex.

In some embodiments, the contacting in (a) and (b) can be conducted under a condition suitable for binding a complementary first nucleotide unit of the first nucleotide conjugate to the first polymerase and the condition suitable for binding a complementary second nucleotide unit of the first nucleotide conjugate to the second polymerase. In some embodiments, the condition can be suitable for inhibiting incorporation of the complementary first nucleotide unit into the 3′ end of the first primer and the condition is suitable for inhibiting incorporation of the complementary second nucleotide unit into the 3′ end of the second primer. In some embodiments, the first nucleotide unit of the first nucleotide conjugate can be bound to the 3′ end of the first primer at a position that is opposite a first complementary nucleotide in the concatemer template molecule, and the second nucleotide unit of the first nucleotide conjugate is bound to the 3′ end of the second primer at a position that is opposite a second complementary nucleotide in the same concatemer template molecule. In some embodiments, the first nucleotide unit of the first nucleotide conjugate can be bound to the 3′ end of the first primer without undergoing nucleotide incorporation, and the second nucleotide unit of the first nucleotide conjugate is bound to the 3′ end of the second primer without undergoing nucleotide incorporation. In some embodiments, the concatemer template molecule, the first and second primer molecules, the first and second polymerases, or a combination thereof can be immobilized to a support or immobilized to a coating on the support. In a similar manner, a second multivalent binding complex can be formed by binding a third and fourth nucleic acid primer, a third and fourth polymerase, and the first nucleotide conjugate to a third and fourth portion of the same concatemer template molecule thereby forming a second binding complex. In some embodiments, the concatemer template molecule can comprise tandem repeat sequences of a sequence of interest and at least one universal sequencing primer binding site. The first, second, third and fourth nucleic acid primers can bind to the same sequencing primer binding site along the concatemer template molecule. The level of valency of the nucleotide units of a given nucleotide conjugate corresponds to the number of nucleotide arms linked to a core. For example, a nucleotide conjugate comprising a streptavidin or avidin core can have a valency of 1, 2, 3 or 4. In some embodiments, a single nucleotide conjugate can essentially simultaneously bind multiple complexed polymerases which are bound to the same template molecule (e.g., a concatemer).

The present disclosure provides methods for forming at least two binding complexes on different template molecules to form an multivalent binding complex, the method comprising: (a) binding a first nucleic acid primer, a first polymerase, and a first nucleotide conjugate to a first template molecule thereby forming a first binding complex, wherein a first nucleotide unit of the first nucleotide conjugate binds to the first polymerase; and (b) binding a second nucleic acid primer, a second polymerase, and the first nucleotide conjugate to a second template molecule thereby forming a second binding complex, wherein a second nucleotide unit of the first nucleotide conjugate binds to the second polymerase, wherein the first and second binding complexes which include the same nucleotide conjugate forms a first multivalent binding complex. In some embodiments, the first and second template molecules can be clonally-amplified template molecules. In some embodiments, the first and second template molecules can be immobilized to a support (or a coating on the support) and are located in close proximity to each other on the support. In some embodiments, the clonally-amplified first and second template molecules can comprise linear template molecules that are generated via bridge amplification and can be immobilized to the same location or feature on a support. The first multivalent binding complex can include the first and second binding complexes on different clonally-amplified template molecules which can be localized in close proximity of each other.

In some embodiments, the contacting in (a) and (b) can be conducted under a condition suitable for binding a complementary first nucleotide unit of the first nucleotide conjugate to the first polymerase and the condition suitable for binding a complementary second nucleotide unit of the first nucleotide conjugate to the second polymerase. In some embodiments, the condition can be suitable for inhibiting incorporation of the complementary first nucleotide unit into the 3′ end of the first primer and the condition can be suitable for inhibiting incorporation of the complementary second nucleotide unit into the 3′ end of the second primer. In some embodiments, the first nucleotide unit of the first nucleotide conjugate can be bound to the 3′ end of the first primer at a position that is opposite a first complementary nucleotide in the first template molecule, and the second nucleotide unit of the first nucleotide conjugate can be bound to the 3′ end of the second primer at a position that is opposite a second complementary nucleotide in the second template molecule. In some embodiments, the first nucleotide unit of the first nucleotide conjugate can be bound to the 3′ end of the first primer without undergoing nucleotide incorporation, and the second nucleotide unit of the first nucleotide conjugate can be bound to the 3′ end of the second primer without undergoing nucleotide incorporation. In some embodiments, the first and second template molecules, the first and second primer molecules, the first and second polymerases, or a combination thereof can be immobilized to a support or immobilized to a coating on the support. In a similar manner, a second multivalent binding complex can be formed by binding a third and fourth nucleic acid primer, a third and fourth polymerase, and the first nucleotide conjugate to a third and fourth template molecule thereby forming a second binding complex. In some embodiments, the linear template molecules can each comprise a sequence of interest and at least one universal sequencing primer binding site. The first, second, third and fourth nucleic acid primers can bind to the sequencing primer binding sites on the first, second, third and fourth template molecules, respectively. The level of valency of the nucleotide units of a given nucleotide conjugate corresponds to the number of nucleotide arms linked to a core. For example, a nucleotide conjugate comprising a streptavidin or avidin core can have a valency of 1, 2, 3 or 4. In some embodiments, a single nucleotide conjugate can essentially simultaneously bind multiple complexed polymerases which are bound to different clonally amplified template molecules.

### Methods for Nucleic Acid Sequencing

The present disclosure provides methods for sequencing one or more nucleic acid template molecules, the method comprising: (a) conducting a sequencing reaction at a position on the template molecule using nucleotide conjugates which bind but do not incorporate; (b) detecting and identifying the bound nucleotide conjugates, (c) removing the nucleotide conjugates from the template molecule, (d) conducting a sequencing reaction at the same position on the template molecule using nucleotides with incorporation; and (e) repeating steps (a)-(d) at the next position on the template molecule. In some embodiments, the binding of the nucleotide conjugates to the template molecule can form at least one multivalent binding complex.

The present disclosure provides methods for sequencing one or more nucleic acid template molecules, the method comprising: (a) contacting a plurality of a first polymerase to (i) a plurality of nucleic acid template molecules and (ii) a plurality of nucleic acid primers, wherein the contacting is conducted under a condition suitable to bind the plurality of first polymerases to the plurality of nucleic acid template molecules and the plurality of nucleic acid primers thereby forming a plurality of first complexed polymerases each comprising a first polymerase bound to a nucleic acid duplex wherein the nucleic acid duplex comprises a nucleic acid template molecule hybridized to a nucleic acid primer.

In some embodiments, in the methods for sequencing one or more nucleic acid template molecules of (a), the plurality of first polymerases can comprise a recombinant polymerase. In some embodiments, the plurality of first polymerases can comprise a wild type or mutant polymerase. In some embodiments, the first polymerases can comprise an amino acid sequence that is at least 80%, 85%, 90%, 95%, 99% identical, or a higher level sequence identity, to any of SEQ ID NOs:1-390, 464, 391-463, or 465. In some embodiments, the primer can comprise a 3′ extendible end or a 3′ non-extendible end. In some embodiments, the plurality of nucleic acid template molecules can comprise amplified template molecules (e.g., clonally amplified template molecules). In some embodiments, the plurality of nucleic acid template molecules can comprise one copy of a target sequence of interest. In some embodiments, the plurality of nucleic acid molecules can comprise two or more tandem copies of a target sequence of interest (e.g., concatemers). In some embodiments, the nucleic acid template molecules in the plurality of nucleic acid template molecules can comprise the same target sequence of interest or different target sequences of interest. In some embodiments, the plurality of nucleic acid template molecules, the plurality of nucleic acid primers, or a combination of the plurality of nucleic acid template molecules and the plurality of nucleic acid primers can be in solution or are immobilized to a support. In some embodiments, when the plurality of nucleic acid template molecules, the plurality of nucleic acid primers, or a combination of the plurality of nucleic acid template molecules and the plurality of nucleic acid primers can be immobilized to a support, the binding with the first recombinant polymerase can generate a plurality of immobilized first complexed polymerases. In some embodiments, the plurality of nucleic acid template molecules, nucleic acid primers, or a combination of the plurality of nucleic acid template molecules and the plurality of nucleic acid primers can be immobilized to 10²-10¹⁵ per mm² different sites on a support. In some embodiments, the binding of the plurality of template molecules and nucleic acid primers with the plurality of first recombinant polymerases can generate a plurality of first complexed polymerases immobilized to 10²-10¹⁵ per mm² different sites on the support. In some embodiments, the plurality of immobilized first complexed polymerases on the support can be immobilized to pre-determined or to random sites on the support. In some embodiments, the plurality of immobilized first complexed polymerases can be in fluid communication with each other to permit flowing a solution of reagents (e.g., enzymes including polymerases, nucleotide conjugates, nucleotides, divalent cations, or a combination thereof) onto the support so that the plurality of immobilized complexed polymerases on the support can be reacted with the solution of reagents in a massively parallel manner.

In some embodiments, the methods for sequencing one or more nucleic acid template molecules can further comprise (b): contacting the plurality of first complexed polymerases with a plurality of nucleotide conjugates to form a plurality of binding complexes. In some embodiments, individual nucleotide conjugates in the plurality of nucleotide conjugates can comprise a core attached to multiple nucleotide arms and each nucleotide arm is attached to a nucleotide (e.g., nucleotide unit). In some embodiments, the contacting of (b) can be conducted under a condition suitable for binding complementary nucleotide units of the nucleotide conjugates to at least two of the plurality of first complexed polymerases thereby forming a plurality of binding complexes. In some embodiments, the nucleotide unit of a nucleotide conjugate can be bound to the 3′ end of the primer at a position that is opposite a complementary nucleotide in the template molecule. In some embodiments, the condition can be suitable for inhibiting incorporation of the complementary nucleotide units into the primers of the plurality of binding complexes. In some embodiments, the contacting of (b) can be conducted in the presence of at least one non-catalytic divalent cation that inhibits polymerase-catalyzed nucleotide incorporation, where the non-catalytic cation can comprise strontium, barium, calcium, or a combination thereof. In some embodiments, at least one of the nucleotide conjugates in the plurality of nucleotide conjugates can be labeled with a detectable reporter moiety. In some embodiments, the detectable reporter moiety can comprise a fluorophore. In some embodiments, the plurality of nucleotide conjugates can comprise at least one nucleotide conjugate having multiple nucleotide arms each attached with a nucleotide analog (e.g., nucleotide analog unit), where the nucleotide analog can include a chain terminating moiety at the sugar 3′ position. In some embodiments, the plurality of nucleotide conjugates can comprise at least one nucleotide conjugate comprising multiple nucleotide arms each attached with a nucleotide unit that lacks a chain terminating moiety. In some embodiments, the template molecule, primer molecule, polymerase, or a combination thereof can be immobilized to a support or immobilized to a coating on the support.

In some embodiments, the methods for sequencing one or more nucleic acid template molecules can further comprise (c): detecting the plurality of binding complexes. In some embodiments, the detecting can include detecting the binding complexes (e.g., nucleotide conjugates that are bound to the complexed polymerases) where the complementary nucleotide units of the nucleotide conjugates are bound to the primers, but incorporation of the complementary nucleotide units is inhibited. In some embodiments, the nucleotide conjugates can be labeled with a detectable reporter moiety to permit detection. In some embodiments, the labeled nucleotide conjugates can comprise a fluorophore attached to the core, the base of the nucleotide unit, or a combination of the core and the base of the nucleotide unit of the nucleotide conjugates. In some embodiments, the detecting can comprise detecting a fluorescent image of the nucleotide conjugate which is bound to the complexed polymerase.

In some embodiments, the methods for sequencing one or more nucleic acid template molecules can further comprise (d): identifying the base of the complementary nucleotide units that are bound to the plurality of first complexed polymerases, thereby determining the sequence of the nucleic acid template. In some embodiments, the nucleotide conjugates can be labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases. In some embodiments, the core of the nucleotide conjugate can be labeled with a fluorophore, and wherein the fluorophore which is attached to a given core of the nucleotide conjugate corresponds to the nucleotide base (e.g., adenine, guanine, cytosine, thymine or uracil) of the nucleotide arm. In some embodiments, at least one of the nucleotide arms of the nucleotide conjugate can comprise a nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments the detecting of (c) and the identifying of (d) can be used to determine the sequence of the nucleic acid template molecules.

In some embodiments, the methods for sequencing one or more nucleic acid template molecules can further comprise (e): dissociating the plurality of binding complexes and removing the plurality of first polymerases and their bound nucleotide conjugates, and retaining the plurality of nucleic acid duplexes.

In some embodiments, the methods for sequencing one or more nucleic acid template molecules can further comprise (f): contacting the plurality of the retained nucleic acid duplexes of (e) with a plurality of second polymerases, wherein the contacting can be conducted under a condition suitable for binding the plurality of second polymerases to the plurality of the retained nucleic acid duplexes, thereby forming a plurality of second complexed polymerases each comprising a second polymerase bound to a nucleic acid duplex. In some embodiments, the plurality of second polymerases can comprise a wild type or mutant polymerase. In some embodiments, the second polymerases can comprise an amino acid sequence that is at least 80%, 85%, 90%, 95%, 99% identical, or a higher-level sequence identity, to any of SEQ ID NOS: 1-390, 464, 391-463, or 465. In some embodiments, the plurality of first polymerases of (a) can have an amino acid sequence that is 100% identical to the amino acid sequence as the plurality of the second polymerases of (f). In some embodiments, the plurality of first polymerases of (a) can have an amino acid sequence that differs from the amino acid sequence of the plurality of the second polymerases of (f).

In some embodiments, the methods for sequencing one or more nucleic acid template molecules can further comprise (g): contacting the plurality of second complexed polymerases with a plurality of nucleotides (e.g., free un-tethered nucleotides), wherein the contacting can be conducted under a condition suitable for binding complementary nucleotides from the plurality of nucleotides to at least two of the second complexed polymerases thereby forming a plurality of nucleotide-complexed polymerases. In some embodiments, the contacting of (g) can be conducted under a condition that is suitable for promoting incorporation of the bound complementary nucleotides into the primers of the nucleotide-complexed polymerases thereby forming a plurality of nucleotide-complexed polymerases. In some embodiments, the incorporating the nucleotide into the 3′ end of the primer in (g) can comprise a primer extension reaction. In some embodiments, the contacting of (g) can be conducted in the presence of at least one catalytic divalent cation that promotes polymerase-catalyzed nucleotide incorporation, where the catalytic cation can comprise magnesium, manganese, or a combination of magnesium and manganese. In some embodiments, the plurality of nucleotides can comprise native nucleotides (e.g., non-analog nucleotides) or nucleotide analogs. In some embodiments, the plurality of nucleotides can comprise a 3′ chain terminating moiety which is removable or is not removable. In some embodiments, the plurality of nucleotides can comprise a plurality of nucleotides labeled with detectable reporter moiety. The detectable reporter moiety can comprise a fluorophore. In some embodiments, the fluorophore can be attached to the nucleotide base. In some embodiments, the fluorophore can be attached to the nucleotide base with a linker which is cleavable/removable from the base or is not removable from the base. In some embodiments, at least one of the nucleotides in the plurality may not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, the methods for sequencing one or more nucleic acid template molecules can further comprise (h): detecting the complementary nucleotides which are incorporated into the primers of the nucleotide-complexed polymerases. In some embodiments, the plurality of nucleotides can be labeled with a detectable reporter moiety to permit detection. In some embodiments, in the methods for sequencing one or more nucleic acid template molecules, the detecting (h) may be omitted.

In some embodiments, the methods for sequencing one or more nucleic acid template molecules can further comprise (i): identifying the bases of the complementary nucleotides which are incorporated into the primers of the nucleotide-complexed polymerases. In some embodiments, the identification of the incorporated complementary nucleotides in (i) can be used to confirm the identity of the complementary nucleotides of the nucleotide conjugates that are bound to the plurality of first complexed polymerases in (d). In some embodiments, the identifying of (i) can be used to determine the sequence of the nucleic acid template molecules. In some embodiments, in the methods for sequencing one or more nucleic acid template molecules, the identifying (i) may be omitted.

In some embodiments, the methods for sequencing one or more nucleic acid template molecules can further comprise (j): removing the chain terminating moiety from the incorporated nucleotide when (g) is conducted by contacting the plurality of second complexed polymerases with a plurality of nucleotides that can comprise at least one nucleotide having a 3′ chain terminating moiety.

In some embodiments, the methods for sequencing one or more nucleic acid template molecules can further comprise (k): repeating (a)-(j) at least once. In some embodiments, the sequence of the nucleic acid template molecules can be determined by detecting and identifying the nucleotide conjugates that bind the polymerases but do not incorporate into the 3′ end of the primer at (c) and (d). In some embodiments, the sequence of the nucleic acid template molecule can be determined (or confirmed) by detecting and identifying the nucleotide that incorporates into the 3′ end of the primer at (h) and (i).

In some embodiments, in the methods for sequencing one or more nucleic acid template molecules, the binding of the plurality of first complexed polymerases with the plurality of nucleotide conjugates forms at least one multivalent binding complex, the method comprises: (a) binding a first nucleic acid primer, a first polymerase, and a first nucleotide conjugate to a first portion of a concatemer template molecule thereby forming a first binding complex, wherein a first nucleotide unit of the first nucleotide conjugate can bind to the first polymerase; and (b) binding a second nucleic acid primer, a second polymerase, and the first nucleotide conjugate to a second portion of the same concatemer template molecule thereby forming a second binding complex, wherein a second nucleotide unit of the first nucleotide conjugate can bind to the second polymerase, wherein the first and second binding complexes which can include the same nucleotide conjugate forms an multivalent binding complex.

In some embodiments, in the methods for sequencing one or more nucleic acid template molecules, the method can include binding the plurality of first complexed polymerases with the plurality of nucleotide conjugates to form at least one multivalent binding complex, the method comprises: (a) contacting the plurality of polymerases and the plurality of nucleic acid primers with different portions of a concatemer nucleic acid template molecule to form at least first and second complexed polymerases on the same concatemer template molecule; (b) contacting a plurality of nucleotide conjugates to the at least first and second complexed polymerases on the same concatemer template molecule, under conditions suitable to bind a single nucleotide conjugate from the plurality to the first and second complexed polymerases, wherein at least a first nucleotide unit of the single nucleotide conjugate can be bound to the first complexed polymerase which can include a first primer hybridized to a first portion of the concatemer template molecule thereby forming a first binding complex (e.g., first ternary complex), and wherein at least a second nucleotide unit of the single nucleotide conjugate can be bound to the second complexed polymerase which can include a second primer hybridized to a second portion of the concatemer template molecule thereby forming a second binding complex (e.g., second ternary complex), wherein the contacting can be conducted under a condition suitable to inhibit polymerase-catalyzed incorporation of the bound first and second nucleotide units in the first and second binding complexes, and wherein the first and second binding complexes which can be bound to the same nucleotide conjugate to form an multivalent binding complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

In some embodiments, in the methods for sequencing one or more nucleic acid template molecules, the binding of the plurality of first complexed polymerases with the plurality of nucleotide conjugates forms at least one multivalent binding complex, the method comprises: (a) binding a first nucleic acid primer, a first polymerase, and a first nucleotide conjugate to a first template molecule thereby forming a first binding complex, wherein a first nucleotide unit of the first nucleotide conjugate can bind to the first polymerase; and (b) binding a second nucleic acid primer, a second polymerase, and the first nucleotide conjugate to a second template molecule thereby forming a second binding complex, wherein a second nucleotide unit of the first nucleotide conjugate can bind to the second polymerase, wherein the first and second binding complexes which include the same nucleotide conjugate can form an multivalent binding complex. In some embodiments, the first and second template molecules can be clonally-amplified template molecules. In some embodiments, the first and second template molecules can be immobilized to a support (or a coating on the support) and can be located in close proximity to each other on the support. In some embodiments, the clonally-amplified first and second template molecules can comprise linear template molecules that are generated via bridge amplification and can be immobilized to the same location or feature on a support. The first multivalent binding complex can include the first and second binding complexes on different clonally-amplified template molecules which can be localized in close proximity of each other.

In some embodiments, in the methods for sequencing one or more nucleic acid template molecules, the method can include binding the plurality of first complexed polymerases with the plurality of nucleotide conjugates to form at least one multivalent binding complex, the method comprising: (a) contacting the plurality of polymerases and the plurality of nucleic acid primers with a first and second nucleic acid template molecule to form at least first and second complexed polymerases on the first and second template molecules; (b) contacting a plurality of nucleotide conjugates to the first and second complexed polymerases, under conditions suitable to bind a single nucleotide conjugate from the plurality to the first and second complexed polymerases, wherein at least a first nucleotide unit of the single nucleotide conjugate can be bound to the first complexed polymerase which can include a first primer hybridized to the first template molecule thereby forming a first binding complex, and wherein at least a second nucleotide unit of the single nucleotide conjugate can be bound to the second complexed polymerase which can include a second primer hybridized to a second template molecule thereby forming a second binding complex, wherein the contacting can be conducted under a condition suitable to inhibit polymerase-catalyzed incorporation of the bound first and second nucleotide units in the first and second binding complexes, and wherein the first and second binding complexes which are bound to the same nucleotide conjugate can form an multivalent binding complex; and (c) detecting the first and second binding complexes on the first and second template molecules, and (d) identifying the first nucleotide unit in the first binding complex thereby determining the sequence of the first template molecule, and identifying the second nucleotide unit in the second binding complex thereby determining the sequence of the second template molecule. In some cases, the first and second template molecules can comprise clonally-amplified first and second template molecules. In some cases, the clonally-amplified template molecules can be generated via bridge amplification. In some cases, the clonally-amplified template molecules can be generated via bridge amplification and are immobilized to the same location or feature on a support. In some cases, the plurality of nucleotide conjugates can be fluorescently labeled.

In some embodiments, in any of the methods described herein, including in the methods for forming a plurality of binding complexes, in the methods for sequencing, and in the methods for forming multivalent binding complexes, individual nucleotide conjugates in the plurality of nucleotide conjugates can comprise: (a) a core; and (b) a plurality of nucleotide arms. In some cases, the nucleotide arms can comprise a core attachment moiety. In some cases, the nucleotide arms can comprise a spacer. In some cases, the nucleotide arms can comprise a linker. In some cases, the nucleotide arms can comprise a nucleotide unit. In some cases, the nucleotide arms can comprise a core attachment moiety, a spacer, a linker, and a nucleotide unit. In some cases, the core can be attached to the plurality of nucleotide arms via their core attachment moiety. In some cases, the spacer can be linked to the linker. In some cases, the linker can be attached to the nucleotide. Nucleotide conjugates are shown in FIGS. 1, 2 and 3.

In some embodiments, in any of the methods described herein, including in the methods for forming a plurality of binding complexes, in the methods for sequencing, and in the methods for forming multivalent binding complexes, the nucleotide arm can comprise a spacer. A non-limited example of a spacer is shown in FIG. 5A. The spacer can have any length, for example the value of m is 1 or at least 2, at least 5, at least 10, at least 20, at least 25, at least 50, at least 75, at least 100, at least 200, at least 500, at least 750, at least 1000, or 2000 or more. In some embodiments, the value of m can be about 20-500, or about 100-110 (e.g., 5,000 g/mol PEG). In some embodiments, in the spacer shown in FIG. 5A (top), the value of o can be 1-50. In some embodiments, the value of o can be about 1-10, or the value of o is about 4. In some embodiments, the spacer can comprise a linear or branched molecule. In some embodiments, the spacer can comprise polyethylene glycol, polypropylene glycol, polyvinyl acetate, polylactic acid, or polyglycolic acid. In some embodiments, the spacer can comprise polyethylene glycol (PEG) having a molecular weight of about 100-200 Daltons (Da), 200-300 Da, 300-400 Da, 400-500 Da, 1 thousand (K) Da, 2K Da, 3K Da, 4K Da, 5K Da, 10K Da, 15 K Da, 20K Da, 30K Da, 40K Da, 50K Da, or larger molecular weight PEG. In some embodiments, the spacer unit of a nucleotide-arm can be attached to a biotin moiety, thereby forming a biotinylated nucleotide-arm which can comprise (i) biotin, (ii) spacer, (iii) linker, and (iv) nucleotide (or nucleoside) (e.g., see FIGS. 7-D).

In some embodiments, in any of the methods described herein, including in the methods for forming a plurality of binding complexes, in the methods for sequencing, and in the methods for forming multivalent binding multivalent binding complexes, the nucleotide arm can comprise a linker which can comprise any of the linker structures shown in FIG. 5A (bottom) or 5B-5F. In some embodiments, the linker can comprise a linear or branched molecule. In some embodiments, the R1 of a linker can comprise any group, for example a nucleotide, nucleoside, or analog thereof. In some embodiments, the R2 of a linker can comprise any group, for example a spacer (e.g., see the top of FIG. 5A). In some embodiments, in the linker, the value of m can be 0-10. In some embodiments, in the linker, the value of n can be 1-6. In some embodiments, the linker can comprise an aliphatic chain having 2-8 units. In some embodiments, the linker can comprise an oligo ethylene glycol moiety having 2-8 units. In some cases, the linker can comprise an aliphatic chain having 2-6 subunits. In some cases, the linker can comprise an oligo ethylene glycol chain having 2-6 subunits. In some embodiments, the linker can comprise an aromatic group. In some embodiments, the linker can comprise an aromatic group and an oligo ethylene glycol moiety having 2-8 units. In some embodiments, the aromatic group can comprise a six-carbon ring group. In some embodiments, the aromatic group can comprise an aryl group (e.g., see N3 Linker and Linkers 1-7 in FIGS. 5A and 5B-F). In some embodiments, the aromatic group can comprise a meta-aminomethylbenzoic acid (also called 3-aminomethylbenzoic acid) group (mAMBA) (e.g., Linkers 8 and 9 of FIG. 5C). In some embodiments, the linker can comprise a fluorenylmethoxycarbonyl protecting group (Fmoc) which can be removed when joining the linker to a spacer. In some embodiments, the linker can comprise an NHS ester group (N-Hydroxysuccinimide) which can be removed when joining the linker to a nucleotide unit.

In some embodiments, in any of the methods described herein, including in the methods for forming a plurality of binding complexes, in the methods for sequencing, and in the methods for forming multivalent binding multivalent binding complexes, a nucleotide-arm can comprise a spacer joined to any of the linkers shown in FIGS. 5A-F, and the linker can be joined to any nucleotide or nucleoside. Nucleotide-arms are shown in FIGS. 6A and 6B. In some embodiments, the nucleotide-arm can comprise any type of nucleotide or nucleoside joined to the linker. Nucleotides can include but are not limited to dATP, dGTP, dCTP, dTTP or dUTP. The nucleotide unit can be joined to the linker via a propargyl amine attachment at the 5 position of a pyrimidine base or the 7 position of a purine base, or at the 1 position of a pyrimidine base or 9 position of a purine base. In some embodiments, certain combinations of linkers and nucleotides can exhibit improved nucleotide discrimination by a polymerase as exhibited by brighter fluorescent signals in nucleotide trapping assays (e.g., see FIGS. 9, 10 and 11, and Example 4). It is noted that the “N3” linker is photolabile. The “N3” moiety may be useful in decreasing residual fluorescent signals seen in cycling assays.

In some embodiments, in any of the methods described herein, including in the methods for forming a plurality of binding complexes, in the methods for sequencing, and in the methods for forming multivalent binding complexes, any of the linkers described herein can be joined to a nucleotide to generate a nucleotide-linker molecule. Nucleotide-linker configurations are shown in FIGS. 7A-G. For example, the nucleotide unit can be joined to the linker via a propargyl amine attachment at the 5 position of a pyrimidine base or the 7 position of a purine base, or at the 1 position of a pyrimidine base or 9 position of a purine base.

In some embodiments, in any of the methods described herein, including in the methods for forming a plurality of binding complexes, in the methods for sequencing, and in the methods for forming multivalent binding complexes, the nucleotide-arm can be a biotinylated nucleotide-arm comprising (i) biotin, (ii) spacer, (iii) linker, and (iv) nucleotide (or nucleoside). Biotinylated nucleotide-arms are shown in FIGS. 8A and 8B. In some embodiments, the nucleotide conjugates can comprise a core attached to a plurality of biotinylated nucleotide-arms, where individual biotinylated nucleotide-arms can comprise (i) a biotin moiety, (ii) a spacer, (iii) a linker, and (iv) a nucleotide unit or a nucleoside unit.

In some embodiments, in any of the methods described herein, including in the methods for forming a plurality of binding complexes, in the methods for sequencing, and in the methods for forming multivalent binding complexes, any of the nucleotide-arms and biotinylated nucleotide-arms described herein can comprise a nucleotide unit that can bind a polymerase which can be complexed with a nucleic acid template and nucleic acid primer (e.g., nucleotide association). The nucleotide unit can also dissociate from the complexed polymerase and can either re-bind the same complexed polymerase or can bind a different complexed polymerase that is proximal to the nucleotide conjugate.

The nucleotide-arm and biotinylated nucleotide-arm can comprise a nucleotide unit, where the nucleotide unit can comprise a heterocyclic base, a sugar and at least one phosphate group. In some embodiments, the nucleotide can comprise 1, 2, 3, 4, 5, 6, 7, 8, 9 or 10 phosphate groups.

The nucleotide-arm and biotinylated nucleotide-arm can comprise a nucleotide unit (or nucleoside unit) which can include a heterocyclic base comprising a purine or pyrimidine base, or analogs thereof.

In some embodiments, the 5 position of the pyrimidine base can be joined to the linker, the 7 position of the purine base can be joined to the linker, the 1 position of the pyrimidine base can be joined to the linker, or the 9 position of the purine base can be joined to a linker.

In some embodiments, the nucleotide-arm and biotinylated nucleotide-arm can comprise a nucleotide unit which is a propargyl-amine (PA) modified nucleotide, where the 5-position of the pyrimidine base or 7-position of the purine base can be joined to the linker via a propargyl-amine group, or where the 1 position of a pyrimidine base or 9 position of a purine base can be joined to the linker via a propargyl-amine group.

In some embodiments, the nucleotide-arm and biotinylated nucleotide-arm can comprise a nucleotide unit having a ribose or deoxyribose sugar moiety. In some embodiments, the nucleotide unit can be selected from a group consisting of adenosine triphosphate (ATP), adenosine diphosphate (ADP), adenosine monophosphate (AMP), deoxyadenosine triphosphate (dATP), deoxyadenosine diphosphate (dADP), and deoxyadenosine monophosphate (dAMP), thymidine triphosphate (TTP), thymidine diphosphate (TDP), thymidine monophosphate (TMP), deoxythymidine triphosphate (dTTP), deoxythymidine diphosphate (dTDP), deoxythymidine monophosphate (dTMP), uridine triphosphate (UTP), uridine diphosphate (UDP), uridine monophosphate (UMP), deoxyuridine triphosphate (dUTP), deoxyuridine diphosphate (dUDP), deoxyuridine monophosphate (dUMP), cytidine triphosphate (CTP), cytidine diphosphate (CDP), cytidine monophosphate (CMP), deoxycytidine triphosphate (dCTP), deoxycytidine diphosphate (dCDP), deoxycytidine monophosphate (dCMP), guanosine triphosphate (GTP), guanosine diphosphate (GDP), guanosine monophosphate (GMP), deoxyguanosine triphosphate (dGTP), deoxyguanosine diphosphate (dGDP), and deoxyguanosine monophosphate (dGMP).

In some embodiments, the nucleotide-arm and biotinylated nucleotide-arm can comprise a nucleotide unit (or nucleoside unit) having a sugar moiety which comprises a ribose, deoxyribose, or analog thereof. In some embodiments, the sugar moiety can comprise a 3′OH group. In some embodiments, a nucleotide unit having a sugar 3′OH group can bind a complexed polymerase which can include a polymerase bound to a nucleic acid template which is hybridized to a nucleic acid primer (or a self-priming template/primer nucleic acid molecule). For example, the nucleotide unit can bind the polymerase, and can bind the terminal 3′ end of the primer at a position that is opposite a complementary nucleotide in the template strand. A nucleotide unit having a sugar 3′OH group can undergo nucleotide incorporation in a polymerase-catalyzed reaction. The sugar 3′OH group on an incorporated nucleotide unit can mediate polymerase-catalyzed incorporation of a subsequent nucleotide, for example in a primer extension reaction.

In some embodiments, the nucleotide-arm and biotinylated nucleotide-arm can comprise a nucleotide unit (or nucleoside unit) having a sugar moiety with a 3′OH group substituted with a blocking group. In some embodiments, a nucleotide unit having a 3′ blocking group can bind a complexed polymerase which can include a polymerase bound to a nucleic acid template which is hybridized to a nucleic acid primer (or a self-priming template/primer nucleic acid molecule). For example, the nucleotide unit can bind the polymerase, and can bind the terminal 3′ end of the primer at a position that is opposite a complementary nucleotide in the template strand. A nucleotide unit having a 3′ blocking group can undergo nucleotide incorporation in a polymerase-catalyzed reaction. The 3′ blocking group on an incorporated nucleotide unit can inhibit/block a polymerase-catalyzed incorporation of a subsequent nucleotide, for example in a primer extension reaction.

In some embodiments, in any of the methods described herein, including in the methods for forming a plurality of binding complexes, in the methods for sequencing, and in the methods for forming multivalent binding complexes, any of the nucleotide-arms and biotinylated nucleotide-arms described herein can comprise a linker that lacks a reactive group, or the linker can include a reactive group located at any position along the linker. For example, the azide moiety of the N3 Linker (e.g., FIG. 5A) can be replaced with a reactive group.

In some embodiments, in any of the methods described herein, including in the methods for forming a plurality of binding complexes, in the methods for sequencing, and in the methods for forming multivalent binding complexes, any of the nucleotide-arms and biotinylated nucleotide-arms described herein can further comprise a linker having a reactive group which can be selected from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group.

The reactive group in the linker can be reactive with a chemical reagent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride.

The reactive group in the linker can be an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, including in the methods for forming a plurality of binding complexes, in the methods for sequencing, and in the methods for forming multivalent binding complexes, any of the nucleotide-arms and biotinylated nucleotide-arms described herein can further comprise a nucleotide unit having a sugar moiety with a 3′OH group substituted with a blocking group, where the sugar 3′ blocking group can be selected from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the sugar 3′ blocking group can comprises a 3′-O-azidomethyl group, 3′-O-methyl group, 3′-O-alkyl hydroxylamino group, a 3′-phosphorothioate group, a 3′-O-malonyl group, or a 3′-O-benzyl group.

In some embodiments, the nucleotide-arm and biotinylated nucleotide-arm can comprise a nucleotide unit having a sugar 3′ blocking group that can be reactive with a chemical reagent. For example, the sugar 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The blocking groups aryl and benzyl can be reactive with Palladium on carbon (Pd/C). The blocking groups amine, amide, keto, isocyanate, phosphate, thiol, disulfide can be reactive with phosphine or with a thiol group including beta-mercaptoethanol or dithiothritol (DTT). The blocking group carbonate can be reactive with potassium carbonate (K₂CO₃) in MeOH, with triethylamine in pyridine, or with Zn in acetic acid (AcOH). The blocking groups urea and silyl can be reactive with tetrabutylammonium fluoride, pyridine-HF, with ammonium fluoride, or with triethylamine trihydrofluoride.

In some embodiments, the nucleotide-arm and biotinylated nucleotide-arm can comprise a nucleotide unit having a sugar 3′ blocking group comprising an azide, azido or azidomethyl group.

In some embodiments, the azide, azido or azidomethyl 3′ blocking group can be reactive with a chemical reagent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, including in the methods for forming a plurality of binding complexes, in the methods for sequencing, and in the methods for forming multivalent binding complexes, the nucleotide-arm and biotinylated nucleotide-arm can comprise a nucleotide unit having a chain of one, two or three phosphorus atoms where the chain can be attached to the 5′ carbon of the sugar moiety via an ester or phosphoramide linkage. In some embodiments, the nucleotide unit can be an analog having a phosphorus chain in which the phosphorus atoms are linked together with intervening O, S, NH, methylene or ethylene. In some embodiments, the phosphorus atoms in the chain can include substituted side groups including O, S or BH₃. In some embodiments, the chain can include phosphate groups substituted with analogs including phosphoramidate, phosphorothioate, phosphordithioate, and O-methylphosphoroamidite groups.

In some embodiments, in any of the methods described herein, including in the methods for forming a plurality of binding complexes, in the methods for sequencing, and in the methods for forming multivalent binding complexes, the core can comprise a bead, particle or nanoparticle. In some embodiments, the core can comprise an alkyl, alkenyl, or alkynyl core such as may be present in a branched polymer or dendrimer. In some embodiments the core can comprise a moiety that mediates conjugation of the core to the nucleotide-arm. FIGS. 1, 2 and 3 show the general architecture of nucleotide conjugates.

In some embodiments the core can comprise a streptavidin-type or avidin-type moiety, and the biotin unit of the biotinylated nucleotide-arm can mediate conjugation of the core to the biotinylated nucleotide-arm (FIG. 2). A streptavidin-type or avidin-type core can be a tetrameric biotin-binding protein that can bind one, two, three or up to four biotinylated nucleotide-arms.

In some embodiments, the core can comprise a streptavidin-type or avidin-type moiety, including streptavidin or avidin protein, as well as any derivatives, analogs and other non-native forms of streptavidin or avidin that can bind to at least one biotin moiety. The streptavidin or avidin moiety can comprise native or recombinant forms, as well as mutant versions and derivatized molecules. Mutant versions of streptavidin and avidin can comprise any one or any combination of two or more of amino acid insertions, deletions, substitutions, truncations, or any combination thereof. Mutant versions can also include fusion polypeptides. Many different forms of streptavidin and avidin are commercially-available.

In some embodiments, in any of the methods described herein, the nucleotide conjugate can comprise a core which comprises a streptavidin moiety having a full-length or truncated amino acid sequence and having a high affinity for binding biotin. For example, the streptavidin moiety can exhibit a dissociation constant (k_{d}) of about 10⁻¹⁴ mol/L, or about 10⁻¹⁵ mol/L. In some embodiments, the streptavidin moiety can comprise a polypeptide having the backbone sequence any of SEQ ID NOs:466-470. The streptavidin moiety can comprise an amino acid sequence having at least 80%, at least 85%, at least 90%, at least 95%, at least 99% or higher levels of identity to any of SEQ ID NOs:466-470. The streptavidin moiety can comprise the core portion of a streptavidin protein which can be truncated at the N-terminal end, the C-terminal end, or a combination of the N-terminal and the C-terminal ends of a streptavidin protein having an amino acid sequence of any of SEQ ID NOs:466-468, 470-471. For example, the streptavidin moiety can lack the N-terminal portion of any of SEQ ID NOS: 466-468, 470-471 (e.g., the underlined N-terminal portions in FIGS. 17-19, 21-22). The streptavidin moiety can lack the C-terminal portion of any of SEQ ID NOs:466-468 (e.g., the underlined C-terminal portions in FIGS. 17-19). The streptavidin moiety can comprise a core portion comprising the amino acid sequence of SEQ ID NO:469 or 470.

In some embodiments, the streptavidin moiety can comprise any amino acid substitution mutation at a site that can be labeled with a dye. For example, the dye-labeling site can comprise lysine at position 121 (e.g., see SEQ ID NO:469) which may overlap with a biotin binding site. In some embodiments, a dye attached to streptavidin at Lys121 may block or inhibit biotin binding to the dye-labeled streptavidin. A nucleotide conjugate comprising a dye labeled streptavidin carrying lysine at position 121 may exhibit dissociation of a biotinylated nucleotide-arm from the streptavidin core. A nucleotide conjugate having increased stability can comprise a dye labeled streptavidin carrying a Lys121Arg mutation which may exhibit reduced dissociation of a biotinylated nucleotide-arm from the streptavidin core.

In some embodiments, the streptavidin moiety can comprise any amino acid substitution that increases the affinity for binding biotin (e.g., increases the K_{d} to about 10⁻¹⁶ mol/L), improves retention of biotin at temperatures up to about 60° C., or about 65° C., or about 70° C. or about 80° C., or a combination of increases the affinity for binding biotin and improves retention of biotin. Amino acid substitutions can comprise His151Lys or His 151Asp of SEQ ID NO:466; His128Lys or His128Asp of SEQ ID NO:467; His127Lys or His127Asp of SEQ ID NO:468; His116Lys or His116Asp of SEQ ID NO:469; or His135Lys or His135Asp of SEQ ID NO:470. The histidine residue that can be substituted with lysine or aspartic acid are bolded and underlined in FIGS. 17-21.

In some embodiments, in any of the methods described herein, the nucleotide conjugate can comprise a core which comprises an avidin moiety having a full-length or truncated amino acid sequence and having a high affinity for binding biotin. For example, the avidin moiety can exhibit a dissociation constant (K_{d}) of about 10⁻¹⁴ mol/L, or about 10⁻¹⁵ mol/L. In some embodiments, the avidin moiety can comprise a polypeptide having the backbone sequence SEQ ID NOs:471 or 472. The avidin moiety can comprise an amino acid sequence having at least 80%, at least 85%, at least 90%, at least 95%, at least 99% or higher levels of identity to SEQ ID NOs:471 or 472. The avidin moiety can comprise the core portion of an avidin protein which can be truncated at the N-terminal end, the C-terminal end, or a combination of the N-terminal and the C-terminal ends of an avidin protein having an amino acid sequence of SEQ ID NO:471. For example, the avidin moiety can lack the N-terminal portion of SEQ ID NO: 471 (e.g., the underlined N-terminal portions in FIG. 22). In some embodiments, the avidin can comprise substitutions of any one or any combination of the eight arginine residues (e.g., underlined and bolded in FIG. 22 or 23).

The avidin can comprise partially de-glycosylated forms and non-glycosylated forms. The avidin moiety can include derivatized forms, for example, N-acyl avidins, e.g., N-acetyl, N-phthalyl and N-succinyl avidin, and the commercially-available products including EXTRAVIDIN, CAPTAVIDIN (selective nitration of tyrosine residues at the four biotin-binding sites to generate avidin that reversibly binds biotin), NEUTRAVIDIN (having chemically de-glycosylated and include modified arginine residues), and NEUTRALITE AVIDIN (five of the eight arginine residues are replaced with neutral amino acids, two of the lysines are replaced with glutamic acid, and Asp17 is replaced with isoleucine).

In some embodiments, in any of the methods described herein, the nucleotide conjugate can comprise a core attached to a plurality of nucleotide-arms, where all of the attached nucleotide-arms can comprise the same type of nucleotide units. For example, a nucleotide conjugate can comprise a core (e.g., streptavidin or avidin core) attached to a plurality of nucleotide arms or biotinylated nucleotide arms, where all of the attached arms can have a nucleotide unit selected from a group consisting of dATP, dGTP, dCTP, dTTP and dUTP.

In some embodiments, in any of the methods described herein, the nucleotide conjugate can comprise a core attached to a plurality of nucleotide-arms, where the attached nucleotide-arms can comprise the different types of nucleotide units. For example, a nucleotide conjugate can comprise a core (e.g., streptavidin or avidin core) attached to a plurality of nucleotide arms or biotinylated nucleotide arms, where at least a first attached arm can have a first nucleotide unit selected from a group consisting of dATP, dGTP, dCTP, dTTP and dUTP, and a second attached arm can have a second nucleotide unit selected from a group consisting of dATP, dGTP, dCTP, dTTP and dUTP, where the first and second nucleotide units are different.

In some embodiments, in any of the methods described herein, the nucleotide conjugate can comprise a core attached to a plurality of nucleotide-arms, where all of the attached nucleotide-arms can comprise the same type of spacer. For example, a nucleotide conjugate can comprise a core (e.g., streptavidin or avidin core) attached to a plurality of nucleotide arms or biotinylated nucleotide arms, where all of the attached arms can have the same spacer. In some embodiments, the spacer can be selected from any of the spacers described herein (e.g., FIG. 5A (top)).

In some embodiments, in any of the methods described herein, the nucleotide conjugate can comprise a core attached to a plurality of nucleotide-arms, where the attached nucleotide-arms can comprise different types of spacers. For example, a nucleotide conjugate can comprise a core (e.g., streptavidin or avidin core) attached to a plurality of nucleotide arms or biotinylated nucleotide arms, where at least a first attached arm can have a first type of spacer, and a second attached arm can have a second type of spacer, where the first and second spacer units are different. In some embodiments, the first and second type of linker can be selected from any of the spacers described herein (e.g., FIG. 5A (top)).

In some embodiments, in any of the methods described herein, the nucleotide conjugate can comprise core attached to a plurality of nucleotide-arms, where all of the attached nucleotide-arms can comprise the same type of linker. For example, a nucleotide conjugate can comprise a core (e.g., streptavidin or avidin core) attached to a plurality of nucleotide arms or biotinylated nucleotide arms, where all of the attached arms can have the same linker. In some embodiments, the linker can be selected from any of the linkers described herein (e.g., FIGS. 5A (bottom) and 5B-F).

In some embodiments, in any of the methods described herein, the nucleotide conjugate can comprise a core attached to a plurality of nucleotide-arms, where the attached nucleotide-arms can comprise different types of linkers. For example, a nucleotide conjugate can comprise a core (e.g., streptavidin or avidin core) attached to a plurality of nucleotide arms or biotinylated nucleotide arms, where at least a first attached arm can have a first type of linker, and a second attached arm can have a second type of linker, where the first and second linker units are different. In some embodiments, the first and second type of linker can be selected from any of the linkers described herein (e.g., FIGS. 5A (bottom) and 5B-F).

In some embodiments, in any of the methods described herein, the nucleotide conjugate can comprise a core attached to a plurality of nucleotide-arms, where all of the attached nucleotide-arms can comprise the same type of spacer and linker. For example, a nucleotide conjugate can comprise a core (e.g., streptavidin or avidin core) attached to a plurality of nucleotide arms or biotinylated nucleotide arms, where all of the attached arms can have the same spacer and linker. In some embodiments, the spacer and linker can be selected from any of the spacers and linkers described herein.

In some embodiments, in any of the methods described herein, the nucleotide conjugate can comprise a core attached to a plurality of nucleotide-arms, where all of the attached nucleotide-arms can comprise the same type of reactive group. For example, a nucleotide conjugate can comprise a core (e.g., streptavidin or avidin core) attached to a plurality of nucleotide arms or biotinylated nucleotide arms, where all of the attached arms can have the same reactive group. In some embodiments, the reactive group can comprise an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group.

In some embodiments, the reactive group in the linker can be reactive with a chemical reagent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride.

In some embodiments, the nucleotide-arms can have the same type of reactive group in the linker where the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP),bis-sulfo triphenyl phosphine (BS-TPP) or Tri(hydroxyproyl)phosphine (THPP).

In some embodiments, in any of the methods described herein, the nucleotide conjugate can comprise a core attached to a plurality of nucleotide-arms, where the attached nucleotide-arms can comprise different types of reactive groups in the linkers. For example, a nucleotide conjugate can comprise a core (e.g., streptavidin or avidin core) attached to a plurality of nucleotide arms or biotinylated nucleotide arms, where at least a first attached arm can have a first type of reactive group in a first linker unit, and a second attached arm can have a second type of reactive group in a second linker unit, where the first and second reactive groups are different.

In some embodiments, the first reactive group in the first linker unit, and the second reactive group in the second linker unit, can be selected in any combination from a group consisting of an alkyl group, alkenyl group, alkynyl group, allyl group, aryl group, benzyl group, azide group, amine group, amide group, keto group, isocyanate group, phosphate group, thio group, disulfide group, carbonate group, urea group, and silyl group.

In some embodiments, the first and second reactive groups can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride.

In some embodiments, the nucleotide-arms can have the different types of reactive groups in the linkers where the reactive group can comprises an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, the nucleotide conjugate can comprise a core attached to a plurality of nucleotide-arms, where all of the attached nucleotide-arms can comprise a nucleotide unit having the same type of sugar 3′OH group. For example, a nucleotide conjugate can comprise a core (e.g., streptavidin or avidin core) attached to a plurality of nucleotide arms or biotinylated nucleotide arms, where all of the attached arms can have a nucleotide unit having the same type of sugar 3′OH group.

In some embodiments, in any of the methods described herein, the nucleotide conjugate can comprise a core attached to a plurality of nucleotide-arms, where all of the attached nucleotide-arms can comprise a nucleotide unit having the same type of sugar 3′ blocking group (e.g., chain terminating moiety). For example, a nucleotide conjugate can comprise a core (e.g., streptavidin or avidin core) attached to a plurality of nucleotide arms or biotinylated nucleotide arms, where all of the attached arms can have a nucleotide unit having the same type of sugar 3′ blocking group. In some embodiments, the sugar 3′ blocking group can comprise an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the sugar 3′ blocking group can comprise a 3′-O-alkyl hydroxylamino group, a 3′-phosphorothioate group, a 3′-O-malonyl group, or a 3′-O-benzyl group. In some embodiments, the sugar 3′ blocking group can comprise an azide, azido or azidomethyl group.

In some embodiments, the sugar 3′ blocking group can be reactive with a chemical reagent. For example, the sugar 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The sugar 3′ blocking groups aryl and benzyl can be reactive with H2 and Palladium on carbon (Pd/C). The sugar 3′ blocking groups amine, amide, keto, isocyanate, phosphate, thiol, disulfide can be reactive with phosphine or with a thiol group including beta-mercaptoethanol or dithiothritol (DTT). The sugar 3′ blocking group carbonate can be reactive with potassium carbonate (K₂CO₃) in MeOH, with triethylamine in pyridine, or with Zn in acetic acid (AcOH). The sugar 3′ blocking groups urea and silyl can be reactive with tetrabutylammonium fluoride, pyridine-HF, with ammonium fluoride, or with triethylamine trihydrofluoride.

In some embodiments, the sugar 3′ blocking group (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, the nucleotide conjugate can comprise a core attached to a plurality of nucleotide-arms, where the attached nucleotide-arms can comprise nucleotide units having different sugar 3′ blocking groups. For example, a nucleotide conjugate can comprise a core (e.g., streptavidin or avidin core) attached to a plurality of nucleotide arms or biotinylated nucleotide arms, where at least a first attached arm can have a first nucleotide unit having a first 3′ blocking group, and a second attached arm can have a second nucleotide unit having a second 3′ blocking group, where the first and second 3′ blocking groups are different.

In some embodiments, the first 3′ blocking group in the first nucleotide unit, and the second 3′ blocking group in the second nucleotide unit, can be selected in any combination from a group consisting of an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the first 3′ blocking group in the first nucleotide unit, and the second 3′ blocking group in the second nucleotide unit, can be selected in any combination from a group consisting of an 3′-O-alkyl hydroxylamino group, a 3′-phosphorothioate group, a 3′-O-malonyl group, or a 3′-O-benzyl group. In some embodiments, the first 3′ blocking group in the first nucleotide unit, and the second 3′ blocking group in the second nucleotide unit, can be selected in any combination from a group consisting of an azide, azido or azidomethyl group.

In some embodiments, the first and second 3′ blocking groups can be reactive with a chemical reagent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride.

In some embodiments, the first and second 3′ blocking groups (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, the nucleotide conjugate can comprise a core attached to a plurality of nucleotide-arms, where the attached nucleotide-arms can comprise at one nucleotide unit having a sugar 3′ OH, at least one nucleotide unit having a first 3′ blocking group and at least another nucleotide unit having a second 3′ blocking group, where the first and second blocking groups differ from each other. For example, a nucleotide conjugate can comprise a core (e.g., streptavidin or avidin core) attached to a plurality of nucleotide arms or biotinylated nucleotide arms, where (a) at least a first arm can comprise a first nucleotide unit having a sugar moiety which includes a 3′OH group, (b) at least second arm can comprise a second nucleotide unit having a first 3′ blocking group, and (c) at least third arm can comprise a third nucleotide unit having a second blocking group, wherein the first and second 3′ blocking groups are different from each other.

In some embodiments, the first 3′ blocking group in the first nucleotide unit, and the second 3′ blocking group in the second nucleotide unit, can be selected in any combination from a group consisting of an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the first 3′ blocking group in the first nucleotide unit, and the second 3′ blocking group in the second nucleotide unit, can be selected in any combination from a group consisting of an 3′-O-alkyl hydroxylamino group, a 3′-phosphorothioate group, a 3′-O-malonyl group, or a 3′-O-benzyl group. In some embodiments, the first 3′ blocking group in the first nucleotide unit, and the second 3′ blocking group in the second nucleotide unit, can be selected in any combination from a group consisting of an azide, azido or azidomethyl group.

In some embodiments, the first and second 3′ blocking groups can be reactive with a chemical reagent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride.

In some embodiments, the first and second 3′ blocking groups (e.g., azide, azido and azido methyl) can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, in any of the methods described herein, the nucleotide conjugate can comprise a core labeled with at least one detectable reporter moiety to form a labeled core. In some embodiments, a labeled core attached to two or more nucleotide-arms can comprise a labeled nucleotide conjugate. In some embodiments, a streptavidin or avidin core can be labeled with 1-6 or more reporter moieties. In some embodiments, the reporter moiety can comprise a fluorophore.

A method using a mixture of nucleotide conjugates having different units in their nucleotide-arms, where distinction between the different nucleotide conjugates may be achieved. In some embodiments, the core of a first nucleotide conjugate can be labeled with a reporter moiety to distinguish it from a second labeled (or non-labeled) nucleotide conjugate. For example, a unit in a nucleotide-arm of the labeled first nucleotide conjugate can differ from a unit in a nucleotide-arm of a labeled second nucleotide conjugate. Any unit in the first nucleotide conjugate (e.g., spacer, linker, reactive group, nucleotide base, sugar 3′OH, 3′ blocking group, or a combination thereof) can differ from a corresponding unit in the second nucleotide conjugate, where the first and second reporter moieties correspond to the differentiating unit. In some embodiments, the first and second reporter moieties can be spectrally distinguishable from each other.

In some embodiments, in any of the methods described herein, the core of a first nucleotide conjugate can be labeled with a first reporter moiety that corresponds to the base (e.g., dATP, dGTP, dCTP, dTTP or dUTP) in the attached nucleotide-arms, and the core of a second nucleotide conjugate can be labeled with a second reporter moiety that corresponds to the base (e.g., dATP, dGTP, dCTP, dTTP or dUTP) in the attached nucleotide-arms, where the base in the first nucleotide conjugate and the base in the second nucleotide conjugate are different. In some embodiments, the first and second reporter moieties can be spectrally distinguishable from each other. In some embodiment, detection of the first reporter moiety can indicate a binding event, an incorporation event, or a combination of a binding event and an incorporation event of the first nucleotide conjugate having the first base, and detection of the second reporter moiety can indicate a binding event, an incorporation event, or a combination of a binding event and an incorporation event of the second nucleotide conjugate having the second base. The binding event can be a nucleotide conjugate binding to a complexed polymerase. The incorporation event can be a nucleotide unit incorporating into the terminal 3′ end of an extendible primer in a complexed polymerase, where the nucleotide unit is part of a nucleotide conjugate.

In some embodiments, any of the methods described herein can employ a mixture of different sub-populations of labeled nucleotide conjugates where each sub-population comprises a different reporter moiety, where a particular reporter moiety can correspond to a particular base in the nucleotide arms. A particular sub-population can be distinguishable from other sub-populations based on the reporter moiety attached to the core. Two, three, four, five or more separate sub-populations can be mixed together to form a plurality of labeled nucleotide conjugates comprising two or more sub-populations of spectrally distinguishable nucleotide conjugates. In some embodiments, at least one sub-population of nucleotide conjugates in the mixture can be non-labeled (e.g., dark nucleotide conjugates).

Disclosed herein is a formulation. In some embodiments, the formulation comprises: at least two of the composition disclosed herein. In some embodiments, the at least two of the composition comprises a first composition and a second composition. In some embodiments, the nucleotide unit of the first composition comprises a different nucleobase than the nucleotide unit of the second composition. In some embodiments, the nucleotide unit of the first composition comprises a first blocking group. In some embodiments, the first blocking group is linked to the 3′ carbon of the sugar moiety. In some embodiments, the first blocking group reacts with a chemical compound to remove the first blocking group. In some embodiments, the first blocking group reacts with a chemical compound to remove the first blocking group and generate a OH moiety on the 3′ carbon of the sugar moiety of the nucleotide unit of the first composition. In some embodiments, the nucleotide unit of the second composition comprises a second blocking group. In some embodiments, the second blocking group is linked to the 3′ carbon of the sugar moiety. In some embodiments, the second blocking group reacts with a chemical compound to remove the second blocking group. In some embodiments, the second blocking group reacts with a chemical compound to remove the second blocking group and generate a OH moiety on the 3′ carbon of the sugar moiety of the nucleotide unit of the second composition. In some embodiments, the nucleotide unit of the first composition differs from the nucleotide unit of the second composition. In some embodiments, the nucleotide unit of the first composition comprises a first blocking group linked to the 3′ carbon of the sugar moiety and the first blocking group reacts with a chemical compound to remove the first blocking group and generate a OH moiety on the 3′ carbon of the sugar moiety of the nucleotide unit of the first composition. In some embodiments, the nucleotide unit of the second composition comprises a second blocking group linked to the 3′ carbon of the sugar moiety and the second blocking group reacts with a chemical compound to remove the second blocking group and generate a OH moiety on the 3′ carbon of the sugar moiety of the nucleotide unit of the second composition. In some embodiments, the nucleotide unit of the first composition differs from the nucleotide unit of the second composition. In some embodiments, the linker of the first composition differs from the linker of the second composition. In some embodiments, the linker of the first composition comprises a first reactive group and the linker of the second composition comprises a second reactive group. In some embodiments, the second reactive group differs from the first reactive group. In some embodiments, the first composition comprise a first fluorophore and the second composition comprise a second fluorophore. In some embodiments, the first fluorophore emits light at a wavelength that differs from the wavelength of light emitted from the second fluorophore. In some embodiments, the first composition comprises a fluorescent label and the second composition is unlabeled. In some embodiments, the at least two of the composition comprises a first composition, a second composition, and a third composition. In some embodiments, the at least two of the composition comprises a first composition, a second composition, a third composition, and a fourth composition. In some embodiments, the first composition comprises a first fluorophore. In some embodiments, the second composition comprise a second fluorophore. In some embodiments, the third composition comprise a third fluorophore. In some embodiments, the fourth composition comprise a fourth fluorophore. In some embodiments, the first fluorophore emits light at a first wavelength. In some embodiments, the second fluorophore emits light at a second wavelength. In some embodiments, the third fluorophore emits light at a third wavelength. In some embodiments, the fourth fluorophore emits light at a fourth wavelength. In some embodiments, at least two of the first wavelength, the second wavelength, and the third wavelength are different from each other. In some embodiments, each of the first wavelength, the second wavelength, and the third wavelength is different from another. In some embodiments, all of the first wavelength, the second wavelength, and the third wavelength are different from each other. In some embodiments, at least two of the first wavelength, the second wavelength, the third wavelength and the fourth wavelength are different from each other. In some embodiments, at least three of the first wavelength, the second wavelength, the third wavelength and the fourth wavelength are all different from each other. In some embodiments, each of the first wavelength, the second wavelength, the third wavelength and the fourth wavelength is different from another. In some embodiments, the first wavelength, the second wavelength, the third wavelength and the fourth wavelength are all different from each other.

In some embodiments, any of the methods described herein employ a mixture of different sub-populations of labeled nucleotide conjugates which comprises a mixture of at least two sub-populations of nucleotide conjugates labeled with different reporter moieties. In some embodiments, the mixture of different subpopulations of labeled nucleotide conjugates can comprise at least a first sub-population of nucleotide conjugates labeled with a first reporter moiety that corresponds to a first nucleotide unit on the nucleotide-arm. In some embodiments, the mixture of different subpopulation of labeled nucleotide conjugates can comprise at least a second sub-population of nucleotide conjugates labeled with a second reporter moiety that corresponds to a second nucleotide unit on the nucleotide-arms. In some embodiments, the mixture of different subpopulation of labeled nucleotide conjugates can comprise the first sub-population and the second sub-population of nucleotide conjugates. In some cases, the first and second reporter moieties from the first sub-population and the second sub-population of nucleotide conjugates. In some embodiments, the mixtures of different sub-populations of labeled nucleotide conjugates further can comprise at least a third sub-population of nucleotide conjugates labeled with a third reporter moiety. In some cases, the first, second and third reporter moieties differ from each other. In some embodiments, the mixture of different sub-populations of labeled nucleotide conjugates can further comprise at least a fourth sub-population of nucleotide conjugates labeled with a fourth reporter moiety. In some cases, the first, second, third and fourth reporter moieties differ from each other. In some embodiments, additional sub-populations (e.g., fifth, sixth, seventh, eighth, ninth, tenth or more) of labeled nucleotide conjugates can be added into the mixture.

In some embodiments, any of the methods described herein can employ a mixture of different sub-populations of labeled nucleotide conjugates which can comprise a mixture of at least two sub-populations of nucleotide conjugates labeled with different reporter moieties. In some embodiments, the mixture of different sub-populations of labeled nucleotide conjugates can comprise at least a first sub-population of nucleotide conjugates. In some cases, the first sub-population of nucleotide conjugates can be labeled with a first reporter moiety that corresponds to a first nucleotide unit on the nucleotide-arms. In some embodiments, the mixture of different sub-populations of labeled nucleotide conjugates can comprise at least a second sub-population of nucleotide conjugates. In some cases, the second sub-population of nucleotide conjugates can be non-labeled (e.g., a dark nucleotide conjugate). In some embodiments, the mixture of different sub-populations of labeled nucleotide conjugates can comprise the first sub-population of nucleotide conjugates and the second sub-population of nucleotide conjugates.

In some embodiments, any of the methods described herein can employ a mixture of different sub-populations of labeled nucleotide conjugates which can comprise a mixture of at least three sub-populations of nucleotide conjugates labeled with different reporter moieties. In some embodiments, the mixture of different sub-populations of labeled nucleotide conjugates can comprise at least a first sub-population of nucleotide conjugates. In some cases, the first sub-population can be labeled with a first reporter moiety that corresponds to a first nucleotide unit on the nucleotide-arms. In some embodiments, the mixture of different sub-populations of labeled nucleotide conjugates can comprise at least a second sub-population of nucleotide conjugates. In some cases, the second sub-population can be labeled with a second reporter moiety that corresponds to a second nucleotide unit on the nucleotide-arms. In some embodiments, the mixture of different sub-populations of labeled nucleotide conjugates can comprise at least a third sub-population of nucleotide conjugates. In some cases, the third sub-population can be non-labeled (e.g., a dark nucleotide conjugate). In some cases, the first and second reporter moieties differ from each other. In some embodiments, the mixture of different sub-populations of labeled nucleotide conjugates can comprise the first subpopulation, the second sub-population, and the third sub-population.

In some embodiments, any of the methods described herein can employ a mixture of different sub-populations of labeled nucleotide conjugates which comprises a mixture of at least four sub-populations of nucleotide conjugates labeled with different reporter moieties. In some embodiments, the mixture of different sub-populations of labeled nucleotide conjugates can comprise at least a first sub-population of nucleotide conjugates. In some cases, the first sub-population can be labeled with a first reporter moiety that corresponds to a first nucleotide unit on the nucleotide-arms. In some embodiments, the mixture of different sub-populations of labeled nucleotide conjugates can comprise at least a second sub-population of nucleotide conjugates. In some cases, the second sub-population can be labeled with a second reporter moiety that corresponds to a second nucleotide unit on the nucleotide-arms. In some embodiments, the mixture of different sub-populations of labeled nucleotide conjugates can comprise at least a third sub-population of nucleotide conjugates. In some cases, the third sub-population can be labeled with a third reporter moiety. In some embodiments, the mixture of different sub-populations of labeled nucleotide conjugates can comprise at least a fourth sub-population of nucleotide conjugates. In some cases, the fourth sub-population can be non-labeled (e.g., a dark nucleotide conjugate). In some embodiments, the mixture of different sub-populations of labeled nucleotide conjugates can comprise the first sub-population, the second sub-population, the third sub-population, and the fourth sub-population. In some cases, the first, second and third reporter moieties differ from each other. An embodiment can comprises: a mixture of four different types of nucleotide conjugates comprising (1) a first sub-population of nucleotide conjugates each comprising a dATP nucleotide unit and a core labeled with a first type of fluorophore, (2) a second sub-population of nucleotide conjugates each comprising a dGTP nucleotide unit and a core labeled with a second type of fluorophore, (3) a third sub-population of nucleotide conjugates each comprising a dCTP nucleotide unit and a core labeled with a third type of fluorophore, and (4) a fourth sub-population of nucleotide conjugates each comprising a dTTP nucleotide unit and a core labeled with a fourth type of fluorophore, where the first, second, third and fourth fluorophores can be spectrally distinguishable. In some embodiments, any one of the sub-populations of nucleotide conjugates can be non-labeled for use as “dark” nucleotide conjugates.

In some embodiments, any of the methods described herein can employ a plurality of nucleotide conjugates, wherein individual nucleotide conjugates in the plurality can comprise a core bound to at least one nucleotide-arm. In some embodiments, individual nucleotide conjugates in the plurality can comprise a core bound to 2-5 nucleotide-arms. In some embodiments, individual nucleotide conjugates in the plurality can comprise a streptavidin or avidin core bound to 2-5 biotinylated nucleotide-arms.

In some embodiments, any of the methods described herein can employ a plurality of nucleotide conjugates, wherein individual nucleotide conjugates in the plurality can comprise a core bound to at least one nucleotide-arm having one type of nucleotide unit comprising dATP, dGTP, dCTP, dTTP or dUTP. In some embodiments, individual nucleotide conjugates in the plurality can comprise a core bound to 2-5 nucleotide-arms, where the nucleotide-arms have one type of nucleotide unit comprising dATP, dGTP, dCTP, dTTP or dUTP. In some embodiments, individual nucleotide conjugates in the plurality can comprise a core bound to 2-5 biotinylated nucleotide-arms, where the biotinylated nucleotide-arms have one type of nucleotide unit comprising dATP, dGTP, dCTP, dTTP or dUTP.

In some embodiments, any of the methods described herein can employ a plurality of nucleotide conjugates comprising a mixture (sub-populations) of two or more different types of nucleotide conjugates, wherein at least a first nucleotide conjugate in the plurality can comprise a core bound to at least one nucleotide-arm having a first type of nucleotide selected from a group consisting of dATP, dGTP, dCTP, dTTP or dUTP, and at least a second nucleotide conjugate comprising a core bound to at least one nucleotide-arm having a second type of nucleotide that differs from the first nucleotide in the first nucleotide conjugate. In some embodiments, the first nucleotide conjugate can comprise a core bound to 2-5 biotinylated nucleotide arms, where the biotinylated-arms can have a first type of nucleotide selected from a group consisting of dATP, dGTP, dCTP, dTTP or dUTP. In some embodiments, the second nucleotide conjugate can comprise a core bound to 2-5 biotinylated nucleotide arms, where the biotinylated-arms can have a second type of nucleotide selected from a group consisting of dATP, dGTP, dCTP, dTTP or dUTP, where the first and second type of nucleotides are different. In some embodiments, the mixture can comprise two, three, four, five, or more different types of nucleotide conjugates having nucleotides selected in any combination from a group consisting of dATP, dGTP, dCTP, dTTP or dUTP.

In some embodiments, any of the methods described herein can employ a plurality of nucleotide conjugates, wherein individual nucleotide conjugates in the plurality can comprise a core bound to at least one nucleotide-arm, wherein all of the nucleotide arms that are bound to a core can have the same spacer. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

In some embodiments, any of the methods described herein can employ a plurality of nucleotide conjugates, wherein individual nucleotide conjugates in the plurality can comprise a core bound to at least one nucleotide-arm, wherein all of the nucleotide arms that are bound to a core can have the same linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

In some embodiments, any of the methods described herein can employ a plurality of nucleotide conjugates, wherein individual nucleotide conjugates in the plurality can comprise a core bound to at least one nucleotide-arm, wherein all of the nucleotide arms that are bound to a core can have the same spacer and linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

In some embodiments, any of the methods described herein can employ a plurality of nucleotide conjugates comprising a mixture (sub-populations) of two or more different types of nucleotide conjugates. In some embodiments, the mixture of two or more different types of nucleotide conjugates can comprise at least a first nucleotide conjugate. In some cases, the first nucleotide conjugate can comprise a core bound to at least one nucleotide-arm having a first type of spacer. In some embodiments, the mixtures of two or more different types of nucleotide conjugates can comprise at least a second nucleotide conjugate. In some cases, the second nucleotide conjugate can comprise a core bound to at least one nucleotide-arm having a second type of spacer. In some cases, the second type of spacer in the second nucleotide conjugate differs from the first spacer in the first nucleotide conjugate. In some embodiments, the mixture of two or more different types of nucleotide conjugates can comprise the first nucleotide conjugate and the second nucleotide conjugate. In some embodiments, the first nucleotide conjugate can comprise a core bound to 2-5 biotinylated nucleotide arms, where the biotinylated-arms can have a first type of spacer. In some embodiments, the second nucleotide conjugate can comprise a core bound to 2-5 biotinylated nucleotide arms, where the biotinylated-arms can have a second type of spacer, where the first and second type of spacers are different.

In some embodiments, any of the methods described herein can employ a plurality of nucleotide conjugates comprising a mixture (sub-populations) of two or more different types of nucleotide conjugates. In some embodiments, the mixture of two or more different types of nucleotide conjugates can comprise at least a first nucleotide conjugate. In some cases, the first nucleotide conjugate can comprise a core bound to at least one nucleotide-arm having a first type of linker. In some embodiments, the mixture of two or more different types of nucleotide conjugates can comprise at least a second nucleotide conjugate. In some cases, the second nucleotide conjugates can comprise a core bound to at least one nucleotide-arm having a second type of linker. In some embodiments, the mixture of two or more different types of nucleotide conjugates can comprise the first nucleotide conjugate and the second nucleotide conjugate. In some cases, the second linker in the second nucleotide conjugate differs from the first linker in the first nucleotide conjugate. In some embodiments, the first nucleotide conjugate can comprise a core bound to 2-5 biotinylated nucleotide arms, where the biotinylated-arms can have a first type of linker. In some embodiments, the second nucleotide conjugate can comprise a core bound to 2-5 biotinylated nucleotide arms, where the biotinylated-arms can have a second type of linker, where the first and second type of spacers are different.

In some embodiments, any of the methods described herein can employ a plurality of nucleotide conjugates, wherein individual nucleotide conjugates in the plurality comprise a core bound to at least one nucleotide-arm. In some cases, all of the nucleotide arms that are bound to a core can have the same reactive group in the linker. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the reactive group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a reactive group that can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the reactive group can comprise an azide, azido or azidomethyl group. In some embodiments, the azide, azido or azidomethyl group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the methods described herein can employ a plurality of nucleotide conjugates comprising a mixture (sub-populations) of two or more different types of nucleotide conjugates. In some embodiments, the mixture of two or more different types of nucleotide conjugates can comprise at least a first nucleotide conjugate. In some cases, the first nucleotide conjugate can comprise a core bound to at least one nucleotide-arm having a first type of reactive group in the linker. In some embodiments, the mixture of two or more different types of nucleotide conjugates can comprise at least a second nucleotide conjugate. In some cases, the second nucleotide conjugate can comprise a core bound to at least one nucleotide-arm having a second type of reactive group in the linker. In some embodiments, the mixture of two or more different types of nucleotide conjugates can comprise the first nucleotide conjugate and the second nucleotide conjugate. In some cases, the first reactive group differs from the second reactive group. In some embodiments, the first nucleotide conjugate can comprise a core bound to 2-5 biotinylated nucleotide arms, where the biotinylated-arms can have a first type of reactive group in the linker. In some embodiments, the second nucleotide conjugate can comprises a core bound to 2-5 biotinylated nucleotide arms, where the biotinylated-arms can have a second type of reactive group in the linker, where the first reactive group differs from the second reactive group.

In some embodiments, the first and second reactive can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second reactive group that can be reactive with a chemical agent. For example, the reactive groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The reactive groups aryl and benzyl can be reactive with H2 and Palladium on carbon (Pd/C). The reactive groups amine, amide, keto, isocyanate, phosphate, thiol, disulfide can be reactive with phosphine or with a thiol group including beta-mercaptoethanol or dithiothritol (DTT). The reactive group carbonate can be reactive with potassium carbonate (K₂CO₃) in MeOH, with triethylamine in pyridine, or with Zn in acetic acid (AcOH). The reactive groups urea and silyl can be reactive with tetrabutylammonium fluoride, pyridine-HF, with ammonium fluoride, or with triethylamine trihydrofluoride. In some embodiments, the first or second reactive can be selected, in any combination, from a group consisting of an azide, azido or azidomethyl group. In some embodiments, the azide, azido or azidomethyl reactive group in the linker can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the methods described herein can employ a plurality of nucleotide conjugates, wherein individual nucleotide conjugates in the plurality can comprise a core bound to at least one nucleotide-arm, wherein all of the nucleotide arms that are bound to a core can have a nucleotide unit having the same sugar 3′OH group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms.

In some embodiments, any of the methods described herein can employ a plurality of nucleotide conjugates, wherein individual nucleotide conjugates in the plurality can comprise a core bound to at least one nucleotide-arm, wherein all of the nucleotide arms that are bound to a core can have a nucleotide unit having the sugar 3′OH group substituted with the same 3′ blocking group. In some embodiments, individual nucleotide conjugates in the plurality can comprise a core bound to 2-5 nucleotide-arms. In some embodiments, individual nucleotide conjugates in the plurality can comprise a core bound to 2-5 biotinylated nucleotide-arms. In some embodiments, the sugar 3′blocking group can comprise alkyl, alkenyl, alkynyl, allyl, aryl, benzyl, azide, amine, amide, keto, isocyanate, phosphate, thiol, disulfide, carbonate, urea, or silyl group. In some embodiments, the individual nucleotide conjugates can comprise a 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the 3′ blocking group can comprise a 3′-O-alkyl hydroxylamino group, a 3′-phosphorothioate group, a 3′-O-malonyl group, or a 3′-O-benzyl group. In some embodiments, the 3′ blocking group can comprise an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the methods described herein can employ a plurality of nucleotide conjugates comprising a mixture (sub-populations) of two or more different types of nucleotide conjugates. In some embodiments, the mixture of two or more different types of nucleotide conjugates can comprise at least a first nucleotide conjugate. In some cases, the first nucleotide conjugate can comprise a core bound to at least one nucleotide-arm having a first nucleotide unit with a first type of sugar 3′OH blocking group. In some embodiments, the mixture of two or more different types of nucleotide conjugates can comprise at least a second nucleotide conjugate. In some cases, the second nucleotide conjugate can comprise a core bound to at least one nucleotide-arm having a second nucleotide unit having a second type of sugar 3′ blocking group. In some embodiments, the mixture of two or more different types of nucleotide conjugates can comprises the first nucleotide conjugate and the second nucleotide conjugate. In some cases, the first 3′ blocking group in the first nucleotide conjugate differs from the second 3′ blocking group in the second nucleotide conjugate. In some embodiments, the first nucleotide conjugate can comprise a core bound to 2-5 biotinylated nucleotide arms, where the biotinylated-arms can have a first type of 3′ blocking group. In some embodiments, the second nucleotide conjugate can comprise a core bound to 2-5 biotinylated nucleotide arms, where the biotinylated-arms can have a second type of 3′ blocking group, where the first 3′ blocking group differs from the second 3′ blocking group.

In some embodiments, the first and second 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first or second 3′ blocking group can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the methods described herein can employ a plurality of nucleotide conjugates comprising a mixture (sub-populations) of two or more different types of nucleotide conjugates. In some embodiments, the mixture of two or more different types of nucleotide conjugates can comprise at least a first nucleotide conjugate. In some cases, the first nucleotide conjugate can comprise a core bound to at least one nucleotide-arm having a first nucleotide unit with a sugar 3′ OH group. In some embodiments, the mixture of two or more different types of nucleotide conjugates can comprise at least a second nucleotide conjugate. In some cases, the second nucleotide conjugate can comprise a core bound to at least one nucleotide-arm having a second nucleotide unit having a first type of sugar 3′ blocking group. In some embodiments, the mixture of two or more different types of nucleotide conjugates can comprise the first nucleotide conjugate and the second nucleotide conjugate. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group.

In some embodiments, the first 3′ blocking group can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first 3′ blocking group can be selected, in any combination, from a group consisting of a 3′-O-alkyl hydroxylamino group, a 3′-phosphorothioate group, a 3′-O-malonyl group, and a 3′-O-benzyl group. In some embodiments, the first 3′ blocking group can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

In some embodiments, any of the methods described herein can employ a plurality of nucleotide conjugates comprising a mixture (sub-populations) of three or more different types of nucleotide conjugates. In some embodiment, the mixture of three or more different types of nucleotide conjugates can comprise at least a first nucleotide conjugate. In some cases, the first nucleotide conjugate can comprise a core bound to at least one nucleotide-arm having a first nucleotide unit with a sugar 3′ OH group. In some embodiments, the mixture of three or more different types of nucleotide conjugates can comprise at least a second nucleotide conjugate. In some cases, the second nucleotide conjugate can comprise a core bound to at least one nucleotide-arm having a second nucleotide unit having a first type of sugar 3′ blocking group. In some embodiments, the mixture of three or more different types of nucleotide conjugates can comprise at least a third nucleotide conjugate. In some cases, the third nucleotide conjugate can comprise a core bound to at least one nucleotide-arm having a third nucleotide unit having a second type of sugar 3′ blocking group. In some embodiments, the mixture of three or more different types of nucleotide conjugates can comprise the first nucleotide conjugate, the second nucleotide conjugate, and the third nucleotide conjugate. In some cases, the first 3′ blocking group from the second nucleotide conjugate and the second 3′ blocking group from the third nucleotide conjugate are different. In some embodiments, the first nucleotide conjugate can comprise a core bound to 2-5 biotinylated nucleotide arms, where the biotinylated-arms can have a sugar 3′ OH group. In some embodiments, the second nucleotide conjugate can comprise a core bound to 2-5 biotinylated nucleotide arms, where the biotinylated-arms can have a first type of 3′ blocking group. In some embodiments, the third nucleotide conjugate can comprise a core bound to 2-5 biotinylated nucleotide arms, where the biotinylated-arms can have a second type of 3′ blocking group.

In some embodiments, the first and second 3′ blocking groups can be selected, in any combination, from a group consisting of alkyl, alkenyl, alkynyl, allyl, aryl, benzyl, azide, amine, amide, keto, isocyanate, phosphate, thiol, disulfide, carbonate, urea, and silyl group. In some embodiments, the individual nucleotide conjugates can comprise a first or second 3′ blocking group that can be reactive with a chemical agent. For example, the 3′ blocking groups alkyl, alkenyl, alkynyl and allyl can be reactive with tetrakis(triphenylphosphine)palladium(0) (Pd(P(C₆H₅)₃)₄) with piperidine, or with 2,3-Dichloro-5,6-dicyano-1,4-benzo-quinone (DDQ). The 3′ blocking groups aryl and benzyl can be reactive with H2 and Palladium on carbon (Pd/C). The 3′ blocking groups amine, amide, keto, isocyanate, phosphate, thiol, disulfide can be reactive with phosphine or with a thiol group including beta-mercaptoethanol or dithiothritol (DTT). The 3′ blocking group carbonate can be reactive with potassium carbonate (K₂CO₃) in MeOH, with triethylamine in pyridine, or with Zn in acetic acid (AcOH). The 3′ blocking groups urea and silyl can be reactive with tetrabutylammonium fluoride, pyridine-HF, with ammonium fluoride, or with triethylamine trihydrofluoride. In some embodiments, the first and second 3′ blocking groups can be selected, in any combination, from a group consisting of a 3′-O-alkyl hydroxylamino group, a 3′-phosphorothioate group, a 3′-O-malonyl group, and a 3′-O-benzyl group. In some embodiments, the first and second 3′ blocking groups can be selected, in any combination, from a group consisting of an azide, azido or azidomethyl group. In some embodiments, the azide, azido or azidomethyl 3′ blocking group can be reactive with a chemical agent. In some embodiments, the chemical agent can comprise a phosphine compound. In some embodiments, the phosphine compound can comprise a derivatized tri-alkyl phosphine moiety or a derivatized tri-aryl phosphine moiety. In some embodiments, the phosphine compound can comprise Tris(2-carboxyethyl)phosphine (TCEP), bis-sulfo triphenyl phosphine (BS-TPP) or Tri(hydroxyproyl)phosphine (THPP).

### Computer-Implemented Methods

Disclosed herein, in some embodiments, are computer-implemented methods of performing a nucleic acid processing and/or analysis utilizing the computer systems disclosed herein. In some embodiments, the one or more processors are configured to perform the methods of the present disclosure. In some embodiments, the memory is configured to store instructions in the form of computer program or software that is executable by the one or more processors disclosed herein.

### Definitions

The headings provided herein are not limitations of the various aspects of the disclosure, which aspects can be understood by reference to the specification as a whole.

Unless defined otherwise, technical and scientific terms used herein have the standard definition, unless defined otherwise. Generally, terminologies pertaining to techniques of molecular biology, nucleic acid chemistry, protein chemistry, genetics, microbiology, transgenic cell production, and hybridization described herein are used in the art. Techniques and procedures described herein are generally performed and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those used in the art. Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of”, “consisting essentially of,” or a combination of “consisting essentially of” and “consisting of” are also provided.

As used herein, the terms “about” and “approximately” refer to a value or composition that is within an acceptable error range for the particular value or composition, which will depend in part on how the value or composition is measured or determined, e.g., the limitations of the measurement system. For example, “about” or “approximately” can mean within one or more than one standard deviation per the practice in the art. Alternatively, “about” or “approximately” can mean a range of up to 10% (e.g., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges, subranges, or a combination of ranges and subranges of values are provided, the ranges, subranges, or a combination of ranges and subranges can include the endpoints of the ranges, subranges, or a combination of ranges and subranges.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar (e.g., five carbon sugar comprising ribose or deoxyribose). A “nucleotide unit” referred to herein refers to a single nucleotide.

Nucleotides (and nucleosides) comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick hydrogen bonds, Hoogstein hydrogen bonds, or a combination of Watson-Crick hydrogen bonds and Hoogstein hydrogen bonds with an appropriate complementary base. Bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional bases can be found in Fasman, 1989, in “Practical Handbook of Biochemistry and Molecular Biology”, pp. 385-394, CRC Press, Boca Raton, Fla.

Nucleotides (and nucleosides) comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2 ′,3′-dideoxyribosyl; 2 ′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides can comprise a chain of one, two, three, or up to ten phosphorus atoms where the chain is attached to the 5′ carbon of the sugar moiety via an ester or phosphoramide linkage. In some embodiments, the nucleotide can be an analog having a phosphorus chain in which the phosphorus atoms are linked together with intervening O, S, NH, methylene or ethylene. In some embodiments, the phosphorus atoms in the chain can include substituted side groups including O, S or BH₃. In some embodiments, the chain can include phosphate groups substituted with analogs including phosphoramidate, phosphorothioate, phosphordithioate, and O-methylphosphoroamidite groups.

The term “detectable moiety”, “detectable reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. Reporter moieties can be selected so that each absorbs excitation radiation, emits fluorescence, or a combination of absorbing excitation radiation and emitting fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A detectable reporter moiety (or label) comprises a fluorescent label or a fluorophore. Fluorescent moieties which may serve as fluorescent labels or fluorophores can include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, quinolinium groups, or any combination thereof separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-41E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carb oxy pentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the detectable reporter moiety can be a FRET pair, such that multiple classifications can be performed under a single excitation and imaging protocol. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but are not limited to: nucleotide transient-binding; nucleotide incorporation; de-blocking; washing; removing; flowing; detecting; imaging, identifying, or any combination thereof. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage can occur intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. The first nucleic acid component can be located 5′ to the second nucleic acid component, or vice versa. For example, linkage between a primer binding sequence and a sequence of interest can form a cojoined nucleic acid molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene can be operably linked to a host cell regulatory sequence (e.g., a promoter sequence) that affects expression of the transgene in the host cell. In some embodiments, the vector can comprise at least one host cell regulatory sequence, including a promoter sequence, enhancer, transcription initiation sequence, translation initiation sequence, or a combination of transcription and translation initiation sequences, transcription termination sequences, translation termination sequences, or a combination of transcription and translation termination sequences, polypeptide secretion signal sequences, and the like. In some embodiments, the host cell regulatory sequence can control expression of the level, timing of the transgenes, location of the transgene, or a combination of timing and location of the transgene.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases, sugars, or a combination of natural or non-natural bases and sugars. Nucleic acids comprise naturally-occurring internucleosidic linkages, for example phosphodiester linkages. Nucleic acids comprise non-natural internucleoside linkages, including phosphorothioate, phosphorothiolate, or peptide nucleic acid (PNA) linkages. In some embodiments, nucleic acids can comprise a one type of polynucleotides or a mixture of two or more different types of polynucleotides.

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The sequence of the template nucleic acid can be partially or wholly complementary to the sequence of the complementary strand. The complementary nucleic acid strand can be generated by conducting a primer extension reaction on a template strand. The primer extension reaction can include amplification and sequencing reactions. The template nucleic acid can be obtained from a naturally-occurring source, recombinant form, or chemically synthesized to include any type of nucleic acid analog. The template nucleic acid can be linear, circular, or other forms. The template nucleic acids can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The template nucleic acid molecules may be isolated from any source including from organisms such as prokaryotes, eukaryotes (e.g., humans, plants and animals), fungus, and viruses; cells; tissues; normal or diseased cells or tissues, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. The template nucleic acid can be subjected to nucleic acid analysis, including sequencing and composition analysis.

The term “primer” and related terms used herein refers to an oligonucleotide, either natural or synthetic, that is capable of hybridizing with a DNA polynucleotide template, a RNA polynucleotide template, or a combination of a DNA and RNA polynucleotide template to form a duplex molecule. Primers may have any length, but typically range from 4-50 nucleotides. A primer comprises a 5′ end and 3′ end. The 3′ end of the primer can include a terminal 3′ OH moiety which serves as a nucleotide polymerization initiation site in a polymerase-catalyzed primer extension reaction. Alternatively, the 3′ end of the primer can lack a terminal 3′ OH moiety, or can include a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a surface capture primer).

When used in reference to nucleic acid molecules, the terms “hybridize” or “hybridizing” or “hybridization” or other related terms refers to hydrogen bonding between two different nucleic acids to form a duplex nucleic acid. Hybridization can also include hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands may not hybridize with each other across their entire length. The complementary base pairing can be the standard A-T or C-G base pairing, or can be other forms of base-pairing interactions. Duplex nucleic acids can include mismatched base-paired nucleotides.

When used in reference to nucleic acids, the terms “extend”, “extending”, “extension” and other variants, refers to incorporation of one or more nucleotides into a nucleic acid molecule. Nucleotide incorporation can comprise polymerization of one or more nucleotides into the terminal 3′ OH end of a nucleic acid strand, resulting in extension of the nucleic acid strand. Nucleotide incorporation can be conducted with natural nucleotides, nucleotide analogs, or a combination of natural nucleotides and nucleotide analogs. Nucleotide incorporation can occur in a template-dependent fashion. Any suitable method of extending a nucleic acid molecule may be used, including primer extension catalyzed by a DNA polymerase or RNA polymerase.

As used herein, the term “clonally amplified” and it variants refers to a nucleic acid template molecule that has been subjected to one or more amplification reactions either in-solution or on-support. In the case of in-solution amplified template molecules, the resulting amplicons can be distributed onto the support. Prior to amplification, the template molecule can comprise a sequence of interest and at least one universal adaptor sequence. In some embodiments, clonal amplification can comprise the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

The term “polymerizing enzyme” and its variants, as used herein, comprises any enzyme or catalytic portion thereof that can catalyze polymerization of nucleotides (including analogs thereof) into a nucleic acid strand. In some embodiments, the polymerizing enzyme is a polymerase. Nucleotide polymerization can occur in a template-dependent fashion. A polymerase can comprise one or more active sites at which nucleotide binding, catalysis of nucleotide polymerization, or a combination of nucleotide binding and catalysis of nucleotide polymerization can occur. In some embodiments, a polymerase can include other enzymatic activities, such as for example, 3′ to 5′ exonuclease activity or 5′ to 3′ exonuclease activity. In some embodiments, a polymerase can have strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “binding complex” refers to a complex formed by binding together a nucleic acid duplex, a polymerase, and a free nucleotide or a nucleotide unit of a nucleotide conjugate, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule.

As used herein, the term “ternary complex” may be an example of a binding complex which can be formed by binding together a nucleic acid duplex, a polymerizing enzyme, and a free nucleotide or nucleotide unit of a nucleotide conjugate, where the free nucleotide or nucleotide unit may be bound to the 3′ end of the nucleic acid primer (as part of the nucleic acid duplex) at a position that can be opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a nucleotide conjugate or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time can be indicative of the stability of the binding complex and strength of the binding interactions. Persistence time can be measured by observing the onset of a binding complex, the duration of a binding complex, or a combination of the onset and the duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One label can be a fluorescent label.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions such as stringent conditions commonly used in the art) to hybridization with non-target sequences during a hybridization reaction. Typically, hybridizable sequences share a degree of sequence complementarity over all or a portion of their respective lengths such as between 25%-100% complementarity, including greater than or equal to about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm. Optimal alignment may be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are commonly known. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “multivalent binding complex” refers to a binding complex formed by a single nucleotide conjugate and two or more substrates (e.g., nucleic acid sequence). In some embodiments, the multivalent binding complex also comprises two or more polymerizing enzymes disclosed herein.

As used herein, the term nucleotide conjugate refers to a conjugate comprising a core; and one or more nucleotide units coupled to the core directly or indirectly.

As used herein, the term “core” as used herein refers to a central member of a nucleotide conjugate that is coupled to one or more nucleotide units directly or indirectly.

As used herein, the term “package” refers to a suitable solid matrix or material such as glass, plastic, paper, foil, and the like, capable of holding the individual kit components. For example, a package may be a glass vial or prefilled syringes used to contain suitable quantities of the pharmaceutical. The packaging material has an external label which indicates the contents and/or purpose of the kit and its components.

In some embodiments, the support can be solid, semi-solid, or a combination of both. In some embodiments, the support can be porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support can comprise a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates may be contemplated.

In some embodiments, the surface of the support can be coated with one or more compounds to produce a passivated layer on the support. In some embodiments, the support can comprise a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides (e.g., surface capture primers) that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may be no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In some cases, the contact angle can be no more than 40 degrees. A given hydrophilic, low-binding support surface of the present disclosure may exhibit a water contact angle having a value of anywhere within this range.

The present disclosure provides a plurality (e.g., two or more) of nucleic acid templates immobilized to a support. In some embodiments, the immobilized plurality of nucleic acid templates can have the same sequence or have different sequences. In some embodiments, individual nucleic acid template molecules in the plurality of nucleic acid templates can be immobilized to a different site on the support. In some embodiments, two or more individual nucleic acid template molecules in the plurality of nucleic acid templates can be immobilized to a site on the support. In some embodiments, the support can comprise a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites can be arranged on the support in an organized fashion. In some embodiments, the plurality of pre-determined sites can be arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support can comprise at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites may be located at pre-determined locations on the support. In some embodiments, a plurality of pre-determined sites on the support (e.g., 10²-10¹⁵ sites or more) can be immobilized with nucleic acid templates to form a nucleic acid template array. In some embodiments, the nucleic acid templates can be immobilized at a plurality of pre-determined sites by hybridization to immobilized surface capture primers, or the nucleic acid templates can be covalently attached to the surface capture primers. In some embodiments, the nucleic acid templates can be immobilized at a plurality of pre-determined sites, for example immobilized at 10²-10¹⁵ sites or more. In some embodiments, the nucleic acid templates that are immobilized at a plurality of sites on the support can comprise linear or circular nucleic acid template molecules or a mixture of both linear and circular molecules. In some embodiments, the immobilized nucleic acid templates can be clonally-amplified to generate immobilized nucleic acid polonies at the plurality of pre-determined sites. In some embodiments, individual immobilized nucleic acid template molecules can comprise one copy of a sequence of interest, or comprise concatemers having two or more tandem copies of a sequence of interest.

In some embodiments, a support comprising a plurality of sites located at random locations on the support is referred to herein as a support having randomly located sites thereon. The location of the randomly located sites on the support may not pre-determined. The plurality of randomly-located sites can be arranged on the support in a disordered, an unpredictable, or a combination of disordered and unpredictable fashion. In some embodiments, the support can comprise at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) can be immobilized with nucleic acid templates to form a support immobilized with nucleic acid templates. In some embodiments, the nucleic acid templates can be immobilized at a plurality of randomly located sites by hybridization to immobilized surface capture primers, or the nucleic acid templates can be covalently attached to the surface capture primer. In some embodiments, the nucleic acid templates can be immobilized at a plurality of randomly located sites, for example immobilized at 10²-10¹⁵ sites or more. In some embodiments, the nucleic acid templates that are immobilized at a plurality of sites on the support can comprise linear or circular nucleic acid template molecules or a mixture of both linear and circular molecules. In some embodiments, the immobilized nucleic acid templates can be clonally-amplified to generate immobilized nucleic acid polonies at the plurality of randomly located sites. In some embodiments, individual immobilized nucleic acid template molecules can comprise one copy of a sequence of interest, or comprise concatemers having two or more tandem copies of a sequence of interest.

In some embodiments, with respect to nucleic acid template molecules immobilized to pre-determined or random sites on the support, the plurality of immobilized nucleic acid template molecules on the support can be in fluid communication with each other to permit flowing a solution of reagents (e.g., enzymes including polymerases, nucleotide conjugates, nucleotides, divalent cations, buffers, or a combination thereof, and the like) onto the support so that the plurality of immobilized nucleic acid template molecules on the support can be reacted with the reagents in a massively parallel manner. In some embodiments, the fluid communication of the plurality of immobilized nucleic acid template molecules can be used to conduct nucleotide binding assays, conduct nucleotide polymerization reactions (e.g., primer extension or sequencing), or a combination of nucleotide bindings assays and nucleotide polymerization reactions on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, where the nucleic acid molecules or enzymes can be attached directly to a support through covalent bond or non-covalent interaction, or the nucleic acid molecules or enzymes are attached to a coating on the support.

As used herein, the term “sequencing” and its variants comprise obtaining sequence information from a nucleic acid strand, by determining the identity of at least some nucleotides (including their nucleobase components) within the nucleic acid template molecule. While in some embodiments, “sequencing” a given region of a nucleic acid molecule can include identifying each and every nucleotide within the region that is sequenced, in some embodiments “sequencing” can comprise methods whereby the identity of some of the nucleotides in the region is determined, while the identity of some nucleotides remains undetermined or incorrectly determined. Any suitable method of sequencing may be used. In an embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing can include massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing can include Heliscope single molecule sequencing, and single molecule real time (SMRT) sequencing. An example of sequence-by-hybridization can include SOLiD sequencing (e.g., from Life Technologies; WO 2006/084132). An example of sequence-by-binding can include Omniome sequencing (e.g., U.S. Pat. No. 10,246,744).

### EXAMPLES

The following examples are included for illustrative purposes only and are not intended to limit the scope of the methods or systems.

### Example 1: Preparation of Nucleotide-Arms

In a 1.5 mL Eppendorf tube, 320 uL of biotin-5k PEG-SVA (from Laysan Bio) was mixed with 33% DMF to produce a concentration of 25 mM of the Biotin-5k PEG-SVA. In a separate tube, add 440 uL buffer (0.2 M NaHCO₃Na₂CO₃ pH 9) and 200 uL of dGTP-PA-NH₂ (10 mM stock, from MyChem), the tube was centrifuged. The dissolved biotin-5k PEG SVA was added to the second tube and incubated at room temperature for 1 hour. The reaction was purified via ion exchange chromatography.

The nucleotide-arm comprising an azido group was synthesized as follows. The FMOC N3 linker was obtained from a commercial source. An NHS ester synthesis reaction was conducted by mixing together one equivalent of the N3 linker, one equivalent of disuccinimidyl carbonate (DSC), one equivalent of 4-dimethylaminopyridine (DMAP), with anhydrous N,N-dimethylformamide (DMF), at room temperature for 1 hour. The conjugation to propargyl-amine dNTPs was conducted by reacting three equivalents of the NHS-ester solution with one equivalent of propargyl-dATP, with reaction buffer, at room temperature, for 1 hour.

### Example 2: Preparation of Streptavidin Core

Ten mg of streptavidin (Anaspec, catalog No. AS-72177) was dissolved in 525 uL of freshly-prepared 1×PBS buffer (pH 8), and centrifuged for 5 minutes at 14,000 rcf at 4° C. to aggregate the protein. The concentration of the mixture was analyzed via Nanodrop at absorption 280 nm, with E=179200 M−1 cm−1 for the tetramer (assuming MW=56,000). The mixture was diluted 1:10 with water.

The fluorophore NHS ester was prepared as a 25 mM stock in DMSO. In a 5 mL Eppendorf tube, DMSO and modified 1×PBS buffer (pH8 with 0.01% Tween) and streptavidin was added. The fluorophore was added slowly, and incubated in the dark at room temperature for 7 hours. The reaction was quenched by adding 100 uL of 1M glycine (pH 9). The mixture was centrifuged for 5 minutes at 14,000 rcf at 4° C., and any precipitate was discarded. Unreacted fluorophore was removed using an Amicon Ultra-15 filter.

### Example 3: Preparation of Nucleotide Conjugates

One type of nucleotide conjugate was prepared by reacting propargylamine dNTPs with Biotin-PEG-NHS. This aqueous reaction was driven to completion and purified to produce a biotin-PEG-dNTP species. In separate reactions, several different PEG lengths were used, corresponding to average molecular weights varying from 1K Da to 20K Da. The Biotin-PEG-dNTP species were mixed with either freshly prepared or commercially-sourced dye-labeled streptavidin (SA) using a Dye: SA ratio of approximately 3-5:1. Mixing of biotin-PEG-dNTP with dye-labeled streptavidin was conducted in the presence of excess biotin-PEG-dNTP to ensure saturation of the biotin binding sites on each streptavidin tetramer. Complete complexes were purified away from excess biotin-PEG-dNTP by size exclusion chromatography. Each type of multivalent nucleotide having either dATP, dGTP, dCTP or dTTP nucleotide units was conjugated and purified separately, then mixed together to create a four base mixture for nucleotide binding, nucleotide incorporation and nucleic acid sequencing reactions.

Another type of nucleotide conjugates was prepared in a single pot by reacting multi-arm PEG NHS with excess dye-NH₂ and propargylamine dNTPs. Various multi-arm PEG NHS variants were used ranging from 4-16 arms and ranging in molecular weight from 5K Da to 40K Da. After reacting, excess small molecule dye and dNTP were removed by size exclusion chromatography. Each type of multivalent nucleotide having either dATP, dGTP, dCTP or dTTP nucleotide units was conjugated and purified separately, then mixed together to create a four base mixture for nucleotide binding, nucleotide incorporation and nucleic acid sequencing reactions.

The single pot method is described herein. In a 2 mL Eppendorf tube, mixed 914.1 uL of water, 150 uL of acetonitrile, 112.5 uL of TEAB, 51.6 uL of the biotinylated nucleotide-arms, and 271.7 uL of the labeled streptavidin core. The mixture was incubated for 15 minutes at room temperature in the dark. Unreacted biotinylated nucleotide-arms were removed with Amicon Ultra-4.

### Example 4: Trapping Assays on Plates

Trapping assays were conducted to determine the capability of a nucleotide unit (as part of a nucleotide conjugate) to bind a complexed polymerase. The trapping assays were conducted under conditions that permit binding of the nucleotide unit to the complexed polymerase but without incorporation. The complexed polymerase included a polymerase bound to a nucleic acid template molecule which is hybridized to a primer.

Wells of 394-well plates were coated with PEG-silane. Single-stranded polonies of template molecules (clonally-amplified) were prepared in the wells. A sequencing primer was hybridized to the polonies.

Trapping assay using nucleotides having a 3′ azido-blocked moiety: The wells were pre-washed once with 20 mM TRIS pH 8.8, 10 mM (NH₄)₂SO₄, 10 mM KCl, 10 mM MgSO₄. Azido-blocked nucleotides were incorporated in 20 mM TRIS pH 8.8, 10 mM (NH₄)₂SO₄, 10 mM KCl, 10 mM MgSO₄, 5 μM dNTP-N3, 600 nM a polymerase at 55° C. for 5 minutes. The wells were washed six times with 50 mM TRIS pH 8.0, 1 mM EDTA pH 7.5, 750 mM NaCl, 0.02% Tween-20.

Trapping assay using nucleotide conjugates: The wells were washed once with 10 mM TRIS pH 8.0, 0.5 mM EDTA, 50 mM NaCl. Trap reactions were performed by adding 10 mM TRIS pH 8.0, 2 M Betaine, 1% Triton X-100, 0.48 μM polymerase, 10 mM CaCl₂, 0.5 mM EDTA, 100 mM NaCl, 20-160 nM fluorescently-labeled nucleotide conjugates, for 45 seconds at 45° C. The wells were washed 5 times with 10 mM TRIS pH 8.0, 2 M betaine, 10 mM CaCl₂, 100 mM NaCl, 0.5 mM EDTA, 1% Triton X-100.

The trapping assay using the nucleotide conjugates were suitable for forming a plurality of multivalent binding complexes on concatemer template molecules (e.g., polonies). For example, the trapping assays comprise: (a) binding a first nucleic acid primer, a first polymerase, and a first nucleotide conjugate to a first portion of a concatemer template molecule thereby forming a first binding complex, wherein a first nucleotide unit of the first nucleotide conjugate binds to the first polymerase; and (b) binding a second nucleic acid primer, a second polymerase, and the first nucleotide conjugate to a second portion of the same concatemer template molecule thereby forming a second binding complex, wherein a second nucleotide unit of the first nucleotide conjugate binds to the second polymerase, wherein the first and second binding complexes which include the same nucleotide conjugate forms a first multivalent binding complex.

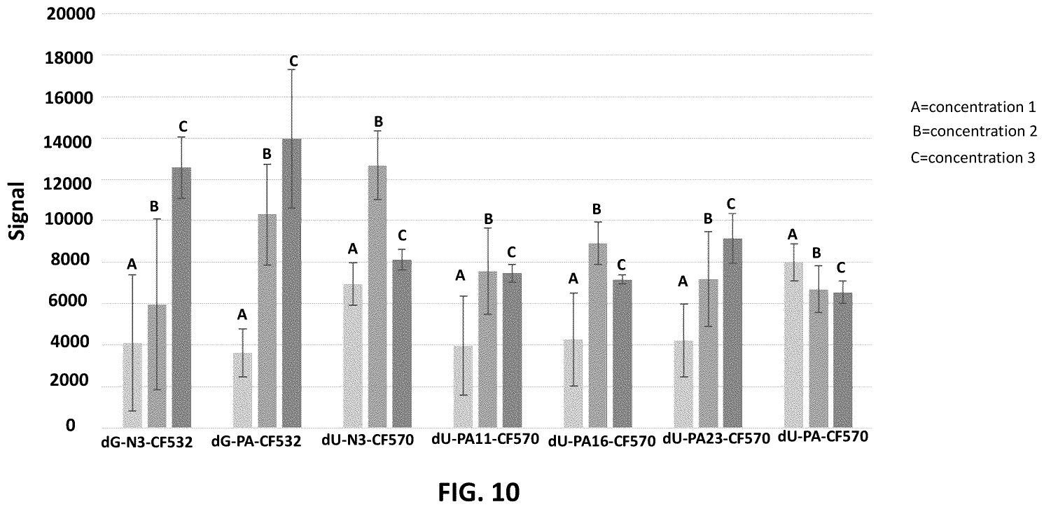

The surfaces were imaged using epifluorescence and the signal intensity was determined using the 90^{th} percentile. The data is shown in FIGS. 9 and 10.

The data in FIG. 9 shows that dA nucleotide conjugates (dATP nucleotide unit) produce optimal signals using PA, PA11, or PA23 linkers. The dC nucleotide conjugates (dCTP nucleotide units) produce an optimal signal when carrying the N3 linker. It is notable that nucleotide conjugates carrying the PA linker produces an optimal signal when linked to a dA (dATP) nucleotide unit, however nucleotide conjugates carrying the same linker and Cy5 dye combination fails to produce an optimal signal when linked to a dC (dCTP) nucleotide unit.

The data in FIG. 10 show that signal intensity varied as a function of linker and concentration.

### Example 5: Trapping Assays on Flow Cells

Trapping assays were conducted to determine the capability of a nucleotide unit (as part of a nucleotide conjugate) to bind a complexed polymerase. The trapping assays were conducted under conditions that permit binding of the nucleotide unit to the complexed polymerase but without incorporation. A complexed polymerase includes a polymerase bound to a nucleic acid template molecule which is hybridized to a primer.

Fluorescently-labeled nucleotide conjugates carrying Linker-6 were prepared. Labeled nucleotide conjugates carrying the N3-Linker, Linker-8 or 11-atom Linker (sometimes called ‘PA’ Linker) were also prepared. The nucleotide conjugates were labeled with fluorophores CF680, CF532, CF570 or AF647.

Mixes of nucleotide conjugates carrying two different color fluorophores were prepared. One mix contained 20 nM or 80 nM of each of dCTP-CF680 and dUTP-CF532 nucleotide conjugates. Another mix contained 20 nM or 80 nM of each of dATP-AF647 and dGTP-CF570 nucleotide conjugates. Each of these mixes were prepared for nucleotide conjugates having a different linker: N3-Linker; Linker-6 (A-linker); Linker-8 (mAMBA-linker); or 11 atom Linker (also called PA Linker). For example, a first mix contained 20 nM of dCTP-CF680 and dUTP-CF532 nucleotide conjugates carrying N3-Linkers. A second mix contained 20 nM of dCTP-CF680 and dUTP-CF532 nucleotide conjugates carrying Linker-6. Twelve different mixes were prepared. Each mix also contained 0.1 μM sequencing polymerase, 5 mM strontium acetate, a buffering compound, EDTA, a salt, detergent and viscosity additives. The strontium acetate was included in the mixes to promote binding of the nucleotide units of the nucleotide conjugates to the complexed polymerases without incorporation. Individual complexed polymerases included a polymerase bound to a template molecule which was hybridized to a primer.

Single-stranded concatemer template molecules were immobilized on a flow cell. The template molecules were hybridized with sequencing primers. The flow cell was loaded into a sequencing apparatus configured to deliver laser excitation to the flow cell and obtain fluorescent images from the flow cell.

Repeat cycles of binding reactions were conducted. Each binding cycle included the following general method: flowing a multivalent mix and incubation; washing; imaging; and washing. The flow cell was pre-washed, then flowed with a mix of labeled nucleotide conjugates and incubated for a different length of time (e.g., 2-180 seconds). The flow cell was washed. The flow cell was imaged using epifluorescence of a red and green channel, and the signal intensity was determined using the 90^{th} percentile. The flow cell was washed. The binding cycles were repeated 62 times for the mixes containing dATP-AF647 and dGTP-CF570 nucleotide conjugates, and 71 times for the mixes containing dCTP-CF680 and dUTP-CF532 nucleotide conjugates.

The trapping assay using the nucleotide conjugates were suitable for forming a plurality of multivalent binding complexes on concatemer template molecules (e.g., polonies). For example, the trapping assays comprise: (a) binding a first nucleic acid primer, a first polymerase, and a first nucleotide conjugate to a first portion of a concatemer template molecule thereby forming a first binding complex, wherein a first nucleotide unit of the first nucleotide conjugate binds to the first polymerase; and (b) binding a second nucleic acid primer, a second polymerase, and the first nucleotide conjugate to a second portion of the same concatemer template molecule thereby forming a second binding complex, wherein a second nucleotide unit of the first nucleotide conjugate binds to the second polymerase, wherein the first and second binding complexes which include the same nucleotide conjugate forms a first multivalent binding complex.

In FIGS. 11 and 12, the data for N3-Linker molecules are in green, Linker-molecules are in blue, Linker-8 molecules are in red, and 11 atom Linker molecules are in purple.

The data in FIG. 11 generally shows that nucleotide conjugates at a concentration of 20 nM or 80 nM, and having dCTP or dUTP nucleotide units, and labeled with CF680 or CF532, the N3-Linker generated the highest signal intensities at all binding times tested, the Linker-6 molecules generated the next highest signal intensities, Linker-8 molecules generated lower signal intensities, and the 11 atom Linker molecules generated the lowest signal intensities.

The data in FIG. 12 generally shows that nucleotide conjugates at a concentration of 20 nM or 80 nM, and having dATP nucleotide units, and labeled with AF647, the N3-Linker generated the highest signal intensities at all binding times tested, the Linker-6 molecules generated the next highest signal intensities, Linker-8 molecules generated lower signal intensities, and the 11 atom Linker molecules generated the lowest signal intensities.

The data in FIG. 12 generally shows that nucleotide conjugates at a concentration of 20 nM or 80 nM, and having dGTP nucleotide units, and labeled with CF570, the N3-Linker generated the highest signal intensities at all binding times tested, and the Linker-8 molecules generated the lowest signal intensities. The Linker-6 and 11 atom Linker molecules generated similar signal intensities that were lower than the intensities of the N3-Linker molecules and higher than the Linker-8 molecules.

The data in FIGS. 11 and 12 indicate that signal intensities generated by labeled nucleotide conjugates binding to complexed polymerases may be impacted by the linker structure, the nucleotide unit, the fluorophore dye, or a combination thereof.

### Example 6: Real-Time Imaging of Trapping on a Microscope

Real-time trapping assays were conducted to determine the binding kinetics of a nucleotide unit (as part of a nucleotide conjugate) to bind a complexed polymerase. The real-time trapping assays were conducted under conditions that permit binding of the nucleotide unit to the complexed polymerase but without incorporation. The complexed polymerase included a polymerase bound to a nucleic acid template molecule which is hybridized to a primer.

A trap mix with quencher was prepared, which included: Tris HCl (pH 8.8), EDTA (pH 7.5), NaCl, Triton X-100, strontium acetate, sucrose, and a combination of reagents that can act as singlet oxygen quenchers. Sequencing polymerase was added to the trap/quencher mix to generate a trap/quencher/enzyme mix. For example, the sequencing polymerase comprises an amino acid sequence of any one of SEQ ID NOS: 1-390, 464, 391-463, or 465. The trap/quencher/enzyme mix was split into twelve separate aliquots, and each aliquot was mixed with one type of nucleotide conjugate at a concentration of 2.5 nM, 7.5 nM or 15 nM (e.g., the nucleotide conjugates included nucleotide units dATP, dGTP, dCTP or dUTP) to generate twelve separate enzyme/nucleotide conjugate mixes. The nucleotide conjugates in each of the twelve separate mixes were labeled with either a red or green fluorophore. Different enzyme/nucleotide conjugate mixes were prepared to test and compare nucleotide conjugates carrying a different linker, including Linker 6, 10, 11, 12, 13, 14, 15 or 16.

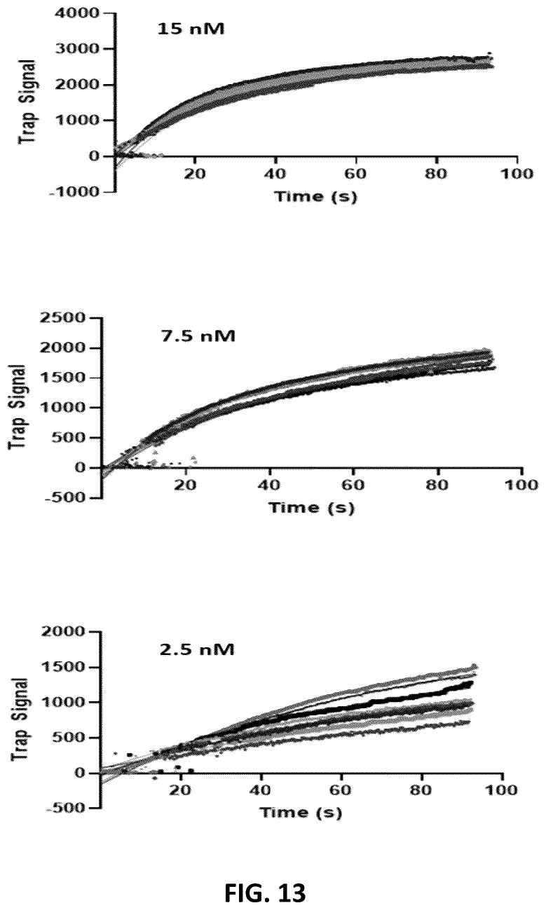

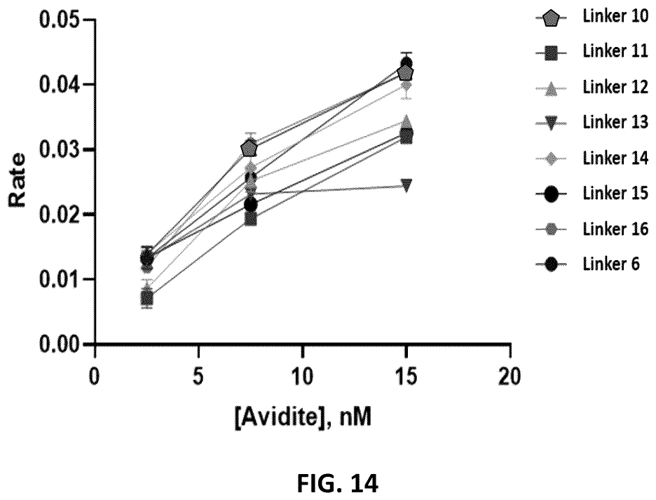

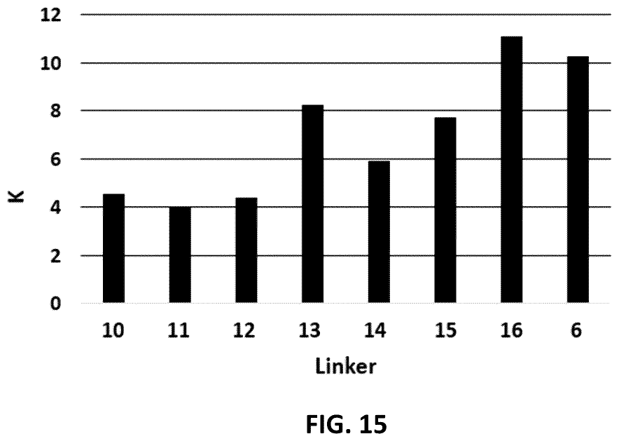

A flow cell having immobilized concatemer template molecules was prepared. The flow cell was loaded into a sequencing apparatus configured to deliver laser excitation to the flow cell and obtain fluorescent images from the flow cell. The enzyme/nucleotide conjugate mixes were flowed onto the flow cell. Images were obtained for 0.25 second exposure time (e.g., 400 images were obtained for 100 seconds). The signal intensities of the images were plotted and fitted to a single-phase exponential curve to determine the K value, and upper and lower limits. The results are shown in FIGS. 13, 14 and 15. The legend shown in FIG. 14 is also applicable to the date in FIG. 13.

### Example 7: Crystallization of a Nucleotide Conjugate Binding a Polymerase

Crystals of a nucleotide conjugate binding a polymerase which is bound to a template-primer duplex (e.g., a binding complex) were obtained by hanging drop diffusion method. A recombinant exonuclease-minus mutant polymerase having a backbone sequence based on SEQ ID NO:1 was co-crystallized with a two-fold excess of template/primer DNA duplex. The template/primer duplex included a 16-mer hybridized to a 14-mer, and the 14-mer oligonucleotide included a 3′ terminal di-deoxynucleotide.

Crystallization was conducted by hanging drop vapor diffusion at room temperature under conditions containing PEG, glycerol and ethylene glycol and utilized seeding. The crystals were soaked overnight in a 1 mM solution of a nucleotide conjugates (e.g., having a streptavidin core joined to biotinylated nucleotide arms comprising a spacer, Linker-6, and dCTP nucleotide unit. The structure was solved by molecular replacement using KLD structure from PDB and refinement and model building was conducted followed by manual inspection and rebuilding. The 2.8 Angstrom model is shown in FIG. 16.

### Example 8: Sequencing Using Nucleotide Conjugates

A two-stage sequencing reaction was conducted on a flow cell having a plurality of concatemer template molecules immobilized thereon.

The first-stage sequencing reaction was conducted by hybridizing a plurality of a soluble sequencing primers to the immobilized concatemers to form immobilized primer-concatemer duplexes. A plurality of a first sequencing polymerase was flowed onto the flow cell (e.g., contacting the immobilized primer-concatemer duplexes) and incubated under a condition suitable to bind the sequencing polymerase to the duplexes to form complexed polymerases. However, any one of the polymerases disclosed herein, see for e.g., having an amino acid sequence of any one of SEQ ID NOs: 1-390, 464, 391-463, or 465, could be used as well. A mixture of fluorescently labeled nucleotide conjugates (e.g., at a concentration of about 20-100 nM) was flowed onto the flow cell in the presence of a buffer that included a non-catalytic cation (e.g., strontium, barium, calcium, or combination thereof) and incubated under conditions suitable to bind complementary nucleotide units of the nucleotide conjugates to the complexed polymerases to form multivalent binding complexes without polymerase-catalyzed incorporation of the nucleotide units. The complexed polymerases were washed. An image was obtained of the fluorescently labeled nucleotide conjugates that remained bound to the complexed polymerases. The first sequencing polymerases and nucleotide conjugates were removed, while retaining the sequencing primers hybridized to the immobilized concatemers (retained duplexes), by washing with a buffer comprising a detergent.

The first stage sequencing reaction was suitable for forming a plurality of multivalent binding complexes on concatemer template molecules (e.g., polonies). For example, the first stage sequencing reaction comprises: (a) binding a first nucleic acid primer, a first polymerase, and a first nucleotide conjugate to a first portion of a concatemer template molecule thereby forming a first binding complex, wherein a first nucleotide unit of the first nucleotide conjugate binds to the first polymerase; and (b) binding a second nucleic acid primer, a second polymerase, and the first nucleotide conjugate to a second portion of the same concatemer template molecule thereby forming a second binding complex, wherein a second nucleotide unit of the first nucleotide conjugate binds to the second polymerase, wherein the first and second binding complexes which include the same nucleotide conjugate forms a first multivalent binding complex.

The second-stage sequencing reaction was conducted by contacting the retained duplexes with a plurality of second sequencing polymerases to form complexed polymerases. However, any one of the polymerases disclosed herein, see for e.g., having an amino acid sequence of any one of SEQ ID NO: 1-390, 464, 391-463, or 465 could be used as well. A mixture of fluorescently labeled nucleotide analogs (e.g., 3′O-methylazido nucleotides) (e.g., about 1-5 μM) was added to the complexed polymerases in the presence of a buffer that included a catalytic cation (e.g., magnesium, manganese, or a combination of magnesium and manganese) and incubated under conditions suitable to bind complementary nucleotides to the complexed polymerases and promote polymerase-catalyzed incorporation of the nucleotides to generate a nascent extended sequencing primer. The complexed polymerases were washed. An image was obtained of the incorporated fluorescently labeled nucleotide analogs as a part of the complexed polymerases. The incorporated fluorescently labeled nucleotide analogs were reacted with a cleaving reagent that removes the 3′ O-methylazido group and generates an extendible 3′OH group.

In an alternative second stage sequencing reaction, a mixture of non-labeled nucleotide analogs (e.g., 3′O-methylazido nucleotides) (e.g., about 1-5 μM) was added to the complexed polymerases in the presence of a buffer that included a catalytic cation (e.g., magnesium, manganese, or a combination of magnesium and manganese) and incubated under conditions suitable to bind complementary nucleotides to the complexed polymerases and promote polymerase-catalyzed incorporation of the nucleotides to generate a nascent extended sequencing primer. The complexed polymerases were washed. No image was obtained. The incorporated non-labeled nucleotide analogs were reacted with a cleaving reagent that removes the 3′ O-methylazido group and generates an extendible 3′OH group.

The second sequencing polymerases were removed, while retaining the nascent extended sequencing primers hybridized to the concatemers (retained duplexes), by washing with a buffer comprising a detergent. Recurring sequencing reactions were conducted by performing multiple cycles of first-stage and second-stage sequencing reactions to generate extended forward sequencing primer strands.

### EMBODIMENTS

Embodiment 1. A plurality of nucleotide conjugates, wherein individual nucleotide conjugates in the plurality comprise:
a) a core; and
b) a plurality of nucleotide arms wherein each nucleotide-arm comprises
c) a core attachment moiety,
d) a spacer,
e) a linker, and
f) a nucleotide unit,
wherein the core is attached to the plurality of nucleotide arms via the core attachment moiety, wherein the spacer is attached to the linker, and wherein the linker is attached to the nucleotide unit.

Embodiment 2. The plurality of nucleotide conjugates of embodiment 1, wherein the core is attached to 2-20 nucleotide-arms.

Embodiment 3. The plurality of nucleotide conjugates of embodiment 1, wherein the core comprises streptavidin or avidin, and wherein the core attachment moiety comprises biotin.

Embodiment 4. The plurality of nucleotide conjugates of embodiment 1, comprising a fluorescently-labeled nucleotide conjugate.

Embodiment 5. The plurality of nucleotide conjugates of embodiment 1, wherein the spacer comprises a structure

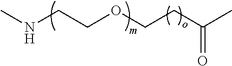

wherein m is 20-500, and
wherein o is 1-10.

Embodiment 6. The plurality of nucleotide conjugates of embodiment 1, wherein the linker comprises an aliphatic chain having 2-6 subunits or an oligo ethylene glycol chain having 2-6 subunits.

Embodiment 7. The plurality of nucleotide conjugates of embodiment 1, wherein the linker comprises any one of the following linkers

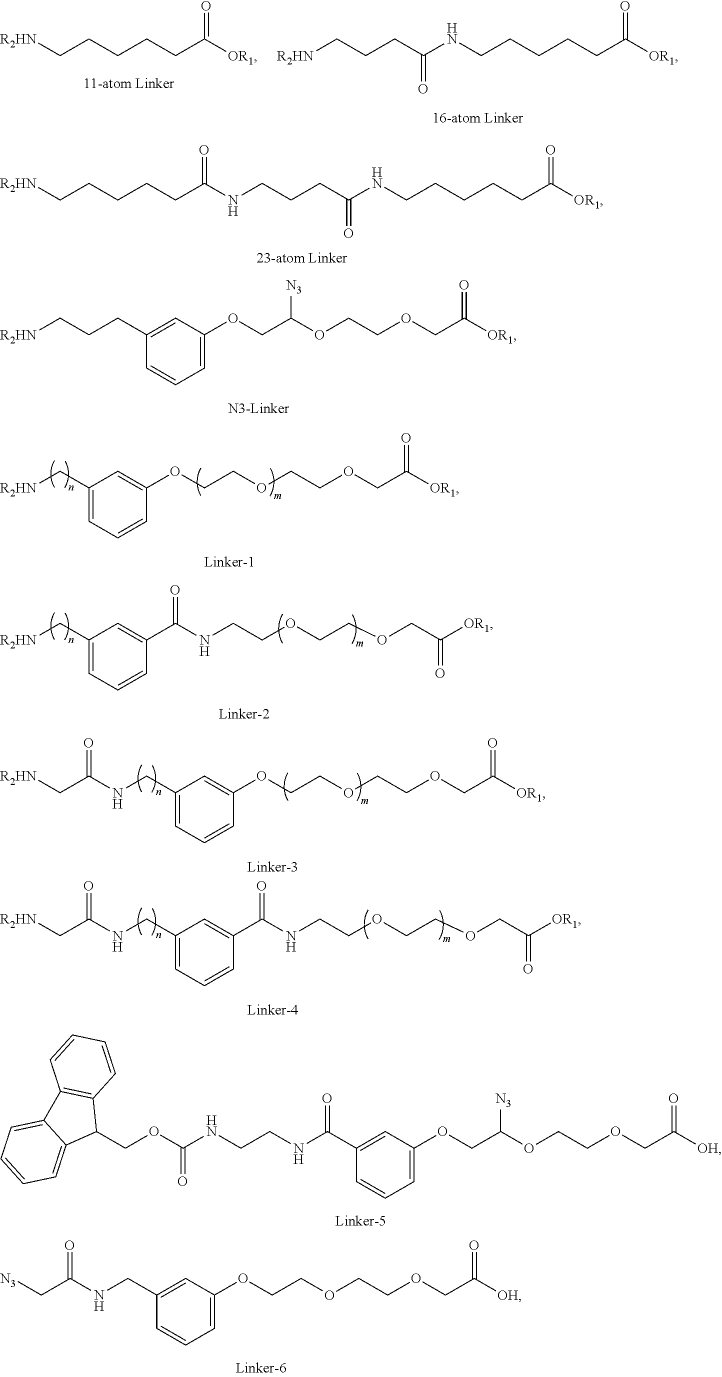

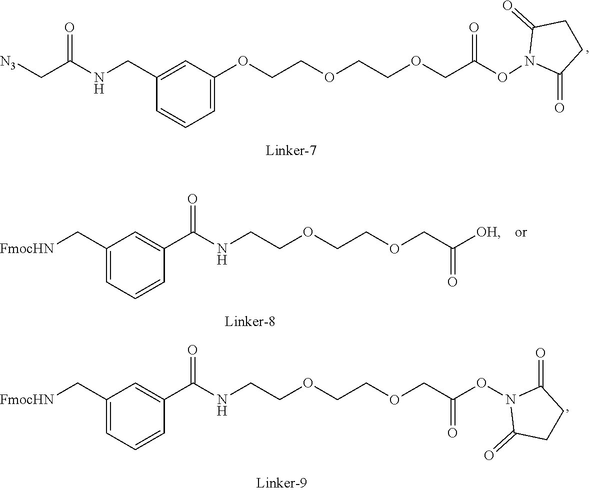

wherein the value of n is 1-6, and wherein the value of m is 0-10.

Embodiment 8. The plurality of nucleotide conjugates of embodiment 1, wherein the plurality of nucleotide arms comprise a reactive group which is reactive with a chemical agent.

Embodiment 9. The plurality of nucleotide conjugates of embodiment 8, wherein the reactive group comprises an alkyl, alkenyl, alkynyl, allyl, aryl, benzyl, azide, amine, amide, keto, isocyanate, phosphate, thio, disulfide, carbonate, urea, or silyl group.

Embodiment 10. The plurality of nucleotide conjugates of embodiment 9, wherein
a) the reactive groups alkyl, alkenyl, alkynyl and allyl are reactive with tetrakis(triphenylphosphine)palladium(0) (Pd(P(C₆H₅)₃)₄) with piperidine, or with 2,3-Dichloro-5,6-dicyano-1,4-benzo-quinone (DDQ);
b) the reactive groups aryl and benzyl are reactive with H2 and Palladium on carbon (Pd/C);
c) the reactive groups amine, amide, keto, isocyanate, phosphate, thio, disulfide are reactive with phosphine or with a thiol group including beta-mercaptoethanol or dithiothritol (DTT);
d) the reactive group carbonate is reactive with potassium carbonate (K₂CO₃) in MeOH, with triethylamine in pyridine, or with Zn in acetic acid (AcOH); and
e) the reactive groups urea and silyl are reactive with tetrabutylammonium fluoride, pyridine-HF, with ammonium fluoride, or with triethylamine trihydrofluoride.

Embodiment 11. The plurality of nucleotide conjugates of embodiment 9, wherein the azide reactive group in the linker comprises an azide, azido or azidomethyl group.

Embodiment 12. The plurality of nucleotide conjugates of embodiment 11, wherein the azide, azido or azidomethyl group in the linker is reactive with a chemical agent which comprises a phosphine compound.

Embodiment 13. The plurality of nucleotide conjugates of embodiment 12, wherein the phosphine compound comprises a derivatized tri-alkyl phosphine moiety or a derivatized tri-aryl phosphine moiety.

Embodiment 14. The plurality of nucleotide conjugates of embodiment 13, wherein the phosphine compound comprises Tris(2-carboxyethyl)phosphine (TCEP) or bis-sulfo triphenyl phosphine (BS-TPP).

Embodiment 15. The plurality of nucleotide conjugates of embodiment 1, wherein the core is attached to the plurality of nucleotide arms having the same type of a nucleotide unit, and wherein the nucleotide unit comprises dATP, dGTP, dCTP, dTTP or dUTP.

Embodiment 16. The plurality of nucleotide conjugates of embodiment 1, wherein the nucleotide unit comprises a chain terminating moiety (blocking group) linked to the 3′ carbon of the sugar moiety, and wherein the chain terminating moiety are reactive with a chemical compound to remove the chain terminating moiety and generate a sugar 3′OH moiety on the nucleotide unit.

Embodiment 17. The plurality of nucleotide conjugates of embodiment 16, wherein the chain terminating moiety comprises an alkyl, alkenyl, alkynyl, allyl, aryl, benzyl, azide, amine, amide, keto, isocyanate, phosphate, thio, disulfide, carbonate, urea, or silyl group.

Embodiment 18. The plurality of nucleotide conjugates of embodiment 17, wherein
a) the chain terminating moieties alkyl, alkenyl, alkynyl and allyl are reactive with tetrakis(triphenylphosphine)palladium(0) (Pd(P(C₆H₅)₃)₄) with piperidine, or with 2,3-Dichloro-5,6-dicyano-1,4-benzo-quinone (DDQ);
b) the chain terminating moieties aryl and benzyl are reactive with H2 and Palladium on carbon (Pd/C);
c) the chain terminating moieties amine, amide, keto, isocyanate, phosphate, thio, disulfide are reactive with phosphine or with a thiol group including beta-mercaptoethanol or dithiothritol (DTT);
d) the chain terminating moiety carbonate is reactive with potassium carbonate (K₂CO₃) in MeOH, with triethylamine in pyridine, or with Zn in acetic acid (AcOH); and
e) the chain terminating moieties urea and silyl are reactive with tetrabutylammonium fluoride, pyridine-HF, with ammonium fluoride, or with triethylamine trihydrofluoride.

Embodiment 19. The plurality of nucleotide conjugates of embodiment 17, wherein the azide chain terminating moiety comprises an azide, azido or azidomethyl group.

Embodiment 20. The plurality of nucleotide conjugates of embodiment 19, wherein the chain terminating moieties azide, azido and azidomethyl group are reactive with a chemical agent which comprises a phosphine compound.

Embodiment 21. The plurality of nucleotide conjugates of embodiment 20, wherein the phosphine compound comprises a derivatized tri-alkyl phosphine moiety or a derivatized tri-aryl phosphine moiety.

Embodiment 22. The plurality of nucleotide conjugates of embodiment 20, wherein the phosphine compound comprises Tris(2-carboxyethyl)phosphine (TCEP) or bis-sulfo triphenyl phosphine (BS-TPP).

Embodiment 23. The plurality of nucleotide conjugates of any of the preceding embodiments, wherein the plurality of nucleotide conjugates comprises a mixture of two or more sub-populations of nucleotide conjugates, wherein the mixture comprises:
a) a first sub-population comprising nucleotide conjugates having a core attached to a plurality of nucleotide arms having the same type of a nucleotide unit, wherein the nucleotide unit comprises dATP, dGTP, dCTP, dTTP or dUTP; and
b) a second sub-population comprising nucleotide conjugates having a core attached to a plurality of nucleotide arms having the same type of a nucleotide unit, wherein the nucleotide unit comprises dATP, dGTP, dCTP, dTTP or dUTP, and wherein the nucleotide unit in the first sub-population differ from the nucleotide unit in the second sub-population.

Embodiment 24. The plurality of nucleotide conjugates of any of the preceding embodiments, wherein the plurality of nucleotide conjugates comprises a mixture of two or more sub-populations of nucleotide conjugates, wherein the mixture comprises:
1. a first sub-population comprising nucleotide conjugates having a core attached to a plurality of nucleotide arms having the same type of a nucleotide unit, wherein the nucleotide unit comprises dATP, dGTP, dCTP, dTTP or dUTP and wherein the nucleotide unit comprises a first chain terminating moiety (blocking group) linked to the 3′ carbon of the sugar moiety, and wherein the first chain terminating moiety is reactive with a chemical compound to remove the first chain terminating moiety and generate a sugar 3′OH moiety on the nucleotide unit; and
2. a second sub-population comprising nucleotide conjugates having a core attached to a plurality of nucleotide arms having the same type of a nucleotide unit, wherein the nucleotide unit comprises dATP, dGTP, dCTP, dTTP or dUTP, and wherein the nucleotide unit in the first sub-population differ from the nucleotide unit in the second sub-population, and wherein the nucleotide unit comprises a second chain terminating moiety (blocking group) linked to the 3′ carbon of the sugar moiety, and wherein the second chain terminating moiety is reactive with a chemical compound to remove the second chain terminating moiety and generate a sugar 3′OH moiety on the nucleotide units.

Embodiment 25. The plurality of nucleotide conjugates of any of the preceding embodiments, wherein the plurality of nucleotide conjugates comprises a mixture of two or more sub-populations of nucleotide conjugates, wherein the mixture comprises:
a) a first sub-population comprising nucleotide conjugates having a core attached to a plurality of nucleotide arms having the same type of a first type of linker; and
b) a second sub-population comprising nucleotide conjugates having a core attached to a plurality of nucleotide arms having the same type of a second type of linker, and wherein the first type of linker in the first sub-population differ from the second type of linker in the second sub-population.

Embodiment 26. The plurality of nucleotide conjugates of any of the preceding embodiments, wherein the plurality of nucleotide conjugates comprises a mixture of two or more sub-populations of nucleotide conjugates, wherein the mixture comprises:
(i) a first sub-population comprising nucleotide conjugates having a core attached to a plurality of nucleotide arms having the same type of a first type of linker with a first reactive group; and
(ii) a second sub-population comprising nucleotide conjugates having a core attached to a plurality of nucleotide arms having the same type of a second type of linker with a second reactive group, and wherein the first reactive group in the first type of linker in the first sub-population differ from the second reactive group in the second type of linker in the second sub-population.

Embodiment 27. The plurality of nucleotide conjugates of any one of embodiments 23, 24, 25 or 26, wherein
1. the first sub-population of nucleotide conjugates are labeled with a first fluorophore; and
2. the second sub-population of nucleotide conjugates are labeled with a second fluorophore that differs from the first fluorophore.

Embodiment 28. The plurality of nucleotide conjugates of any one of embodiments 23, 24, 25 or 26, wherein
1. the first sub-population of nucleotide conjugates are labeled with a first fluorophore; and
2. the second sub-population of nucleotide conjugates are non-labeled.

Embodiment 29. A system comprising a plurality of binding complexes, which comprises a plurality of the nucleotide conjugates of any of embodiments 1-22, and further comprising a plurality of polymerases, a plurality of nucleic acid template molecules, and a plurality of nucleic acid primer molecules, wherein an individual binding complex in the plurality of binding complexes include: a polymerase bound to a nucleic acid template molecule which is hybridized to a nucleic acid primer, and a nucleotide conjugate which is bound to the nucleic acid primer.

Embodiment 30. The system of embodiment 29, wherein the plurality of nucleic acid template molecules and/or the plurality of nucleic acid primer molecules are immobilized to a support, thereby generating a plurality of binding complexes immobilized on the support.

Embodiment 31. The system of embodiment 30, wherein a density of the immobilized binding complexes on the support is 10²-10⁹ per mm².

Embodiment 32. The system of embodiment 30, wherein the plurality of immobilized binding complexes on the surface are in fluid communication with each other to permit flowing a solution of reagents onto the support so that the plurality of immobilized binding complexes on the support react with the solution of reagents in a massively parallel manner.

Embodiment 33. The system of embodiment 30, wherein the support comprises a surface coating comprising at least one hydrophilic polymer coating layer and a plurality of surface capture primers.

Embodiment 34. The system of embodiment 33, wherein the at least one hydrophilic polymer coating layer comprises PEG.

Embodiment 35. The system of embodiment 33, wherein the at least one hydrophilic polymer coating layer comprises a branched PEG having at least 4 branches.

Embodiment 36. The system of embodiment 33, wherein the at least one hydrophilic polymer coating layer has a water contact angle of no more than 45 degrees.

Embodiment 37. The system of embodiment 30, wherein a fluorescent image of the plurality of binding complexes immobilized on the support has a contrast to noise ratio is greater than 20.

Embodiment 38. An multivalent binding complex, comprising a plurality of the nucleotide conjugates of any of embodiments 1-22, and further comprising a plurality of nucleic acid primer molecules, a plurality of polymerases, and a nucleic acid concatemer template molecule, wherein
a) a first primer, a first polymerase, and a first nucleotide conjugate are bound to a first portion of a concatemer template molecule thereby forming a first binding complex, wherein a first nucleotide unit of the nucleotide conjugate is bound to the first polymerase, and
b) a second primer, a second polymerase, and the first nucleotide conjugate are bound to a second portion of the concatemer template molecule thereby forming a second binding complex, wherein a second nucleotide unit of the nucleotide conjugate is bound to the second polymerase, and wherein the first binding complex and second binding complex which include the first nucleotide conjugate forms an multivalent binding complex.

Embodiment 39. An multivalent binding complex, comprising a plurality of the nucleotide conjugates of any of embodiments 1-22, and further comprising a plurality of nucleic acid primer molecules, a plurality of polymerases, and a plurality of clonally-amplified nucleic acid template molecules, wherein
a) a first primer, a first polymerase, and a first nucleotide conjugate are bound to a first clonally-amplified template molecule thereby forming a first binding complex, wherein a first nucleotide unit of the nucleotide conjugate is bound to the first polymerase, and
b) a second primer, a second polymerase, and the first nucleotide conjugate are bound to a second clonally-amplified template molecule thereby forming a second binding complex, wherein a second nucleotide unit of the nucleotide conjugate is bound to the second polymerase, wherein the first and second binding complexes which include the first nucleotide conjugate forms an multivalent binding complex, wherein the first cloncally-amplified template molecule and second clonally-amplified template molecule are generated via bridge amplification and are immobilized to a same location or feature on a support.

Embodiment 40. A method for forming at least one binding complex, the method comprising:
a) providing at least one complexed polymerase which comprises a polymerase bound to a nucleic acid template molecule which is hybridized to a nucleic acid primer, and
b) contacting the at least one complexed polymerase with a plurality of the nucleotide conjugates of any of embodiments 1-22, wherein the contacting is performed under conditions suitable for binding the nucleotide unit of the nucleotide conjugate to the nucleic acid primer, thereby forming at least one binding complex.

Embodiment 41. The method of embodiment 40, wherein the conditions are suitable for binding the nucleotide unit of the nucleotide conjugate to the nucleic acid primer but inhibit polymerase-catalyzed nucleotide incorporation into the nucleic acid primer.

Embodiment 42. The method of embodiment 40, wherein the nucleic acid template molecule comprises a concatemer template molecule having two or more tandem copies of a sequence of interest and at least one universal adaptor sequence.

Embodiment 43. The method of embodiment 40, wherein the nucleic acid template molecule comprises a clonally-amplified template molecule having one copy of a sequence of interest and at least one universal adaptor sequence, and wherein the clonally-amplified template molecule is generated via bridge amplification.

Embodiment 44. A method for forming an multivalent binding complex, comprising a plurality of the nucleotide conjugates of any of embodiments 1-22, the method comprising:
a) a first primer, a first polymerase, and a first nucleotide conjugate are bound to a first portion of a concatemer template molecule thereby forming a first binding complex, wherein a first nucleotide unit of the nucleotide conjugate is bound to the first polymerase, and
b) a second primer, a second polymerase, and the first nucleotide conjugate are bound to a second portion of the concatemer template molecule thereby forming a second binding complex, wherein a second nucleotide unit of the nucleotide conjugate is bound to the second polymerase, and wherein the first and second binding complexes which include the first nucleotide conjugate forms an multivalent binding complex.

Embodiment 45. A method for sequencing one or more nucleic acid template molecules, the method comprising:
a) contacting a plurality of polymerases and a plurality of nucleic acid primers with different portions of a concatemer nucleic acid template molecule to form at least a first complexed polymerase and a second complexed polymerase on the concatemer nucleic acid template molecule, wherein the first complexed polymerase comprises a first polymerase bound to a first portion of the concatemer nucleic acid template molecule which is hybridized to a first primer, and wherein the second complexed polymerase comprises a second polymerase bound to a second portion of the concatemer nucleic acid template molecule which is hybridized to a second primer (e.g., the first primer and the second primer hybridize to different locations on the concatemer nucleic acid template molecule);
b) contacting a plurality of fluorescently-labeled nucleotide conjugates of any of embodiment 1-22 to the at least first complexed polymerase and the second complexed polymerase on the concatemer nucleic acid template molecule, under conditions suitable to bind a single nucleotide conjugate from the plurality to the at least first complexed polymerase and second complexed polymerase, wherein at least a first nucleotide unit of the single nucleotide conjugate is bound to the first complexed polymerase which includes the first primer hybridized to a first portion of the concatemer nucleic acid template molecule thereby forming a first binding complex, and wherein at least a second nucleotide unit of the single nucleotide conjugate is bound to the second complexed polymerase which includes the second primer hybridized to a second portion of the concatemer nucleic acid template molecule thereby forming a second binding complex, wherein the contacting is conducted under a condition suitable to inhibit polymerase-catalyzed incorporation of the bound first nucleotide unit and second nucleotide unit in the first binding complex and second binding complex, and wherein the first binding complex and second binding complex which are bound to the single nucleotide conjugate forms an multivalent binding complex;
c) detecting the first binding complex and second binding complex on the concatemer nucleic acid template molecule, and
d) identifying the at least first nucleotide unit in the first binding complex thereby determining the sequence of the first portion of the concatemer nucleic acid template molecule, and identifying the at least second nucleotide unit in the second binding complex thereby determining the sequence of the second portion of the concatemer nucleic acid template molecule.

Embodiment 46. An multivalent binding complex, comprising a plurality of the nucleotide conjugates of any of embodiments 1-22, and further comprising a plurality of nucleic acid primer molecules, a plurality of polymerases, and a plurality of clonally-amplified nucleic acid template molecules, wherein
a) a first primer, a first polymerase, and a first nucleotide conjugate are bound to a first clonally-amplified template molecule thereby forming a first binding complex, wherein a first nucleotide unit of the first nucleotide conjugate is bound to the first polymerase, and
b) a second primer, a second polymerase, and the first nucleotide conjugate are bound to a second clonally-amplified template molecule thereby forming a second binding complex, wherein a second nucleotide unit of the first nucleotide conjugate is bound to the second polymerase, wherein the first and second binding complexes which include the first nucleotide conjugate forms an multivalent binding complex, wherein the first clonally-amplified template molecule and the second clonally-amplified template molecule are generated via bridge amplification and are immobilized to a same location or feature on a support.

Embodiment 47. A method for sequencing one or more nucleic acid template molecules, the method comprising:
a) contacting a plurality of polymerases and a plurality of nucleic acid primers with a first clonally-amplified template molecule and a second first clonally-amplified template molecule template molecule to form at least a first complexed polymerase and a second complexed polymerase on the first clonally-amplified template molecule and second clonally-amplified template molecule template molecule, wherein the first complexed polymerase comprises a first polymerase bound to the first clonally-amplified template molecule which is hybridized to a first primer, and wherein the second complexed polymerase comprises a second polymerase bound to the second clonally-amplified template molecule which is hybridized to a second primer;
b) contacting a plurality of fluorescently-labeled nucleotide conjugates of any of embodiment 1-22 to the first complexed polymerase and the second complexed polymerase, under conditions suitable to bind a single nucleotide conjugate from the plurality to the first complexed polymerase and the second complexed polymerase, wherein at least a first nucleotide unit of a single nucleotide conjugate is bound to the first complexed polymerase which includes the first primer hybridized to the first clonally-amplified template molecule thereby forming a first binding complex, and wherein at least a second nucleotide unit of the single nucleotide conjugate is bound to the second complexed polymerase which includes the second primer hybridized to the second clonally-amplified template molecule thereby forming a second binding complex, wherein the contacting is conducted under a condition suitable to inhibit polymerase-catalyzed incorporation of the bound first nucleotide unit and the second nucleotide unit in the first binding complex and the second binding complex, and wherein the first binding complex and the second binding complex which are bound to the single nucleotide conjugate forms an multivalent binding complex;
c) detecting the first binding complex and the second binding complex on the first clonally-amplified template molecule and the second clonally-amplified template molecule, and
d) identifying the first nucleotide unit in the first binding complex thereby determining the sequence of the first clonally-amplified template molecule, and identifying the second nucleotide unit in the second binding complex thereby determining the sequence of the second template molecule, wherein the first template molecule and the second template molecule comprise the first clonally-amplified template molecule and the second clonally-amplified template molecule that are generated via bridge amplification and are immobilized to a same location or feature on a support.

Embodiment 48. The method of embodiment 44 or 45, wherein the concatemer nucleic acid template molecule, the first primer and/or the second primer are immobilized to a support, thereby immobilizing the first binding complex and the second binding complex to the support.

Embodiment 49. The method of embodiment 48, wherein the immobilized first binding complex and the second binding complex are in fluid communication with each other to permit flowing a solution of reagents onto the support so that the immobilized first binding complex and second binding complex on the support react with the solution of reagents in a massively parallel manner.

Embodiment 50. The method of embodiment 48, wherein the support comprises a surface coating comprising at least one hydrophilic polymer coating layer and a plurality of surface capture primers.

Embodiment 51. The method of embodiment 50, wherein the at least one hydrophilic polymer coating layer comprises PEG.

Embodiment 52. The method of embodiment 50, wherein the at least one hydrophilic polymer coating layer comprises a branched PEG having at least 4 branches.

Embodiment 53. The method of embodiment 50, wherein the at least one hydrophilic polymer coating layer has a water contact angle of no more than 45 degrees.

Embodiment 54. The method of embodiment 50, wherein the detecting step comprises obtaining a fluorescent image of a plurality of binding complexes immobilized to the support where the fluorescent image exhibits a contrast to noise ratio that is greater than 20.

Embodiment 55. The method of embodiment 46 or 47, wherein the first clonally-amplified template molecule, the first primer, the second clonally-amplified template, the first primer and/or the second primer are immobilized to a support, thereby immobilizing the first binding complex and the second binding complex to the support.

Embodiment 56. The method of embodiment 55, wherein the immobilized first binding complex and second binding complex are in fluid communication with each other to permit flowing a solution of reagents onto the support so that the immobilized first binding complex and second binding complex on the support react with the solution of reagents in a massively parallel manner.

Embodiment 57. The method of embodiment 55, wherein the support comprises a surface coating comprising at least one hydrophilic polymer coating layer and a plurality of surface capture primers.

Embodiment 58. The method of embodiment 57, wherein the at least one hydrophilic polymer coating layer comprises PEG.

Embodiment 59. The method of embodiment 57, wherein the at least one hydrophilic polymer coating layer comprises a branched PEG having at least 4 branches.

Embodiment 60. The method of embodiment 57, wherein the at least one hydrophilic polymer coating layer has a water contact angle of no more than 45 degrees.

Embodiment 61. The method of embodiment 57, wherein the detecting step comprises obtaining a fluorescent image of a plurality of binding complexes immobilized to the support where the fluorescent image exhibits a contrast to noise ratio that is greater than 20.

1.-6. (canceled)

7. A method for processing a nucleic acid molecule, the method comprising:
(a) introducing a synthetic polypeptide and a detectable nucleotide to a nucleic acid sequence hybridized to a primer nucleic acid sequence under conditions sufficient to form a binding complex comprising the synthetic polypeptide, the detectable nucleotide and the nucleic acid sequence,
wherein the detectable nucleotide is complementary to a nucleotide of the nucleic acid sequence, and
wherein the synthetic polypeptide has an amino acid sequence that comprises a D141A mutation or a D143A mutation with reference to SEQ ID NO: 391 and has at least 85% sequence identity to SEQ ID NO: 391; and
(b) detecting the binding complex to identify the nucleotide of the nucleic acid sequence.

8. The method of claim 7, wherein the amino acid sequence further comprises the D141A mutation and the D143A mutation.

9. The method of claim 7, wherein the amino acid sequence further comprises a Y410A mutation, a L409S mutation, a Y261A mutation, a P411G mutation, an F406I mutation, a P411A mutation, a Y7A mutation, a Y493I mutation, a Y493T mutation, a V513I mutation, a L409A mutation, an A485S mutation, a Y410G mutation, an I521H mutation, or a K507L mutation, or any combination thereof, with reference to SEQ ID No: 391.

10. The method of claim 7, wherein the amino acid sequence further comprises:
(a) a Y410A mutation, with reference to SEQ ID No: 391;
(b) a L409S mutation, a Y410A mutation, and a Y261A mutation, with reference to SEQ ID No: 391;
(c) a L409S mutation, a Y410A mutation, a P411G mutation, and a Y261A mutation, with reference to SEQ ID No: 391;
(d) a Y261A mutation, an F406I mutation, a L409S mutation, a Y410A mutation, and a P411A mutation, with reference to SEQ ID No: 391;
(e) a Y7A mutation, a Y261A mutation, and a Y410A mutation, with reference to SEQ ID No: 391;
(f) a Y261A mutation, a Y410A mutation, and a Y493I mutation, with reference to SEQ ID No: 391;
(g) a Y261A mutation, a Y410A mutation, and a Y493T mutation, with reference to SEQ ID No: 391;
(h) a Y261A mutation, a Y410A mutation, and a V513I mutation, with reference to SEQ ID No: 391;
(i) a Y7A mutation, a Y261A mutation, a L409S mutation, and a Y410A mutation, with reference to SEQ ID No: 391;
(j) a Y261A mutation, a L409A mutation, a Y410A mutation, and a P411A mutation, with reference to SEQ ID No: 391;
(k) a Y261A mutation, a L409S mutation, a Y410A mutation, and a P411G mutation, with reference to SEQ ID No: 391;
(l) a Y261A mutation, a L409S mutation, and a Y410A mutation, with reference to SEQ ID No: 391;
(m) a Y261A mutation, a L409S mutation, and a Y410G mutation, with reference to SEQ ID No: 391;
(n) a Y261A mutation, a L409A mutation, a Y410A mutation, a P411A mutation, and an A485S mutation, with reference to SEQ ID No: 391;
(o) a Y261A mutation, a L409S mutation, a Y410A mutation, a P411G mutation, and an A485S mutation, with reference to SEQ ID No: 391;
(p) a Y261A mutation, a L409S mutation, a Y410A mutation, and an A485S mutation, with reference to SEQ ID No: 391;
(q) a Y261A mutation, a L409S mutation, a Y410G mutation, and an A485S mutation, with reference to SEQ ID No: 391;
(r) a Y261A mutation, a L409A mutation, a Y410A mutation, a P411A mutation, and an I521H mutation, with reference to SEQ ID No: 391;
(s) a Y261A mutation, a L409S mutation, a Y410A mutation, a P411G mutation, and an I521H mutation, with reference to SEQ ID No: 391;
(t) a Y261A mutation, a L409S mutation, a Y410A mutation, and an I521H mutation, with reference to SEQ ID No: 391;
(u) a Y261A mutation, a L409S mutation, a Y410G mutation, and an I521H mutation, with reference to SEQ ID No: 391; or
(v) a Y261A mutation, a L409S mutation, a Y410A mutation, an A485S mutation, a K507L mutation, and an I521H mutation, with reference to SEQ ID No: 391.

11. The method of claim 7, wherein the amino acid sequence has at least 90% sequence identity to SEQ ID NO: 391.

12. The method of claim 7, wherein the amino acid sequence has at least 99% sequence identity to SEQ ID NO: 391.

13. The method of claim 7, wherein the amino acid sequence comprises any one of SEQ ID NOs: 392-413.

14. The method of claim 7, wherein the detectable nucleotide comprises a blocking group.

15. The method of claim 14, wherein the blocking group is coupled directly or indirectly to a 3′ carbon of the sugar of the detectable nucleotide, and wherein the blocking group is removable from the detectable nucleotide to generate a hydroxyl group on the 3′ carbon.

16. The method of claim 14, wherein the blocking group comprises a 3′-O-azidomethyl group, 3′-O-methyl group, a 3′-O-alkyl hydroxylamino group, a 3′-phosphorothioate group, a 3′-O-malonyl group, or a 3′-O-benzyl group.

17. The method of claim 14, wherein the blocking group comprises a 3′-O-azidomethyl group.

18. The method of claim 7, wherein the detectable nucleotide comprises a label coupled thereto directly or indirectly.

19. The method of claim 7, wherein a 3′ terminal nucleotide of the primer nucleic acid sequence comprises a blocking group.

20. The method of claim 19, wherein the blocking group comprises a 3′-O-azidomethyl group, 3′-O-methyl group, a 3′-O-alkyl hydroxylamino group, a 3′-phosphorothioate group, a 3′-0-malonyl group, or a 3′-O-benzyl group.

21. The method of claim 7, further comprising incorporating the detectable nucleotide into a 3′ terminus of the primer nucleic acid sequence.

22. The method of claim 7, wherein the detecting the binding complex does not comprise incorporating the detectable nucleotide into a 3′ terminus of the primer nucleic acid sequence.

23. The method of claim 7, wherein binding complex is a multivalent binding complex comprising at least two of the synthetic polypeptide, at least two copies of the detectable nucleotide, and at least two of the nucleic acid sequence.

24. The method of claim 23, wherein the at least two copies of the detectable nucleotide are coupled directly or indirectly to a common core to form a nucleotide-conjugate.

25. The method of claim 24, wherein the nucleotide-conjugate further comprises at least two nucleotide arms, wherein a nucleotide arm of the at least two nucleotide arms comprises:
(i) a core attachment moiety coupled to the common core;
(ii) a spacer coupled to the core attachment moiety;
(iii) a linker coupled to the spacer; and
(iv) the detectable nucleotide coupled to the linker.

26. The method of claim 25, wherein the linker comprises: 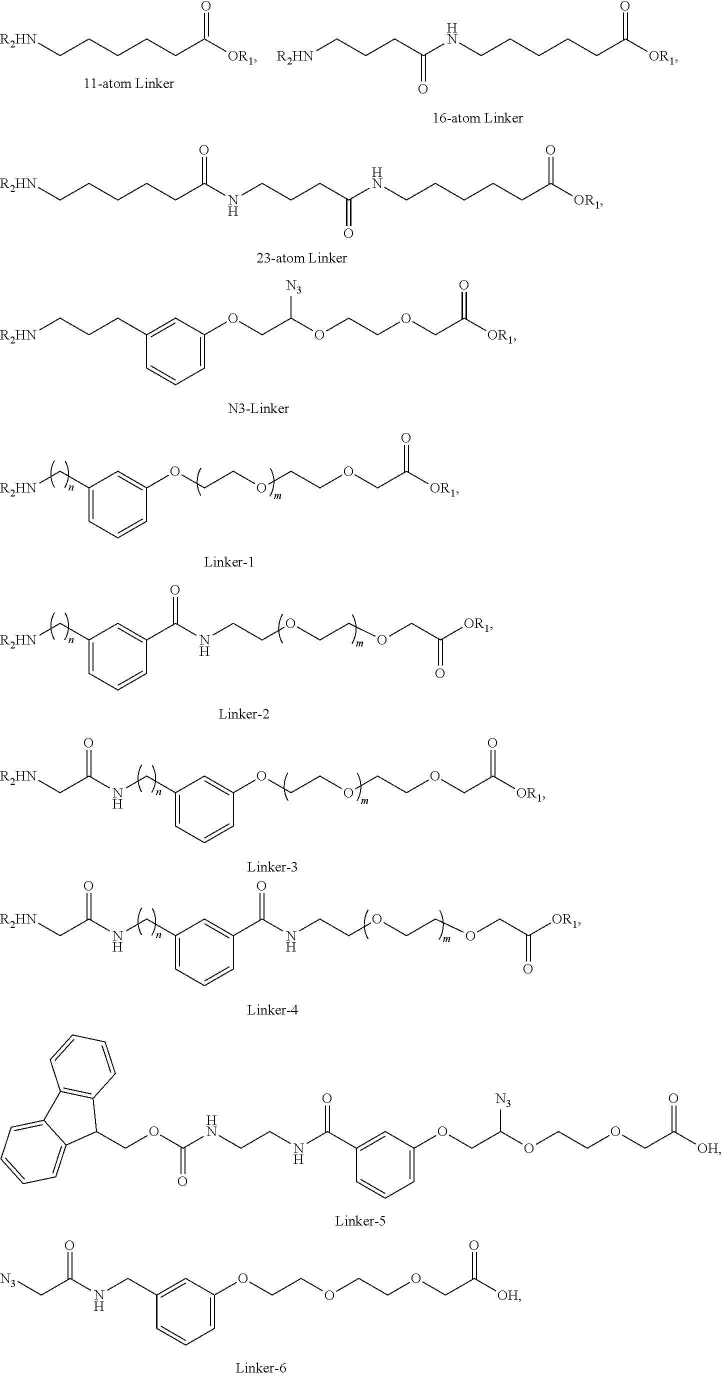 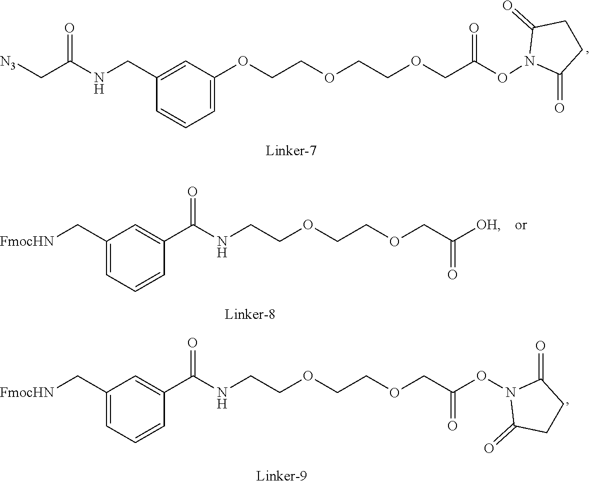
wherein n is 1 to 6 and m is 0 to 10.

27. The method of claim 25, wherein the at least two nucleotide arms comprises 3 to 20 nucleotide arms.

28. The method of claim 25, wherein the common core comprises streptavidin or avidin.

29. The method of claim 25, wherein the core attachment moiety comprises biotin.

30. The method of claim 25, wherein the spacer comprises a structure: 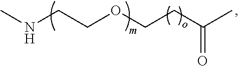
wherein m is 20 to 500 and o is 1 to 10.

31. The method of claim 23, wherein the at least two of the nucleic acid sequence is comprised in a concatemer nucleic acid molecule.
